(19) United States
(12) Patent Application Publication
(10) Pub. No.: US 2026/0263529 A1
(43) Pub. Date: Sep. 10, 2026

(54) BIOENGINEERED COLON-SPECIFIC IMUNE NICHE FOR THE TREATMENT OF ULCERATIVE COLITIS

(71) Applicant: THE BOARD OF REGENTS OF THE UNIVERSITY OF TEXAS SYSTEM, Austin, TX (US)

(72) Inventors: Andrew WANG, Dallas, TX (US); Kin Man AU, Dalls, TX (US)

(21) Appl. No.: 19/166,162

(22) PCT Filed: Mar. 15, 2024
(86) PCT No.: PCT/US2024/020202

§ 371 Date: Sep. 17, 2025

Related U.S. Application Data
(60) Provisional application No. 63/491,359, filed on Mar. 21, 2023.
(60) Provisional application No. 63/587,661, filed on Oct. 3, 2023.

(51) Int. Cl. A61K 35/37; A61K 9/70; A61K 31/5377; A61K 47/69; A61P 1/04; C07K 16/28

(52) U.S. Cl. CPC A61K 35/37; A61K 9/70; A61K 31/5377; A61K 47/6953; A61P 1/04; C07K 16/2827

(57) ABSTRACT

The present disclosure generally relates to compositions and methods comprising colon-specific immune niche. Further provided is a method of treating a condition or disease of large intestinal tract tissue using the colon-specific immune niche. A method of making a colon-specific immune niche is also described.

### CROSS REFERENCE TO RELATED APPLICATIONS

This application claims priority to U.S. Provisional Patent Application Ser. No. 63/491,359, filed Mar. 21, 2023, and titled “BIOENGINEERED COLON-SPECIFIC IMMUNE NICHE FOR THE TREATMENT OF ULCERATIVE COLITIS,” and U.S. Provisional Patent Application Ser. No. 63/587,661, filed Oct. 3, 2023, and titled “BIOENGINEERED COLON-SPECIFIC IMMUNE NICHE FOR THE TREATMENT OF ULCERATIVE COLITIS,” which are incorporated by reference herein in their entireties.

### BACKGROUND

### 1. Field

The present disclosure relates generally to treatment of ulcerative colitis using an engineered colon niche with an immune suppressive microenvironment.

### 2. Discussion of Related Art

Ulcerative colitis (UC) is a chronic autoinflammatory bowel disease in which autoreactive T cells and macrophages attack healthy colon (COL) epithelial (EPI) cells in the colonic mucosa, leading to chronic inflammation and ulceration. Symptoms of UC can be debilitating and can lead to life-threatening complications, such as bowel perforation and toxic megacolon. While the causes of UC are not fully understood, it is widely believed to be triggered by genetic mutations, immune system malfunction, and environmental factors. Despite intense efforts in treatment development, UC remains incurable. Current treatment strategies generally involve immunosuppressive therapies and surgical resection. However, long-term immune suppression has significant side effects, including infection and cancer development, and surgery has a high morbidity and long-term health complications.

Therefore, there is a great need for novel and curative treatment development for UC such as a disease-specific treatment that eliminate autoimmunity without causing systemic immunosuppression.

### SUMMARY

In one aspect, the disclosure encompasses a method of making a colon-specific immune niche (COIN), the method comprising combining diblock copolymer nanofibers (NFs), with colon epithelial (EPI) cells, and decellularized colon extracellular matrix (ECM). In some aspects, the NFs, EPI, ECM, or any combination thereof is functionalized with an immunomodulator to form the COIN.

In some aspects, the diblock copolymer comprises carboxylic acid-functionalized poly(2-(methacryloyloxy)ethyl phosphorylcholine)-poly(2-hydroxypropyl methacrylate) (PMPC-PHPMA). In some aspects, the NFs are further functionalized with sulfo DBCO-amine via amine-NHS ester coupling reaction (DBCO-NFs) or anionic carboxylic acid (COOH-NFs).

In some aspects, the immunomodulator comprises a PD-L1 antibody, a CD86 antibody, leflunomide (LEF), HVEM (Herpesvirus entry mediator) or BTLA (B and T lymphocyte attenuator) agonist, Fas ligand, FGL-1, CD47, Gal-9, TGF-β1 mimetic peptide, or any combination thereof.

In some aspects, the immunomodulator comprises a PD-L1 antibody, a CD86 antibody, or any combination thereof, and the immunomodulator is functionalized onto NFs by strain-promoted azide-alkyne cycloaddition with the azide-functionalized antibody at about 1-20 μg per mg of DBCO-NFs. In some aspects, the immunomodulator comprises LEF, and the immunomodulator is functionalized onto NFs by combining azide-functionalized LEF-encapsulated poly(ethylene glycol)-poly(lactide-co-glycolide) nanoparticles (LEF-NPs) with DBCO-NFs at about 1-400 μg of azide-functionalized LEF NPs per milligram of DBCO-NFs. In certain aspects, the immunomodulator is functionalized onto the ECM, EPI, or any combination thereof. In some aspects, the immunomodulator comprises TGF-β1 mimetic peptide, and the immunomodulator is functionalized onto NFs by ionic adsorption of the oligomerized cationic TGF-β1 mimetic peptide onto COOH-NFs at about 1 μg to about 100 μg mimetic peptide per milligram of COOH-NFs.

In some aspects, the colon EPI cells and/or decellularized colon ECM are syngeneic, allogenic, or autologous. In some aspects, the combining comprises mixing together, NFs at a concentration of about 0.1 mg to about 100 mg, decellularized colon ECM at concentration of about 0.05 mg to about 5 mg, and colon EPI cells at a concentration of about 1×10² to about 1×10¹⁰ cells. In some aspects, the combining comprises mixing together, NFs at a concentration of about 1 mg, decellularized colon ECM at a concentration of about 0.5 mg, and colon EPI cells at a concentration of about 1×10⁶ cells.

In further aspects, provided herein is a composition comprising the disclosed COIN. In further aspects, the COIN comprises a combination of NFs functionalized with a PD-L1 antibody, a CD86 antibody, leflunomide (LEF), and a TGF-β1 mimetic peptide. In some aspects, the disclosed composition is formulated for infusion, implantation, subcutaneous injection, or intraperitoneal injection.

The disclosure further encompasses a method of preventing, treating, or reducing the risk of a condition or disease of large intestinal tract tissue in a subject comprising: administering to a subject in need thereof an effective amount of a composition comprising COIN disclosed herein.

In some aspects, provided herein is a method of preventing, treating, or reducing the risk of a condition or disease of large intestinal tract tissue associated cancer in a subject, comprising administering to a subject in need thereof an effective amount of a composition comprising the disclosed COIN.

In certain aspects, a method of preventing, treating, or reducing the risk of a condition or disease of large intestinal tract tissue associated with administration of a Phosphoinositide 3-kinase (PI3K) inhibitor in a subject, comprising: administering to a subject in need thereof an effective amount of a composition comprising the disclosed COIN is further provided. In such aspects, the PI3K inhibitor is copanlisib, idelalisib, umbralisib, duvelisib, alpelisib, anti-PI3K antibody, CAL-120/GS-9820, Wortmannin, demethoxyviridin, LY294002, perifosine, CAL-101, PX-866, IPI-145, BAY 80-6946, BEZ-235, RP-6503, TGR-1202, SF-1126, INK-1117, GDC-0941, BKM-120, XL-147, XL-765, Palomid 529, GSK-1059615, ZSTK-474, PWT-33597, IC-87114, TG100-115, CAL-263, RP-6530, PI-103, GNE-477, CUDC-907, AEZS-136, or any combination thereof.

In some aspects, the disclosure further encompasses a method of preventing, treating, or reducing the risk of a condition or disease of large intestinal tract tissue associated with immune-related adverse events (irAEs) during immune checkpoint blockade therapy in a subject, comprising administering to a subject in need thereof an effective amount of a composition comprising the disclosed COIN. In such aspects, the subject is undergoing or planning to undergo cancer treatment with immune checkpoint blockade therapy. In some aspects, the immune checkpoint blockade therapy comprises administration of an inhibitor of PD-1, PD-L1, TIM-3, LAG-3, CTLA-4, CSF-1R, or any combinations thereof.

In some aspects of the method, the effective amount of the disclosed COIN is about 1 μg to about 100 mg. In some aspects, the administration results in reduced CD8+ T cell activation, pro-inflammatory cytokine expression, hematochezia, colitis-induced anemia, lymphocytic colitis, inflammation-induced colon shortening, epithelium damage immune cell infiltration into the lamina propria, tumor burden, rectum prolapse, microbiome dysbiosis, or any combination thereof. In some aspects, the administration results in enhanced M1 Mφ differentiation into M2 Mφ, pro-regulatory cytokine expression, repair of epithelium damage, recovery of body weight, colon length maintenance, commensal microbiomes, or any combination thereof.

In some aspects, the condition or disease of large intestinal tract tissue comprises colitis, inflammatory bowel disease, Crohn's Disease, ulcerative colitis, familial adenomatous polyposis, Hirschsprungs, proctitits, celiac disease, gastrointestinal mucositis, or fistula.

Further provided is a kit comprising COIN disclosed herein.

Other technical features may be readily apparent to one skilled in the art from the following figures, descriptions, and claims.

### BRIEF DESCRIPTION OF THE DRAWINGS

FIG. 1A-1I show bioengineering and characterization of the immunosuppressive COMBO COIN. FIG. 1A is a schematic of the proposed COMBO COIN mechanism of action. COMBO COIN exhaust anergize autoreactive CD8+ T cells and polarizes M1 Mφs to M2 Mφs. COMBO COIN promotes the recovery of ulcerated colon epithelium by producing a non-inflammatory environment for colon stem cells at crypts to replace apoptotic colon epithelial cells. FIG. 1B shows representative TEM image of unmodified carboxylic acid-functionalized PMPC-PHPMA NFs. FIG. 1C shows background-subtracted solution SAXS profiles of the unmodified and functionalized immunosuppressive NFs. The open dots represent the collected angle-dependent scattering intensities, and the solid lines represent the best fit to the cylinder scattering model. FIG. 1D shows in vitro binding of PE-labeled anti-PD-L1 and A488-labeled anti-CD86 to PD-L1 and/or CD86 NFs, as quantified via FACS. FIG. 1E shows representative H&E-stained images of the mouse colon before and after decellularization. FIG. 1F shows representative dark-field optical microscopy image of the ball-milled decellularized COL ECM. FIG. 1G shows representative SEM image of the ball-milled decellularized colon ECM. FIG. 1H shows volcano plots showing proteomic data for a näive mouse colon and a decellularized colon ECM. (n=3 biologically independent samples) FIG. 1I shows analysis of growth factors and cytokines present in mouse colon before and after decellularization via sandwich-type antibody microarray assay. (n=4 biologically independent samples) Data are presented as the mean±standard error of the mean (s.e.m.). All P values were analyzed using two-way ANOVA with Tukey's HSD multiple comparisons post-hoc test. * denotes p<0.05, hence statistically significant.

FIG. 2 shows self-assembly of PMPC-PHPMA diblock copolymers. Phase diagram of PMPC-PHPMA diblock copolymers with different PMPC and PHMPA block lengths. All PMPC-PHPMA diblock copolymers were prepared via aqueous RAFT polymerization at 25 wt/wt % solid content. High solid content facilitates the diblock copolymers self-assembly into different nano-objects.

FIG. 3 shows synthesis of PMPC-PHPMA diblock copolymer NFs through an optimized two-step RAFT polymerization. Carboxylic acid α-end-functionalized PMPC27 macro-chain transfer agent (macro-CTA) synthesized through RAFT polymerization. Chain extension of the PMPC27 macro-CTA with HPMA at 25 wt/wt % solid content yielded wormlike PMPC27-PHPMA280 (COOH-functionalized) NFs. The as-prepared COOH-functionalized NFs were then activated with EDC/sulfo-NHS before being functionalized with sulfo-DBCO amine to produce DBCO-functionalized NFs. MPC=2-methacryloyloxyethyl phosphorylcholine (monomer); CPAD=4-cyano-4-(phenylcarbonothioylthio) pentanoic acid (chain transfer agent); VA044=2,2′-azobis [2-(2-imidazolin-2-yl) propane]dihydrochloride (thermal initiator); HPMPA=2-hydroxypropyl methacrylate (monomer); EDC=1-ethyl-3-(3-dimethylaminopropyl) carbodiimide; sulfo-NHS=N-hydroxysulfosuccinimide. A family of PMPC27-PHPMAx (where x=140, 210, 350, and 420) diblock polymers were prepared using the same method, except that different PMPC27 macro-CTA and HPMA (monomer) molar ratios were used while the solid content was maintained at 25 wt/wt %.

FIG. 4 shows ¹H NMR spectroscopy of purified diblock copolymer NFs. ¹H NMR (400 MHz, in CD₃OD) spectra of (i) purified PMPC27 macro-CTA; (ii) unmodified COOH-functionalized PMPC27-PHPMA280 diblock copolymer; (iii) purified DBCO-functionalized PMPC27-PHPMA280 diblock copolymer; and (iv) unmodified COOH-functionalized PMPC27-PHPMA280 diblock copolymer after adjusted to pH 8.5 (in 1×PBS) and dialysis against water. The COOH-functionalized PMPC27-PHPMA280 diblock copolymer was lyophilized before being redissolved in CD₃OD for the ¹H NMR study. The DBCO-functionalized PMPC27-PHPMA280 diblock copolymer was purified via equilibrium dialysis against deionized water and lyophilized before being redissolved in CD₃OD for the ¹H NMR study. The control sample, dialyzed unmodified COOH-functionalized PMPC27-PHPMA280 diblock copolymer, was first incubated in 1×PBS at pH 8.5 for 12 h before dialysis against deionized water. The control diblock polymer was lyophilized before being redissolved in CD₃OD for the ¹H NMR study. The thiocarbonylthio RAFT end group was hydrolyzed under basic conditions (pH 8.5) during amine-NHS coupling.

FIG. 5 shows ¹H NMR spectroscopy of as-polymerized (non-purified) PMPC-PHPMA diblock copolymers. ¹H NMR (400 MHz) spectra of HPMA (monomer, recorded in CD₃OD); and the as-synthesized PMPC27-PHPMA280 diblock copolymer. The diblock polymer was prepared at 25 wt/wt % solid content. The spectrum of the as-prepared diblock copolymer was recorded in 1:10 H₂O/CD₃OD. The disappearance of methacrylate protons (CH₂═C(CH₃)C(═O)O—R) at 5.7 and 6.2 ppm (highlighted red regions) in all diblock copolymers confirmed the complete polymerization of HPMA.

FIG. 6 shows representative TEM images of the PMPC27-PHPMA280 diblock copolymer prepared at different solid contents. The preparation of the PMPC27-PHPMA280 diblock polymer at 25 wt/wt % solid content yielded the formation of monophasic rod-like NFs and thus suggested further use for in vitro and in vivo studies.

FIG. 7 shows physiological properties of PMPC27-PHPMA280 diblock copolymer NFs. Variable-temperature oscillatory rheology data were recorded for 12.5 wt/wt % and 5 wt/wt % of unmodified PMPC27-PHPMA280 diblock copolymer NFs. The oscillatory rheology study confirmed that the diblock copolymer is thermally stable between 5° C. and 37° C.

FIG. 8 shows bioengineering of COMBO ISM NFs and COMBO COIN. Bioengineering of the immune niche for UC treatment. The COL-specific immune niche was constructed from immunosuppressive NFs, COL EPI cells, and a decellularized COL biomatrix. Different immunomodulator-functionalized NFs were constructed from COOH alpha-end-functionalized PMPC-PHPMA diblock copolymer NFs. COOH-functionalized NFs were converted into DBCO-functionalized NFs through sulfo-NHS-EDC coupling with sulfo DBCO-amine. PD-L1 immunoglobulin (PD-L1-lg) and CD86 immunoglobulin (CD86-Ig) were converted into azide-functionalized PD-L1-Ig and CD86-Ig through amine-NHS ester coupling. The azide-functionalized fusion proteins were then conjugated to the DBCO-functionalized NFs through strain-promoted azide-alkyne cycloaddition (SPAAC). Azide-functionalized LEF-encapsulated NPs (LEF NPs) were conjugated to DBCO-functionalized NFs via SPAAC. Cationic thiol-rich TGF-β1 mimetic peptide was first oligomerized by incubation at 20° C. for 4 h before physiosorbed onto the anionic COOH-functionalized NFs to give TGF-β1 mimetic peptide-functionalized NFs (TGF-β1 mimic NFs). In this proof-of-concept study, decellularized COL biomatrix and COL EPI cells were isolated from healthy C57BL/6 mice. In practice, healthy COL tissue and COL EPI cells can be isolated from healthy COL tissue collected through biopsy or obtained from deceased donors. The decellularized COL was lyophilized and ball-milled to fine powder.

FIG. 9A-9B show characterization of azide-functionalized PD-L1-Ig and CD86-Ig. FIG. 9A shows UV-visible spectra of 1 mg/mL azide-functionalized PD-L1-Ig and CD86-Ig. The number of conjugated azide ligands was quantified by reacting with a large excess (50 molar equivalents per Ig) of MB488-DBCO dye at 37° C. for 1 h. Unreacted MB488-DBCO was removed by desalting columns (3 times). The target degree of conjugation was 40, and the actual degree of conjugation was approximately 8 for both proteins. The azide ligands were randomly conjugated to both fusion proteins. FIG. 9B shows UV-visible spectra of 1 mg/mL rhodamine B/azide dual-functionalized PD-L1-Ig and CD86-Ig. Both proteins were prepared using the same amine/NHS ester coupling (target degree of functionalization=50) in the presence of 4 molar equivalents of rhodamine B isocyanate. It was calculated that each protein was labeled with an average of 1 rhodamine B molecule.

FIG. 10A-10C show characterization of PD-L1 NFs, CD86 NFs and PD-L1/CD86 NFs. Azide-functionalized PD-L1-Ig and/or CD86-Ig were conjugated to the DBCO-functionalized NFs through SPAAC at a target loading of 10 μg of protein per 1 mg of DBCO-functionalized NFs, e.g., 5 μg of PD-L1 plus 5 μg of CD86 per 1 mg of NFs for the dual-functionalized NFs. FIG. 10A shows digital photographs of rhodamine B-labeled azide-functionalized PD-L1-Ig after conjugation to DBCO-functionalized NFs recorded before and after purification. FIG. 10B shows Conjugation efficiencies for the preparation of rhodamine B-labeled PD-L1 and/or CD86 mono-/dual-functionalized NFs determined by fluorescence spectroscopy (to quantify the amount of conjugated rhodamine B-labeled PD-L1 and/or CD86). The conjugation efficiencies of mono- and/or dual-functionalized NFs were above 92%. FIG. 10C shows UV-visible absorption spectra of 1 mg/mL non-fluorescently labeled PD-L1 and/or CD86 mono-/dual-functionalized NFs. The reduction in absorbance at 310 nm is a result of the change of cyclooctyne into triazole.

FIG. 10D shows representative TEM images of DBCO-functionalized NFs, PD-L1 NFs, CD86 NFs, and PD-L1/CD86 NFs.

FIG. 11A-11C show characterization of LEF NFs. Azide-functionalized LEF-encapsulated NPs (LEF NPs) were conjugated to the DBCO-functionalized NFs at a target loading of 0.1 mg LEF NPs for each 1 mg of DBCO-functionalized NFs. FIG. 11A shows Quantification of Cy5-labeled LEF NPs conjugated to DBCO-functionalized NFs via fluorescence spectroscopy. It was calculated that the conjugation efficiency was 59±4% (target loading: 200 μg of NPs per 1 mg of NFs); i.e., approximately 118 μg of LEF NPs were conjugated to 1 mg of DBCO-functionalized NFs. Since each 1 mg of the LEF NPs contained 8.7±1.5 wt/wt % of LEF (target loading=10 wt/wt %), each 1 mg of the LEF NFs encapsulated approximately 10 μg of the encapsulated LEF. FIG. 11B shows representative TEM images of LEF NPs, DBCO-functionalized NFs, LEF NFs (before and after purification), and LEF NFs after the in vitro drug release study. FIG. 11C shows In vitro LEF release kinetics under physiological conditions.

FIG. 12A-12D show characterization of TGF-β1 mimic NFs. Cationic TGF-β1 mimetic peptide was adsorbed onto the anionic COOH-functionalized NFs through electrostatic interactions at a loading of 50 μg of peptide per 1 mg of NFs. FIG. 12A shows representative TEM images of unmodified COOH-functionalized NFs, TGF-β1 mimic NFs, and NFs after an in vitro release study under different physiological conditions. Physiosorbed rhodamine-labeled TGF-β1 mimetic peptide was gradually released from NFs and taken up by CD8⁺ T cells in vitro. FIG. 12B shows in vitro TGF-β1 mimetic peptide release kinetics under reducing and non-reducing physiological conditions. FIG. 12C shows time-dependent FACS histograms (rhodamine channel) of CD8⁺ T cells after being cultured with rhodamine-labeled TGF-β1 mimic NFs for up to 48 h. FIG. 12D shows representative CLSM images of CD8⁺ T cells after being cultured with rhodamine-labeled TGF-31 mimic NFs for up to 48 h.

FIG. 13 shows solution SAXS patterns recorded for LEF NPs and a 1:1 mixture of PD-L1-Ig/CD86-Ig.

FIG. 14 shows biofunctionalized NFs show low in vitro toxicities when co-cultured with COL EPI cells. In vitro toxicities of different functionalized NFs against COL EPI cells. The viabilities of the COL EPI cells (1×10⁴ cells per well, 1/100 times the dose used in the in vivo studies) were quantified by MTS assay after being cultured with 10 μg of NFs per well or 10 μg of PD-L1/CD86 NFs plus 10 μg of LEF NFs and 10 μg of TGF-β1 mimic NFs per well for 4 days.

FIG. 15 shows spin-decellularization effectively decellularized the mouse colon. Quantification of DNA content in fresh and spin-decellularized mouse colon. Data are presented as the mean±s.e.m. P value was analyzed using one-way ANOVA with Tukey's HSD multiple comparisons post-hoc test.

FIG. 16A-16B show decellularized colon ECM provides nutrients for the proliferation of COL EPI cells in low growth factor growth media. The proliferation of COL EPI cells after being co-cultured with different concentrations of decellularized colon ECM in complete tissue culture medium supplemented with 2% FBS and 5 ng/mL recombinant epithelial growth factor (rEGF) and/or incomplete tissue culture medium supplemented with 1% FBS and 2.5 ng/mL rEGF. FIG. 16A shows representative optical microscope images of colon EPI cells after being cultured in vitro in the presence of different concentrations of decellularized colon ECM for 8 days. FIG. 16B shows relative viabilities of COL EPI cells (vs COL EPI cells cultured in ECM-free complete medium in gelatin-coated plates) after being cultured with different concentrations of decellularized colon ECM for 8 days. The in vitro study was performed in a non-adhesive 96-well plate. Unless specified, the wells were coated with gelatin before the in vivo study. The relative viabilities were quantified via MTS proliferation assay.

FIG. 17A-17E show COMBO ISM NFs effectively inhibit antigen-specific CD8⁺ T cell activation in vitro. FIG. 17A shows T cell inhibition (exhaustion) markers and proliferation indices of CD8⁺ T cells cultured with different functionalized NFs in the presence of anti-CD3/anti-CD28-functionalized T cell activation beads (Dynabeads) at a 1:1 ratio for 48 h, as quantified through FACS. The proliferation study was performed on CFSE-labeled CD8⁺ T cells. (n=4). FIG. 17B shows T cell activation and exhaustion markers on naïve and TNP-sensitized CD8⁺ T cells (in splenocytes) after co-culture with TNP-modified COL EPI cells in the presence or absence of COMBO ISMs or COMBO ISM NFs (48 h). (n=4). FIG. 17C shows ELISpot assay of IFN-gamma in splenocytes after culture with unmodified or TNP-modified COL EPI cells in the presence or absence of COMBO ISMs or COMBO ISM NFs (n=4). FIG. 17D-17E show cytotoxicity of splenocytes against COL EPI cells in the presence or absence of COMBO ISMs or COMBO ISM NFs (n=7). Data are presented as the mean±s.e.m. All P values were analyzed using two-way ANOVA with Tukey's HSD multiple comparisons post-hoc test.

FIG. 18A-18B show immunosuppressive NFs induce CD8⁺ T cell exhaustion. FIG. 18A shows representative flow histograms showing TIM-3 expressions on stimulated CD8⁺ T cells after incubation with different immunosuppressive NFs. FIG. 18B shows representative flow histograms showing LAG-3 expressions on stimulated CD8⁺ T cells after incubation with different immunosuppressive NFs.

FIG. 19A-19G show COMBO ISM NFs and colon ECM effectively inhibit antigen-specific CD8⁺ T cell activation and promote M1 Mφ differentiation into M2 Mφs in vitro. FIG. 19A-19B show T cell inhibition (exhaustion) markers and proliferation indices, respectively of CD8⁺ T cells cultured with COMBO ISM NFs, colon ECM and their mixture in the presence of anti-CD3/anti-CD28-functionalized T cell activation beads (Dynabeads) at a 1:1 ratio for 48 h, as quantified through FACS. The proliferation study was performed on CFSE-labeled CD8⁺ T cells. (n=4). FIG. 19C show T cell activation and exhaustion markers expressed on TNP-sensitized CD8⁺ T cells (in splenocytes) after co-culturing with TNP-modified colon epithelial cells in the presence or absence of COMBO ISM NFs, colon ECM, and their mixture for (48 h). (n=4). FIG. 19D shows ELISpot assay of IFN-γ in splenocytes after culture with TNP-modified colon epithelial cells in the presence or absence of COMBO ISM NFs, colon ECM, and their mixture (n=4). FIG. 19E shows cytotoxicity of TNP-sensitized splenocytes against TNP-modified colon epithelial cells in the presence or absence of COMBO ISM NFs, colon ECM, and their mixture (n=7). FIG. 19F shows ratio of M1 Mφs to M2 Mφs after the IFN-γ- and LPS-induced M1 Mφs were co-cultured with COMOBO ISM NFs, colon ECM, and their mixture, as determined by CLSM. M1 Mφs are characterized by roundish cell bodies with a few cytoplasmic extensions on the cell surface, and M2 Mφs are characterized by its elongated cell body with cytoplasmic extensions on their apical ends. (n=80-100 cells per group) FIG. 19G shows quantification of TNP-modified colon epithelial cells killed by induced M1 Mφs in the presence of COMBO ISM NFs, COL ECM, and their combination. (n=7) Data are presented as the mean±s.e.m. All P values were analyzed using two-way ANOVA with Tukey's HSD multiple comparisons post-hoc test.

FIG. 20 shows the combination of COMBO ISM NFs and COL ECM is more effective than COMBO ISM NFs alone in exhausting stimulated CD8⁺ T cells. Representative flow histograms show the TIM-3 and LAG-3 expressions on stimulated CD8⁺ T cells after incubation with different immunosuppressive NFs.

FIG. 21 shows immunosuppressive NFs inhibit the proliferation of stimulated CD8⁺ T cells. Representative CFSE profiles (and their best fit to the proliferation model) recorded for the CFSE-labeled CD8⁺ T cells cultured in the presence of different immunosuppressive NFs under stimulated conditions for 72 h. The CFSE-labeled CD8⁺ T cells were stimulated using anti-CD3/anti-CD28 T cell activation beads (Dynbeads) at a 1:1 ratio.

FIG. 22 shows the combination of COMBO ISM NFs and COL ECM is more effective than COMBO ISM NFs in inhibiting the proliferation of stimulated CD8⁺ T cells. Representative CFSE profiles (and their best fit to the proliferation model) recorded for the CFSE-labeled CD8⁺ T cells cultured in the presence of different immunosuppressive NFs under stimulated conditions for 72 h. The CFSE-labeled CD8⁺ T cells were stimulated using anti-CD3/anti-CD28 T cell activation beads (Dynbeads) at a 1:1 ratio.

FIG. 23A-23C show COMBO ISM NFs inhibit antigen-specific CD8⁺ T cell activation against TNP-modified COL EPI cells in vitro. FIG. 23A shows Gating strategy for the quantification of intracellular IFN-gamma and granzyme B expression in CD8⁺ T cells (among cocultured splenocytes) via FACS. FIG. 23B-23C show FACS dot plots showing the IFN-gamma and granzyme B, respectively expressions of TNP-sensitized and non-sensitized (naïve) CD8⁺ T cells after incubation with TNP-modified COL EPI cells (or unmodified COL EPI cells, as a control) in the presence or absence of COMBO ISM NFs (or COMBO ISMs) for 48 h. The effector cells are TNF-sensitized or non-sensitized splenocytes. The E:T ratio was 20:1 (2×10⁵ splenocytes:1×10⁴ COL EPI cells).

FIG. 24 shows the combination of COMBO ISM NFs and COL ECM is more effective than COMBO ISM NFs in inhibiting antigen-specific CD8⁺ T cell activation against TNP-modified COL EPI cells in vitro. FACS dot plots showing the IFN-expression of TNP-sensitized and non-sensitized (naïve) CD8⁺ T cells after incubation with TNP-modified COL EPI cells (or unmodified COL EPI cells, as a control) in the presence or absence of COMBO ISM NFs and COL ECM for 48 h. The effector cells are TNF-sensitized or non-sensitized splenocytes. The E:T ratio was 20:1 (2×10⁵ splenocytes:1×10⁴ COL EPI cells).

FIG. 25A-25B show COMBO ISM NFs inhibit antigen-specific CD8⁺ T cell activation triggered by TNP-modified COL EPI cells in vitro by inducing T cell exhaustion. FIG. 25A shows Gating strategy for the quantification of T cell inhibition markers on CD8⁺ T cells (among the co-cultured splenocytes) via FACS. FIG. 25B shows FACS dot plots showing the LAG-3 and TIM-3 expressions of TNP-sensitized and non-sensitized (naïve) CD8⁺ T cells after incubation with TNP-modified COL EPI cells in the presence or absence of COMBO ISM NFs (or COMBO ISMs) for 48 h. The effector cells are TNF-sensitized or non-sensitized splenocytes. The E:T ratio was 20:1 (2×10⁵ splenocytes:1×10⁴ COL EPI cells). Data are presented as the mean±standard error of the mean (s.e.m.). All P values were analyzed using two-way ANOVA with Tukey's HSD multiple comparisons post-hoc test.

FIG. 26 shows the combination of COMBO ISM NFs and COL ECM is more effective in inducing antigen-specific T cell exhaustion in vitro. FACS dot plots showing the LAG-3 and TIM-3 expression in TNP-sensitized and non-sensitized (naïve) CD8⁺ T cells after incubation with TNP-modified COL EPI cells in the presence or absence of COMBO ISM NFs and COL ECM for 48 h. The effector cells are TNF-sensitized or non-sensitized splenocytes. The E:T ratio was 20:1 (2×10⁵ splenocytes:1×10⁴ COL EPI cells). Data are presented as the mean±standard error of the mean (s.e.m.). All P values were analyzed using two-way ANOVA with Tukey's HSD multiple comparisons post-hoc test.

FIG. 27 shows COMBO ISM NFs inhibit the secretion of proinflammatory IFN-gamma from antigen-specific splenocytes when co-cultured with TNP-modified COL EPI cells in vitro. Digital photograph of IFN-gamma ELISpot wells after TNP-modified COL EPI cells were co-cultured with TNP-sensitized or naïve splenocytes in the presence or absence of COMBO ISM NFs or COMBO ISMs for 48 h.

FIG. 28 shows the combination of COMBO ISM NFs and COL ECM effectively inhibits the secretion of proinflammatory IFN-gamma from antigen-specific splenocytes when co-cultured with TNP-modified COL EPI cells in vitro. Digital photograph of IFN-gamma ELISpot wells after TNP-modified COL EPI cells were co-cultured with TNP-sensitized or naïve splenocytes in the presence COMBO ISM NFs and/or COL ECM for 48 h.

FIG. 29 shows the combination of COMBO ISM NFs and COL ECM induces M1 Mφs differentiation into M2 Mφs in vitro. Representative CLSM images of IFN-γ- and LPS-induced M1 Mφs after cultured with different ISM NFs, COL ECM and their combination for 48 h. The round-shape M1 Mφs (with cytoplasmic extension on the cellular surface) differentiated into elongated M2 Mφs with cytoplasmic extension on the apical ends after co-cultured with COMBO ISM NFs and COL ECM.

FIG. 30A-30T show COMBO COIN effectively ameliorated colitis and promoted recovery in established mouse UC models. FIG. 30A-301 show optimization of COIN formulations in the DSS-induced colitis model. FIG. 30A depicts colitis treatment schedule with each colitis mouse received a single s.c. inoculation of COIN at day 6 (onset of colitis symptoms). Bodyweight changes and disease activity index (DAI) scores were monitored daily for 13 days. The colons were preserved at the study endpoint (day 13). FIG. 30B shows DAI score after colitis treatment with different immune checkpoint molecule-functionalized COIN. (n=8). All P values were analyzed using two-way ANOVA with Tukey's HSD multiple comparisons post-hoc test. FIG. 30C shows length of colon preserved at the study endpoint after colitis treatment with different immune checkpoint molecule-functionalized COIN. (n=8). All P values were analyzed using one-way ANOVA with Tukey's HSD multiple comparisons post-hoc test. FIG. 30D shows colon damage score from colitis mice after different treatments with immune checkpoint molecule-functionalized COINs. All P values were analyzed using two-way ANOVA with Tukey's HSD multiple comparisons post-hoc test. FIG. 30E shows representative H&E-stained colon sections preserved from colitis mice after different treatments with immune checkpoint molecule-functionalized COINs. FIG. 30F shows DAI score after colitis treatment with different non-immune checkpoint molecule-functionalized COIN and COMBO COIN. (n=8). All P values were analyzed using two-way ANOVA with Tukey's HSD multiple comparisons post-hoc test. FIG. 30G shows length of colon preserved at the study endpoint after colitis treatment with different non-immune checkpoint molecule-functionalized COIN and COMBO COIN. (n=8). All P values were analyzed using one-way ANOVA with Tukey's HSD multiple comparisons post-hoc test. FIG. 30H shows colon damage score from colitis mice after different treatments with immune checkpoint molecule-functionalized COINs. All P values were analyzed using two-way ANOVA with Tukey's HSD multiple comparisons post-hoc test. FIG. 30I shows representative H&E-stained colon sections preserved from colitis mice after different treatments with immune checkpoint molecule-functionalized COINs. FIG. 30J-30N show evaluation of the optimized COMBO COIN formulation for colitis treatment in a TNP-induced colitis model. FIG. 30J depicts colitis induction and treatment schedule. Each colitis mouse received a single s.c. inoculation of TNP-modified COMBO COIN (engineered with TNP-modified colon epithelial cells) at day 15 (onset of colitis symptoms). The colon was preserved at the endpoint (day 20) for mechanistic study. FIG. 30K shows DAI score of TNP-induced colitis mice after colitis treatment with TNP-modified COMBO COIN. All P values were analyzed using two-way ANOVA with Tukey's HSD multiple comparisons post-hoc test. FIG. 30L shows length of colon preserved at the experiment endpoint. All P values were analyzed using one-way ANOVA with Tukey's HSD multiple comparisons post-hoc test. FIG. 30M shows colon damage score from TNP-induced colitis mice after treatment with TNP-modified COMBO COIN. (n=8, 1 mouse in the non-treatment group died shortly before euthanization, and the colon was not preserved for histological study.). All P values were analyzed using two-way ANOVA with Tukey's HSD multiple comparisons post-hoc test. FIG. 30N shows representative H&E-stained colon sections preserved from TNP-induced colitis mice after treatment with TNP-modified COMBO COIN. (n=8, 1 mouse in the non-treatment group died shortly before euthanization, and the colon was not preserved for histological study.) FIG. 30O-FIG. 30T show evaluation of the optimized COMBO COIN formulation for colitis treatment in IL10 KO mice that spontaneously developed colitis at an early age. IL10 KO mice developed colitis symptoms at 10-11 weeks of age and received COMBO COIN treatment at weeks 11.5, 13.5 and 15.5. FIG. 30O shows DAI score of colitis IL10 KO mice after receiving COMBO COIN treatments. FIG. 30P shows blood hemoglobin counts recorded for IL10 KO mice after colitis treatments with COMBO COIN. All P values were analyzed using two-way ANOVA with Tukey's HSD multiple comparisons post-hoc test. FIG. 30Q shows lymphocyte counts recorded for IL10 KO mice after colitis treatments with COMBO COIN. All P values were analyzed using one-way two-way ANOVA with Tukey's HSD multiple comparisons post-hoc test. FIG. 30R shows length of the colon of IL10 KO mice preserved at the study endpoint (17 weeks old). All P values were analyzed using one-way ANOVA with Tukey's HSD multiple comparisons post-hoc test. FIG. 30S shows colon damage score from 17 week old IL10 KO mice after treatment with COMBO COIN. (n=6). All P values were analyzed using two-way ANOVA with Tukey's HSD multiple comparisons post-hoc test. FIG. 30T shows representative H&E-stained colon sections preserved from 17 week old IL10 KO mice after treatment with COMBO COIN. (n=6) Data are presented as the mean±s.e.m. * denotes p<0.05, hence statistically significant. In the DSS-induced colitis model, DAI scores were compared based on data recorded up to day 10, i.e., before the non-treated mice started to recover.

FIG. 31A-31C show therapeutic treatment with immune checkpoint molecule-bioengineered COINs prevents colitis-associated weight loss, and colon shortening and reduces immune cell infiltration into the LP. FIG. 31A shows bodyweight changes throughout the study. FIG. 31B shows digital photographs of colons preserved at the study endpoint, i.e., 7 days after therapeutic treatments. FIG. 31C shows representative H&E-stained section of each colon preserved at the study endpoint after different treatments. The inserted label in each image represents the corresponding colon epithelial damage score and inflammatory cell infiltration score. P values were analyzed using one-way ANOVA with Tukey's HSD multiple comparisons post-hoc test.

FIG. 32A-32D show treatment with s.c.-inoculated COINs or i.p.-administered COMBO ISM NFs inhibits the production of colitis-associated MPO and proinflammatory cytokines in the colon. FIG. 32A shows Colonic MPO activity in the preserved COL specimens at the study endpoint (5 days after different therapeutic treatments) after different therapeutic treatments (n=5). FIG. 32B shows pro-inflammatory cytokine in the preserved COL specimens at the study endpoint (5 days after different therapeutic treatments) after different therapeutic treatments (n=5). FIG. 32C shows pro-regulatory cytokine expression in the preserved COL specimens at the study endpoint (5 days after different therapeutic treatments) after different therapeutic treatments (n=5). FIG. 32D shows representative immunofluorescence images of colons preserved from DSS colitis mice, captured 5 days after different therapeutic treatments, reveal PL-infiltrated IFN-gamma⁺ CD8⁺ T cells. Data are presented as the mean±s.e.m. All P values were analyzed using one-way ANOVA with Tukey's HSD multiple comparisons post-hoc test.

FIG. 33A-33C show therapeutic treatment with non-immune checkpoint molecule-functionalized COINs and COMBO COIN prevents colitis-associated colon shortening and reduces immune cell infiltration into the LP. FIG. 33A shows bodyweight changes throughout the study. FIG. 33B shows digital photographs of colons preserved at the study endpoint, i.e., 7 days after different therapeutic treatments. FIG. 33C shows representative H&E-stained section of each colon preserved at the study endpoint after different treatments. The inserted label in each image represents the corresponding colon epithelial damage score and inflammatory cell infiltration score. P values were analyzed using one-way ANOVA with Tukey's HSD multiple comparisons post-hoc test.

FIG. 34 shows colitis treatment with s.c.-administered COMBO COIN or i.p-administered COMBO ISM NFs resolved imbalance of the pathogenic and commensal fecal microbiome in DSS colitis mice. Heatmap of the relative abundance of the microbiome (rows) for each DSS colitis mouse (column) after different treatments. The abundance is shown as a relative percentage. Gut microbiome analysis (by 16S rRNA sequencing) was performed on feces collected 5 days after colitis treatment.

FIG. 35A-35E show COMBO COIN effectively ameliorates DSS-induced colitis in male C57BL/6 mice. FIG. 35A-35D show evaluation of the therapeutic treatment efficiency of COMBO COIN in the DSS-induced colitis mouse model. FIG. 35A shows the therapeutic treatment schedule with colitis mice receiving a single s.c. treatment with the combo immune niche at day 6. FIG. 35B shows bodyweight change monitored for up to 12 days. P values were analyzed using two-way ANOVA with Tukey's HSD multiple comparisons post-hoc test. FIG. 35C shows DAI score monitored for up to 12 days. P values were analyzed using two-way ANOVA with Tukey's HSD multiple comparisons post-hoc test. FIG. 35D shows digital images and length of colon preserved at the study endpoint. P values were analyzed using one-way ANOVA with Tukey's HSD multiple comparisons post-hoc test. FIG. 35E shows H&E-stained sections of each colon preserved at the study endpoint and their disease scores after therapeutic treatment with the combo immune niche. The inserted label in each image represents the corresponding colon epithelial damage score and inflammatory cell infiltration score. P values were analyzed using one-way ANOVA with Tukey's HSD multiple comparisons post-hoc test. Data are presented as the mean±standard error of the mean (s.e.m.).

FIG. 36A-36F show the combination of COL EPI cells and COMBO ISM NFs, or COL ECM alone are not as effective as COMBO COIN (the combination of COL EPI cells, COL ECM, COMBO ISM NFs) in relieving the colitis symptoms in a DSS-induced colitis model. FIG. 36A shows bodyweight changes after different control colitis treatments with different control COINs. FIG. 36B shows DAI scores after different control colitis treatments with different control COINs. FIG. 36C shows bodyweight changes after different control colitis treatments with different control COINs. FIG. 36D shows DAI scores after different control colitis treatments with different control COINs. FIG. 36E shows bodyweight changes after different control colitis treatments with different control COINs. FIG. 36F shows DAI scores after different control colitis treatments with different control COINs. Statistical analysis was performed 4 days after different colitis treatments. (n=6 or 7) Data are presented as the mean±s.e.m. All P values were analyzed using two-way ANOVA with Tukey's HSD multiple comparisons post-hoc test.

FIG. 37A-37B show the combination of COL EPI cells and COMBO ISM NFs, or COL ECM alone are not as effective as COMBO COIN (the combination of COL EPI cells, COL ECM, COMBO ISM NFs) in preventing inflammation-associated colon shortening, colitis-induced colon epithelial damage and immune cell infiltration into the colon in the DSS colitis model. FIG. 37A shows digital photographs and the lengths of colons preserved at the study endpoint after different colitis treatments. All P values were analyzed using one-way ANOVA with Tukey's HSD multiple comparisons post-hoc test. FIG. 37B shows representative H&E-stained sections of each colon and colon damage scores after different colitis treatments. The inserted label in each image represents the corresponding colon epithelial damage score and inflammatory cell infiltration score. (n=6 or 7) All P values were analyzed using two-way ANOVA with Tukey's HSD multiple comparisons post-hoc test.

FIG. 38A-38D show pancreas-specific COMBO immune niche (COMBO PAN-IN) cannot relieve colitis symptoms in DSS colitis mice. FIG. 38A shows analysis of growth factors and cytokines present in decellularized colon ECM and decellularized mouse pancreas via sandwich-type antibody microarray assay. (n=4 biologically independent samples). All P values were analyzed using two-way ANOVA with Tukey's HSD multiple comparisons post-hoc test. FIG. 38B shows bodyweight change after colitis treatment with COMBO COIN or COMBO PAN-IN. All P values were analyzed using two-way ANOVA with Tukey's HSD multiple comparisons post-hoc test. FIG. 38C shows DAI score after colitis treatment with COMBO COIN or COMBO PAN-IN. All P values were analyzed using two-way ANOVA with Tukey's HSD multiple comparisons post-hoc test. FIG. 38D shows digital photographs of colons preserved at the study endpoint (5 days after therapeutic treatment), and their lengths. (n=7 or 8) All P values were analyzed using one-way ANOVA with Tukey's HSD multiple comparisons post-hoc test.

FIG. 39A-39D show i.p.-administered COMBO ISM NFs are as effective as s.c.-inoculated COMBO COIN to treat DSS colitis. FIG. 39A shows bodyweight change after colitis treatment with COMBO COIN (s.c.) or COMBO ISM NFs (i.p.). All P values were analyzed using two-way ANOVA with Tukey's HSD multiple comparisons post-hoc test. FIG. 39B shows DAI score after colitis treatment with COMBO COIN (s.c.) or COMBO ISM NFs (i.p.). All P values were analyzed using two-way ANOVA with Tukey's HSD multiple comparisons post-hoc test. FIG. 39C shows digital photographs of colons preserved at the study endpoint (5 days after therapeutic treatment), and their lengths. All P values were analyzed using one-way ANOVA with Tukey's HSD multiple comparisons post-hoc test. FIG. 39D shows representative H&E-stained sections of each colon and colon damage scores after different colitis treatments. The inserted label in each image represents the corresponding colon epithelial damage score and inflammatory cell infiltration score. (n=8) All P values were analyzed using two-way ANOVA with Tukey's HSD multiple comparisons post-hoc test.

FIG. 40A-40D show s.c.-inoculated COMBO COIN is retained at the inoculation site, but i.p.-administered COMBO ISM NFs diffuse throughout the abnormal cavity after administration. FIG. 40A shows ex vivo fluorescent images of inoculation site and grafts preserved 3 days after s.c. inoculation of COMBO COIN constructed from rhodamine-labeled NFs in DSS-induced colitis mice. FIG. 40B shows ex vivo fluorescent images of spleen and liver preserved 3 days after s.c. inoculation of COMBO COIN. There is no indication for the clearance of NFs by the immune cells that would accumulate in the liver and spleen. FIG. 40C-40D show in vivo and ex vivo fluorescence images recorded for DSS colitis mice 3 days after therapeutic treatment with i.p.-administered rhodamine-labeled COMBO ISM NFs.

FIG. 41A-41B show TNP-modified COMBO COIN effectively ameliorates TNP-induced colitis. FIG. 41A shows digital photographs of colons preserved at the study endpoint. FIG. 41B shows representative H&E-stained sections of each colon and colon damage scores after different colitis treatments. The inserted label in each image represents the corresponding colon epithelial damage score and inflammatory cell infiltration score.

FIG. 42A-42C show COMBO COIN effectively ameliorates colitis in IL10 KO mice that spontaneously develop colitis. FIG. 42A shows bodyweight change. FIG. 42B shows digital photographs of colons preserved at the study endpoint. FIG. 42C shows representative H&E-stained sections of each colon and colon damage scores after different colitis treatments. The inserted label in each image represents the corresponding colon epithelial damage score and inflammatory cell infiltration score. (n=6).

FIG. 43A-43J show mechanistic insight: COMBO COIN ameliorated DSS-induced colitis by inhibiting COL-specific CD8⁺ T cell activation and polarizing M1 Mφs into M2 Mφs. FIG. 43A-43B show immune profiling of colons preserved from DSS colitis mice after different COIN treatments. FIG. 43A shows stacked bar graph representing the frequencies of each type of LP-infiltrated lymphocyte at different time points after different COIN treatments. FIG. 43B shows frequencies of proinflammatory CD86^{high} F4/80⁺ CD11b⁺ M1 Mφs, anti-inflammatory CD₂₀₆^{high} F4/80⁺ CD11b⁺ M2 Mφs, and proinflammatory IFN-γ^{high} and/or TNF-α^{high} CD8⁺ T cells in the LP after different colitis treatments. (n=5). All P values were analyzed using two-way test ANOVA with Tukey's HSD multiple comparisons post-hoc test. FIG. 43C shows representative immunofluorescence images of colons preserved from DSS colitis mice, captured four days after different therapeutic treatments, reveal PL-infiltrated CD4⁺ T cells, CD8⁺ T cells and F4/80⁺ Mφs. FIG. 430-43E show evaluation of the therapeutic efficiencies of COMBO COIN in T_{reg} cell-, CD8⁺ T cell- and Mφ-depleted DSS colitis mice. T_{reg} cells and CD8⁺ T cells were depleted (De.) by i.p. administration of anti-CD25 or anti-CD8a at days 4, 6 and 8. Mφs were depleted by a single i.v. administration of Clodrosome (containing encapsulated clodronate) on day 5. All mice received colitis treatment (T) with COMBO COIN at day 6. FIG. 43D shows DAI scores of different immune cell-depleted mice after colitis treatment with COMBO COIN. FIG. 43E shows length of the colon of different immune cell-depleted mice measured at the study endpoint. (n=6 or 7). All P values were analyzed using one-way test ANOVA with Tukey's HSD multiple comparisons post-hoc test. FIG. 43F shows representative TUNEL-stained colon sections preserved from healthy and colitis mice 4 days after treatment with different COINs. The TUNEL staining identified TUNEL⁺ apoptotic colon epithelial cells. (n=5). All P values were analyzed using two-way test ANOVA with Tukey's HSD multiple comparisons post-hoc test. FIG. 43G shows Representative Ki67-stained colon sections preserved from health and colitis mice 4 days after treatment with different COINs. The Ki67 stain highlights the proliferating colon epithelial stem cells in the colonic crypts. FIG. 43H-43J show immune profiling of COMBO COIN at different time points after s.c. inoculation in DSS colitis mice. FIG. 43H shows stacked bar graph representing the frequencies of each type of infiltrated lymphocytes at different time points after inoculation. (n=5). FIG. 43I shows frequencies of proinflammatory CD₈₆^{high} F4/80⁺ CD11b⁺ M1 Mφs and TNF-α^{high} CD8⁺ T cells infiltrated into the COIN at different time points after inoculation. All P values were analyzed using two-way test ANOVA with Tukey's HSD multiple comparisons post-hoc test. FIG. 43J shows representative immunofluorescence images of s.c. inoculated COMBO COIN preserved 3, 5 or 12 days after inoculation. Data are presented as the mean±s.e.m. In the DSS-induced colitis model, the DAI scores were compared based on data recorded up to day 10 before the non-treated mice started to recover.

FIG. 44 shows gating strategy for immune profiling of immune cells at the colonic lamina propria through flow mass cytometry.

FIG. 45A-45D show COIN and ISM NF treatment significantly increased the anti-inflammatory M2 MOs, CCR7⁺ CD4⁺ memory T cells and exhausted CD8⁺ T cells at the LP. FIG. 45A shows ratio of colonic proinflammatory M1 Mφs to anti-inflammatory M2 Mφs after different colitis treatments. FIG. 45B shows frequency of pro-regulatory colonic T_{reg} after different colitis treatments. FIG. 45C shows frequency of memory T cells after different colitis treatments. FIG. 45D shows frequency of exhausted colonic CD8⁺ T cells after different colitis treatments. (n=5) All P values were analyzed using two-way ANOVA with Tukey's HSD multiple comparisons post-hoc test.

FIG. 46A-46B show treatment with COMBO COIN relieves colitis symptoms through the inhibition of CD8⁺ T cell activation and macrophage polarization. FIG. 46A shows bodyweight change after treatment with COMBO COIN in CD8⁺ T cell, T_{reg} cell-, and macrophage-depleted DSS colitis mice. FIG. 46B shows digital photographs of colons preserved at the study endpoint after treatment with COMBO COIN in CD8⁺ T cell or T_{reg} cell-, and macrophage-depleted colitis mice. All P values were analyzed using two-way ANOVA with Tukey's HSD multiple comparisons post-hoc test.

FIG. 47A-47D show single therapeutic treatment with COMBO COIN effectively ameliorates colitis symptoms in a chronic DSS-colitis model. FIG. 47A shows DAI score change after treatment(s) with COMBO COIN. FIG. 47B shows bodyweight change after treatment(s) with COMBO COIN. FIG. 47C shows digital photographs and length of colons preserved at the study endpoint (48 days after the initial colitis induction) after one or three therapeutic treatments with the combo immune niche. FIG. 47D shows representative H&E-stained images, colon epithelial damage score, inflammatory cell infiltration score, and colonic damage score of colons preserved at the study endpoint (48 days after the first induction of colitis) after one or three therapeutic treatments with COMBO COIN.

FIG. 48 shows gating strategy for immune profiling of immune cells at the s.c.-inoculated COMBO COIN through imaging mass cytometry.

FIG. 49A-49M show COMBO COIN effectively ameliorated colitis-associated colorectal cancer and PI3K inhibition associated colitis in cell signaling pathway-targeted cancer treatment. FIG. 49A shows schematic of the treatment schedule for the evaluation of COMBO COIN in preventing colitis-associated colorectal cancer through the AOM/DSS colitis-associated cancer model. FIG. 49B shows time-dependent DAI scores. Data are presented as mean±s.e.m. All P values were analyzed using the two-way ANOVA with Tukey's HSD multiple comparisons post-hoc test. FIG. 49C shows length of colons preserved at the study endpoint after different treatments, and representative digital photographs of colons from different control and treatment group mice harvested at the study endpoint. The preserved colons were stained with Alcian blue to identify the tumor boundaries. Data are presented as mean±s.e.m. All P values were analyzed using the one-way ANOVA with Tukey's HSD multiple comparisons post-hoc test. FIG. 49D shows colorectal cancer tumor burdens of AOM/DSS colitis mice after different treatments. Data are presented as mean±s.e.m. All P values were analyzed using the one-way ANOVA with Tukey's HSD multiple comparisons post-hoc test. FIG. 49E shows H&E-stained histological images of preserved colons at the study endpoint after different treatments against AOM/DSS colitis. (n=5, except AOM/DSS colitis-associated cancer groups with 8 mice per group.) FIG. 49F shows schematic of cancer and colitis treatment schedules for the evaluation of COMBO COIN in relieving copanlisib-induced colitis in B16OVA tumor model. FIG. 49G shows tumor growth curves recorded for B16OVA tumor-bearing mice after cancer treatment with copanlisib and colitis treatment with copanlisib. (n=7). Data are presented as mean±s.e.m. All P values were analyzed using the two-way ANOVA with Tukey's HSD multiple comparisons post-hoc test. FIG. 49H shows DAI scores recorded for B16OVA tumor-bearing mice after cancer treatment with copanlisib and colitis treatment with copanlisib. (n=7). Data are presented as mean±s.e.m. All P values were analyzed using the two-way ANOVA with Tukey's HSD multiple comparisons post-hoc test. FIG. 49I shows length of colon preserved at the experimental endpoint (day 22 post-inoculation). Data are presented as mean±s.e.m. All P values were analyzed using the one-way ANOVA with Tukey's HSD multiple comparisons post-hoc test. FIG. 49J shows colon damage score preserved from copanlisib-treated B16OVA tumor-bearing mice after cancer and colitis treatments. Data are presented as mean±s.e.m. All P values were analyzed using the two-way ANOVA with Tukey's HSD multiple comparisons post-hoc test. FIG. 49K shows representative H&E-stained colon sections preserved from copanlisib-treated B16OVA tumor-bearing mice after cancer and colitis treatments. FIG. 49L shows representative TUNEL-stained colon sections preserved from healthy and colitis mice 4 days after treatment (day 22 post-inoculation of B16OVA tumor) with COMBO COINs. TUNEL staining identified TUNEL⁺ apoptotic colon epithelial cells. Data are presented as mean±s.e.m. All P values were analyzed using the two-way ANOVA with Tukey's HSD multiple comparisons post-hoc test. FIG. 49M shows representative Ki67-stained colon sections from copanlisib-treated B16OVA tumor-bearing mice preserved 4 days after colitis treatment with COMBO COIN. Ki67 staining highlights proliferating colon epithelial stem cells in the colonic crypts. Data are presented as mean±s.e.m.

FIG. 50A-50B show therapeutic treatment with COMBO COIN prevents the development of colorectal cancer. FIG. 50A shows bodyweight change after treatment with COMBO COIN in the AOM/DSS colitis-induced colorectal cancer tumor model. FIG. 50B shows H&E-stained colon sections preserved at the study endpoint (65 days after the initial study) after therapeutic treatment with COMBO COIN in the AOM/DSS colitis-induced colorectal cancer tumor model.

FIG. 51A-51C show COMBO COIN effectively ameliorates COP-induced colitis in B16OVA murine tumor model. FIG. 51A shows in vitro toxicity of COP against B16OVA cells, as determined by MTS assay. FIG. 51B shows digital photographs of colons preserved at the study endpoint. FIG. 51C shows representative H&E-stained sections of each colon and colon damage scores after different colitis treatments. The inserted label in each image represents the corresponding colon epithelial damage score and inflammatory cell infiltration score. (n=7).

FIG. 52A-52E show dual immune checkpoint blockade worsens colitis symptoms in a DSS-induced colitis model. FIG. 52A shows colitis induction schedule. FIG. 52B shows bodyweight change recorded after colitis induction with and without immune checkpoint blockade. All P values were analyzed using two-way ANOVA with Tukey's HSD multiple comparisons post-hoc test. FIG. 52C shows DAI score recorded after colitis induction with and without immune checkpoint blockade. All P values were analyzed using two-way ANOVA with Tukey's HSD multiple comparisons post-hoc test. FIG. 52D shows digital photographs of colons and their corresponding lengths preserved at the study endpoint. All P values were analyzed using one-way ANOVA with Tukey's HSD multiple comparisons post-hoc test. FIG. 52E shows H&E images of preserved colons and corresponding disease scores. Data are presented as the mean±standard error of the mean (s.e.m.). All P values were analyzed using one-way ANOVA with Tukey's HSD multiple comparisons post-hoc test.

FIG. 53A-53N show s.c.-administered COMBO COIN and i.p.-administered COMBO ISM NFs are equally effective in ameliorating immune-related colitis without affecting the anticancer activities of ICB. FIG. 53A shows an ex vivo binding study quantified the binding affinities of splenetic CD8⁺ T cells to FITC-labeled PD-L1-Ig and Rhod-labeled CD86-Ig after ICB, as determined using FACS. All P values were analyzed using one-way ANOVA with Tukey's HSD multiple comparisons post-hoc test. FIG. 53B-53D show evaluation of s.c.-inoculated COMBO COIN and i.p.-administered COMBO ISM NFs ameliorating immune-related colitis after ICB with anti-PD-1 (100 μg/injection) and anti-CTLA-4 (100 μg/injection) in a B16F10 tumor model. FIG. 53B shows tumor growth curves after cancer ICB and colitis treatment with s.c.-administered COMBO COIN and i.p.-administered COMBO ISM NFs (n=8). All P values were analyzed using two-way ANOVA with Tukey's HSD multiple comparisons post-hoc test. FIG. 53C shows DAI scores after cancer ICB and colitis treatment with s.c.-administered COMBO COIN and i.p.-administered COMBO ISM NFs (n=8). All P values were analyzed using two-way ANOVA with Tukey's HSD multiple comparisons post-hoc test. FIG. 53D shows colon length at the endpoint after cancer ICB and colitis treatment with s.c.-administered COMBO COIN and i.p.-administered COMBO ISM NFs (n=8). All P values were analyzed using two-way ANOVA with Tukey's HSD multiple comparisons post-hoc test. FIG. 53E shows survival of B16F10 tumor-bearing mice after receiving ICB treatment with or without colitis treatment. FIG. 53F shows colon damage scores of colons preserved at the endpoint. All P values were analyzed two-way ANOVA with Tukey's HSD multiple comparisons post-hoc test. FIG. 53G shows representative H&E-stained colon sections. FIG. 53H shows representative TUNEL-stained colon sections preserved from B16F10 tumor bearing mice after receiving cancer and colitis treatments. TUNEL staining identified TUNEL⁺ apoptotic COL EPI cells. All P values, were analyzed using two-way ANOVA with Tukey's HSD multiple comparisons post-hoc test. FIG. 53I shows representative Ki67-stained colon sections preserved from B16F10 tumor-bearing mice after receiving cancer and colitis treatments. Ki67 staining highlights proliferating colon epithelial stem cells in the colonic crypts. FIG. 53J-53K show evaluation of the impact of colitis treatment with s.c.-inoculated COMBO COIN or i.p.-administered COMBO ISM NFs on ICB treatment with anti-PD-I and anti-CTLA-4 in the B16OVA tumor model. FIG. 53J shows tumor growth curve. All P values, were analyzed using two-way ANOVA with Tukey's HSD multiple comparisons post-hoc test. FIG. 53K shows frequencies of tumor-infiltrated OVA-specific CD8⁺ T cells and TIM-3^{high} OVA-specific CD8⁺ (exhausted) T cells 3 days after colitis treatment with COMBO COIN or COMBO ISM NFs. All P values were analyzed using one-way ANOVA with Tukey's HSD multiple comparisons post-hoc test. FIG. 53L-53N show evaluation of COMBO COIN and COMBO ISM NFs ameliorating IMC after ICB with anti-PD-1 and anti-CTLA-4 in the MC38 synergetic colorectal cancer model and the impact of colitis treatment on subsequent maintenance ICB treatment. FIG. 53L shows tumor growth curves of MC38 tumor-bearing mice after receiving ICB cancer treatment and colitis treatment with COMBO COIN or COMBO ISM NFs (n=8). All P values were analyzed using two-way ANOVA with Tukey's HSD multiple comparisons post-hoc test. FIG. 53M shows DAI scores of MC38 tumor-bearing mice after receiving ICB cancer treatment and colitis treatment with COMBO COIN or COMBO ISM NFs (n=8). All P values, were analyzed using two-way ANOVA with Tukey's HSD multiple comparisons post-hoc test. FIG. 53N shows survival curves of MC38 tumor-bearing mice after receiving ICB cancer treatment and colitis treatment with COMBO COIN or COMBO ISM NFs (n=8). All P values were analyzed using two-way ANOVA with Tukey's HSD multiple comparisons post-hoc test. Data are presented as the mean±s.e.m. P values for the survival data were analyzed using the log-rank (Mantel-Cox) test.

FIG. 54A-54C show anti-PD-1 and anti-CTLA-4 slowly detach from CD8⁺ T cells after ICB treatment, allowing PD-L1 and CD86 binding to CD8⁺ T cells to induce colon-specific immunotolerance. FIG. 54A shows ex vivo binding study treatment schedule. FIG. 54B shows FACS gating strategy. FIG. 54C shows individual dot scatter plots of CD8⁺ T cells after binding to FITC-labeled PD-L1-Ig and Rhod-labeled CD86-Ig.

FIG. 55A-55C show colitis treatment with s.c.-inoculated COMBO COIN or i.p.-administered COMBO ISM NFs ameliorates severe colitis symptoms triggered by dual immune checkpoint blockade in B16F10 tumor-bearing mice by reducing immune cell infiltration into the colon epithelium. FIG. 55A shows Bodyweight change. All P values were analyzed using two-way ANOVA with Tukey's HSD multiple comparisons post-hoc test. FIG. 55B shows digital photographs of colons preserved at the study endpoint (23 days after the inoculation of B16F10 xenograft tumor/10 days after colitis treatment). FIG. 55C shows representative H&E-stained section of each colon preserved at the study endpoint after different treatments. The inserted label in each image represents the corresponding colon epithelial damage score and inflammatory cell infiltration score. All P values were analyzed using two-way ANOVA with Tukey's HSD multiple comparisons post-hoc test.

FIG. 56A-56C show colitis treatment with s.c.-inoculated COMBO COIN or i.p.-administered COMBO ISM NFs does not exhaust tumor-specific antigen-specific CD8⁺ T cells in the B16OVA tumor model. FIG. 56A shows in vivo treatment schedule. FIG. 56B shows FACS gating strategy and individual dot-scatter plots showing the populations of OVA-specific CD8⁺ T cells. FIG. 56C shows FACS gating strategy and individual dot-scatter plots showing TIM-3 expression in OVA-specific CD8⁺ T cells.

FIG. 57 shows colitis treatment with s.c.-inoculated COMBO COIN or i.p.-administered COMBO ISM NFs does not affect ICB cancer treatment in the MC38 tumor model. Bodyweight change after cancer and colitis treatment. All P values were analyzed using two-way ANOVA with Tukey's HSD multiple comparisons post-hoc test.

The drawing figures do not limit the present disclosure to the specific embodiments disclosed and described herein. The drawings are not necessarily to scale, emphasis instead being placed on clearly illustrating principles of certain embodiments of the present disclosure.

### DETAILED DESCRIPTION

The following detailed description references the accompanying drawings that illustrate various aspects of the present disclosure. The drawings and description are intended to describe aspects of the present disclosure in sufficient detail to enable those skilled in the art to practice the present disclosure. Other components can be utilized, and changes can be made without departing from the scope of the present disclosure. The following description is, therefore, not to be taken in a limiting sense.

Provided herein are compositions and methods of making a colon-specific immune niche (COIN). Further provided is a method of preventing, treating, or reducing the risk of a condition or disease of large intestinal tract tissue by administering an effective amount of a composition comprising a COIN. The present disclosure is partly based on the surprising determination that colon-specific immune niche (COIN) constructed from immunomodulator-functionalized nanofibers, colon epithelial cells, and a decellularized colon extracellular matrix (ECM) provided a highly suppressive immune microenvironment, was able to engage with autoimmune cells and provided intestinal immunotolerance, ameliorating inflammation in the large intestinal tract. The COIN disclosed herein engineered with immune suppressive programmed death-ligand 1 (PD-L1), cluster of differentiation 86 (CD86), transforming growth factor-beta 1 (TGF-β1) mimic peptide leflunomide (LEF), or any combination thereof can simultaneously anergize autoreactive T cells and polarize proinflammatory macrophages (M(s) through multiple immunosuppression pathways. Further, the bioengineered COIN could effectively reduce colitis symptoms by inhibiting autoreactive cytotoxic T cell activation and polarizing classically activated M1 Mφs into anti-inflammatory alternatively activated M2 Mφs, preventing immune cell infiltration into the colon lamina propria, and promoted epithelial damage recovery, in vivo. Additionally, COIN was identified to be able to prevent colitis-associated colorectal cancer and eliminate immune-related colitis triggered by immune checkpoint and cell signaling pathway inhibitors.

### 1. Terminology

For the purposes of promoting an understanding of the principles of the present disclosure, reference will now be made to preferred aspects and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

As used in the specification, articles “a” and “an” are used herein to refer to one or to more than one (i.e., at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result. The term “about” in association with a numerical value means that the numerical value can vary plus or minus by 5% or less of the numerical value.

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements or steps but not the exclusion of any other integer or step or group of integers or steps.

As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the disclosure states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder, or condition.

As used herein, “prevent” or “prevention” refers to eliminating or delaying the onset of a particular disease, disorder or physiological condition, or to the reduction of the degree of severity of a particular disease, disorder or physiological condition, relative to the time and/or degree of onset or severity in the absence of intervention.

The term “effective amount” or “therapeutically effective amount” refers to an amount sufficient to effect beneficial or desirable biological and/or clinical results.

As used herein, “individual”, “subject”, “host”, and “patient” can be used interchangeably herein and refer to any mammalian subject for whom diagnosis, treatment, prophylaxis or therapy is desired, for example, humans, pets, livestock, horses or other animals. As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. In some aspects, the subject can be a human.

As used herein “extracellular matrix” or “ECM” refers to three-dimensional network of proteins and other molecules that surround, support, and give structure to cells and tissues in the body. The extracellular matrix helps cells attach to, and communicate with, nearby cells, and plays an important role in cell growth, cell movement, and other cell functions. The extracellular matrix may include the interstitial matrix and the basement membrane. ECM is a complex mixture of structural and non-structural biomolecules, including, but not limited to, collagens, elastins, laminins, glycosaminoglycans, proteoglycans, antimicrobials, chemoattractants, cytokines, and growth factors. In mammals, ECM often comprises about 90% collagen, in its various forms. The composition and structure of ECM varies depending on the source of the tissue. For example, small intestine submucosa (SIS), urinary bladder matrix (UBM), liver stroma ECM, and dermal ECM each differ in their overall structure and composition due to the unique cellular-niche needed for each tissue.

As used herein “inflammatory bowel disease” or “IBD” is a chronic disorder of the gastrointestinal tract. The disease is characterized by inflammation of the small intestine and/or the large intestine (colon). Symptoms of IBD include abdominal pain, persistent diarrhea, and colorectal bleeding. The two main subcategories of IBD are ulcerative colitis and Crohn's disease. Both disorders cause inflammation of the digestive tract, but they differ as to the nature and location of the inflammatory reactions in the guts. Ulcerative colitis is restricted to the colon mucosa and may involve the entire colon or only parts thereof of, such as the rectum. In contrast, Crohn's disease may affect any part of the gastrointestinal tract, i.e., from mouth to anus, although it commonly affects the lower part of the small intestine (ileum). Further, in contrast to the inflammation caused by ulcerative colitis, the inflammation caused by Crohn's disease extends deep into the intestinal wall.

Ulcerative colitis is a chronic inflammatory disease characterized by diffuse mucosal inflammation of the colon. The disease is characterized by amongst other features bloody diarrhea, often with symptoms of rectal urgency and tenesmus. Ulcerative colitis can include diverticulitis, pouchitis, proctitis, mucositis, diversion colitis, ischemic colitis, infectious colitis, chemical colitis, radiation-induced colitis, microscopic colitis (including collagenous colitis and lymphocytic colitis), atypical colitis, pseudomembranous colitis, fulminant colitis, autistic enterocolitis, indeterminate colitis, Behcet's disease, jejunoiletis, ileitis, ileocolitis and granulomatous colitis.

As used herein “familial adenomatous polyposis” or “FAP” is an inherited disorder characterized by cancer of the large intestine (colon) and rectum. People with the classic type of familial adenomatous polyposis may begin to develop multiple noncancerous (benign) growths (polyps) in the colon as early as their teenage years.

As used herein “Hirschsprung's disease” or “congenital aganglionic megacolon” is a condition of the large intestine (colon) that causes difficulty passing stool. Hirschsprung's disease involves missing nerve cells in the muscles of part or all of the large intestine (colon). Present at birth, it causes difficulty passing stool.

As used herein “Proctitis” refers to the inflammation of the lining of the rectum. The rectum is a muscular tube that's connected to the end of your colon. Stool passes through the rectum on its way out of the body. Proctitis can cause rectal pain, diarrhea, bleeding and discharge, as well as the continuous feeling that you need to have a bowel movement. Proctitis symptoms can be short-lived, or they can become chronic.

As used herein “Celiac disease”, or “celiac sprue” or “gluten-sensitive enteropathy”, refers to an immune reaction to eating gluten, a protein found in wheat, barley and rye. In subjects with celiac disease, eating gluten triggers an immune response in the small intestine. Over time, this reaction damages the small intestine's lining and prevents it from absorbing some nutrients (malabsorption). The intestinal damage often causes diarrhea, fatigue, weight loss, bloating and anemia, and can lead to serious complications.

As used herein “gastrointestinal mucositis” refers to serious pathologies along the alimentary tract characterized by mucosal damage, ranging from mild inflammation to deep ulcerations and affecting one or more parts of the alimentary tract from the mouth to the anus. Mucositis usually occurs as an adverse effect of treatment of diseases such as cancer. As a result of cell death in reaction to chemotherapy, the mucosal lining of the alimentary tract becomes thin, may be inflamed, and ulcerated.

As used herein “colon cancer”, “colorectal cancer”, or “bowel cancer”, is a cancer from uncontrolled cell growth in the colon or rectum. Colon cancer can also originate in the colorectal epithelium. Colorectal cancer is thought to result from the clonal expansion of a single mutant cell in the epithelial lining of the colon or rectum.

As used herein “fistula” refers to an abnormal conduit or connection between bodily organs or vessels that do not usually connect. Fistulas or fistulae can form in many parts of the body. Anal fistula and rectal fistula are conditions in which tubes form between rectum and intestines, or other internal organs, or between rectum and the external skin adjacent to the anus. For example, fistulas situated high in the anus (high anal fistula) may connect with the urinary tract, and fistulas situated low in the anus (low anal fistula) may, in women, pass into the vagina. In addition to significant pain, rectal and anal fistulas commonly become infected and accumulate pus.

As used herein “nanofiber” or “NF” refers to a nanostructure in the form of continuous filaments or discrete elongated pieces of material typically characterized by at least one physical dimension less than about 1000 nm, less than about 500 nm, less than about 250 nm, less than about 150 nm, less than about 100 nm, less than about 50 nm, less than about 25 nm or even less than about 10 nm or 5 nm. In many cases, the region or characteristic dimension will be along the smallest axis of the structure.

As used herein “immune checkpoint molecule” is a molecule involved in immune checkpoints. These checkpoints are a normal part of the immune system and keep immune responses from being too strong. Non-limiting examples of immune checkpoint molecules include PD-1, PD-L1, TIM-3, LAG-3, CTLA-4, and CSF-1R and any combination thereof. The immune checkpoint receptors may be on tumor cells or immune cells such as T cells, monocytes, microglia, and macrophages, without limitation. In some aspects immune checkpoint molecule may be any immune checkpoint molecule known in the art, including but not limited to, CTLA-4, PDL1, PDL2, PD1, B7-H3, B7-H4, BTLA, HVEM, TIM3, GAL9, LAG3, CSF-1R, VISTA, KIR, 2B4, CD160, CGEN-15049, CHK1, CHK2, A2aR, CD28, CD86, CD69, CD48, CD113, CEACAM-1, Galectin-1, TIGIT, GPR56, CD48, GARP, PD1H, LAIR1, TIM1, TIM4 and the B-7 family of ligands. As disclosed herein, inhibitors of immune checkpoint molecules may be small chemical entities or polymers, antibodies, antibody fragments, single chain antibodies or other antibody constructs, including, but not limited to, bispecific antibodies and diabodies. Immune checkpoint inhibitors which may be used according to the disclosure include any that disrupt the inhibitory interaction of cytotoxic T cells. These include but are not limited to PD-1 antibody, PD-L1 antibody, CTLA4 antibody, LAG-3 antibody, TIM-3 antibody. The inhibitor need not be an antibody but can be a small molecule or other polymer. If the inhibitor is an antibody, it can be a polyclonal, monoclonal, fragment, single chain, or other antibody variant construct. Combinations of inhibitors for a single target immune checkpoint or different inhibitors for different immune checkpoints may be used. Illustrative examples of immune checkpoint inhibitors include CTLA-4 blocking antibodies (Ipilimumab (Yervoy), Tremelimumab (Imjuno)), PD-1 inhibitors (Pembrolizumab (Keytruda), Nivolumab (Opdivo), Cemiplimab (Libtayo), CT-011 (Pidilizumab), AMP224), PD-L1 inhibitors (Atezolizumab (tecentriq), Avelumab (Bavencio), Durvalumab (Imfinzi), BMS-936559), Lag3 inhibitors (Relatlimab), combination of Lag3 and PD1 inhibitor (PD-1 inhibitor nivolumab (Opdualag) OX40 inhibitor (MEDI6469), CD160 inhibitor (BY55). Non-limiting examples of inhibitors of CSF-1R include PLX3397, PLX486, RG7155, AMG820, ARRY-382, FPA008, IMC-CS4, JNJ-40346527, and MCS 110.

As used herein “immune-related adverse event” or “irAE” refers to diverse toxicities, side-effects or problems associated with cancer immunotherapy. Immunotherapy include therapy using immunotoxins, T-cell transfer, chimeric antigen receptors, antibodies, immune system modulators and immune checkpoint inhibitors, and/or other immunotherapies known to those of skill in the art. In some aspects, irAE can be a condition or disease of the gastrointestinal system, for e.g., colitis, diarrhea, or celiac disease.

As used herein “cancer” may be one or more neoplasm or cancer. The neoplasm may be malignant or benign, the cancer may be primary or metastatic; the neoplasm or cancer may be early stage or late stage. Non-limiting examples of neoplasms or cancers include acute lymphoblastic leukemia, acute myeloid leukemia, adrenocortical carcinoma, AIDS-related cancers, AIDS-related lymphoma, anal cancer, appendix cancer, astrocytoma (childhood cerebellar or cerebral), basal cell carcinoma, bile duct cancer, bladder cancer, bone cancer, brainstem glioma, brain tumors (cerebellar astrocytoma, cerebral astrocytoma/malignant glioma, ependymoma, medulloblastoma, supratentorial primitive neuroectodermal tumors, visual pathway and hypothalamic gliomas), breast cancer, bronchial adenomas/carcinoids, Burkitt lymphoma, carcinoid tumors (childhood, gastrointestinal), carcinoma of unknown primary, central nervous system lymphoma (primary), cerebellar astrocytoma, cerebral astrocytoma/malignant glioma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma in the Ewing family of tumors, extracranial germ cell tumor (childhood), extragonadal germ cell tumor, extrahepatic bile duct cancer, eye cancers (intraocular melanoma, retinoblastoma), gallbladder cancer, gastric (stomach) cancer, gastrointestinal carcinoid tumor, gastrointestinal stromal tumor, germ cell tumors (childhood extracranial, extragonadal, ovarian), gestational trophoblastic tumor, gliomas (adult, childhood brain stem, childhood cerebral astrocytoma, childhood visual pathway and hypothalamic), gastric carcinoid, hairy cell leukemia, head and neck cancer, hepatocellular (liver) cancer, Hodgkin lymphoma, hypopharyngeal cancer, hypothalamic and visual pathway glioma (childhood), intraocular melanoma, islet cell carcinoma, Kaposi sarcoma, kidney cancer (renal cell cancer), laryngeal cancer, leukemias (acute lymphoblastic, acute myeloid, chronic lymphocytic, chronic myelogenous, hairy cell), lip and oral cavity cancer, liver cancer (primary), lung cancers (non-small cell, small cell), lymphomas (AIDS-related, Burkitt, cutaneous T-cell, Hodgkin, non-Hodgkin, primary central nervous system), macroglobulinemia (Waldenström), malignant fibrous histiocytoma of bone/osteosarcoma, medulloblastoma (childhood), melanoma, intraocular melanoma, Merkel cell carcinoma, mesotheliomas (adult malignant, childhood), metastatic squamous neck cancer with occult primary, mouth cancer, multiple endocrine neoplasia syndrome (childhood), multiple myeloma/plasma cell neoplasm, mycosis fungoides, myelodysplastic syndromes, myelodysplastic/myeloproliferative diseases, myelogenous leukemia (chronic), myeloid leukemias (adult acute, childhood acute), multiple myeloma, myeloproliferative disorders (chronic), nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer (surface epithelial-stromal tumor), ovarian germ cell tumor, ovarian low malignant potential tumor, pancreatic cancer, pancreatic cancer (islet cell), paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors (childhood), pituitary adenoma, plasma cell neoplasia, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma (childhood), salivary gland cancer, sarcoma (Ewing family of tumors, Kaposi, soft tissue, uterine), Sezary syndrome, skin cancers (nonmelanoma, melanoma), skin carcinoma (Merkel cell), small cell lung cancer, small intestine cancer, soft tissue sarcoma, squamous cell carcinoma, squamous neck cancer with occult primary (metastatic), stomach cancer, supratentorial primitive neuroectodermal tumor (childhood), T-Cell lymphoma (cutaneous), testicular cancer, throat cancer, thymoma (childhood), thymoma and thymic carcinoma, thyroid cancer, thyroid cancer (childhood), transitional cell cancer of the renal pelvis and ureter, trophoblastic tumor (gestational), unknown primary site (adult, childhood), ureter and renal pelvis transitional cell cancer, urethral cancer, uterine cancer (endometrial), uterine sarcoma, vaginal cancer, visual pathway and hypothalamic glioma (childhood), vulvar cancer, and Wilms tumor (childhood).

As used herein “nanoparticle” or “ultrafine particle” is defined as a particle of matter that is between 1 and 100 nanometers (nm) in diameter. Nanoparticles are small particles, for e.g., clusters of metal or semiconductor atoms. The term is also applied for larger particles, up to 500 nm, or fibers and tubes that are less than 100 nm in only two directions. Nanoparticles can occur in a variety of shapes, including as nanospheres, nanorods, nanochains, nanostars, nanoflowers, nanoreefs, nanowhiskers, nanofibers, and nanoboxes. In certain aspects, the nanoparticles and/or nanoparticle cores contemplated herein have a generally polyhedral or spherical geometry. References to “diameter” of a nanoparticle or a nanoparticle core a generally taken to mean the longest dimension of the nanoparticle or nanoparticle core, respectively. As used herein, “corona” refers to a layer or coating, which may partially or completely cover the exposed surface of the nanoparticle core. The corona includes a plurality of ligands covalently attached to the core of the nanoparticle. Thus, the corona may be considered to be an organic layer that surrounds or partially surrounds the metallic core. In certain aspects, the corona provides and/or participates in passivating the core of the nanoparticle. Thus, in certain cases the corona may include a sufficiently complete coating layer substantially to stabilize the core. In certain cases, the corona facilitates solubility, such as water solubility, of the nanoparticles.

### II. Methods of Making a Colon-Specific Immune Niche

In some aspects, the present disclosure provides a method of making a colon-specific immune niche. In some aspects, the method can comprise combining diblock copolymer nanofibers (NFs), with colon epithelial (EPI) cells, and decellularized colon extracellular matrix (ECM). In some aspects of the method, the nanofibers can be further functionalized with an immunomodulator. In such aspects, the method can comprise combining diblock copolymer nanofibers (NFs) functionalized with an immunomodulator, with colon epithelial (EPI) cells, and decellularized colon extracellular matrix (ECM). The COIN fabricated using methods disclosed herein provide a colon-like tissue microenvironment, that can be used for treating a condition or disease associated with lower intestinal tract tissue.

### Nanofibers (NFs)

In some aspects, the present disclosure provides nanofibers for use in fabricating COIN. In some aspects, the nanofibers can be further functionalized with immunomodulators.

In some aspects, NFs disclosed herein can be engineered using a polymer including but not limited to potassium and calcium alginates, agar, gelatin, polylactic acid (PLA), poly(lactic-co-glycolic acid) (PGLA), polyethylene glycol (PEG), polyethylene oxide (PEO), acrylnitril-butadien-styrol (ABS), polyvinyl pyrrolidone (PVP), polyvinyl alcohol (PVA), polycarbonate (PC), nylon, acrylnitril-styrol-acrylester (ASA), silicone, polycaprolactone (PCL), poly(vinyl pyrrolidone) (PVP), poly (methacrylic acid) (PMAA), poly(2-hydroxyethyl) methacrylate (pHEMA), poly(acrylamide) (PA), poly(methacrylamide) (PMA), poly(2-hydroxyethyl) methacrylamide (PHEMAA), poly(methacryoylaminoalkyl) phosphorylcholines (PMAmPCs), poly(sulfopropyl) acrylates (PSPAs), poly (sulfopropyl) methacrylates (PSPMAs), poly(methacrylates) (PMAs), hydrophilic polyether, polysaccharide, hydrophilic polyurethane, polyhydroxyethylacrylate, polymethacrylate, dextran, natural gums, and homopolymers and copolymers of N-vinylpyrrolidone, N-vinyllactam, N-vinyl butyrolactam, N-vinyl caprolactam, other vinyl compounds having polar pendant groups, acrylate and methacrylate having hydrophilic esterifying groups, acrylamide, hydroxyethylacrylate, acrylic acid, methyl cellulose, ethyl cellulose, hydroxypropyl cellulose, carboxymethyl cellulose, hydroxypropyl methyl cellulose, hydroxyethyl methyl cellulose, hydroxyethyl cellulose, xanthan gum, guar gum, pectin, gum Arabic, locust bean gum, carrageenan gum, or copolymers thereof. Additionally, polymers of methacryloyloxyethyl phosphorylcholine (MPC), methacryloyloxypropyl phosphorylcholine, methacryloyloxybutyl phosphorylcholine, methacryloylaminoalkyl phosphorylcholine or similar phosphorylcholine containing polymers may be employed. As will be obvious to those skilled in the art, mixtures of the various polymeric materials may also be used.

In some aspects, the NFs disclosed herein can be engineered using a diblock copolymer. In such aspects, the first or second block can comprise poly(dimethylacrylamide)(PDMA), poly(ethylene glycol) (PEG), poly(PEG), poly(methyl oxazoline)(PMOX), poly(ethyl oxazoline), polysarcosine, poly(4-acryloylmorpholine), poly(glycerol monomethacrylate), poly(propylene sulfoxide), poly(2-(methylsulfinyl)ethyl acrylate)(PMSEA), poly(vinyl alcohol)(PVA), poly(glycidol), poly(thioglycidyl glycerol)(PTGG), poly(2-methacryloyloxyethyl phosphorylcholine)(PMPC), poly(vinyl pyrrolidone)(PVP), poly(N-(2-hydroxypropyl)methacrylamide)(PHPMA), poly(trimethylamine N-oxide), poly(lysine-methacrylamide), poly(lysine-acrylamide), poly(carboxybetaine), poly(sulfobetaine), Heparosan, and any combination thereof. In some aspects, the NFs disclosed herein can be engineered using a diblock copolymer wherein the first block comprises poly(2-(methacryloyloxy)ethyl phosphorylcholine (PMPC), and second block comprises poly(hydroxypropyl methacrylate) (PHPMA).

In some aspects, copolymers can be synthesized using any known method in the art, non-limiting examples of which include controlled radical polymerization such as reversible addition fragmentation chain transfer (RAFT), atom transfer radical polymerization (ATPR), stable free radical mediated polymerization (e.g. nitroxide mediated polymerization or verdazyl mediated polymerization) or iniferter polymerization, or (uncontrolled) free radical polymerization. In some aspects, the diblock copolymer can be synthesized through a two-step reversible addition-fragmentation chain transfer (RAFT) polymerization

In further aspects, NFs can comprise a functionalized polymer. In some aspects, the NFs can comprise a functionalized diblock copolymer functionalized. In some aspects, the diblock copolymer is functionalized with moieties including but not limited to hydroxyl, hydroxy, hydroxyalkyl, halogen, ═O (oxo), ═S (thioxo), cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycle, cycloalkylalkyl, heteroarylalkyl, arylalkyl, hydroxy, hydroxyalkyl, alkoxy, alkoxyalkyl, alkylene, aryloxy, phenoxy, benzyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, carboxylic acid (—COOH), ketone, amide, carbamate, acyl, a crosslinking agent, phenyl, naphthyl, pyridinyl, pyrrole, imidazolyl, pyrazolyl, indolyl, furyl, dibenzocyclooctyne (DBCO), OCT (cyclooctyne), MOFO (monofluorinated cyclooctyne), ALO (aryl-less octyne), DIFO (difluorocy-clooctyne), DIF02 (difluorocyclooctyne), DIF03 (difluorocyclooctyne), DIMAC (dimethoxyazacyclooctyne), DIBO (dibenzocyclooctyne), DIBAC (dibenzoazacyclooctyne), BARAC (biarylazacyclooctynone), BCN (bicyclononyne), Sondheimer diyne, TMDIBO (2,3,6,7-tetramethoxy-DIBO), S-DIBO (sulfonylated DIBO), COMBO (carboxymethyl-monobenzocyclooctyne), PYRROC (pyrrolocyclooctyne), or any combination thereof. In some aspects, a crosslinking agent can be N-hydroxysuccinimidyl ester (eg, including, but not limited to, disuccinimidyl ester, dithiobis(succinimidylpropionate), 3,3′-dithiobis(sulfosuccinimidylpropionate), suberate Disuccinimidyl Bis(sulfosuccinimidyl)suberate, Disuccinimidyl Tartarate, Disulfosuccinimidyl Tartarate, Bis[2-(succinimidyloxycarbonyl)ethyl]sulfone, Bis[2-(sulfosuccinimidooxycarbonyl)ethyl]sulfone, Ethylene Glycol Bis(succinimidyl Succinate), Ethylene Glycol Bis(sulfosuccinimidyl Succinate), Glutarate disuccinimidyl, N,N′-disuccinimidylcarbonate, N-succinimidyl 3-(2-pyridyldithio)propionate, succinimidyl 6-(3-[2-pyridithio]-propionamido)hexanoate, sulfosuccinimidyl 6-(3′-[2-pyridithio]-propionamido)hexanoate, succinimidyloxycarbonyl-a-methyl-a-(2-pyridithio)toluene, sulfosuccinimidyl-6-[a-methyl-a-(2-pyridyldithio)toluamido]hexanoate, succinimidyl 4-(N-maleimidomethyl)cyclohexane-1-carboxylate, sulfosuccinimidyl 4-(N-maleimidomethyl)cyclohexane-1-carboxylate, m-maleimidobenzoyl-N-hydroxysuccinimide ester, m-Maleimidobenzoyl-N-hydroxy-sulfosuccinimide, N-succinimidyl(4-iodoacetyl)aminobenzoate, sulfo-succinimidyl(4-iodoacetyl)aminobenzoate, succinimidyl-4-(p-maleimidophenyl)butyrate, sulfosuccinimidyl-4-(p-maleimidophenyl)butyrate, N-(y-maleimidobutyloxy)succinimide ester, N-(ymaleimidobutyloxy)sulfosuccinimide ester, succinimidyl hexanoate 6-((iodo acetyl)amino)hexanoate, succinimidyl hexanoate 6-((4-iodoacetyl)amino)hexanoyl)amino, succinimidyl 4-(((iodoacetyl)amino)methyl) cyclohexane-1-carboxylate, succinimidyl 6-((((4-iodoacetyl)amino)methyl)cyclohexane-1-carbonyl)amino)-hexanoate and p-nitrophenyl iodoacetate). In some aspects, crosslinking agent can be a photoactivatable crosslinking agent including, but are not limited to, N-((2-pyridyldithio)ethyl)-4-azidosalicylamide aryl azide, 4-azido-2,3,5,6-tetrafluorobenzoic acid, succinimidyl ester, 4-Azido-2,3,5,6-Tetrafluorobenzoic Acid, Benzophenone STP Maleimide Ester, 4-Benzoylbenzoic Acid Succinimidyl Ester, N-5azido-2-nitrobenzoyloxysuccinimide, N-hydroxysulfo succinimidyl-4-azidobenzoate, N-hydroxysuccinimidyl-4-azidosalicylic acid, and (4-[p-azidosalicylamido]butylamine).

In further aspects, the functional moiety can be conjugated to the NFs using a linker moiety. In some aspects, the linker moiety can comprise sulfhydryl, amino, alkenyl, alkynyl, azidyl, aldehyde, carboxyl, maleimidyl, succinimidyl, or hydroxylamino groups. In some aspects, the linker can comprise N-hydroxysuccinimidyl (NHS) or N-hydroxysulfosuccinimidyl (sulfo-NHS) ester. In some aspects, the linker and functional group can comprise or be NHS-sulfo-DBCO (dibenzocyclooctyne-sulfo-N-hydroxysuccinimidyl ester) or NHS-DBCO (diben-zocyclooctyne-N-hydroxysuccinimidyl ester), wherein the NHS and DBCO may optionally be linked via a linker or spacer group.

In some aspects, the NFs disclosed herein can comprise a weight-averaged length of about 1-50 μm. In some aspects, the NFs can comprise a weight-averaged length of about 1 μm, about 2 μm, about 3 μm, about 4 μm, about 5 μm, about 6 μm, about 7 μm, about 8 μm, about 9 μm, about 10 μm, about 11 μm, about 12 μm, about 13 μm, about 14 μm, about 15 μm, about 16 μm, about 17 μm, about 18 μm, about 19 μm, about 20 μm, about 21 μm, about 22 μm, about 23 μm, about 24 μm, about 25 μm, about 26 μm, about 27 μm, about 28 μm, about 29 μm, about 30 μm, about 31 μm, about 32 μm, about 33 μm, about 34 μm, about 35 μm, about 36 μm, about 37 μm, about 38 μm, about 39 μm, about 40 μm, about 41 μm, about 42 μm, about 43 μm, about 44 μm, about 45 μm, about 46 μm, about 47 μm, about 48 μm, about 49 μm, or about 50 μm. In some aspects, the NFs can comprise a weight-averaged length of about 3 μm.

In some aspects, the NFs disclosed herein can comprise a core weight-averaged radius of 1-50 nm. In some aspects, the NFs can comprise a core weight-averaged radius of about 1 nm, about 2 nm, about 3 nm, about 4 nm, about 5 nm, about 6 nm, about 7 nm, about 8 nm, about 9 nm, about 10 nm, about 11 nm, about 12 nm, about 13 nm, about 14 nm, about 15 nm, about 16 nm, about 17 nm, about 18 nm, about 19 nm, about 20 nm, about 21 nm, about 22 nm, about 23 nm, about 24 nm, about 25 nm, about 26 nm, about 27 nm, about 28 nm, about 29 nm, about 30 nm, about 31 nm, about 32 nm, about 33 nm, about 34 nm, about 35 nm, about 36 nm, about 37 nm, about 38 nm, about 39 nm, about 40 nm, about 41 nm, about 42 nm, about 43 nm, about 44 nm, about 45 nm, about 46 nm, about 47 nm, about 48 nm, about 49 nm, or about 50 nm. In some aspects, the NFs can comprise a core weight-averaged radius of about 16 nm.

In some aspects, the NFs disclosed herein can be functionalized with a carboxylic acid moiety. In some aspects, the NFs disclosed herein can be functionalized with a sulfo DBCO-amine moiety. In some aspects, the NFs disclosed herein can be functionalized with a carboxylic acid moiety and a sulfo DBCO-amine moiety. In some aspects, the NFs can comprise a diblock copolymer comprising PMPC and PHPMA. In some aspects, the diblock copolymer comprising PMPC and PHPMA can be functionalized with carboxylic acid. In further aspects, the diblock copolymer comprising PMPC and PHPMA can be functionalized with sulfo

DBCO-amine. In some aspects, the diblock copolymer comprising PMPC and PHPMA functionalized with carboxylic acid can be further functionalized with sulfo DBCO-amine. In some aspects, carboxylic acid-functionalized NFs can be functionalized with sulfo DBCO-amine using any know methods in the art, for e.g., an amine-NHS ester coupling reaction and click chemistry.

In further aspects, NFs disclosed herein can further comprise a label or a fluorescent moiety. In some aspects, the label or fluorescent moiety can be a dye such as azo dyes, triarylmethane dyes, anthraquinone dyes, and heterocyclic compounds. In some aspects, the label can be rhodamine.

In some aspects, the NFs can further comprise or be functionalized with a biologically active agent. In such aspects, the biologically active agent can include but are not limited to proteins, peptides, antibodies, antibody fragments, nucleic acid, DNA, RNA, and other known biologic substances, particularly those that have therapeutic use. In certain aspects, the agent can be a drug or therapeutic agent (for e.g., a small molecule) (for e.g., analgesic, growth factor, anti-inflammatory, signaling molecule, cytokine, antimicrobial (for e.g., antibacterial, antibiotic, antiviral, and/or antifungal), hemostatic agent (for e.g., blood clotting agent, factor, or protein), pain medications (for e.g., anesthetics), etc.), or an anti-cancer agent. In some aspects, the agent can enhance tissue regeneration, tissue growth, and wound healing for e.g., immunomodulators, or growth factors. In some aspects, the agent can treat or prevent infections (for e.g., antimicrobials such as antibacterials, antivirals and/or antifungals).

### Epithelial (EPI) Cells

In some aspects, the disclosure further encompasses epithelial cells isolated from an intestinal tissue. In some aspects, the intestinal tissue can be obtained from a resected intestinal tissue or an endoscopic biopsy tissue, In some aspects, the intestinal tissue can be derived from histologically normal duodenal, jejunal, and ileal endoscopic biopsies. In some aspects, the intestinal epithelial cells can be obtained from induced pluripotent stem cells. In some aspects, the intestinal epithelial cells can be mammalian intestinal epithelial cells. In some aspects, the intestinal epithelial cells can be human intestinal epithelial cells. In some aspects, the intestinal epithelial cells can be mouse intestinal epithelial cells. In some aspects, the intestinal epithelial cells can be cell line-derived cells. In some aspects, the intestinal epithelial cells can be colon epithelial cells.

The epithelial (EPI) cells disclosed herein, can be isolated using any known methods in the art. In some aspects, EPI cells can be syngeneic, allogenic, or autologous. In some aspects, EPI cells can be isolated using colon biopsy. In some aspects, EPI cells can be isolated using biomarker based assays from collected samples, for e.g., using flow cytometric isolation or cell sorting. In some aspects, EPI cells can be EPI cells from an established colon cell line (for e.g., HcoEpiC from ScienCell Research Laboratories isolated from human colonic tissue, Catalog #2950). In some aspects, I EPI cells can be further cultured using an epithelial cell medium and passaged. In some aspects, the method of isolation can comprise: washing a resected intestinal tissue or an endoscopic biopsy tissue and removing any associated muscle or mucosa layers; contacting the tissue with one or more extracellular-matrix degrading enzymes; removing an intestinal crypt from the supernatant resulting from the contacting step, and isolating the EPI cells.

In further aspects, the EPI cells disclosed herein can be isolated from the subject planning to or undergoing treatment. In some aspects, the EPI cells can be isolated from a donor for e.g., a human Leucocyte Antigen (HLA) compatible donor. In such aspects, EPI cells can be isolated from a donor individual or individuals who are HLA compatible. EPI cells can be isolated from a sample provided by the donor individual, or can be pooled samples from multiple donor individuals who are HLA compatible. In some aspects, the EPI cells disclosed herein encompasses an allogenic EPI cells. In such aspects, EPI cells can be isolated from a donor individual or multiple individuals. In such aspects, EPI cells can be isolated from a sample provided by the donor individual, or pooled samples from multiple donor individuals. In some aspects, the EPI cells can be fresh EPI cells. In some aspects, the EPI cells can be previously frozen EPI cells.

In further aspects, the EPI cells can be suspended in a solution such as buffered solution, physiological saline, Ringer's solution, phosphate solution, or Phosphate Buffered Saline (PBS). In some aspects, the colon EPI cells can be suspended in PBS. In some aspects, the colon EPI cells can be suspended in 1×PBS.

In some aspects, the EPI cells can be further modified, for e.g., genetically modified to alter expression of one or more specific genes, markers or peptides or to secrete or not secrete specific cytokines.

### Colon Extracellular Matrix (ECM)

Colon ECM disclosed herein can be isolated and processed using any known methods in the art. The colon for isolating the ECM or colon ECM can be isolated from syngeneic, allogenic, or autologous sources. In some aspects, colon ECM can be isolated from preserved donor colon (for e.g., from colonic disease-free deceased donor) followed by detergent-facilitated decellularization and sterilization. In some aspects, the colon for isolating the ECM or colon ECM disclosed herein can be isolated from the subject planning to or undergoing treatment. In some aspects, the colon for isolating the ECM or colon ECM can be isolated from a donor e.g., a HLA compatible donor. In such aspects, colon for isolating the ECM or colon ECM can be isolated from a donor individual or individuals who are HLA compatible. The colon for isolating the ECM or colon ECM can be isolated from a sample provided by the donor individual, or pooled samples from multiple donor individuals who are HLA compatible. In some aspects, the colon for isolating the ECM or colon ECM disclosed herein can encompass an allogenic colon or colon ECM. In such aspects, colon for isolating the ECM or colon ECM can be isolated from a donor individual or individuals. In such aspects, colon for isolating the ECM or colon ECM can be isolated from a sample provided by the donor individual, or pooled samples from multiple donor individuals. In some aspects, the colon or colon ECM can be fresh colon or colon ECM. In some aspects, the colon or colon ECM can be previously frozen colon or colon ECM.

In some aspects, the methods for preparing colon ECM can comprise harvesting the excess connective tissue, removing the residual waste from the colon, and optionally freezing the tissue. Colonic submucosa can be obtained by any suitable method, for example by manually isolating from the surrounding tissue.

In some aspects, the colonic tissue, such as the colonic submucosa, can be first delipidized using a combination of chloroform and lower alcohols, such as a C₁-C₄ alcohol, non-limiting examples of which can include methanol, ethanol, n-propanol, isopropanol, n-butanol, isobutanol, 2-butanol, or mixtures thereof. The tissue can be incubated in chloroform, or a mixture of chloroform and a C1-C4 alcohol, such as for e.g., chloroform:methanol, and, in one aspect, can be then rinsed in a lower alcohol, for e.g., a C 1-C4 alcohol, such as ethanol. The delipidized tissue can be then washed and incubated with a protease, such as trypsin, and then subsequently washed with a deoxycholic acid or salt thereof, such as sodium deoxycholate. Alternative proteases to trypsin include, for example, other proteases active at a neutral or near-neutral pH, such as, without limitation: chymotrypsin, thermolysin, pepsin, Arg-C, Lys-N, Asp-N, and Lys-C. The tissue can then be disinfected or sterilized, for e.g., with a peroxyacid, such as peracetic acid, thereby producing a colon ECM composition. In some aspects, the colon tissue cannot be crosslinked or dialyzed at any step, such that the ECM material is an intact colon ECM material.

In one aspect, the decellularized colonic extracellular matrix material can be further dried (for e.g., lyophilized (freeze-dried) or air dried). The colon ECM material can be optionally comminuted, for example prior to enzymatic digestion, or prior to or after drying. Dried colon ECM material produced, when comminuted can be comminuted by any effective method, including, but not limited to, tearing, milling, cutting, grinding, and shearing. The comminuted colon ECM can also be further processed into a powdered form by methods, for example and without limitation, such as grinding or milling in a frozen or freeze-dried state.

In some aspects, decellularized colon ECM can be isolated using through detergent-facilitated spin-decellularization. Briefly, freshly preserved colon can be washed at least three times with 1×PBS before being incubated in 1% sodium dodecyl sulfate with gentle shaking, followed by incubation with 1% Triton-X100. The acellular colons can then be washed at least six times with 1×PBS. The acellular colons can then be incubated in 1×PBS supplemented with 1× Anti-Anti to destroy the residual microbes. The decellularized colons can then be washed and lyophilized. Lyophilized colons can then be ball-milled into fine powders. The lyophilized colons can be stored at −80° C. for up to 6 months before being used.

In some aspects, decellularized colon ECM of the present disclosure can retain activity of at least a portion of its structural and non-structural biomolecules, including, but not limited to, collagens, elastins, laminins, glycosaminoglycans, proteoglycans, antimicrobials, chemoattractants, cytokines, and/or growth factors. In some aspects, the disclosed decellularized colon ECM can retain one or more of the immunomodulatory growth factors (for e.g., granulocyte colony-stimulating factor (GCSF), granulocyte-macrophage colony-stimulating factor (GM-CSF), platelet-derived growth factor (PDGF), pro-regulatory cytokines (for e.g., TGF-μ1), or any combination thereof.

In some aspects, the colon ECM can comprise a synthetic or artificial ECM. In such aspects, biomaterials, such as matrices, scaffolds, and culture substrates that can mimic or present key environment and/or regulatory signals of a natural colon ECM can be used. Said biomaterials can comprise natural, semi-synthetic and synthetic biomaterials, and/or mixtures thereof. A scaffold can provide a two-dimensional or three-dimensional network. Suitable synthetic materials for said scaffold can comprise polymers selected from porous solids, nanofibers, and hydrogels such as, for example, peptides including self-assembling peptides, hydrogels composed of polyethylene glycol phosphate, polyethylene glycol fumarate, polyacrylamide, polyhydroxyethyl methacrylate, polycellulose acetate, and/or copolymers thereof. In some aspects, ECM can be a synthetic hydrogel extracellular matrix. In some aspects, synthetic ECM can be obtained commercially, for e.g., Matrigel™ (BD Biosciences), and can comprise comprises laminin, entactin, and collagen IV.

In further aspects, the lyophilized and/or milled colon ECM can be suspended in a solution such as buffered solution, physiological saline, Ringer's solution, phosphate solution, or Phosphate Buffered Saline (PBS). In some aspects, the colon ECM can be suspended in PBS. In some aspects, the colon ECM can be suspended in 1×PBS.

### Immunomodulator

In some aspects, the NFs, EPI cells, colon ECM, or any combination thereof can be functionalized with an immunomodulator.

In some aspects, the immunomodulator can be a peptide, polypeptide, mimetic, antibody, small molecule, large molecule, or a chemical compound that prevent or treat large intestinal tract tissue pathogenesis. In some aspects, the disclosed immunomodulator can be a peptide, polypeptide, mimetic, antibody, small molecule, large molecule, or a chemical compound that can anergize autoreactive T cells, polarize proinflammatory macrophages, or any combination thereof.

In some aspects, the immunomodulator can be an inhibitor of immune checkpoint molecule. In some aspects, the immune checkpoint molecule can be an inhibitor of CTLA-4, PDL1, PDL2, PD1, B7-H3, B7-H4, BTLA, HVEM, TIM3, GAL9, LAG3, CSF-1R, VISTA, KIR, 2B4, CD160, CGEN-15049, CHK1, CHK2, A2aR, CD28, CD86, CD69, CD48, CD113, CEACAM-1, Galectin-1, TIGIT, GPR56, CD48, GARP, PD1H, LAIR1, TIM1, TIM4, and the B-7 family of ligands. In some aspects, the immunomodulator can be an inhibitor of CD40, CD3, IL-2, IL-6, IL-12, TNFα, and IFNγ, and/or CD20. In some aspects, the inhibitor can comprise vedolizumab (Entyvio), ustekinumab (Stelara), 38ethotrexate (Skyrizi), adalimumab (Humira), golimumab (Simponi), certolizumab (Cimzia), vedolizumab (Entyvio), and/or Infliximab (Remicade). In some aspects, the inhibitor disclosed herein can be an antibody, or a biologically active fragment thereof. In some aspects, antibody used herein can be an antibody derived from any non-human animals such as mice, rats, rabbits, hamsters, dogs, sheep, and monkeys, human antibody, and the like. Recombinant antibodies, non-limiting examples of which include antibody artificially modified to reduce antigenicity in humans or for other purposes, such as chimeric antibody and humanized antibody can also be used. Antibodies as disclosed herein, can also include modified antibodies obtained by conjugating antibodies with various molecules. The modified antibodies can include antibodies conjugated with various molecules such as cytotoxic agents or polyethylene glycol (PEG). Non-limiting examples of cytotoxic agents include radioisotopes, chemotherapeutic agents, cellular toxins, etc. Such modified antibodies can be obtained by chemically modifying the antibodies produced as above. Methods for modifying antibodies have already been established in this field of art. In some aspects, antibody used herein, can include small antibody fragments. Small antibody fragment can include antibody fragments obtained by removing a part of whole antibodies (e.g., whole IgG, etc.) and are not specifically limited so far as they retain antigen-binding ability. Antibody fragments of the present disclosure are not specifically limited so far as they form a part of whole antibodies. In some aspects, antibody fragments contain a heavy chain variable region (VH) or a light chain variable region (VL), or both VH and VL. Non-limiting examples of antibody fragments include, e.g., Fab, Fab′, F(ab′)2, Fv, scFv (single-chain Fv), etc. Such antibody fragments can be obtained by treating antibodies with an enzyme such as papain or pepsin to produce antibody fragments or by constructing genes encoding these antibody fragments and introducing them into an expression vector and then expressing them in a suitable host cell. In some aspects, diabody is a dimer consisting of two fragments, each having variable regions joined together via a linker. In some aspects, the small antibody fragment comprises diabodies. In some aspects, diabody-forming fragments can include those consisting of VL and VH, VL and VL, or VH and VH. In some aspects, diabody-forming fragments can be joined via a linker to form single-chain diabodies (sc(Fv)₂).

In some aspects, the immunomodulator can be a small molecule. Small molecules can comprise an organic or inorganic molecule that is not a polymer, that has a therapeutic activity, and has a molecular weight less than 1 kDa. In some aspects, a small peptide or nucleic acid analog can also be considered a small molecule. Small molecules can be derived synthetically, semi-synthetically (for e.g., from naturally occurring precursors), or biologically. Non-limiting examples of small molecules that can be used in the disclosure described herein include Tofacitinib, Upadacitinib, SHR0302, Peficitinib, PF-06651600, PF-06700841, Deucravacitinib, TD-1473, OST-122, Ozanimod, Etrasimod, Ontamalimab, Tetomilast, Apremilast, Mongersen, Vidofludimus calcium, ABX464, BT-11, prednisone, budesonide, mesalamine, or any combination thereof.

In some aspects, the immunomodulator can be large molecule biologic. Large molecules can comprise a polymeric protein having a molecular weight greater than 1 kDa. In some aspects, mutant proteins that can reduce pathogenesis or inflammation of the large intestinal tract tissue can be used as an immunomodulator.

In some aspects, the immunomodulator can be a chemical compound, non-limiting examples of which can include, leflunomide (LEF), azathioprine (Imuran), mercaptopurine (6-MP, Purinethol), methotrexate, cyclosporine, or any combination thereof. Other immunosuppressive drugs are also contemplated for use as immunomodulator in the disclosure described herein.

In some aspects, the immunomodulator can be a peptide, polypeptide or mimetic. In some aspects, the immunomodulator can be a peptide, polypeptide or mimetic of IL-10 and/or TGF-β1. In some aspects, the sequence of IL-10 of various species are available on the world wide web from the NCBI, including human, mouse, and rat. For example, the nucleotide sequence encoding a variant of human IL-10 is available at NCBI under Accession No. NM_000572.3 and corresponding amino acid sequence under Accession No. NP_000563.1. The nucleotide sequence encoding a human TGF-β1 is available at NCBI under Accession No. NM_000660.7 and corresponding amino acid sequence under Accession No. NP_000651.3. IL-10 and/or TGF-β1 may be isolated from naturally occurring sources (e.g., mammalian cells that naturally produce the protein), produced in eukaryotic or prokaryotic cells using recombinant expression technology, or chemically synthesized. Soluble, biologically active IL-10 and/or TGF-β1 may be prepared in purified form using methods known in the art.

In some aspects, the immunomodulator can be a HVEM (Herpesvirus entry mediator) or a BTLA (B and T lymphocyte attenuator) agonist. In such aspects, the immunomodulator can be a BTLA-HVEM agonist capable of increasing BTLA activity in a cell having BTLA on its surface, for e.g., a lymphocyte, a T cell, a CD4⁺ T cell, a T_{H}1 cell, a CD8⁺ T cell, a B cell, a plasma cell, a macrophage, or an NK cell. In some aspects, suitable agonists can include, but not limited to BTLA agonistic antibodies (e.g., monoclonal, polyclonal, single chain, and/or bispecific antibodies as well as Fab and F(ab)₂ fragments, variants and derivatives thereof), fragments and truncated forms of HVEM proteins, fusion proteins, and the like, such as proteins and polypeptides comprising or consisting essentially of an HVEM CRD1 domain or fragment thereof capable of binding to a BTLA Ig domain and increasing BTLA activity, and/or lacking a CRD2 and/or CRD3 domain, and small molecule chemicals.

In some aspects, the immunomodulator can be a Fas ligand. In some aspects, the Fas ligand can be a polypeptide, which is a Fas ligand or a fragment thereof, or a mimetic. In some aspects, amino acid sequence of Fas ligand of various species are available on the world wide web from the NCBI, including human, mouse, and rat. For example, the nucleotide sequence encoding a variant of human Fas ligand is available at NCBI under Accession No. NM_000639.3 and amino acid sequence under Accession No. AAC50071.1. Fas ligand may be isolated from naturally occurring sources (e.g., mammalian cells that naturally produce the protein), produced in eukaryotic or prokaryotic cells using recombinant expression technology, or chemically synthesized. Soluble, biologically active Fas ligand may be prepared in purified form using methods known in the art.

In some aspects, the immunomodulator can be FGL-1. In some aspects, the FGL-1 can be a polypeptide, or a fragment thereof, or a mimetic. In some aspects, the sequence of FGL-1 of various species are available on the world wide web from the NCBI, including human, mouse, and rat. For example, the nucleotide sequence encoding a variant of human FGL-1 is available at NCBI under Accession No. NM_004467.4 and amino acid sequence under Accession No. NP_004458.3. FGL-1 may be isolated from naturally occurring sources (e.g., mammalian cells that naturally produce the protein), produced in eukaryotic or prokaryotic cells using recombinant expression technology, or chemically synthesized. Soluble, biologically active FGL-1 may be prepared in purified form using methods known in the art.

In some aspects, the immunomodulator can be CD47. In some aspects, the CD47 can be a polypeptide, or a fragment thereof, or a mimetic. In some aspects, the sequence of CD47 of various species are available on the world wide web from the NCBI, including human, mouse, and rat. For example, the nucleotide sequence encoding a variant of human CD47 is available at NCBI under Accession No. NM_001777.4 and corresponding amino acid sequence under Accession No. NP_001768.1. CD47 may be isolated from naturally occurring sources (e.g., mammalian cells that naturally produce the protein), produced in eukaryotic or prokaryotic cells using recombinant expression technology, or chemically synthesized. Soluble, biologically active CD47 may be prepared in purified form using methods known in the art.

In some aspects, the immunomodulator can be Gal9. In some aspects, the Gal9 can be a polypeptide, or a fragment thereof, or a mimetic. In some aspects, the sequence of Gal9 of various species are available on the world wide web from the NCBI, including human, mouse, and rat. For example, the nucleotide sequence encoding a variant of human Gal9 is available at NCBI under Accession No. NM_001777.4 and corresponding amino acid sequence under Accession No. NP_001768.1. Gal9 may be isolated from naturally occurring sources (e.g., mammalian cells that naturally produce the protein), produced in eukaryotic or prokaryotic cells using recombinant expression technology, or chemically synthesized. Soluble, biologically active Gal9 may be prepared in purified form using methods known in the art.

In some aspects, the immunomodulator can be functionalized onto NFs. In some aspects, the NFs can be functionalized with an immunomodulator. In some aspects, the NFs can be functionalized with a nanoparticle encapsulating an immunomodulator.

In some aspects, the immunomodulator can be a PD-L1 antibody, a CD86 antibody, leflunomide (LEF), a TGF-β1 mimetic peptide, or any combination thereof. In some aspects, the NFs can comprise or can be functionalized with a PD-L1 antibody. In some aspects, the NFs can comprise or can be functionalized with a CD86 antibody. In some aspects, the NFs can comprise or can be functionalized with a leflunomide (LEF). In some aspects, the NFs can comprise or can be functionalized with a TGF-β1 mimetic peptide. In some aspects, the NFs can comprise or can be functionalized with a PD-L1 antibody, and a CD86 antibody. In some aspects, the NFs can comprise or can be functionalized with a PD-L1 antibody, a CD86 antibody, a leflunomide (LEF), and a TGF-β1 mimetic peptide.

In some aspects, the NFs can be functionalized with an immunomodulator by conjugating the immunomodulator to the NFs. In such aspects, the immunomodulator can be further modified, for e.g., functionalized with azide. Methods of preparing immunomodulator functionalized NFs non-limiting examples of which can include for e.g., strain-promoted azide-alkyne cycloaddition (SPAAC) of DBCO functionalized NFs with a corresponding azide-functionalized immunomodulator. In certain aspects, DBCO functionalized NFs can be further functionalized with an azide-functionalized PD-L1 antibody. In certain aspects, DBCO functionalized NFs can be further functionalized with an azide-functionalized CD86 antibody. In certain aspects, DBCO functionalized NFs can be further functionalized with an azide-functionalized PD-L1 antibody and an azide-functionalized CD86 antibody. In such aspects, the degree of functionalization of the immunomodulator can be about 1-50 μg per mg of DBCO-NFs. In some aspects, the degree of functionalization of immunomodulator can be about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 41 μg, about 42 μg, about 43 μg, about 44 μg, about 45 μg, about 46 μg, about 47 μg, about 48 μg, about 49 μg, or about 50 μg per mg of DBCO-NFs. In some aspects, the degree of functionalization of the immunomodulator can be about 1-20 μg per mg of DBCO-NFs. In some aspects, the degree of functionalization of the immunomodulator can be about 5 μg per mg of DBCO-NFs. In some aspects, the degree of functionalization of the immunomodulator can be about 10 μg per mg of DBCO-NFs.

In some aspects, the NFs can be functionalized with an immunomodulator by conjugating a nanoparticle encapsulating the immunomodulator to the NFs. In such aspects, the immunomodulator may be a chemical compound. In some aspects, the immunomodulator can be LEF.

In some aspects, the nanoparticle disclosed herein can be prepared using a suitable polymer including, but are not limited to, biodegradable α-hydroxy polyesters and biocompatible polyethers. In some aspects, exemplary polyesters include, for example, PLA, PLGA, PEG, PEO, PEGylated polymers and copolymers of lactide and glycolide (e.g., PEGylated PLA, PLGA, PEGylated PGA, PEGylated PLGA), and derivatives thereof. In other aspects, suitable polymers include, for example, polyanhydrides, poly(ortho ester) PEGylated poly(ortho ester), poly(caprolactone), PEGylated poly(caprolactone), polylysine, PEGylated polylysine, poly(ethylene inline), PEGylated poly(ethylene imine), poly(L-lactide-co-L-lysine), poly(serine ester), poly(4-hydroxy-L-proline ester), poly[a-(4-aminobutyl)-L-glycolic acid], and combinations and derivatives thereof.

In some aspects, nanoparticles can comprise a polymer matrix which may comprise one or more acrylic polymers. Exemplary acrylic polymers include, for example, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid polyacrylamide) copolymer, aminoalkyl methacrylate copolymer, glycidyl methacrylate copolymers, polycyanoacrylates, and combinations thereof. The matrix may include dextran, acylated dextran, chitosan (e.g., acetylated to various levels), poly(vinyl) alcohol (for example, hydrolyzed to various degrees), and/or alginate, for e.g., alginate complexed to bivalent cations such as a calcium alginate complex.

In some aspects, nanoparticles disclosed herein can include one, two, three or more biocompatible and/or biodegradable polymers. For example, a contemplated nanoparticle may include about 10 to about 99 weight percent of one or more block copolymers that include a biodegradable polymer and polyethylene glycol, and about 0 to about 50 weight percent of a biodegradable homopolymer. Exemplary nanoparticles may include about 40 to 90 weight percent PLGA-PEG copolymer and about 33% weight percent PLGA. In further aspects, the exemplary nanoparticle can comprise PLGA-PEG copolymer further functionalized with azide, for e.g., PLGA-PEG-N3. In some aspects, the nanoparticles disclosed herein can comprise about 33% weight percent PLGA-PEG copolymer, about 33% weight percent PLGA, and about 33% PLGA-PEG-N3.

In some aspects, disclosed nanoparticles can comprise about 1 weight % to 25 weight % of the immunomodulator. In some aspects, the nanoparticle can comprise about 1 weight %, about 2 weight %, about 3 weight %, about 4 weight %, about 5 weight %, about 6 weight %, about 7 weight %, about 8 weight %, about 9 weight %, about 10 weight %, about 11 weight %, about 12 weight %, about 13 weight %, about 14 weight %, about 15 weight %, about 16 weight %, about 17 weight %, about 18 weight %, about 19 weight %, about 20 weight %, about 21 weight %, about 22 weight %, about 23 weight %, about 24 weight %, or about 25 weight % of the immunomodulator. In some aspects, the nanoparticles can comprise about 10 weight % of immunomodulator. In some aspects, the nanoparticles can comprise about 8 weight % of immunomodulator. In some aspects, the nanoparticles can comprise about 10 weight % of LEF. In some aspects, the nanoparticles can comprise about 8 weight % of LEF.

In some aspects, the nanoparticle can further comprise other therapeutic agents such as, but are not limited to, chemotherapeutic agents (for e.g., anti-cancer agents), diagnostic agents (for e.g., contrast agents, radionuclides, and fluorescent, luminescent, and magnetic moieties) or imaging agents.

The nanoparticles disclosed herein can be prepared using any suitable methods available in the art. Non-limiting examples of methods that may be used for preparing nanoparticle disclosed herein can include emulsion-solvent evaporation, emulsification-evaporation, nanoprecipitation, or solvent displacement, solvent diffusion, and phase-inversion. In some aspects, nanoparticles disclosed herein can be prepared using nanoprecipitation.

Methods of preparing NFs functionalized with a nanoparticle encapsulating an immunomodulator can include for e.g., stain-promoted 1,3-dipolar cycloaddition of DBCO functionalized NFs with corresponding azide functionalized nanoparticles. In some aspects, DBCO functionalized NFs is functionalized with a nanoparticle encapsulating LEF. In such aspects, the degree of functionalization of the nanoparticle encapsulating an immunomodulator can be about 1-400 μg per mg of DBCO-NFs. In some aspects, the degree of functionalization of nanoparticle encapsulating an immunomodulator is about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 25 μg, about 50 μg, about 75 μg, about 100 μg, about 120 μg, about 150 μg, about 175 μg, about 200 μg, about 225 μg, about 250 μg, about 300 μg, about 325 μg, about 350 μg, about 375 μg, or about 400 μg per mg of DBCO-NFs. In some aspects, the degree of functionalization of the nanoparticle encapsulating an immunomodulator can be about 200 μg per mg of DBCO-NFs. In some aspects, the degree of functionalization of the nanoparticle encapsulating an immunomodulator can be about 120 μg per mg of DBCO-NFs.

In some aspects, the NFs can be functionalized with an immunomodulator by physical adsorption onto the COOH-functionalized NFs (COOH-NFs). In such aspects, the immunomodulator can be a peptide, or a mimetic peptide. In certain aspects, COOH-functionalized NFs can be further functionalized with an TGF-β1 mimetic peptide. In some aspects, the peptide or mimetic peptide can be dissolved in buffer solution (for e.g., PBS) and can be incubated to allow oligomerization. The solution can then be added to NF solution for functionalizing the NFs with the peptide or mimetic peptide. In such aspects, the loading of the immunomodulator can be about 1-100 μg peptide or mimetic peptide per milligram of COOH-NFs. In some aspects, the loading of immunomodulator can be about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 41 μg, about 42 μg, about 43 μg, about 44 μg, about 45 μg, about 46 μg, about 47 μg, about 48 μg, about 49 μg, about 50 μg per, about 60 μg mg, about 70 μg, about 80 μg, about 90 μg, or about 100 μg per mg of COOH-NFs. In some aspects, the loading of the immunomodulator can be about 50 μg per mg of COOH-NFs.

In some aspects, the functionalized NFs can be further processed, such as sterilization (for e.g., by X-ray irradiation, or chemical sterilization).

In some aspects, the immunomodulator can be functionalized onto the EPI cells disclosed herein. In such aspects, the EPI cells can be genetically modified to express an immunomodulator disclosed herein such as a peptide, polypeptide, mimetic, antibody, small molecule, large molecule, or a chemical compound that prevent or treat large intestinal tract tissue pathogenesis. In such aspects, a polynucleotide encoding the immunomodulator, or enzymes/proteins involved in the production of the immunomodulator, can be constructed, introduced into an expression vector, and then can be expressed in suitable EPI cells. Standard molecular biology techniques can be used to prepare the recombinant expression vector, transfect the EPI cells, select for transformants, and culture the transformed EPI cells.

In some aspects, the expression vectors can include, for example, transposons and other site-specific mobile elements, viral vectors, e.g., adenovirus, adeno-associated virus (AAV), poxvirus, papillomavirus, lentivirus, herpesvirus, foamivirus and retrovirus vectors, and including pseudotyped viruses, liposomes and other lipid-containing complexes, and other macromolecular complexes capable of mediating delivery of a polynucleotide to a host cell, e.g., DNA coated gold particles, polymer-DNA complexes, liposome-DNA complexes, liposome-polymer-DNA complexes, virus-polymer-DNA complexes, e.g., adenovirus-polylysine-DNA complexes, and antibody-DNA complexes.

In some aspects, the expression vector can include a promoter necessary for expression in EPI cells, for example, an SV40 promoter, MMLV-LTR promoter, EF1a promoter, or CMV promoter. In addition to the nucleic acid sequence encoding the immunomodulator, or enzymes/proteins involved in the production of the immunomodulator, the recombinant expression vector may carry additional sequence, such as sequences that regulate replication of the vector in host cells (e.g., origins of replication) and selectable marker genes. The selectable marker gene facilitates selection of host cells into which the vector has been introduced. For example, typically the selectable marker gene confers resistance to drugs, such as G418, hygromycin, or methotrexate, on a cell into which the vector has been introduced. Examples of vectors with selectable markers include pMAM, pDR2, pBK-RSV, pBK-CMV, pOPRSV, and pOP13.

In some aspects, the EPI cells can be chemically conjugated to one or more immunomodulator. In such aspects, the one or more immunomodulator can comprise one or more affinity tags. In some aspects, the affinity tag can be a His tag, an Avi-tag, a hemagglutinin (HA) tag, a FLAG tag, a Myc tag, a GST tag, a MBP tag, a chitin binding protein tag, a calmodulin tag, a V5 tag, a streptavidin binding tag, a green fluorescent protein (GFP), YFP, RFP, CFP, mCherry, tdTomato, SUMO tag, Ubiquitin tag, or any combination thereof.

In some aspects, the immunomodulator can be a PD-L1 antibody, a CD86 antibody, leflunomide (LEF), a TGF-β1 mimetic peptide, or any combination thereof. In some aspects, the EPI cells can express or be functionalized with a PD-L1 antibody. In some aspects, the EPI cells can express or can be functionalized with a CD86 antibody. In some aspects, the EPI cells can express or can be functionalized with a leflunomide (LEF). In some aspects, the EPI cells can express or can be functionalized with a TGF-β1 mimetic peptide. In some aspects, the EPI cells can express or can be functionalized with a PD-L1 antibody, and a CD86 antibody. In some aspects, the EPI cells can express or can be functionalized with a PD-L1 antibody, a CD86 antibody, a leflunomide (LEF), and a TGF-β1 mimetic peptide.

In some aspects, the loading of immunomodulator can be about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 41 μg, about 42 μg, about 43 μg, about 44 μg, about 45 μg, about 46 μg, about 47 μg, about 48 μg, about 49 μg, about 50 μg per, about 60 μg mg, about 70 μg, about 80 μg, about 90 μg, or about 100 μg per about 1×10⁶ cells of EPI.

In some aspects, the immunomodulator can be functionalized onto ECM disclosed herein. In some aspects, the ECM can be functionalized with a nanoparticle encapsulating an immunomodulator. In such aspects, the ECM can be functionalized with an immunomodulator disclosed herein, such as a peptide, polypeptide, mimetic, antibody, small molecule, large molecule, or a chemical compound that can prevent or treat large intestinal tract tissue pathogenesis.

In some aspects, the ECM can be functionalized with an immunomodulator by using an immunomodulator functionalized with a reactive chemical group complimentary to the reactive chemical group functionalized onto extracellular matrix. In such aspects, complementary reactive chemical group can be an azide (—N₃), an alkyne, a nitrone, an isocyanide, a 47ethotrexate or a tetrazine. In some aspects, the alkyne is an aliphatic alkyne or a cyclooctyne. In some aspects, cyclooctyne can be dibenzocyclooctyne (DBCO), difluorobenzocyclooctyne (DIFBO), biarylazacyclooctynone (BARAC), dibenzocyclooctyne (DIBO), difluorinated cyclooctyne (DIFO), monofluorinated cyclooctyne (MOFO), dimethoxyazacyclooctyne (DIMAC) or aryl-less octyne (ALO).

In some aspects, non-limiting examples of methods for functionalizing ECM can include crosslinking chemistry utilizing the native amino acid residues within the biomaterials, such as lysine residues, for e.g., EDC/HNS crosslinking chemistry, and chemoselective click chemistry, for e.g., copper-catalyzed or copper-free click chemistry.

In some aspects, the ECM can be functionalized with an immunomodulator by conjugating a nanoparticle encapsulating the immunomodulator to the ECM. Nanoparticles can be prepared using methods disclosed herein. In such aspects, nanoparticles can be functionalized with a group that can conjugate or bind to the ECM. In some aspects, non-limiting examples of functional groups can include —NH, —NH₂, —COOH, or other functional groups disclosed herein.

In some aspects, the immunomodulator can be a PD-L1 antibody, a CD86 antibody, leflunomide (LEF), a TGF-β1 mimetic peptide, or any combination thereof. In some aspects, the ECM can be functionalized with a PD-L1 antibody. In some aspects, the ECM can be functionalized with a CD86 antibody. In some aspects, the ECM can be functionalized with a leflunomide (LEF). In some aspects, the ECM can be functionalized with a TGF-β1 mimetic peptide. In some aspects, the ECM can be functionalized with a PD-L1 antibody, and a CD86 antibody. In some aspects, the ECM can be functionalized with a PD-L1 antibody, a CD86 antibody, a leflunomide (LEF), and a TGF-β1 mimetic peptide.

In some aspects, the loading of immunomodulator can be about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 41 μg, about 42 μg, about 43 μg, about 44 μg, about 45 μg, about 46 μg, about 47 μg, about 48 μg, about 49 μg, about 50 μg per, about 60 μg mg, about 70 μg, about 80 μg, about 90 μg, or about 100 μg per about 1 mg of the ECM.

### Colon-Specific Immune Niche

In some aspects, the method of making a colon-specific immune niche (COIN) can comprise combining NFs, with EPI cells, and ECM to form COIN. In some aspects, the method of making COIN can comprise combining NFs functionalized with an immunomodulator, with colon EPI cells, and decellularized colon ECM to form COIN. In some aspects, the method of combining can comprise gentle mixing of the NFs, ECM and EPI cells. In some aspects, the method of combining can comprise gentle mixing of the NFs functionalized with an immunomodulator molecule, milled colon ECM and colon EPI cells.

In some aspects, NFs functionalized with an immunomodulator can be combined with colon EPI cells, and decellularized colon ECM at a concentration of about 0.001 mg to about 100 mg. In some aspects, NFs functionalized with an immunomodulator can be present in the colon-specific immune niche at a concentration of about 0.001 mg to about 10 mg, about 0.01 mg to about 10 mg, or about 0.01 mg to about 1 mg. In some aspects, NFs functionalized with an immunomodulator can be present in the in the colon-specific immune niche at a concentration of about 0.001 mg, about 0.01 mg, about 0.1 mg, about 0.5 mg, about 1 mg, about 2 mg, about 3 mg, about 4 mg, about 5 mg, about 6 mg, about 7 mg, about 8 mg, about 9 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, 80 mg, about 85 mg, about 90 mg, about 95 mg, or about 100 mg. In some aspects, colon-specific immune niche can comprise one or more NFs functionalized with an immunomodulator each of which can be at a concentration of about 0.01 mg. In some aspects, colon-specific immune niche can comprise one or more NFs functionalized with an immunomodulator each of which can be at a concentration of about 1 mg.

In some aspects, decellularized colon ECM can be combined with NFs functionalized with an immunomodulator, and colon EPI cells at a concentration at about 0.005 mg to about 50 mg. In some aspects, decellularized colon ECM can be present in the colon-specific immune niche at a concentration of about 0.005 mg to about 50 mg, about 0.05 mg to about 10 mg, or about 0.5 mg to about 1 mg. In some aspects, decellularized colon ECM can be present in the in the colon-specific immune niche at a concentration of about 0.005 mg, about 0.05 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 5 mg, about 10 mg, about 15 mg, about 20 mg, about 25 mg, about 30 mg, about 35 mg, about 40 mg, about 45 mg, or about 50 mg. In some aspects, colon-specific immune niche can comprise decellularized colon ECM at a concentration of about 0.5 mg.

In some aspects, the colon EPI cells can be combined with decellularized colon ECM and NFs functionalized with an immunomodulator at a concentration of about 1×10² to about 1×10¹⁰ cells. In some aspects, the colon EPI cells can be present in the colon-specific immune niche at a concentration of about 1×10² cells, about 1×10³ cells, about 1×10⁴ cells, about 1×10⁵ cells, about 1×10⁶ cells, about 1×10⁷ cells, about 1×10⁸ cells, about 1×10⁹ cells, or about 1×10¹⁰ cells. In some aspects, the colon EPI cells can be present in the colon-specific immune niche at a concentration of about 1×10⁴ cells. In some aspects, the colon EPI cells can be present in the colon-specific immune niche at a concentration of about 1×10⁶ cells.

In some aspects, a colon-specific immune niche can be prepared by gently mixing about 1 mg of one or more NFs functionalized with an immunomodulator molecule with about 0.5 mg of decellularized colon ECM, and colon EPI cells at about 1×10⁶ cells. In some aspects, a colon-specific immune niche can be made by gently mixing of about 1 mg of one or more NFs functionalized with an immunomodulator molecule with about 0.5 mg of decellularized colon ECM suspended in PBS, and colon EPI cells at about 1×10⁶ cells suspended in PBS. In some aspects, a colon-specific immune niche can be made by gently mixing about 1 mg of one or more of NFs functionalized with an immunomodulator molecule with about 0.5 mg of decellularized colon ECM, and colon EPI cells at about 1×10⁶ cells, and suspending the mixture in PBS.

In some aspects, the colon-specific immune niche of the present disclosure can comprise about 1 mg of NFs functionalized with PD-L1 antibody, about 0.5 mg of decellularized colon ECM, and colon EPI cells at about 1×10⁶ cells. In some aspects, the colon-specific immune niche can comprise about 1 mg of NFs functionalized with CD86 antibody, about 0.5 mg of decellularized colon ECM, and colon EPI cells at about 1×10⁶ cells. In some aspects, the colon-specific immune niche can comprise about 1 mg of NFs functionalized with LEF, about 0.5 mg of decellularized colon ECM, and colon EPI cells at about 1×10⁶ cells. In some aspects, the colon-specific immune niche can comprise about 1 mg of NFs functionalized with TGF-β1 mimetic peptide, about 0.5 mg of decellularized colon ECM, and colon EPI cells at about 1×10⁶ cells. In some aspects, the colon-specific immune niche can comprise about 1 mg of NFs functionalized with PD-L1 antibody, about 1 mg of NFs functionalized with CD86 antibody, about 0.5 mg of decellularized colon ECM, and colon EPI cells at about 1×10⁶ cells. In some aspects, the colon-specific immune niche can comprise about 1 mg of NFs functionalized with PD-L1 antibody, about 1 mg of NFs functionalized with CD86 antibody, about 1 mg of NFs functionalized with LEF, about 0.5 mg of decellularized colon ECM, and colon EPI cells at about 1×10⁶ cells. In some aspects, the colon-specific immune niche can comprise about 1 mg of NFs functionalized with PD-L1 antibody, about 1 mg of NFs functionalized with CD86 antibody, about 1 mg of NFs functionalized with LEF, about 1 mg of NFs functionalized with TGF-β1 mimetic peptide, about 0.5 mg of decellularized colon ECM, and colon EPI cells at about 1×10⁶ cells.

### III. Compositions

In some aspects, the present disclosure provides a composition comprising colon-specific immune niche (COIN). The composition of the present disclosure can comprise NFs, colon EPI cells, and ECM. The composition of the present disclosure can comprise NFs conjugated with an immunomodulator molecule, colon EPI cells, and decellularized colon ECM.

In some aspects, the composition can be formulated by gentle mixing of NFs functionalized with an immunomodulator molecule, milled colon ECM and colon EPI cells, disclosed herein. In some aspects, the composition can comprise NFs functionalized with an immunomodulator molecule at a concentration of about 0.1 mg to about 100 mg. In some aspects, the composition can comprise NFs functionalized with an immunomodulator molecule at a concentration of about 1 mg. In some aspects, the composition can comprise decellularized colon ECM at a concentration at about 0.05 mg to about 5 mg. In some aspects, the composition can comprise decellularized colon ECM at a concentration at about 0.5 mg. In some aspects, the composition can comprise colon EPI cells at a concentration of about 1×10² to about 1×10¹⁰ cells. In some aspects, the composition can comprise colon EPI cells at a concentration of about 1×10⁶ cells. In some aspects, the composition comprising a colon-specific immune niche can be formulated by gently mixing about 1 mg of NFs functionalized with an immunomodulator molecule with 0.5 mg of decellularized colon ECM, and colon EPI cells at about 1×10⁶ cells. In some aspects, the composition comprising a colon-specific immune niche can be formulated by gently mixing about 1 mg of NFs functionalized with an immunomodulator molecule with about 0.5 mg of decellularized colon ECM suspended in PBS, and colon EPI cells at about 1×10⁶ cells suspended in PBS. In some aspects, a colon-specific immune niche can be formulated into a composition by gently mixing about 1 mg of NFs functionalized with an immunomodulator molecule with about 0.5 mg of decellularized colon ECM, and colon EPI cells at about 1×10⁶ cells, and suspending the mixture in PBS.

In some aspects, the composition of the present disclosure can comprise about 1 mg of NFs functionalized with PD-L1 antibody, about 0.5 mg of decellularized colon ECM, and colon EPI cells at about 1×10⁶ cells. In some aspects, the composition can comprise about 1 mg of NFs functionalized with CD86 antibody, about 0.5 mg of decellularized colon ECM, and colon EPI cells at about 1×10⁶ cells. In some aspects, the composition can comprise about 1 mg of NFs functionalized with LEF, about 0.5 mg of decellularized colon ECM, and colon EPI cells at about 1×10⁶ cells. In some aspects, the composition can comprise about 1 mg of NFs functionalized with TGF-β1 mimetic peptide, about 0.5 mg of decellularized colon ECM, and colon EPI cells at about 1×10⁶ cells. In some aspects, the composition can comprise about 1 mg of NFs functionalized with PD-L1 antibody, about 1 mg of NFs functionalized with CD86 antibody, about 0.5 mg of decellularized colon ECM, and colon EPI cells at about 1×10⁶ cells. In some aspects, the composition can comprise about 1 mg of NFs functionalized with PD-L1 antibody, about 1 mg of NFs functionalized with CD86 antibody, about 1 mg of NFs functionalized with LEF, about 0.5 mg of decellularized colon ECM, and colon EPI cells at about 1×10⁶ cells. In some aspects, the composition can comprise about 1 mg of NFs functionalized with PD-L1 antibody, about 1 mg of NFs functionalized with CD86 antibody, about 1 mg of NFs functionalized with LEF, about 1 mg of NFs functionalized with TGF-β1 mimetic peptide, about 0.5 mg of decellularized colon ECM, and colon EPI cells at about 1×10⁶ cells.

In some aspects, the disclosed composition can be a pharmaceutical composition. In such aspects, the composition can further comprise one or more pharmaceutically acceptable diluent, excipient, and/or carrier. Pharmaceutically acceptable diluents, carriers, and excipients can include, but are not limited to, physiological saline, Ringer's solution, phosphate solution or buffer, buffered saline, and other carriers known in the art. Pharmaceutically acceptable carriers include any and all solvents, adjuvants, dispersion media, coatings, surfactants, antioxidants, preservatives (e.g., antibacterial agents, antifungal agents), isotonic agents, absorption delaying agents, salts, drugs, drug stabilizers, gels, binders, excipients, disintegration agents, lubricants, sweetening agents, flavoring agents, dyes, colorants, other medicinal or pharmaceutical agents, wetting agents, emulsifying agents, solution promoters, solubilizers, antifoaming agents, and such like materials and any combinations thereof, as would be known to one of ordinary skill in the art. Examples, without limitation, of excipients include calcium carbonate, calcium phosphate, various sugars and types of starch, cellulose derivatives, gelatin, vegetable oils and polyethylene glycols. Techniques for formulation and administration of drugs may also be found for example in Remington's Pharma. Sci. 18^{th} ed. 1990. Except insofar as any conventional carrier is incompatible with the active ingredient, its use in the therapeutic or pharmaceutical compositions is contemplated.

In certain aspects, compositions described herein may be an aqueous suspension comprising one or more polymers as suspending agents. In some aspects, polymers that may comprise pharmaceutical compositions described herein include: water-soluble polymers such as cellulosic polymers, for e.g., hydroxypropyl methylcellulose; water-insoluble polymers such as cross-linked carboxyl-containing polymers; mucoadhesive polymers, selected from, for example, carboxymethylcellulose, carbomer (acrylic acid polymer), poly(methylmethacrylate), polyacrylamide, polycarbophil, acrylic acid/butyl acrylate copolymer, sodium alginate, and dextran; or a combination thereof. In some aspects, compositions disclosed herein may comprise at least 5%, at least 10%, at least 20%, at least 25%, at least 30%, at least 35%, at least 40%, at least 45%, or at least 50% total amount of polymers as suspending agent(s) by total weight of the composition. In some aspects, compositions disclosed herein may comprise about 5% to about 99%, about 10%, about 95%, or about 15% to about 90% total amount of polymers as suspending agent(s) by total weight of the composition.

In certain aspects, compositions disclosed herein may comprise a viscous formulation. In some aspects, viscosity of composition herein may be increased by the addition of one or more gelling or thickening agents. In some aspects, compositions disclosed herein may comprise one or more gelling or thickening agents in an amount to provide a sufficiently viscous formulation to remain on treated tissue. In some aspects, compositions disclosed herein may comprise at least 5%, at least 10%, at least 20%, at least 25%, at least 30%, at least 35%, at least 40%, at least 45%, or at least 50% total amount of gelling or thickening agent(s) by total weight of the composition. In some aspects, pharmaceutical compositions disclosed herein may comprise about 5% to about 99%, about 10%, about 95%, or about 15% to about 90% total amount of gelling or thickening agent(s) by total weight of the composition. In some aspects, suitable thickening agents for use herein can be hydroxypropyl methylcellulose, hydroxyethyl cellulose, polyvinylpyrrolidone, carboxymethyl cellulose, polyvinyl alcohol, sodium chondroitin sulfate, sodium hyaluronate. In other aspects, viscosity enhancing agents can be acacia (gum 52ethot), agar, aluminum magnesium silicate, sodium alginate, sodium stearate, bladderwrack, bentonite, carbomer, carrageenan, Carbopol, xanthan, cellulose, microcrystalline cellulose (MCC), 52ethotrex, chitin, carboxymethylated chitosan, chondrus, dextrose, furcellaran, gelatin, Ghatti gum, guar gum, hectorite, lactose, sucrose, maltodextrin, mannitol, sorbitol, honey, maize starch, wheat starch, rice starch, potato starch, gelatin, sterculia gum, xanthum gum, gum tragacanth, ethyl cellulose, ethylhydroxyethyl cellulose, ethylmethyl cellulose, methyl cellulose, hydroxyethyl cellulose, hydroxyethylmethyl cellulose, hydroxypropyl cellulose, poly(hydroxyethyl methacrylate), oxypolygelatin, pectin, polygeline, povidone, propylene carbonate, methyl vinyl ether/maleic anhydride copolymer (PVM/MA), poly(methoxyethyl methacrylate), poly(methoxyethoxyethyl methacrylate), hydroxypropyl cellulose, hydroxypropylmethyl-cellulose (HPMC), sodium carboxymethyl-cellulose (CMC), silicon dioxide, polyvinylpyrrolidone (PVP: povidone), Splenda® (dextrose, maltodextrin and sucralose), or any combination thereof.

In certain aspects, compositions disclosed herein may comprise additional agents or additives selected from a group including surface-active agents, detergents, solvents, acidifying agents, alkalizing agents, buffering agents, tonicity modifying agents, ionic additives effective to increase the ionic strength of the solution, antimicrobial agents, antibiotic agents, antifungal agents, antioxidants, preservatives, electrolytes, antifoaming agents, oils, stabilizers, enhancing agents, and the like. In some aspects, compositions disclosed herein may comprise at least 5%, at least 10%, at least 20%, at least 25%, at least 30%, at least 35%, at least 40%, at least 45%, or at least 50% total amount of one or more agents by total weight of the composition. In some aspects, pharmaceutical compositions disclosed herein may comprise about 5% to about 99%, about 10%, about 95%, or about 15% to about 90% total amount of one or more agents by total weight of the composition. In some aspects, one or more of these agents may be added to improve the performance, efficacy, safety, shelf-life and/or other property of the muscarinic antagonist composition of the present disclosure. In some aspects, additives may be biocompatible, without being harsh, abrasive, and/or allergenic.

In certain aspects, compositions disclosed herein may comprise a buffer system. As used herein, a “buffer system” is a composition comprised of one or more buffering agents wherein “buffering agents” are compounds used to resist change in pH upon dilution or addition of acid or alkali. Buffering agents include, by way of example and without limitation, potassium metaphosphate, potassium phosphate, monobasic sodium acetate and sodium citrate anhydrous and dihydrate and other materials known to one of ordinary skill in the art. In some aspects, any pharmaceutically acceptable organic or inorganic buffer can be used. In some aspects, compositions disclosed herein may comprise at least 5%, at least 10%, at least 20%, at least 25%, at least 30%, at least 35%, at least 40%, at least 45%, at least 50% total amount of one or more buffering agents by total weight of the composition. In some aspects, compositions disclosed herein may comprise about 5% to about 99%, about 10%, about 95%, or about 15% to about 90% total amount of one or more buffering agents by total weight of the composition.

In some aspects, the amount of one or more buffering agents may depend on the desired pH level of a composition. In some aspects, the compositions disclosed herein may have a pH of about 6 to about 9. In some aspects, the compositions disclosed herein may have a pH greater than about 8, greater than about 7.5, greater than about 7, greater than about 6.5, or greater than about 6.

In certain aspects, compositions disclosed herein may comprise one or more preservatives. As used herein, “preservatives” refers to agents or combination of agents that inhibits, reduces or eliminates bacterial growth in a pharmaceutical dosage form. Non-limiting examples of preservatives include Nipagin, Nipasol, isopropyl alcohol and a combination thereof. In some aspects, any pharmaceutically acceptable preservative can be used. In some aspects, compositions disclosed herein may comprise at least 5%, at least 10%, at least 20%, at least 25%, at least 30%, at least 35%, at least 40%, at least 45%, at least 50% total amount of one or more preservatives by total weight of the composition. In some aspects, compositions disclosed herein may comprise about 5% to about 99%, about 10%, about 95%, or about 15% to about 90% total amount of one or more preservatives by total weight of the composition.

In certain aspects, compositions disclosed herein may comprise one or more surface-acting reagents or detergents. In some aspects, surface-acting reagents or detergents may be synthetic, natural, or semi-synthetic. In some aspects, compositions disclosed herein may comprise anionic detergents, cationic detergents, zwitterionic detergents, ampholytic detergents, amphoteric detergents, nonionic detergents having a steroid skeleton, or a combination thereof. In some aspects, compositions disclosed herein may comprise at least 5%, at least 10%, at least 20%, at least 25%, at least 30%, at least 35%, at least 40%, at least 45%, at least 50% total amount of one or more surface-acting reagents or detergents by total weight of the composition. In some aspects, compositions disclosed herein may comprise about 5% to about 99%, about 10%, about 95%, or about 15% to about 90% total amount of one or more surface-acting reagents or detergents by total weight of the composition.

In certain aspects, compositions disclosed herein may comprise one or more stabilizers. As used herein, a “stabilizer” refers to a compound used to stabilize an active agent against physical, chemical, or biochemical process that would otherwise reduce the therapeutic activity of the agent. Suitable stabilizers include, by way of example and without limitation, succinic anhydride, albumin, sialic acid, creatinine, glycine and other amino acids, niacinamide, sodium acetyltryptophonate, zinc oxide, sucrose, glucose, lactose, sorbitol, mannitol, glycerol, polyethylene glycols, sodium caprylate and sodium saccharin and others known to those of ordinary skill in the art. In some aspects, compositions disclosed herein may comprise at least 5%, at least 10%, at least 20%, at least 25%, at least 30%, at least 35%, at least 40%, at least 45%, at least 50% total amount of one or more stabilizers by total weight of the composition. In some aspects, compositions disclosed herein may comprise about 5% to about 99%, about 10%, about 95%, or about 15% to about 90% total amount of one or more stabilizers by total weight of the composition.

In some aspects, compositions disclosed herein may comprise one or more tonicity agents. As used herein, a “tonicity agents” refers to a compound that can be used to adjust the tonicity of the liquid formulation. Suitable tonicity agents include, but are not limited to, glycerin, lactose, mannitol, dextrose, sodium chloride, sodium sulfate, sorbitol, trehalose and others known to those or ordinary skill in the art. Osmolarity in a composition may be expressed in milliosmoles per liter (mOsm/L). Osmolarity may be measured using methods commonly known in the art. In some aspects, a vapor pressure depression method is used to calculate the osmolarity of the compositions disclosed herein. In some aspects, the amount of one or more tonicity agents comprising a composition disclosed herein may result in a composition osmolarity of about 150 mOsm/L to about 500 mOsm/L, about 250 mOsm/L to about 500 mOsm/L, about 250 mOsm/L to about 350 mOsm/L, about 280 mOsm/L to about 370 mOsm/L or about 250 mOsm/L to about 320 mOsm/L. In some aspects, a composition herein may have an osmolality ranging from about 100 mOsm/kg to about 1000 mOsm/kg, from about 200 mOsm/kg to about 800 mOsm/kg, from about 250 mOsm/kg to about 500 mOsm/kg, or from about 250 mOsm/kg to about 320 mOsm/kg, or from about 250 mOsm/kg to about 350 mOsm/kg or from about 280 mOsm/kg to about 320 mOsm/kg. In some aspects, a composition described herein may have an osmolarity of about 100 mOsm/L to about 1000 mOsm/L, about 200 mOsm/L to about 800 mOsm/L, about 250 mOsm/L to about 500 mOsm/L, about 250 mOsm/L to about 350 mOsm/L, about 250 mOsm/L to about 320 mOsm/L, or about 280 mOsm/L to about 320 mOsm/L. In some aspects, compositions disclosed herein may comprise at least 5%, at least 10%, at least 20%, at least 25%, at least 30%, at least 35%, at least 40%, at least 45%, at least 50% total amount of one or more tonicity modifiers by total weight of the composition. In some aspects, compositions disclosed herein may comprise about 5% to about 99%, about 10%, about 95%, or about 15% to about 90% total amount of one or more tonicity modifiers by total weight of the composition.

In certain aspects, the composition disclosed herein can be formulated for infusion, implantation, subcutaneous injection, or intraperitoneal injection. In certain aspects, the disclosed composition can be formulated for administration by injection. In some aspects, compositions comprising COIN for e.g., intradermal, subcutaneous or intramuscular administration, can be prepared in sterile suspension, solutions or emulsions. In some aspects, the composition comprising COIN can be formulated for administration by or during colonoscopy. In some aspects, the composition comprising COIN can be directly administered to identified inflamed intestinal tissue. In some aspects, In some aspects, the COIN can be formulated for administration as a suspension in PBS. In some aspects, the compositions for administration comprises about 1 μL, about 5 μL, about 10 μL, about 50 μL, about 100 μL, about 200 μL, about 250 μL, about 300 μL, about 350 μL, about 400 μL, about 450 μL, about 500 μL, about 550 μL, about 600 μL, about 650 μL, about 700 μL, about 750 μL, about 800 μL, about 850 μL, about 900 μL, about 950 μL or about 1000 μL. In some aspects, the composition can comprise COIN in concentration range of about 0.1 mg/mL to 1000 mg/mL, or about 1 to 200 mg/mL, or about 10 mg/mL to 100 mg/mL.

In some aspect, the composition may comprise one or more active agents in addition to the colon-specific immune niche, provided herein. Generally, the therapeutic agents can include any substance that can be coated on, embedded into, absorbed into, adsorbed to, or otherwise attached to or incorporated onto or into the composition described herein or incorporated into a drug product that would provide a therapeutic benefit to a patient. Non-limiting examples of such additional active agents include: growth factors, chemoattractants, cytokines, antimicrobial agents, emollients, retinoids, steroids, anti-inflammatories, hormones, angiogenic factors, analgesics, chemotherapeutic agents, or any other known agents used for treating a condition or disease of large intestinal tract tissue.

Non-limiting examples of growth factors include basic fibroblast growth factor (bFGF), acidic fibroblast growth factor (aFGF), vascular endothelial growth factor (VEGF), hepatocyte growth factor (HGF), insulin-like growth factors 1 and 2 (IGF-1 and IGF-2), platelet derived growth factor (PDGF), stromal derived factor 1 alpha (SDF-1 alpha), nerve growth factor (NGF), ciliary neurotrophic factor (CNTF), neurotrophin-3, neurotrophin-4, neurotrophin-5, pleiotrophin protein (neurite growth-promoting factor I), midline protein (neurite growth-promoting factor 2), brain-derived neurotrophic factor (BDNF), tumor angiogenesis factor (TAF), corticotrophin releasing factor (CRF), transforming growth factors α and β (TGF-α and TGF-β), interleukin-8 (IL-8), granulocyte-macrophage colony stimulating factor (GM-CSF), interleukins, and interferons.

Non-limiting examples of an antimicrobial agent, include, without limitation, isoniazid, ethambutol, pyrazinamide, streptomycin, clofazimine, rifabutin, fluoroquinolones, ofloxacin, sparfloxacin, rifampin, azithromycin, clarithromycin, dapsone, tetracycline, erythromycin, ciprofloxacin, doxycycline, ampicillin, amphotericin B, ketoconazole, fluconazole, pyrimethamine, sulfadiazine, clindamycin, lincomycin, pentamidine, atovaquone, paromomycin, diclazuril, acyclovir, trifluorouridine, foscarnet, penicillin, gentamicin, ganciclovir, itraconazole, miconazole, Zn-pyrithione, and silver salts such as chloride, bromide, iodide and periodate.

Non-limiting examples of an anti-inflammatory agent include, without limitation, an NSAID, such as salicylic acid, indomethacin, sodium indomethacin trihydrate, salicylamide, naproxen, colchicine, fenoprofen, sulindac, diflunisal, diclofenac, indoprofen, sodium salicylamide; an anti-inflammatory cytokine; an anti-inflammatory protein; a steroidal anti-inflammatory agent; or an anti-clotting agent, such as heparin.

Non-limiting examples of chemotherapeutic agents include antimetabolites, such as methotrexate, DNA cross-linking agents, such as cisplatin/carboplatin; alkylating agents, such as canbusil; topoisomerase I inhibitors such as dactinomicin; microtubule inhibitors such as taxol (paclitaxol), and the like. Other chemotherapeutic agents include, for example, a vinca alkaloid, mitomycin-type antibiotic, bleomycin-type antibiotic, antifolate, colchicine, demecoline, etoposide, taxane, anthracycline antibiotic, doxorubicin, daunorubicin, carminomycin, epirubicin, idarubicin, mithoxanthrone, 4-dimethoxy-daunomycin, 11-deoxydaunorubicin, 13-deoxydaunorubicin, adriamycin-14-benzoate, adriamycin-14-octanoate, adriamycin-14-naphthaleneacetate, amsacrine, carmustine, cyclophosphamide, cytarabine, etoposide, lovastatin, melphalan, topetecan, oxalaplatin, chlorambucil, methtrexate, lomustine, thioguanine, asparaginase, vinblastine, vindesine, tamoxifen, or mechlorethamine. While not wanting to be limiting, therapeutic antibodies include antibodies directed against the HER2 protein, such as trastuzumab; antibodies directed against growth factors or growth factor receptors, such as bevacizumab, which targets vascular endothelial growth factor, and OSI-774, which targets epidermal growth factor; antibodies targeting integrin receptors, such as Vitaxin (also known as MEDI-522), and the like. Classes of anticancer agents suitable include, but are not limited to: 1) alkaloids, including, microtubule inhibitors (e.g., Vincristine, Vinblastine, and Vindesine, etc.), microtubule stabilizers (e.g., Paclitaxel [Taxol], and Docetaxel, Taxotere, etc.), and chromatin function inhibitors, including, topoisomerase inhibitors, such as, epipodophyllotoxins (e.g., Etoposide [VP-16], and Teniposide [VM-26], etc.), and agents that target topoisomerase I (e.g., Camptothecin and Isirinotecan [CPT-11], etc.); 2) covalent DNA-binding agents [alkylating agents], including, nitrogen mustards (e.g., Mechlorethamine, Chlorambucil, Cyclophosphamide, Ifosphamide, and Busulfan [Myleran], etc.), nitrosoureas (e.g., Carmustine, Lomustine, and Semustine, etc.), and other alkylating agents (e.g., Dacarbazine, Hydroxymethylmelamine, Thiotepa, and Mitocycin, etc.); 3) noncovalent DNA-binding agents [antitumor antibiotics], including, nucleic acid inhibitors (e.g., Dactinomycin [Actinomycin D], etc.), anthracyclines (e.g., Daunorubicin [Daunomycin, and Cerubidine], Doxorubicin [Adriamycin], and Idarubicin [Idamycin], etc.), anthracenediones (e.g., anthracycline analogues, such as, [Mitoxantrone], etc.), bleomycins (Blenoxane), etc., and plicamycin (Mithramycin), etc.; 4) antimetabolites, including, antifolates (e.g., Methotrexate, Folex, and Mexate, etc.), purine antimetabolites (e.g., 6-Mercaptopurine [6-MP, Purinethol], 6-Thioguanine [6-TG], Azathioprine, Acyclovir, Ganciclovir, Chlorodeoxyadenosine, 2-Chlorodeoxyadenosine [CdA], and 2′-Deoxycoformycin [Pentostatin], etc.), pyrimidine antagonists (e.g., fluoropyrimidines [e.g., 5-fluorouracil (Adrucil), 5-fluorodeoxyuridine (FdUrd) (Floxuridine)] etc.), and cytosine arabinosides (e.g., Cytosar [ara-C] and Fludarabine, etc.); 5) enzymes, including, L-asparaginase; 6) hormones, including, glucocorticoids, such as, antiestrogens (e.g., Tamoxifen, etc.), nonsteroidal antiandrogens (e.g., Flutamide, etc.), and aromatase inhibitors (e.g., anastrozole [Arimidex], etc.); 7) platinum compounds (e.g., Cisplatin and Carboplatin, etc.); 8) monoclonal antibodies conjugated with anticancer drugs, toxins, and/or radionuclides, etc.; 9) biological response modifiers (e.g., interferons [e.g., IFN-.alpha., etc.] and interleukins [e.g., IL-2, etc.], etc.); 10) adoptive immunotherapy; 11) hematopoietic growth factors; 12) agents that induce tumor cell differentiation (e.g., all-trans-retinoic acid, etc.); 13) gene therapy techniques; 14) antisense therapy techniques; 15) tumor vaccines; 16) therapies directed against tumor metastases (e.g., Batimistat, etc.); and 17) inhibitors of angiogenesis.

The actual dosage amount of a composition according to the present disclosure, and the selection of any combination treatment to be administered to a subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

### IV. Methods of Treatment

The present disclosure further provides a method of treating a condition or disease of large intestinal tract tissue of a subject. The method can comprise administering an effective amount of colon-specific immune niche (COIN) disclosed herein, to the subject in need thereof.

In some aspects, the disclosure encompasses a method of preventing, treating, or reducing the risk of a condition or disease of large intestinal tract tissue in a subject. In such aspects, the method can comprise administering to a subject in need thereof an effective amount of a composition comprising COIN.

In some aspects, further disclosed is a method of preventing, treating, or reducing the risk of a condition or disease of large intestinal tract tissue associated cancer in a subject. In such aspects the method can comprise administering to a subject in need thereof an effective amount of a composition comprising COIN.

In some aspects, provided herein is a method of preventing, treating, or reducing the risk of a condition or disease of large intestinal tract tissue associated with Phosphoinositide 3-kinase (PI3K) inhibitor in a subject. In such aspects the method comprises administering to a subject in need thereof, an effective amount of a composition comprising COIN. In some aspects, the disclosed PI3K inhibitor can be copanlisib, idelalisib, umbralisib, duvelisib, alpelisib, anti-PI3K antibody, CAL-120/GS-9820, Wortmannin, demethoxyviridin, LY294002, perifosine, CAL-101, PX-866, IPI-145, BAY 80-6946, BEZ-235, RP-6503, TGR-1202, SF-1126, INK-1117, GDC-0941, BKM-120, XL-147, XL-765, Palomid 529, GSK-1059615, ZSTK-474, PWT-33597, IC-87114, TG100-115, CAL-263, RP-6530, PI-103, GNE-477, CUDC-907, AEZS-136, or any combination thereof. In some aspects, PI3K inhibitor can be copanlisib.

In some aspects, further provided is a method of preventing, treating, or reducing the risk of a condition or disease of large intestinal tract tissue associated with an immune-related adverse event (irAE) during immune checkpoint blockade therapy in a subject. In such aspects, the method can comprise administering to a subject in need thereof, an effective amount of a composition comprising COIN. In such aspects, the subject can be undergoing or planning to undergo cancer treatment with immune checkpoint blockade therapy. In some aspects, the immune checkpoint blockade therapy comprises administration of an inhibitor of PD-1, PD-L1, TIM-3, LAG-3, CTLA-4, CSF-1R, or any combinations thereof.

In some aspects, a condition or disease of lower intestinal tract tissue as disclosed herein can comprise inflammatory bowel disease. In some aspects, condition or disease of lower intestinal tract tissue can be Crohn's Disease, Ulcerative Colitis, familial adenomatous polyposis, Hirschsprungs, proctitits, celiac disease, gastrointestinal mucositis, colorectal cancer, or fistula. In some aspects of the method of treatment, the subject can be a subject, undergoing or planning to undergo cancer treatment (for e.g., chemotherapy or radiation). One skilled in the art can use any known methods for diagnosing the condition or disease of lower intestinal tract tissue, non-limiting examples of which are physical exams, imaging tests (x-rays, computerized tomography (CT) scans, magnetic resonance imaging (MRI) scans, ultrasounds, positron emission tomography (PET) scans, etc.), endoscopy exams, biopsies or exploratory surgery.

In some aspects, the method of the present disclosure can comprise administering an effective amount of a composition comprising about 1 mg of NFs functionalized with PD-L1 antibody, about 0.5 mg of decellularized colon ECM, and colon EPI cells at about 1×10⁶ cells. In some aspects, the method can comprise administering an effective amount of a composition comprising about 1 mg of NFs functionalized with CD86 antibody, about 0.5 mg of decellularized colon ECM, and colon EPI cells at about 1×10⁶ cells. In some aspects, the method can comprise administering an effective amount of a composition comprising about 1 mg of NFs functionalized with LEF, about 0.5 mg of decellularized colon ECM, and colon EPI cells at about 1×10⁶ cells. In some aspects, the method can comprise administering an effective amount of a composition comprising about 1 mg of NFs functionalized with TGF-β1 mimetic peptide, about 0.5 mg of decellularized colon ECM, and colon EPI cells at about 1×10⁶ cells. In some aspects, the method comprises administering an effective amount of a composition comprising about 1 mg of NFs functionalized with PD-L1 antibody, about 1 mg of NFs functionalized with CD86 antibody, about 0.5 mg of decellularized colon ECM, and colon EPI cells at about 1×10⁶ cells. In some aspects, the method comprises administering an effective amount of a composition comprising about 1 mg of NFs functionalized with PD-L1 antibody, about 1 mg of NFs functionalized with CD86 antibody, about 1 mg of NFs functionalized with LEF, about 0.5 mg of decellularized colon ECM, and colon EPI cells at about 1×10⁶ cells. In some aspects, the method comprises administering an effective amount of a composition comprising about 1 mg of NFs functionalized with PD-L1 antibody, about 1 mg of NFs functionalized with CD86 antibody, about 1 mg of NFs functionalized with LEF, about 1 mg of NFs functionalized with TGF-β1 mimetic peptide, about 0.5 mg of decellularized colon ECM, and colon EPI cells at about 1×10⁶ cells.

In some aspects, administering an effective amount of COIN can include, but are not limited to, parenteral administration (e.g., intradermal, intramuscular, intracoronary, intraperitoneal, intravenous and subcutaneous), epidural, and mucosal (e.g., intranasal, inhaled, and oral routes). The suspension comprising of COIN may be administered by any convenient route, for example, by infusion or bolus injection, by absorption through epithelial or mucocutaneous linings (e.g., oral mucosa, rectal and intestinal mucosa, etc.), or implantation, and may be administered together with other biologically active agents. In some aspects, the COIN is administered through infusion, injection or implantation. In some aspects, the colon-like tissue microenvironment is administered through subcutaneous injection.

In some aspects, the effective amount of COIN can be about 1 μL to about 1000 μL. In some aspects, the COIN is administrated at a volume of about 1 μl, about 2 μl, about 3 μl, about 4 μl, about 5 μl, about 6 μl, about 7 μl, about 8 μl, about 9 μl, about 10 μl, about 15 μl, about 20 μl, about 25 μl, about 30 μl, about 35 μl, about 40 μl, about 45 μl, about 50 μl, about 55 μl, about 60 μl, about 65 μl, about 70 μl, about 75 μl, about 80 μl, about 85 μl, about 90 μl, about 95 μl, about 100 μl, about 125 μl, about 150 μl, about 200 μl, about 250 μl, about 300 μl, about 350 μl, about 400 μl, about 450 μl, about 500 μl, about 550 μl, about 600 μl, about 650 μl, about 700 μl, about 750 μl, about 800 μl, about 850 μl, about 900 μl, about 950 μl or about 1 ml. In some aspects, the effective amount is about 200 μl.

In some aspects, the composition comprising COIN can be administered to the subject to provide a total daily dose of COIN of from 0.1 ml/kg to about 10 ml/kg; for example from about 0.5 ml/kg to about 5 ml/kg, from about 0.5 ml/kg to about 2 ml/kg or about 0.5 ml/kg to about 1.5 ml/kg, for example about 1 ml/kg.

The composition comprising COIN described herein, can be administered to the subject daily or more than once daily. In some aspects, the disclosed composition comprising COIN can be administered every 2, 3, 4, 5, 6, 7, 14, or every 30 days. The disclosed composition comprising COIN can be administered over a period ranging from about 1 day to about 1 year, from about 1 day to about 1 week, from about 3 days to about 1 month, from about 2 weeks to about 6 months, or from about 2 months to about 4 months. The disclosed composition comprising COIN composition comprising COIN can also be administered over a period of about 1 day, about 7 days, about 30 days, about 60 days, about 120 days, or about 180 days or more. In some aspects, the disclosed composition comprising COIN is administered over a period of about 57 weeks, about 148 weeks, about 208 weeks, or indefinitely.

Dosage amount and interval may be adjusted individually to provide COIN, which are sufficient to maintain therapeutic or prophylactic effect. For example, the COIN may be administered once per week, several times per week (e.g., every other day), once per day or multiple times per day, depending upon, among other things, the mode of administration, the specific indication being treated and the judgment of the prescribing physician. Skilled artisans will be able to optimize effective dosages without undue experimentation.

In some aspects, the administration of the composition comprising COIN can be combined with other therapeutic agents. In some aspects, administration of the composition comprising COIN can be combined with other therapies, as required by the subject's condition such as chemotherapy, radiation therapy, surgery, etc. or agents that stimulate the immune system or reduce inflammation. In some aspects, the composition comprising COIN can be combined with antibiotics to treat infections, corticosteroids (for e.g., aclometasone, aclometasone dipropionate, aldosterone, amcinonide, beclomethasone, beclomethasone dipropionate, betamethasone, betamethasone dipropionate, betamethasone sodium phosphate, betamethasone valerate, budesonide, clobetasone, clobetasone butyrate, clobetasol propionate, cloprednol, cortisone, cortisone acetate, cortivazol, deoxycortone, desonide, desoximetasone, dexamethasone, dexamethasone sodium phosphate, dexamethasone isonicotinate, difluorocortolone, fluclorolone, flumethasone, flunisolide, fluocinolone, fluocinolone acetonide, fluocinonide, fluocortin butyl, fluorocortisone, fluorocortolone, fluocortolone caproate, fluocortolone pivalate, fluorometholone, fluprednidene, fluprednidene acetate, flurandrenolone, fluticasone, fluticasone propionate, halcinonide, hydrocortisone, hydrocortisone acetate, hydrocortisone butyrate, hydrocortisone aceponate, hydrocortisone buteprate, hydrocortisone valerate, icomethasone, icomethasone enbutate, meprednisone, methylprednisolone, mometasone paramethasone, mometasone furoate monohydrate, prednicarbate, prednisolone, prednisone, tixocortol, tixocortol pivalate, triamcinolone, triamcinolone acetonide, triamcinolone alcohol), immune modifiers to repress autoimmune response, cyclosporin (for e.g., Neoral™, Sandimmun™), immunosuppressant (for e.g., azathioprine, mercaptopurine) aminosalicylates, steroid, anti-TNF antibody (for e.g., infliximab, adalimumab or golimumab), an integrin inhibitor antibody (for e.g., vedolizumab) or a non-steroidal anti-inflammatory drug, NSAID but not limited to diclofenac potassium, diclofenac sodium, etodolac, indomethicin, ketorolac tromethamine, sulindac, tometin sodium, celecoxib, meloxicam, valdecoxib, fructafenine, mefenamic acid, Nabumetone, meloxicam, piroxicam, tenoxicam, fenoprofen calcium, flubiprofen, ibuprofen, ketoprofen, naproxen, naproxen sodium, oxaprozin, thiaprofenic acid, acetylsalicylic acid, diflunisal, choline magnesium trisalicylic acid, choline salicylic acid, triethanolamine salicylic acid, COX1 Inhibitors, COX2 inhibitors (eg Vioxx™ and Celebrex™. In some aspects, the composition comprising COIN may be combined with any other therapy intended to benefit the subject, such as nutrients, vitamins and supplements. For example, vitamin A, vitamin D, vitamin E, vitamin C, vitamin B complex, selenium, zinc, coenzyme Q10, beta carotene, fish oil, curcumin, green tea, bromelain, resveratrol, powdered linseed, garlic, lycopene, maria thistle, Melatonin, other antioxidants, cimetidine, or indomethacin) may also be administered to the subject. Such treatments can be administered concurrently, or sequentially.

The amount of COIN in the compositions of the disclosure may vary depending on factors such as the disease state, age, sex and weight of the subject. Dosage regimens may be adjusted to provide the optimum therapeutic response. For example, a single dose may be administered, several divided doses may be administered over time, or the dose may be reduced or increased proportionally as indicated by the urgency of the treatment situation.

In some aspects, the administration of the composition comprising COIN can reduce CD8+ T cell activation in the subject. In some aspects, the administration can reduce CD8+ T cell activation by about 5% to about 99% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%) compared to CD8+ T cell activation in the absence of COIN, or before treatment. CD8+ T cell activation can be quantified using any known method in the art including quantification of the T cell activation markers or staining with labeled anti-mouse CD8 antibody.

In some aspects, the administration of the composition comprising COIN can reduce pro-inflammatory cytokine production in the subject. In some aspects, pro-inflammatory cytokine can be interleukin-6 (IL-6) TNF-α, or any combination thereof. In some aspects, the administration can reduce pro-inflammatory cytokine production by about 5% to about 99% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%) compared to pro-inflammatory cytokine production in the absence of COIN, or before treatment. Any known method in the art can be used for measuring the production of pro-inflammatory cytokine, for e.g., antibody-based assays.

In some aspects, the administration of the composition comprising COIN can reduce hematochezia in the subject. In some aspects, the administration can reduce hematochezia by about 5% to about 99% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%) compared to hematochezia in the absence of COIN, or before treatment. Hematochezia can be quantified using any known method in the art including visual scoring or examination.

In some aspects, the administration of the composition comprising COIN can reduce colitis-induced anemia in the subject. In some aspects, the administration can reduce colitis-induced anemia by about 5% to about 99% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%) compared to colitis-induced anemia in the absence of COIN, or before treatment. Colitis-induced anemia can be quantified using any known method in the art including hemoglobin counting.

In some aspects, the administration of the composition comprising COIN can reduce lymphocytic colitis in the subject. In some aspects, the administration can reduce lymphocytic colitis by about 5% to about 99% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%) compared to lymphocytic colitis in the absence of COIN, or before treatment. Lymphocytic colitis can be quantified using any known method in the art including using lymphocyte counts.

In some aspects, the administration of the composition comprising COIN can reduce inflammation-induced colon shortening in the subject. In some aspects, the administration can reduce inflammation-induced colon shortening by about 5% to about 99% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%) compared to inflammation-induced colon shortening in the absence of COIN, or before treatment.

In some aspects, the administration of the composition comprising COIN can reduce epithelium damage in the colon of the subject. In some aspects, the administration can reduce epithelium damage in the colon by about 5% to about 99% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%) compared to epithelium damage in the colon in the absence of COIN, or before treatment. Epithelium damage can be quantified using any known method in the art including using histological analysis.

In some aspects, the administration of the composition comprising COIN can reduce immune cell infiltration into the lamina propria in the colon of the subject. In some aspects, the administration can reduce immune cell infiltration into the lamina propria in the colon by about 5% to about 99% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%) compared to immune cell infiltration into the lamina propria in the colon in the absence of COIN, or before treatment. Immune cell infiltration into the lamina propria can be quantified using any known method in the art including using immune cell profiling using mass cytometry.

In some aspects, the administration of the composition comprising COIN can reduce tumor burden in the colon of the subject. In some aspects, the administration can reduce tumor burden in the colon by about 5% to about 99% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%) compared to tumor burden in the colon in the absence of COIN, or before treatment. Tumor burden can be quantified using any known method in the art including using histological analysis.

In some aspects, the administration of the composition comprising COIN can reduce rectum prolapse in the subject. In some aspects, the administration can reduce rectum prolapse by about 5% to about 99% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%) compared to rectum prolapse in the absence of COIN, or before treatment. Rectum prolapse can be quantified using any known method in the art including using anorectal examination.

In some aspects, the administration of the composition comprising COIN can reduce microbiome dysbiosis in the subject. In some aspects, the administration can reduce microbiome dysbiosis by about 5% to about 99% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%) compared to microbiome dysbiosis in the absence of COIN, or before treatment. Microbiome dysbiosis can be quantified using any known method in the art including using gut microbiome analysis by 16S rRNA sequencing.

In some aspects, the administration of the composition comprising COIN can enhance M1 Mφ differentiation into M2 Mφ in the subject. In some aspects, the administration can enhance M1 Mφ differentiation into M2 Mφ by about 5% to about 99% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%) compared to M1 Mφ differentiation into M2 Mφ in the absence of COIN, or before treatment. M1 Mφ differentiation into M2 Mφ can be quantified using any known method in the art including using cell staining.

In some aspects, the administration of the composition comprising COIN can enhance pro-regulatory cytokine production in the subject. In some aspects, the pro-regulatory cytokine can be interleukin-10 (IL-10), TGF-β1, or any combination thereof. In some aspects, the administration can enhance pro-regulatory cytokine production by about 5% to about 99% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%) compared to pro-regulatory cytokine production in the absence of COIN, or before treatment. Pro-regulatory cytokine production can be quantified using any known method in the art including for e.g., antibody based assays.

In some aspects, the administration of the composition comprising COIN can promote repair of epithelium damage in the colon of the subject. In some aspects, the administration can promote repair of epithelium damage by about 5% to about 99% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%) compared to repair of epithelium damage in the absence of COIN, or before treatment. Repair of epithelium damage can be quantified using any known method in the art including for e.g., H&E staining and histological analysis.

In some aspects, the administration of the composition comprising COIN can enhance recovery of body weight in the subject. In some aspects, the administration can enhance recovery of body weight by about 5% to about 99% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%) compared to recovery of body weight in the absence of COIN, or before treatment.

In some aspects, the administration of the composition comprising COIN can promote colon length maintenance in the subject. In some aspects, the administration can promote colon length maintenance by about 5% to about 99% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%) compared to colon length maintenance in the absence of COIN, or before treatment.

In some aspects, the administration of the composition comprising COIN can enhance commensal microbiomes in the subject. In some aspects, the administration can enhance commensal microbiomes by about 5% to about 99% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%) compared to commensal microbiomes in the absence of COIN, or before treatment. Commensal microbiomes can be quantified using any known method in the art including using gut microbiome analysis by 16S rRNA sequencing.

In further aspects, the administration of the composition comprising COIN can result in inhibition of disease progression, reduction in autoimmunity, induction of tolerance, reduction in inflammation, or any combination thereof. In some aspects, the administration of the composition comprising COIN can reduce, minimize or stop one or more symptoms associated with condition or disease of large intestinal tract tissue. In some aspects, the administration of the composition comprising COIN can reduce, minimize, or stop diarrhea, rectal bleeding, bodyweight loss, abdominal pain, cramping, blood in stools, reduced appetite, tiredness, anemia, perforations, obstruction of the bowel, and/or fever.

In some aspects, the reduction in one or more symptoms is 10%, 20%, 25%, 30%, 35%, 40%, 45%, 50%, 55%, 60%, 65%, 70%, 75% or more compared to baseline assessment in the subject, before treatment, or a control subject not receiving the treatment.

In some aspects, the treatment disclosed herein may result in disease remission. In one example, the time to disease remission can be less than that achieved in a subject who is not treated by the method described herein. In another example, the duration of remission can be greater than that achieved in a subject who is not treated by the method described herein. For example, the duration of remission may be for at least 24 weeks, such as for at least 48 weeks.

In some aspects the method, can further comprise modifying one or more of steps of administration. For example, in an aspect, the method can be altered by changing the amount of COIN, thereof administered to a subject, or by changing the frequency of administration of COIN, or by changing the duration of time of COIN administered to a subject.

### V. Kits

In further aspects, COIN described herein can be provided in a kit. The kit can include the COIN, for e.g., a composition that includes NFs functionalized with an immunomodulator molecule, colon ECM and colon EPI cells, disclosed herein, and informational material. The informational material can be descriptive, instructional, marketing or other material that relates to the methods described herein and/or the use of the COIN, for the methods described herein. For example, the informational material describes methods for administering the COIN, to treat or prevent a subject from the development of a condition or disease of lower intestinal tract tissue, or at least one symptom of the condition or disease of lower intestinal tract tissue.

In some aspects, the informational material can include instructions to administer the colon-like tissue microenvironment, in a suitable manner, e.g., in a suitable dose, dosage form, or mode of administration (e.g., a dose, dosage form, or mode of administration described herein). The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In some aspects, the informational material of the kit is a link or contact information, e.g., a physical address, email address, hyperlink, website, or telephone number, where a user of the kit can obtain substantive information about the COIN, and/or its use in the methods described herein.

In addition to the COIN, the composition of the kit can include other ingredients, such as a solvent or buffer, a stabilizer or a preservative, and/or a second agent for treating condition or disease of lower intestinal tract tissue, described herein. Alternatively, the other ingredients can be included in the kit, but in different compositions or containers than the COIN. In such aspects, the kit can include instructions for admixing the COIN, and the other ingredients, or for using the COIN, together with the other ingredients.

In some aspects, the COIN, can be provided in any form, e.g., liquid, dried or lyophilized form. In some aspects, the COIN, is substantially pure and/or sterile. When the colon-like tissue microenvironment is provided in a liquid solution, the liquid solution preferably is an aqueous solution, with a sterile aqueous solution. When the COIN, is provided as a dried form, reconstitution generally is by the addition of a suitable solvent. In some aspects, the solvent, e.g., sterile water or buffer (e.g., PBS), can be provided in the kit.

The kit can include one or more containers for the composition containing the COIN. In some aspects, the kit contains separate containers, dividers or compartments for COIN (e.g., in a composition) and informational material. For example, the COIN (e.g., in a composition) can be contained in a bottle, vial, or syringe, and the informational material can be contained in a plastic sleeve or packet. In other aspects, the separate elements of the kit are contained within a single, undivided container. For example, the COIN (e.g., in a composition) is contained in a bottle, vial or syringe that has attached thereto the informational material in the form of a label. In some aspects, the kit includes a plurality (e.g., a pack) of individual containers, each containing one or more unit dosage forms (e.g., a dosage form described herein) of the agent (e.g., in a composition). For example, the kit can include a plurality of syringes, ampules, or foil packets, each containing a single unit dose of the COIN. The containers of the kits can be airtight and/or waterproof.

Having described several aspects, it will be recognized by those skilled in the art that various modifications, alternative constructions, and equivalents may be used without departing from the spirit of the present disclosure. Additionally, a number of well-known processes and elements have not been described in order to avoid unnecessarily obscuring the present disclosure. Accordingly, this description should not be taken as limiting the scope of the present disclosure.

Those skilled in the art will appreciate that the presently disclosed aspects teach by way of example and not by limitation. Therefore, the matter contained in this description or shown in the accompanying drawings should be interpreted as illustrative and not in a limiting sense. The following claims are intended to cover all generic and specific features described herein, as well as all statements of the scope of the method and assemblies, which, as a matter of language, might be said to fall there between.

### EXAMPLES

The following examples are included to demonstrate preferred aspects of the disclosure. It should be appreciated by those of skill in the art that the techniques disclosed in the examples that follow represent techniques discovered by the inventor to function well in the practice of the present disclosure, and thus can be considered to constitute preferred modes for its practice. However, those of skill in the art should, in light of the present disclosure, appreciate that many changes can be made in the specific aspects which are disclosed and still obtain a like or similar result without departing from the spirit and scope of the present disclosure.

### Materials, and Methods

### Materials

Unless specified, all reagents were purchased from Fisher Scientific or Millipore Sigma.

### Synthesis of Carboxylic Acid-Functionalized Diblock Copolymer Nanofibers

Carboxylic acid-functionalized poly(2-(methacryloyloxy)ethyl phosphorylcholine)-poly(hydroxypropyl methacrylate) (PMPC-PHPMA) amphiphilic diblock-copolymer nanofibers were prepared via an optimized two-step reversible addition-fragmentation chain-transfer (RAFT) polymerization. An idealized morphology-based phase diagram for PMPC-PHPMA was constructed from four PMPC macro-CTAs with different PHPMA block lengths. Briefly, 4 different PMPC macro-CTAs (building blocks) were synthesized via RAFT polymerization with actual degrees of polymerization (DPs, determined by ¹H NMR spectroscopy) of 13, 21, 27, and 50. The PMPC macro-CTAs were chain extended with HPMA in H₂O through RAFT aqueous dispersion polymerization with target DPs (for the HPMA block) varying from 140 to 490 at 25% solid content. The morphologies of all diblock copolymers were inspected via TEM.

The diblock copolymer NFs used in this study have a DP of 27 for the hydrophilic PMPC block and an actual DP of 280 for the hydrophobic PHPMA block. Briefly, a well-defined PMPC₂₇ macro-CTA was first prepared via RAFT polymerization of MPC (catalog number: 67881-98-5; TCI Chemicals) using 4-cyano-4-(phenylcarbonothioylthio)pentanoic acid (CPDB; catalog number: 722995; Sigma) as a RAFT chain-transfer agent and 2,2′-azobis(2-methylpropionamidine) dihydrochloride (VA044; Fujifilm) as a thermal initiator at a target [MPC]:[CPDB]:[VA044] molar ratio of 30:1:0.25. The polymerization was performed at 40% solid content in a 1:9 water:methanol mixture at 50° C. for 150 min (approximately 90% conversion). The PMPC macro-CTA was isolated and purified by repeated precipitation into a 20-fold excess of a 1:20 methanol:acetone mixture (3 times). The collected polymer powders were first dried under vacuum before being redissolved in deionized water and lyophilized to remove residual solvents. The mean DP of the PMPC homopolymer calculated from the aromatic RAFT agent end group was 27, as determined by ¹H NMR spectroscopy. The PMPC₂₇ macro-CTA was chain extended via RAFT aqueous polymerization of HPMA (mixture of isomers, purity >97%; catalog number: A17951.AK; Alfa Aesar) at a target [HPMA]:[PMPC₂₇ CTA]:[VA044] molar ratio of 270:1:0.33. The polymerization proceeded at 50° C. for 180 min to yield the desired carboxylic acid-functionalized PMPC₂₇-PHPMA₂₈₀ diblock copolymer. To induce the diblock copolymer to self-assemble into nanofibers (“wormlike” micelles), polymerization was carried out at a solid content of 25 wt/wt %. ¹H NMR spectroscopy (in CD₃OD) confirmed the complete polymerization of the HPMA monomer under the optimized conditions. The pH of the as-prepared COOH-PMPC₂₇-PHPMA₂₈₀ diblock copolymer was determined to be approximately 4.0 after being diluted to 5 wt/wt % with deionized water. Rhodamine-labeled PMPC₂₇-(PHPMA₂₈₀-stat-RhodMA_{0.2}) diblock copolymer was prepared using the same method except that 0.2 molar equivalents of methacryloxyethyl thiocarbamoyl rhodamine B (Polysciences, Inc.) was added to the HPMA during the second chain extension step.

Functionalization of Carboxylic Acid-Functionalized NFs with Sulfo DBCO-Amine

Carboxylic acid-functionalized NFs were functionalized with sulfo DBCO-amine via an amine-NHS ester coupling reaction. Briefly, a measured amount of carboxylic acid-functionalized NFs was diluted to 5 wt/wt % with deionized water before being activated with molar equivalents (vs. the COOH end-group) of 1-ethyl-3-(3-dimethylaminopropy)carbodiimide (EDC, 40 mg/mL in water) and N-hydroxysulfosuccinimide (sulfo-NHS, 40 mg/mL in water) at 20° C. for 1 h. The activated NFs were purified via 3 centrifugation (20,000 g, 20 min)-redispersion cycles to remove the unconsumed reagents. At the final centrifugation-redispersion cycle, the sulfo-NHS-activated NFs were suspended in 1×PBS. The sulfo-NHS-activated NFs were allowed to react with 50 molar equivalents (vs. the COOH end-group) of sulfo DBCO-amine (catalog number: 1227; Click Chemistry Tools) (20 mg/mL in water) at pH 8.5 (in 0.1 M PBS) and 20° C. in the dark for 12 h. The DBCO-functionalized NFs were purified via equilibrium dialysis against water for 6 cycles.

### Preparation of Azide-Functionalized PD-L1-Ig, CD86-Ig and Isotype IgG

PD-L1-Ig fusion protein (UniProt accession number: Q9EP73, mouse IgG1; catalog number: Pr00112-1.9; Absolute Antibody NA), CD86-Ig fusion protein (UniProt accession number: P42082, mouse IgG1; catalog number: Pr00226-1.9; Absolute Antibody NA) and mouse IgG1 isotype control (clone: MOPC-21, catalog number: BE083, BioXCell) were functionalized with azide-PEG8-NHS ester (catalog number: QBD10503; Sigma) via amine-NHS ester coupling reaction. The target degree of functionalization was 40, and the actual degree of functionalization was approximately 8 after purification via Pierce Polyacrylamide Spin Desalting Columns (7K MWCO; ThermoFisher), as determined by UV-visible spectroscopy after reaction with 50 molar equivalents of sulfo-DBCO MB488 (catalog number: 11905; Click Chemistry Tools) and purification via spin desalting columns. Rhodamine- or fluorescein-labeled fusion proteins were also prepared for quantitative studies in the presence of 4 molar equivalents (vs. the fusion protein) of rhodamine B isothiocyanate (catalog number: 283923; Sigma) or fluorescein isothiocyanate (catalog number: 46950; Sigma) during functionalization with azide-PEG8-NHS.

Fabrication of PD-L1 and/or CD86 NFs

PD-L1, CD86, and PD-L1/CD86 NFs were prepared via stain-promoted 1,3-dipolar cycloaddition. The target degree of functionalization for mono-functionalized NFs was 10 μg of azide-functionalized PD-L1-Ig or CD86-Ig (mono-functionalized NFs) per 1 mg of DBCO-functionalized NFs. The target degree of functionalization for dual-functionalized NFs was 5 μg of azide-functionalized PD-L1-Ig plus 5 μg of azide-functionalized CD86-Ig per milligram of DBCO-functionalized NFs. The bioconjugation was carried out at 5 wt/wt % of NF solid content at 37° C. for 1 h, followed by 20° C. for another 1 h. Unconjugated fusion proteins were removed via 3 repeated centrifugations (20,000 g, 20 min)-redispersion cycles. Rhodamine-labeled azide-functionalized PD-L1-Ig and CD86-Ig were used for the quantification of ligand-conjugation efficiencies. The functionalized NFs were sterilized by 100 Gy of X-ray irradiation (via an XRD320 X-ray irradiator operated at 320 kV and 12.5 mA at a dose rate of 364 cGy/min) before in vitro and in vivo studies were conducted. Isotype mouse IgG-functionalized NFs were prepared via the sample method, except azide-functionalized isotype control IgG was used.

### Preparation of LEF NPs

Azide-functionalized LEF-encapsulated PEG-PLGA NPs were prepared via the nanoprecipitation method. The NPs were constructed from a polymer blend composed of 33.3 wt/wt % PLGA (Mn=35,000-45,000 Da; catalog number: AP045; Akina, Inc.), 33.3 wt/wt % mPEG-PLGA (Mn=3,000+36,000 Da; catalog number: AK029; Akina, Inc.), and 33.3 wt/wt % PLGA-PEG-N3 (Mn=15,000+5,000 Da; catalog number: A1085; Akina, Inc.). The target LEF (EP Reference Standard; catalog number: Y0000654; Sigma) loading was 10 wt/wt %, and the actual LEF loading was 8.7±1.5 wt/wt %, as determined by fluorescence spectroscopy. Cy5-labeled LEF NPs were prepared using a polymer blend composed of Cy5-labeled PLGA (Mn=30,000-55,000 Da; catalog number: AV034; Akina, Inc.) instead of non-fluorescently labeled PLGA for the quantification of LEF NPs conjugated to the DBCO-functionalized NFs. Control drug-free azide-functionalized PEG-PLGA NPs were prepared via the same method, except no drug was added to the polymer blend for the preparation of NPs.

### Fabrication of LEF NFs

NFs were fabricated via stain-promoted 1,3-dipolar cycloaddition. The target degree of functionalization was 200 μg of azide-functionalized LEF NPs per milligram of DBCO-functionalized NFs. The bioconjugation was carried out at 5 wt/wt % solid content at 37° C. for 1 h, followed by 20° C. for another 1 h. Unconjugated LEF NPs were removed via 3 repeated centrifugations (20,000 g, 20 min)-redispersion cycles. NFs functionalized with Cy5-labeled LEF NPs were prepared via the same method for quantification of LEF NP conjugation efficiency via fluorescence spectroscopy. The actual quantity of LEF NPs conjugated to the NFs was 118±8 μg pf NPs per milligram of NFs, as quantified via fluorescence spectroscopy. Thus, each milligram of LEF NFs contained approximately 10 μg of encapsulated LEF. The functionalized NFs were sterilized by 100 Gy of X-ray irradiation (via an XRD320 X-ray irradiator operated at 320 kV and 12.5 mA at a dose rate of 364 cGy/min) before in vitro and in vivo studies were conducted. Control drug-free NP-functionalized NFs were prepared via the same method, except drug-free azide-functionalized PEG-PLGA NPs were used instead of LEF NPs.

### Fabrication of TGF-β1 Mimic NFs

Cationic TGF-β1 mimetic peptide (ACESPLKRQCGGG; purity >95%; LifeTein, LLC) was physically adsorbed onto the anionic (unmodified) COOH-functionalized NFs at a target loading of 50 μg peptide per milligram of NFs. Prior to adsorption, the peptide was first dissolved in 1×PBS at 20 mg/mL and incubated at 20° C. for 4 h to allow oligomerization of the thiol-rich peptide. The peptide solution was then added to the NF solution (final concentration=50 mg/mL) and incubated at 20° C. for 2 h. Rhodamine-labeled TGF-β1 mimetic peptide (ACESPLKRQCGGGS-Lys(Rhodamine B); purity >95%; LifeTein, LLC) was adsorbed onto the non-labeled NFs through the same method for in vitro release and T cell uptake studies. The functionalized NFs were sterilized by 100 Gy of X-ray irradiation (via an XRD320 X-ray irradiator operated at 320 kV and 12.5 mA at a dose rate of 364 cGy/min) before in vitro and in vivo studies were conducted.

### Mouse Colon Tissue Decellularization

Decellularized colon ECM was isolated from healthy C57BL/6 mice (female, 6-7 weeks old) through a detergent-facilitated spin-decellularization protocol as previously reported. Briefly, freshly preserved colons were washed three times with 1×PBS before being incubated in 1% sodium dodecyl sulfate (SDS; ThermoFisher) in deionized water for 4 h with gentle shaking, during which the SDS solution was changed once. The colons were then incubated with 1% Triton-X100 (Thermo Fisher) for another 1 h with gentle shaking. The acellular colons were then washed six times (20 min per wash) with 1×PBS (Gibco) supplemented with 1× Anti-Anti (Gibco). The acellular colons were than incubated in 1×PBS supplemented with 1× Anti-Anti at 37° C. for 24 h to destroy the residual microbes. The decellularized colons were then washed another 6 times with 1×PBS (5 min per wash) before being lyophilized. Lyophilized colons were then ball-milled (50 MHz, 5 min, 4° C.) into fine powders. The lyophilized colons were stored at −80° C. for up to 6 months before being used in the in vitro and in vivo studies. The DNA contents of freshly preserved mouse colon and decellularized COL ECM were determined by Promega QuantiFluor dsDNA binding dye (catalog number: E2671, Promega) according to the manufacturer's protocol. Prior to all in vitro and in vivo studies, the decellularized colon ECM was suspended in serum-free high-glucose DMEM (for in vitro study) or 1×PBS (for in vivo study) and sterilized via 100 Gy of X-ray irradiation (via an XRD320 X-ray irradiator operated at 320 kV and 12.5 mA at a dose rate of 364 cGy/min) before further use in in vitro and in vivo studies. Three batches of mouse colons, with each batch pooled from 40 mice, were preserved for the whole study. Decellularized mouse pancreas ECM was isolated through a similar spin-decellularization method as previously reported.

### NMR Spectroscopy

¹H NMR spectra of crude and purified samples were recorded in a Bruker AN400 NMR spectrometer in the Biochemistry Nuclear Magnetic Resonance Core at the University of Texas Southwestern Medical Center (UTSW).

### Transmission Electron Microscopy (TEM) and Scanning Electron Microscopy (SEM)

The morphologies of all NF samples were inspected through a JEOL 1400+ TEM microscope in the Electron Microscopy Core Facility at UTSW. An imaging study was performed on glow-discharged carbon-coated copper grids in which all samples were negatively stained with 1% phosphotungstic acid. The morphologies of decellularized COL ECM were inspected by SEM via an FEI Tecnai Spirit transmission electron microscope in the Electron Microscopy Core Facility at UTSW.

### Dynamic Light Scattering (DLS) and Aqueous Electrophoresis

Sphere-equivalent intensity-average diameters (Dh) and zeta potentials of all NF samples were measured in a Malvern Zetasizer Pro instrument.

### SAXS Measurements

Small-angle X-ray scattering measurements were performed in a Xenocs Ganesha 300-XL Small Angle Scattering Instrument in the Texas Materials Institute at the University of Texas at Austin. The SAXS instrument was equipped with a microfocus Cu-Kα radiation source operated at 50 kV and 0.6 mA. X-ray diffraction patterns were recorded by a Dectris 300K detector to record extremely small-angle scattering data. The scattering intensity I(q) was recorded in the interval 0.005 Å<q<0.015 Å⁻¹, where q is defined as q=4π/λ·sin(θ/2), in which θ is the scattering angle and A is the wavelength of the X-ray source. Solution samples were analyzed in sealed thin-walled capillary tubes under vacuum conditions at ambient temperature (20° C.). The acquisition time for each sample was approximately 90 min. Data were corrected to give absolute intensities by measuring I₀ directly on a Dectris detector. Two-dimensional scattering patterns were azimuthally integrated to a one-dimensional profile of intensity versus scattering vector using the two-dimensional data reduction utility SAXSGUI. Scattering patterns of the capillary tubes and PBS buffer or water (background solution) were collected and subtracted from the corresponding solution data. No attempt was made to convert the data to an absolute scale. Data analysis was performed by fitting the scattering curve to a polydisperse core-shell cylinder model using the least squares method in the IGOR Pro software pack (NIST, version 9.0; https://usaxs.xray.aps.anl.gov/software/irena) equipped with the Irena macro data analysis package.

### Proteomic Study

Matrisome and non-matrisome proteins in the native mouse colon samples, and decellularized colon ECM were analyzed using a Thermo Orbitrap Fusion™ Lumos™ Tribrid™ mass spectrometer in the UTSW Proteomic Core. Three biologically independent mouse colon specimens preserved at different times and three batches of decellularized colon ECM were analyzed. For quantitative comparison, the protein contents were determined by Pierce BCA Proteins Assay Kit (Thermo Fisher) prior to the proteomic study.

### Growth Factor Antibody Array

The relative levels of growth factors and cytokines in the non-decellularized mouse colon, decellularized mouse colon and decellularized mouse pancreas were quantified via the RayBio Human Growth Factor Antibody Array G-Series 1 by RayBiotech (Norcross, GA).

### Oscillatory Rheology

An oscillatory rheology study of unmodified PMPC₂₇PHPMA₂₈₀ diblock copolymer NFs (12.5 wt/wt % and 5 wt/wt %, in H₂O) was performed in a TA Instrument AR2000 rheometer from Akina, Inc. The sample was equilibrated at a desired temperature from 45 to 5° C. (step size: 4° C.) for 5 min before each temperature-dependent measurement. Each measurement was performed at an angular frequency of 1 rad/s⁻¹ and a stain amplitude of 1.0%.

### In Vitro Binding Study

To confirm that NF-conjugated PD-L1 and CD86 remained active, we performed an in vitro binding study with PE-labeled anti-PD-L1 (clone: MIH6; catalog number: 153611; BioLegend) and A488-labeled anti-CD86 (clone: GL-1; catalog number: 153611; BioLegend). Briefly, 0.5 mg of unmodified and functionalized NFs were incubated with 5 μg of PE-labeled anti-mouse PD-L1 antibody (clone: 10F.9G2; catalog number: 124308; BioLegend) and 5 μg of A488-labeled anti-mouse CD86 antibody (clone: GL-1; catalog number: 105018; BioLegend) in FACS buffer at 20° C. for 30 min. After removal of unbound antibodies via 3 centrifugation-redispersion cycles, the degree of labeling was quantified by a Calibur flow cytometer at the University Flow and Mass Cytometry Facility at UTSW.

### In Vitro Release Studies

The release of encapsulated LEF and physiosorbed rhodamine-labeled TGF-β1 mimetic peptide from the NFs was quantified via fluorescence spectroscopy (LEF: λex=280±15 nm, λem=410±15 nm; rhodamine: λex=550±15 nm, λem=590±15 nm). Briefly, 30 mg/mL of the NFs were incubated against 4 times their volume in 1×PBS (in the presence or absence of 5 mM of dithiothreitol (DTT)) through Slide-A-Lyzer MINI Dialysis Units (20,000 MWCO; ThermoFisher) at 37° C. in the dark. At the desired time points, a known volume of the sample was collected from the dialysis device and diluted to 5 mg/mL. The amount of LEF and rhodamine-labeled TGF-β1 retained in the NFs was quantified and compared with the fluorescent intensities of LEF and rhodamine in the crude NF samples via fluorescence spectroscopy.

The in vitro CD8⁺ T cell uptake of the released rhodamine-labeled TGF-β1 mimetic peptide from the TGF-β1 NFs was quantified by FACS and CLSM. CD8⁺ T cells were harvested from the splenocytes of healthy C57BL/6 mice through the EasySep™ Mouse CD8⁺ T Cell Isolation Kit (STEMCELL Technologies, Cambridge, MA) according to the manufacturer's instructions. Briefly, isolated CD8⁺ T cells (50,000 cells per well in a 96-well plate) were cultured with 10 μg of NFs containing 0.5 μg of TGF-β1 mimetic peptide in T cell culture medium (100 μL of medium per well) under physiological conditions for up to 48 h. At desired time points, the samples were removed, washed 3 times with FACS buffer, and fixed before FACS or CLSM studies were conducted. FACS samples were analyzed in a Calibur flow cytometer at the University Flow and Mass Cytometry Facility at UTSW. The CLSM images were recorded with a Zeiss LSM880 confocal microscope at the Quantitative Light Microscopy Core at UTSW.

### Cell Culture

Mouse primary colonic epithelial cells (catalog number: C57-6047, passage 1) isolated from healthy C57BL/6 mice were purchased from Cell Biologics, Inc. Primary mouse colonic epithelial cells and fibroblasts were cultured in Complete Epithelial Cell Medium with Supplement Kit (supplemented with 2% FBS, 5 ng/mL EGF, and 1× insulin-transferrin-selenium; catalog number: M6621-Kit; Cell Biologics, Inc.) in gelatin-coated tissue culture flasks. COL EPI cells at passage 4 were used for in vitro and in vivo studies. Mouse macrophages (catalog number: C57-2313F) isolated from C57BL/6 mice were purchased from Cell Biologics, Inc. Macrophages were cultured in RPMI-1640 tissue culture medium (Gibco) supplemented with 10% FBS, and 1× Anti-Anti (Gibco). The primary cells were cultured in plasma-treated tissue culture flasks or well plates. MCDs were detached and bypassed using scraping method.

B16F10 cell lines were obtained from the Tissue Culture Facility at the University of North Carolina (UNC) Lineberger Comprehensive Cancer Center at the UNC School of Medicine under the Material Transfer Agreement. B16F10 cells were cultured in high-glucose DMEM (4.5 g/L glucose; Gibco), 10% FBS (Corning), and 1× Anti-Anti (Gibco). Passage 13 or 14 B16F10 cells was used in the in vitro and in vivo studies.

MC38 cells were purchased from Kerafast. These cells were cultured in high-glucose DMEM supplemented with 10% FBS, 1× Anti-Anti (Gibco), 0.1 mM of non-essential amino acids (Gibco), 1 mM of sodium pyruvate, 2 mM GlutaMAX (Gibco), 10 mM HEPES buffer (Gibco), and 0.5 mg/mL gentamicin (Gibco) for positive selection. MC38 cells at passage 8 or 9 were used in the in vitro and in vivo studies.

B16OVA cells were kindly provided by Dr. Jonathan Serody in the UNC Lineberger Comprehensive Cancer Center at the UNC School of Medicine. B16OVA cells were cultured under positive selection conditions in RPMI 1640 culture medium (Gibco) supplemented with 10% FBS (Corning), 1× Anti-Anti (Gibco), 2 mM of GlutaMAX (Gibco), 0.1 mM non-essential amino acid (Gibco), 1 mM sodium pyruvate (Gibco), and 0.5 mg/mL Geneticin (Gibco) for positive selection. B16OVA cells at passage 15 or 16 were used in the in vitro and in vivo studies.

MIN6 cells were purchased from American Type Culture Collection. These cells were cultured in high-glucose DMEM supplemented with 20% FBS (Corning) and 1× Anti-Anti (Gibco). MIN6 cells at passage 29 were used in the in vivo study.

Primary colon epithelial cells were bypassed using TrypLE cell dissociation buffer (Gibco) and detached according to the manufacturer's protocol. All cell lines were bypassed using 0.05% trypsin (Gibco) according to the manufacturer's protocol. Mφs were detached and bypassed using the scraping method.

Unless specified, red blood cells were depleted from splenocytes via ACK Lysing Buffer (Gibco) prior to conducting all in vitro studies. Red blood cell-depleted splenocytes and isolated CD8⁺ T cells were cultured in RPMI 1640 medium (Gibco) supplemented with 10% FBS (Sigma), 1× Anti-Anti (Gibco), and 2 mM of GlutaMAX (Gibco).

### In Vitro Cell Proliferation Studies

The in vitro toxicities of different functionalized NFs to COL EPI cells were evaluated using a 3-(4,5-dimethylthiazol-2-yl)-5-(3-carboxymethoxyphenyl)-2-(4-sulfophenyl)-2H-tetrazolium (MTS) assay. Briefly, COL EPI cells (10,000 cells per well, 1/100 time used in the in vivo study) were cultured with unconjugated PD-L1 (0.1 μg/well, or 0.05 μg/well for the combo formulation), unconjugated CD86 (0.1 μg/well, or 0.05 μg/well for the combo formulation), small-molecule LEF (0.1 μg/well), free TGF-β1 mimetic peptide (0.5 μg/well) or 10 μg/well of each functionalized NF (or a total of 30 μg/well combining all three different functionalized NFs) in complete COL EPI cell culture medium (100 μL of medium per well) in a gelatin-coated 96-well plate for 3 days. The viabilities of COL EPI cells were evaluated using the Promega CellTiter 96™ AQueous One Solution Cell Proliferation Assay (MTS assay) according to the manufacturer's protocol. The in vitro toxicity of copanlisib against B16OVA cells was determined by MTS assay after co-cultured with 0.1-10 μM copanlisib (catalog number: HY15346A100MG, Med Cehem Express, LLC.) in complete medium for 48 h.

The proliferation of COL EPI cells cultured in different quantities of decellularized colon ECM was evaluated by MTS assay. Briefly, a desired concentration of decellularized colon ECM suspended in phenol red-free high-glucose DMEM was added to non-treated 96-well plates (with half of the wells pre-coated with gelatin) to achieve 0-6 μg of ECM per well (50 μL of medium per well) and air-dried in a laminar airflow tissue culture hood at 20° C. for 48 h. The plate was then sterilized via 100 Gy of X-ray irradiation (via an XRD320 X-ray irradiator operated at 320 kV and 12.5 mA at a dose rate of 364 cGy/min) before being briefly washed twice with 1×PBS. COL EPI cells, either suspended in complete COL EPI cell culture medium (supplemented with 2% FBS and growth factors) or suspended in COL EPI cell culture medium with half of the supplements, were added to the ECM-coated plate at a cell density of 2,000 cells per well and cultured for 8 days. Representative optical microscope images were recorded at day 8 through a Nikon Digital Sight optical microscope to record the growth conditions. Cell proliferation was evaluated using the Promega CellTiter 96™ AQueous One Solution Cell Proliferation Assay (MTS assay) according to the manufacturer's protocol. The viability of COL EPI cell growth in gelatin-coated wells in complete cell culture medium (in the absence of colon ECM) was used as a 100% viable reference.

### In Vitro T Cell Assays

Naïve mouse CD8⁺ T cells were isolated from healthy C57BL/6 mice (female, 8-10 weeks old) using EasySep™ Mouse CD8⁺ T Cell Isolation Kit (STEMCELL Technologies, Cambridge, MA) according to the manufacturer's instructions. In the T cell proliferation study, naïve CD8⁺ T cells were further labeled with carboxyfluorescein succinimidyl ester (CFSE) via CellTracer CFSE (Invitrogen) according to the manufacturer's protocol. TNP-specific (sensitized) CD8⁺ T cells were generated via an established method. Briefly, naïve mouse splenocytes were preserved from healthy C57BL/6 mice (female, 8 to 10 weeks old). To obtain TNP-sensitized splenocytes, TNP-modified splenocytes were first engineered by incubating naïve splenocytes with 2,4,6-trinitrobenzene sulfonic acid in HBBS buffer for 15 min according to the published protocol. Native splenocytes were sensitized by incubation with TNP-modified splenocytes at a 1:20 ratio for 4 days. TNP-sensitized splenocytes were used for in vitro studies, and sensitized CD8⁺ T cells were further isolated. TNP-modified COL EPI cells for antigen-specific studies were generated by incubating TNBS in HBBS buffer for 15 min according to the published protocol. To quantify the viability of the COL EPI cells after being co-cultured with splenocytes, the COL EPI cells were loaded with calcein via calcein acetoxymethyl cell-permeant dye (Thermo Fisher), according to the manufacturer's protocol, prior to the viability study.

To evaluate the inhabitation efficiencies of different ISM NFs and COL ECM in naïve CD8⁺ T cells, CFSE-labeled naïve CD8⁺ T cells (5×10⁴ cells per well in a 96-well plate) were stimulated using Dynbeads Mouse T-Activator CD3/CD28 (T cell activation beads; Gibco) at a 1:1 molar ratio with 1 U/well of recombinant mouse IL-2 (Gibco) in complete T cell culture medium (phenol red-free RPMI 1640 culture medium supplemented with 10% FBS, 1× Anti-Anti, and 1× GlutaMAX Supplement) in the presence of 0.1 μg (total amount of Ig, e.g., 0.05 μg of PD-L1-Ig plus 0.05 μg of CD86-Ig)/well of free or NF-conjugated PD-L1-Ig and/or CD86-Ig, 0.1 μg/well of small-molecule or NF-loaded LEF, 0.5 μg/well of free/NF-bound TGF-β1 mimetic peptide, and/or 5 μg/well of COL ECM. Further in vitro study was performed at one-fourth doses of COMBO ISM NFs and COL ECM, i.e., each well contained the combination of PD-L1/CD86 NFs containing 12.5 ng of NF-conjugated PD-L1-Ig and 12.5 ng of NF-conjugated CD86-Ig, LEF NFs containing 25 ng of encapsulated LEF and TGF-31 mimic NFs containing 125 ng of TGF-β1 mimetic peptide, and/or 1.25 μg/well of colon ECM. The cells were allowed to culture for 48 h. The stimulated T cells were then analyzed in a Calibur flow cytometer or LSRII flow cytometer at the University Flow and Mass Cytometry Facility at the UTSW. Each FACS analysis contained 20,000 singlet cells.

To evaluate the expression of inhibitory markers (exhaustion markers) on the surface of CD8⁺ T cells after being cultured with different ISM NFs, naïve CD8⁺ T cells (5×10⁴ cells per well in a 96-well plate) were stimulated using Dynbeads Mouse T-Activator CD3/CD28 (T cell activation beads; Gibco) at a 1:1 molar ratio with 1 U/well of recombinant mouse IL-2 (Gibco) in complete T cell culture medium (phenol red-free RPMI 1640 culture medium supplemented with 10% FBS, 1× Anti-Anti, and 1× GlutaMAX Supplement) in the presence of 0.1 μg (total amount of Ig, e.g., 0.05 μg of PD-L1-Ig plus 0.05 μg of CD86-Ig)/well of free or NF-conjugated PD-L1-Ig and/or CD86-Ig, 0.1 μg/well of small-molecule or NF-loaded LEF, 0.5 μg/well of free/NF-bound TGF-β1 mimetic peptide, and/or 5 μg/well of colon ECM. The cells were allowed to culture for 48 h. The T cells were then stained with PE-labeled anti-mouse TIM-3 antibody (clone: RMT3-23; catalog number: 119704; BioLegend) and APC-labeled anti-mouse LAG-3 antibody (clone: C9B7W; catalog number: 125210; BioLegend) to quantify the inhibitory markers. Stained T cells were then analyzed in a Calibur flow cytometer or LSRII flow cytometer at the University Flow and Mass Cytometry Facility at UTSW. Each FACS analysis contained 20,000 singlet cells.

To demonstrate that the COMBO ISM NFs and COL ECM inhibit colon antigen-specific CD8⁺ T cell activation, non-sensitized and TNP-sensitized splenocytes (1.5×10⁵ cells per well in a 96-well plate) were cultured with pre-seeded TNP-modified COL EPI cells (1×10⁴ cells per well in a 96-well plate) (or pre-seeded unmodified COL EPI cells as a control) in the presence of (i) COMBO ISM NFs: 10 μg/well of PD-L1/CD86 NFs (containing 0.05 μg of PD-L1-Ig and 0.05 μg of CD86-Ig), 10 μg/well of LEF NFs (containing 0.1 μg of encapsulated LEF), and 10 μg/well TGF-β1 mimic NFs (containing 0.5 μg of TGF-β1 mimetic peptide), (ii) COMBO ISMs: 0.05 μg/well of unconjugated PD-L1-Ig, 0.05 μg/well of unconjugated CD86-Ig, 0.1 μg/well of small-molecule LEF, and 0.5 μg/well of TGF-β1 mimetic peptide, (iii) 5 μg/well of COL ECM, and (iv) the combination of COMBO ISM NFs and colon ECM. The cells were cultured for 48 h and then stained first with A488-labeled anti-mouse CD8a antibody (clone: 53-6.7; catalog number: 100723; BioLegend), fixed with 10% neutral buffered formalin, and permeabilized with Intracellular Staining Permeabilization Wash Buffer (BioLegend) before being stained with PE-labeled anti-mouse IFN-gamma antibody (clone: XMG1.2; catalog number: 505808; BioLegend) and APC-labeled anti-human/mouse granzyme B recombinant antibody (clone: OA16A02; catalog number: 372204; BioLegend) to quantify the T cell activation markers. Alternatively, cells were stained with A488-labeled anti-mouse CD8a antibody (clone: 53-6.7; catalog number: 100723; BioLegend), PE-labeled anti-mouse TIM-3 antibody (clone: RMT3-23; catalog number: 119704; BioLegend), and APC-labeled anti-mouse LAG-3 antibody (clone: C9B7W; catalog number: 125210; BioLegend) to evaluate the T cell inhibitory markers on the surface of the sensitized CD8⁺ T cells. Stained cells were then analyzed in a BD LSR II flow cytometer at the University Flow and Mass Cytometry Facility at UTSW. Each FACS analysis contained 20,000 singlet cells.

To demonstrate that the COMBO ISM NFs and COL ECM inhibit colon antigen-specific CD8⁺ T cell activation, non-sensitized and TNP-sensitized splenocytes (1.5×10⁵ cells per well in a 96-well plate) were cultured with pre-seeded TNP-modified COL EPI cells (1×10⁴ cells per well in a 96-well plate) (or pre-seeded unmodified COL EPI cells as a control) in the presence of (i) COMBO ISM NFs: 10 μg/well of PD-L1/CD86 NFs (containing 0.05 μg of PD-L1-Ig and 0.05 μg of CD86-Ig), 10 μg/well of LEF NFs (containing 0.1 μg of encapsulated LEF), and 10 μg/well TGF-β1 mimic NFs (containing 0.5 μg of TGF-β1 mimetic peptide), (ii) COMBO ISMs: 0.05 μg/well of unconjugated PD-L1-Ig, 0.05 μg/well of unconjugated CD86-Ig, 0.1 μg/well of small-molecule LEF, and 0.5 μg/well of TGF-β1 mimetic peptide, (iii) 5 μg/well of COL ECM, and (iv) the combination of COMBO ISM NFs and colon ECM. The cells were cultured for 48 h and then stained first with A488-labeled anti-mouse CD8a antibody (clone: 53-6.7; catalog number: 100723; BioLegend), fixed with 10% neutral buffered formalin, and permeabilized with Intracellular Staining Permeabilization Wash Buffer (BioLegend) before being stained with PE-labeled anti-mouse IFN-gamma antibody (clone: XMG1.2; catalog number: 505808; BioLegend) and APC-labeled anti-human/mouse granzyme B recombinant antibody (clone: OA16A02; catalog number: 372204; BioLegend) to quantify the T cell activation markers. Alternatively, cells were stained with A488-labeled anti-mouse CD8a antibody (clone: 53-6.7; catalog number: 100723; BioLegend), PE-labeled anti-mouse TIM-3 antibody (clone: RMT3-23; catalog number: 119704; BioLegend), and APC-labeled anti-mouse LAG-3 antibody (clone: C9B7W; catalog number: 125210; BioLegend) to evaluate the T cell inhibitory markers on the surface of the sensitized CD8⁺ T cells. Stained cells were then analyzed in a BD LSR II flow cytometer at the University Flow and Mass Cytometry Facility at UTSW. Each FACS analysis contained 20,000 singlet cells.

The change in the number of IFN-gamma secreting cells cultured in the presence of COMBO ISM NFs was quantified via a mouse IFN-gamma ELISpot PLUSE Kit (ALP) (Mabtech, Pittsburgh, PA). In an initial study, non-sensitized and TNP-sensitized splenocytes (1.5×10⁵ cells per well in a 96-well ELISpot plate) were cultured with TNP-modified COL EPI cells (1×10⁴ cells per well in a 96-well ELISpot plate) (or pre-seeded unmodified COL EPI cells as a control) in the presence of (i) COMBO ISM NFs: 10 μg/well of PD-L1/CD86 NFs (containing 0.05 μg of PD-L1-Ig and 0.05 μg of CD86-Ig), 10 μg/well of LEF NFs (containing 0.1 μg of encapsulated LEF), and 10 μg/well TGF-β1 mimic NFs (containing 0.5 μg of TGF-β1 mimetic peptide) or (ii) COMBO ISMs: 0.05 μg/well of unconjugated PD-L1-Ig, 0.05 μg/well of unconjugated CD86-Ig, 0.1 μg/well of small-molecule LEF, and 0.5 μg/well of TGF-β1 mimetic peptide. The cells were cultured in the IFN-gamma ELISpot plate for 48 h. The number of IFN-gamma-positive spots were generated via the manufacturer's protocol and counted through a Zeiss SteREO Discovery microscope. In further studies, TNP-sensitized splenocytes (1.5×10⁵ cells per well in a 96-well ELISpot plate) were cultured with TNP-modified COL EPI cells (1×10⁴ cells per well in a 96-well ELISpot plate) in the presence of one-fourth of immunosuppressants, i.e., COMBO ISM NFs containing PD-L1/CD86 NFs containing 12.5 ng of NF-conjugated PD-L1-Ig and 12.5 ng of NF-conjugated CD86-Ig, LEF NFs containing 25 ng of encapsulated LEF and TGF-β1 mimic NFs containing 125 ng of TGF-β1 mimetic peptide, and/or 1.25 μg/well of COL ECM.

The viabilities of calcein-loaded TNP-modified COL EPI cells (1×10⁴ cells per well in a black 96-well plate) after being co-cultured with naïve and TNP-specific splenocytes (1.5×10⁵ cells per well in a black 96-well plate) in the absence or presence of COMBO ISM NFs (or COMBO ISMs), and/or COL ECM (5 μg/well) were determined by quantifying the calcein retained in the COL EPI cells 48 h after co-treatment according to the manufacturer's protocol. The specific cell killing of each treatment group was calculated according to a published method⁷⁶. To demonstrate that the co-cultured COMBO ISM NFs induced CD8⁺ T cell exhaustion, the co-cultured splenocytes were further stained with APC-labeled anti-mouse CD8a antibody (clone: 53-6.7; catalog number: 100712; BioLegend) and PE-labeled anti-mouse TIM-3 antibody (clone: RMT3-23; catalog number: 119704; BioLegend). The stained cells were then mounted with ProLong™ Diamond Antifade Mountant with DAPI and imaged through a Zeiss LSM880 confocal microscope at the Quantitative Light Microscopy Core at UTSW.

### In Vitro Mφ Assays

M1 Mφs were obtained by culturing mouse M0 Mφs in complete Mφ culture medium supplemented with 50 ng/mL recombinant IFN-γ (catalog number: RP8617, Invitrogen) and 1 μg/mL LPS (catalog number: 497603, Invitrogen) for 36 h, as previously reported77, 78. The induced M1 Mφs were washed three times with RPMI-1640 (Gibco) before detachment via the scraping method for further studies.

To demonstrate that COMBO ISM NFs and COL ECM can directly polarize M1 Mφs to M2 Mφs, induced M1 MCs (1×10⁴ cells per well in a coverglass chamber (catalog number: GBL112358, Grace Bio-Lab), same surface area as in 96-well plate) were cultured with 0.1 μg (total amount of Ig, e.g., 0.05 μg of PD-L1-Ig plus 0.05 μg of CD86-Ig)/well of NF-conjugated PD-L1-Ig and/or CD86-Ig, 0.1 μg/well of NF-loaded LEF, 0.5 μg/well of NF-bound TGF-β1 mimetic peptide, and/or 5 μg/well of colon ECM for 48 h. The cells were washed and stained with PE-labeled anti-mouse CD86 antibody (clone: GL-1, catalog number: 105007). The washed and stained cells were then fixed with 10% neutral buffered formalin, and permeabilized with Intracellular Staining Permeabilization Wash Buffer (BioLegend) before being stained with Alexa Fluor 488-labeled anti-mouse Arginase 1 antibody (clone: 658922; catalog number: IC8026G; R&D Systems). The stained cells were then mounted with ProLong™ Diamond Antifade Mountant with DAPI and imaged through a Zeiss LSM880 confocal microscope at the Quantitative Light Microscopy Core at UTSW.

The viabilities of calcein-loaded TNP-modified COL EPI cells (1×104 cells per well in a black 96-well plate) after being co-cultured with M1 Mφs (1×104 cells per well in a black 96-well plate) in the absence or presence of COMBO ISM NFs (0.05 μg/well of NF-conjugated PD-L1-lg, 0.05 μg/well of NF-conjugated CD86-Ig, 0.1 μg/well of NF-loaded LEF and 0.5 μg/well of NF-bound TGF-β1 mimetic peptide), colon ECM (5 μg/well), or their combination were determined by quantifying the calcein retained in the COL EPI cells 48 h after co-treatment according to the manufacturer's protocol. The cell killing of each treatment group was calculated according to a previously published method.

### In Vivo Studies

Animals were maintained in the Animal Resource Center at UTSW. All procedures involving the experimental animals were performed following the protocol approved by UTSW Institutional Animal Care and Use Committee and conformed to the Guide for the Care and Use of Laboratory Animals (NIH publication no. 86-23, revised 1985). Wild-type C57BL/6 mice (nomenclature: C57BL/6NCrl) were purchased from Charles River Laboratories, Inc. (Houston, TX). IL10 KO mice (B6 background, nomenclature: B6.129P2-Il10tm1Cgn/J) and wild-type C57BL/6 mice (also known as B6 WT, nomenclature: C57BL/6J) were purchased from the Jackson Laboratory for the IL10 KO mouse study. Mice were randomized, eartagged, and grouped on arrival. Grouped mice were housed in the conventional non-sterilized mouse facility for at least 1 week before all in vivo studies were conducted.

### Induction and Assessment of DSS-Induced Colitis

Three mouse colitis models were used in this study. For the DSS-induced model, DSS colitis was induced according to a previously established protocol. Mice were housed in groups of 4 or 5 and acclimatized for at least 7 days before the study. To induce colitis, C57BL/6 mice (7 to 8 weeks old) were fed 3% DSS (molecular weight c.a. 40,000; catalog number: J63606.22; Thermo Scientific) supplemented with deionized water (catalog number: W12-4; Fisher Scientific) for 6 days. Mouse cages were changed before and after the induction of colitis. Unless specified, mouse cages were changed weekly afterward. The bodyweight and disease activity index (DAI) scores were monitored before the induction of colitis and then daily from day 4 after the induction of colitis.

For the TNP-induced model, colitis was induced according to a previously published protocol. Mice were housed in groups of 4 or 5 and acclimatized for at least 7 days before the study. To induce colitis, mice received two intrarectal injections of 100 μL of 2.5% (w/v) trinitrobenzene sulfonate (TNBS) in 1:1 water/ethanol at day 0 (sensitization) and day 14 (colitis induction). Mice were starved for 18 h prior to each intrarectal injection. Mouse cages were changed weekly or the day after the intrarectal injection. The bodyweight and DAI scores for the TNP-induced colitis mice were monitored every two days from day 0 and daily from day 13 of the study.

IL10 KO mice spontaneously develop colitis. 4-week s old IL10 KO mice and B6 WT (control) mice were purchased from the Jackson Laboratory. All mice were born within 3 days. Mice were housed in groups of 4 or 5 and were ear tagged for identification. Mouse cages were changed weekly. Once onset, colitis mice house with colitis mice. The bodyweight and DAI scores were monitored every 2 or 3 days after the study began. Whole blood (~100 μL) was collected from the facial vein at 7, 9, 11, 13, 15 and 17 weeks of age for complete blood count. IL10 KO mice that showed colitis symptoms at 10-11 weeks of age were used in this study.

The DAI score was calculated by combining the score of percentage of bodyweight loss (0=no weight loss from baseline, 1=less than 5% weight loss, 2=5-10% weight loss, 3=10-20% weight loss, and 4=more than 20% weight loss), diarrhea (0=normal stool, 2=semi-formed stool, 4=liquid that adheres to the anus), and rectal bleeding (0=no bleeding, 2=positive hemoccult test, and 4=gross bleeding). For statistical analysis, the DAI scores and bodyweights of DSS colitis mice were compared based on data recorded up to day 10 (4 days after the therapeutic treatment). Unless specified, DSS colitis mice were euthanized at days 12-14, and colons were preserved by trained veterinary technicians at the UTSW ARC Diagnostic Laboratory without prior knowledge of the treatment. For the TNP-induced colitis mice, DAI scores were compared daily from day 16 to day 20. TNP colitis mice were euthanized at day 20, and colons were preserved by trained veterinary technicians at the UTSW ARC Diagnostic Laboratory without prior knowledge of the treatment. For the IL10 KO mice, DAI scores were compared based on data collected up to 17 weeks of age (endpoint). IL10 KO mice were euthanized at 17 weeks of age, and colons were preserved by trained veterinary technicians at the UTSW ARC Diagnostic Laboratory without prior knowledge of the treatment. Unless specified, the lengths of preserved colons were measured, and fixed with 10% NBF for 24 h. Fixed specimens were kept in 70% ethanol and transferred to the UTSW Histo Pathology Core or IDEXX BioAnalytics (Columbia, MO) for paraffin embedding before further histological studies.

### In Vivo Evaluation of Different Immune Niche Formulations for Amelioration of DSS Colitis in a Mouse Model

Therapeutic colitis treatment was performed at day 6 after the induction of colitis. COINs were engineered by gently mixing immunosuppressive NFs (1 mg/mouse PD-L1/CD86 NFs, 1 mg/mouse LEF NFs and/or 1 mg/mouse TGF-β1 mimic NFs in 1×PBS), ball-milled decellularized colon ECM (0.5 mg/mouse in 1×PBS), and COL EPI cells (1×10⁶ cells/mouse, in 1×PBS) together prior to conducting the in vivo studies. COIN suspensions (200 μL per mouse) were s.c. inoculated into the neck fat pocket via a 1 mL syringe equipped with a 21-gauge needle. In the control studies, DSS colitis mice received s.c. treatment with: (i) COL EPI cells (1×10⁶ cells/mouse), colon ECM (0.5 mg/mouse) and isotype control NFs (1 mg/mouse of IgG isotype control NFs, 1 mg/mouse of drug-free azide PEG-PLGA NP-functionalized NF and 1 mg/mouse non-functionalized NFs); (ii) COL EPI cells (1×10⁶ cells/mouse) and COMBO ISM NFs (1 mg/mouse of PD-L1/CD86 NFs, 1 mg/mouse of LEF NF and 1 mg/mouse TGF-β1 mimic NFs); (iii) colon ECM (0.5 mg/mouse); (iv) colon ECM (2 mg/mouse); (v) free immunosuppressants (5 μg of PD-L1-Ig, 5 μg of CD86-Ig, 120 μg/mouse of LEF NPs and 50 μg of TGF-β1 mimetic peptide); (vi) MIM6 cells (1×10⁶ cells/mouse), pancreas ECM (0.5 mg/mouse) and COMBO ISM NFs (1 mg/mouse of PD-L1/CD86 NFs, 1 mg/mouse of LEF NF and 1 mg/mouse TGF-β1 mimic NFs). In another control study, DSS colitis mice received an i.p. treatment with COMBO ISM NFs (1 mg/mouse PD-L1/CD86 NFs, 1 mg/mouse LEF NFs and 1 mg/mouse TGF-β1 mimic NFs). The therapeutic effect was compared with that of s.c. treatment with COMBO COIN.

Unless specified, DSS colitis mice were euthanized on days 13-14, colons were preserved by trained veterinary technicians at the UTSW ARC Diagnostic Laboratory without prior knowledge of the treatment. Unless specified, the lengths of preserved colons were measured, and fixed with 10% NBF for 24 h. Fixed specimens were kept in 70% ethanol and transferred to the UTSW Histo Pathology Core or IDEXX BioAnalytics (Columbia, MO) for paraffin embedding before further histological studies. Subsequent H&E staining was performed by UTSW Histo Pathology Core or IDEXX BioAnalytics (Columbia, MO). Ki67 and TUNEL staining was performed by UTSW Histo Pathology Core. Further immunohistological studies were performed by the Pathology Services Core in the UNC Lineberger Comprehensive Cancer Center at the UNC School of Medicine or iHisto, Inc. (Salem, MA). Colon damage in each mouse was assessed from representative H&E-stained colon sections. Briefly, colon epithelial damage was assigned scores as follows: 0=normal; 1=hyperproliferation, irregular crypts; 2=mild to moderate crypt loss (10-50%); 3=severe crypt loss (50-90%); 4=complete crypt loss, surface epithelium intact; 5=small to medium size ulcer (<10 crypt widths); and 6=large ulcer (>10 crypt widths). An inflammatory cell infiltration score was assigned separately for the mucosa (0=normal, 1=mild, 2=modest, 3=severe), submucosa (0=normal, 1=mild to modest, 2=severe), and muscle/serosa (0=normal, 1=moderate to severe). Scores for epithelial damage and inflammatory cell infiltration were added, resulting in a total score ranging from 0 to 12. For selected experimental groups, feces were collected from mice 5 days after colitis treatment before euthanization. Preserved feces were submitted to the UTSW Microbiome Research Lab for 16S rRNA sequencing. Preserved colons were homogenized in (i) TrisHCl/Triton X-100-based lysis buffer in the presence of Thermo Scientific™ Halt™ Protease Inhibitor Cocktail (1×; ThermoFisher) for the quantification of mouse IL-6, mouse TNF-alpha, mouse IL-10, and mouse TGF-β1 expression in the preserved colon tissues in the Genomic and Microarray Core Facility at UTSW or (ii) hexadecyltrimethylammonium bromide buffer for the quantification of MPO expression through a Cayman Chemical Neutrophil Myeloperoxidase Activity Assay Kit according to the manufacturer's protocol.

For the immune cell depletion study, three doses of anti-mouse CD8a antibody (clone: YTS169.4; catalog number: BE0117; BioXCell) and anti-mouse CD25 antibody (clone: PC-61.5.3; catalog number: BE0012; BioXCell) were i.p. administered on days 4, 6, and 8 to deplete CD8⁺ T cells and regulatory T cells, respectively. The antibody dose was 200 μg/mouse per injection. A single dose of Clodrosome® (140 μL, containing 700 μg of encapsulated clodronate; Encapsula NanoSciences, Brentwood, TN) was i.v. tail vein-administered at day 5 to deplete macrophages.

The chronic DSS colitis study was performed as previously reported. Mice were subjected to 4 cycles of 3% DSS treatment for 5 to 6 days, followed by 14 days of normal drinking water. Mice in the single treatment group received a single s.c. treatment with the combo immune niche at day 6. Mice in the multiple (triple) treatment group received s.c. treatment of COMBO COIN at days 6, 25, and 44. Each s.c. injection was composed of 1 mg of PD-L1/CD86 NFs, 1 mg of LEF NFs, 1 mg of TGF-β1 mimic NFs, 0.5 mg of decellularized colon ECM, and 1×10⁶ COL EPI cells. Mice were euthanized at day 48. Colons were preserved for further histological study.

IDSSAn ex vivo biodistribution study was performed to investigate the biodistribution of s.c. administered COMBO COIN and i.p. administered COMBO ISM NFs in DSS colitis mice via ex vivo fluorescence imaging. Similar to the therapeutic study, rhodamine-labeled COMBO COIN (constructed from rhodamine-labeled COMBO ISM NFs) and rhodamine-labeled COMBO ISM NFs were administered to DSS colitis mice at day 6 after the initial study. Mice were euthanized by an overdose of carbon dioxide 3 days after colitis treatment (day 9 after the initial study). The biodistribution of rhodamine-labeled NFs was quantified by an AMI HTX optical imaging system (Spectra Instrument Imaging). Selected organs were preserved to quantify the uptake of rhodamine-labeled NFs.

Immune profiling studies were performed on colon and s.c. immune niches preserved 3 (for immune niche), 7 (for colon and immune niche), and 12 days (for colon) after colitis treatment via mass cytometry. For the preserved colons, immune cells infiltrated into lamina propria cells were isolated using a previously reported method. Freshly isolated cells were transferred to the University Flow and Mass Cytometry Facility at UTSW for staining and subsequent flow mass cytometry study. For the preserved s.c. inoculated COMBO COIN, the preserved niches were fixed for 24 h before transfer to the UTSW Histo Pathology Core and University Flow and Mass Cytometry Facility at UTSW for imaging mass cytometry study. The antibodies used in the mass cytometry studies are summarized in Table 5.

### Table 5: Antibody Lists for Flow and Imaging Mass Cytometry Studies.

TABLE 5
Antibody lists for flow and imaging mass cytometry studies.
List of antibodies for Helios flow mass
cytometry

| Mouse Helios | | |
|---|---|---|
| Abs | | |
| Item Name * | Clone | metal |
| CD3e 152Sm | 145-2C11 | 152Sm |
| CD4 145Nd | RM4-5 | 145Nd |
| CD8a 168Er | 53-6.7 | 168Er |
| CD11b 148Nd | M1/70 | 148Nd |
| CD11c 142Nd | N418 | 142Nd |
| CD44 171Yb | IM7 | 171Yb |
| CD45 89Y | 30-F11 | 89Y |
| CD69 143Nd | H1.2F3 | 143Nd |
| CD86 172Yb | GL1 | 172Yb |
| CD206 175Lu | C068C2 | 175Lu |
| IA/IE 209Bi | m5/114.15.2 | 209Bi |
| Ly-6G 141Pr | 1A8 | 141Pr |
| NK1.1 170ER | PK136 | 170Er |
| F4/80 146Nd | BM8 | 146Nd |
| Foxp3 158Gd | FJK-16S | 158Gd |
| PD-1 159Tb | 29F.1A12 | 159Tb |
| IFNy 165Ho | XMG1.2 | 165Ho |
| TNFa 162Dy | MP6-T22 | 162Dy |
| CCR7 164Dy | 4B12 | 164Dy |

List of antibodies for Hyperion imaging
mass cytometry

| Mouse | | |
|---|---|---|
| Hyperion Abs | | |
| Item Name * | Clone | metal |
| CD4 159Tb | BLR167J | 159Tb |
| CD8 152Sm | EPR21769 | 146Nd |
| CD11b 149Sm | EPR1344 | 149Sm |
| CD11c 154Sm | 3.9 | 154Sm |
| CD45 141Pr | 30-F11 | 141Pr |
| CD69 161Dy | poly | 161Dy |
| CD44 144Nd | IM7 | 144Nd |
| CD86 156Gd | GL-1 | 156Gd |
| F4/80 153Eu | D2S9R | 153Eu |
| IA-IE 147Sm | M5/114.15.2 | 147Sm |
| Gr-1 169Tm | RB6805 | 169TM |
| FoxP3 155Gd | FJK-16s | 155Gd |
| TNFa 162Dy | MP6-T22 | 162Dy |

### In Vivo Evaluation of TNP-Modified COMBO COIN for Amelioration of TNP-Induced Colitis in a Mouse Model

To demonstrate that the s.c. inoculated COMBO COIN can ameliorate TNP-induced colitis, therapeutic treatment was performed 1 day after the second intrarectal injection of TNP (15 days after the initial study). Briefly, TNP-modified COMBO COIN composed of TNP-modified COL EPI cells (1×10⁶ cells per mouse), COL ECM (0.5 mg per mouse) and COMBO ISM NFs (containing 1 mg/mouse PD-L1/CD86 NFs, 1 mg/mouse LEF NFs and 1 mg/mouse TGF-β1 mimic NFs) was s.c. administered to the neck fat pocket of the TNP-induced colitis mice. Mice were monitored daily after the treatment. Mice were euthanized on day 20. Colons were preserved by trained veterinary technicians at the UTSW ARC Diagnostic Laboratory without prior knowledge of the treatment. The lengths of preserved colons were measured and fixed with 10% NBF for 24 h. Fixed specimens were kept in 70% ethanol and transferred to the UTSW Histo Pathology Core for histological study.

### In Vivo Evaluation of COMBO COIN for Amelioration of Colitis in IL10 KO Mice

To demonstrate that s.c administration of COMBO COIN can ameliorate colitis in IL10 KO mice that spontaneously develop colitis, IL10 KO mice that developed colitis at 10-11 weeks of age received repeated colitis treatment with COMBO COIN at 11.5, 13.5 and 15.5 weeks of age. Each treatment involved s.c. administration of COMBO COIN engineered from COL EPI cells (1×10⁶ cells per mouse), COL ECM (0.5 mg per mouse) and COMBO ISM NFs (containing 1 mg/mouse PD-L1/CD86 NFs, 1 mg/mouse LEF NFs and 1 mg/mouse TGF-β1 mimic NFs). Bodyweight and DAI scores were monitored every 2 or 3 days after treatment. Whole blood samples were collected every 2 weeks for complete blood count at the UTSW ARC Diagnostic Laboratory. Mice were euthanized at 17 weeks of age. Colons were preserved by trained veterinary technicians at the UTSW ARC Diagnostic Laboratory without prior knowledge of the treatment. The lengths of preserved colons were measured and fixed with 10% NBF for 24 h. Fixed specimens were kept in 70% ethanol and transferred to the UTSW Histo Pathology Core for histological study.

### In Vivo Evaluation of Immune Niches for Ameliorating Colitis-Associated Colorectal Cancer in an AOM/DSS Model of Colitis-Associated Cancer

The ability of the immunosuppressive immune niche to ameliorate colitis-associated colorectal cancer was evaluated in the established AOM/DSS colitis mouse model. At the beginning of the study, AOM (Sigma) pre-dissolved in 1×PBS (Gibco) was i.p. administered to a healthy group of C57BL/6 mice (female, 8 to 9 weeks old) at a dose of 10 mg/kg. Mice in the DSS colitis and AOM/DSS colitis groups were fed 3% DSS at days 7, 27, and 47 for 6 days. Mice in the treatment group received s.c. therapeutic treatment with COMBO COIN at days 13, 32, and 52. Each immune niche treatment was composed of 1 mg of PD-L1/CD86 NFs, 1 mg of LEF NFs, 1 mg of TGF-β1 mimic NFs, 0.5 mg of ball-milled decellularized COL ECM, and 1×10⁶ COL EPI cells. Bodyweight and DAI scores were monitored every 1 to 2 days for up to 65 days. Mice were then euthanized. Colons were preserved and washed with saline before being stained with 1% Alcian blue (Electron Microscopy Sciences) to label tumor lesions. The length of the colon was measured, and the number of tumors was counted. The colon was then washed 2 to 3 times with saline to remove the Alcian blue before being fixed with 10% NBF for further histological study at the Histo Pathology Core at UTSW.

### In Vivo Immune-Mediated Colitis Treatment in Syngeneic Tumor Models

B16F10, B16OVA, and MC38 xenograft tumors were inoculated into healthy C₅₇BL/6 mice (female, 7 to 8 weeks old) through s.c. inoculation of B16F10 cells (2.5×10⁵ cells/mouse) in serum-free DMEM, B16OVA cells (2.5×10⁵ cells/mouse) in serum-free RPMI 1640 culture medium, and MC38 cells (5×10⁵ cells/mouse) in serum-free DMEM in the right flank. Tumor volume (volume=0.5×a×b², where a is the large diameter and b is the small diameter) was measured every 2 to 3 days from day 7 after the tumor was inoculated.

For the COP-triggered colitis study in the B16OVA tumor model, COP treatment was provided by i.p. administration of COP (25 mg/kg, in saline) at days 6, 8, 10, 12, 14, 16 and 18 after tumor inoculation. Colitis was induced by feeding the tumor-bearing mice 3% DSS at day 14 for 4 days. For the colitis treatment group, mice received a single s.c. treatment with COMBO COIN at day 18. Bodyweight and DAI scores were monitored every 2 or 3 days. Mice were euthanized at 22 days post-inoculation. Colons were preserved by trained veterinary technicians at the UTSW ARC Diagnostic Laboratory without prior knowledge of the treatment. The lengths of preserved colons were measured and fixed with 10% NBF for 24 h. Fixed specimens were kept in 70% ethanol and transferred to the UTSW Histo Pathology Core for histological study (H&E, Ki67 and TUNEL stains).

For the B16F10 and B16OVA tumor models, ICB treatment was provided by i.p. administration of 100 μg of anti-mouse CTLA-4 antibody (clone: 9H10; catalog number: BE0131; BioXCell) and 100 μg of anti-mouse PD-1 antibody (clone: RMP1-14; catalog number: BP0146; BioXCell) at days 7 and 9 after the tumor was inoculated. For the MC38 tumor model, mice received ICB treatments at days 7 and 9, followed by maintenance treatments at days 19, 22, and 25. Colitis was induced by feeding the tumor-bearing mice 3% DSS at day 7 for 6 days. For the colitis treatment group, mice received a single s.c. treatment with COMBO COIN or i.p. treatment with COMBO ISM NFs at day 13. Each immune niche treatment was composed of 1 mg of PD-L1/CD86 NFs, 1 mg of LEF NFs, 1 mg of TGF-β1 mimic NFs, 0.5 mg of ball-milled decellularized COL ECM, and 1×10⁶ COL EPI cells.

For the B16F10 tumor model, mice were euthanized when the large diameter of the tumor was more than 2 cm, if they had suffered 10% weight loss in 2 days, or 21 days after tumor inoculation. Colons were preserved for further histological study. For the B16OVA tumor model, mice were euthanized at day 16 (3 days after colitis treatment). Xenograft tumors were preserved for mechanistic study to investigate the phenotypes of the tumor-infiltrated T cells. Briefly, a preserved tumor was digested with 2 mg/mL collagenase D (Roche) and 0.2 mg/mL DNase I (Roche) in HBBS buffer (10 mL per tumor). The dead cells were first stained with Live/Dead Fixable Green Dead Cell Stain Kit (catalog number: L23101; Invitrogen) before being incubated with PE-labeled OVA(257-264)-specific tetramer (catalog number: TB50011; MBL International Corporation) at 20° C. for 1 h. The stained cells were then divided into two portions before being stained with (i) PE/Cy5-labeled anti-mouse CD3 antibody (clone: 17A2; catalog number: 100274; BioLegend) and APC-labeled anti-mouse CD8a antibody (clone: 53-6.7; catalog number: 100712; BioLegend) or (ii) APC-labeled anti-mouse CD8a antibody (clone: 53-6.7; catalog number: 100712; BioLegend) and PE/Cy5-labeled anti-TIM-3 antibody (clone: F38-2E2; catalog number: 345052; BioLegend). Stained cells were analyzed in a Calibur flow cytometer at the University Flow and Mass Cytometry Facility at UTSW. For the MC38 tumor model, mice were euthanized when the large diameter of the tumor was greater than 2 cm, if they suffered 10% weight loss in 2 days, or 42 days after the tumor inoculation.

In a control study, B16F10 tumor-bearing mice received ICB treatments at days 7 and 9 and were fed 3% DSS drinking water from days 7 to 13. Mice were euthanized at day 11 or 13. Spleens were preserved. The splenocytes were then stained with FITC-labeled PD-L1-Ig, rhodamine-labeled CD86, and APC-labeled anti-mouse CD8a antibody (clone: 53-6.7; catalog number: 100712; BioLegend) to confirm that the CD8+ T cells can bind to PD-L1 and CD86. The stained cells were analyzed in a Calibur flow cytometer at the University Flow and Mass Cytometry Facility at UTSW.

### Statistical Analysis

No statistical methods were used to pre-determine the sample size of the experiment. The sample sizes were based on similar published in vitro and in vivo studies. No collected experimental data were excluded from the quantitative analysis. Quantitative data are expressed as the mean±standard error of the mean (SEM). One-way or two-way analysis of variance (ANOVA), followed by Tukey's honestly significant difference (HSD) multiple comparison post hoc test was used to test differences among groups. Statistical analyses were performed in Graph Pad Prism 6 software pack. A p-value <0.05 was considered statistically significant and a p-value >0.05 was considered statistically insignificant.

### Example 1. Bioengineering of Organ-Specific Immune Niches

To create a colon-like tissue microenvironment for modulating autoreactive immune cells, COINs were bioengineered from colon epithelial cells and decellularized colon ECM. The niche was further engineered with NFs containing immunosuppressive molecules (ISMs). The colon epithelial cells and decellularized colon ECM recapitulate a colon-like microenvironment to attract and interact with autoreactive immune cells involved in UC. The decellularized colon ECM also acts as a depot that gradually releases pro-regulatory growth factors and cytokines to modulate the activation of autoreactive T cells and Mφs. ISM NFs were engineered to simultaneously anergize autoreactive T cells and polarize Mφs through multiple immunosuppressive signaling pathways by incorporating immune checkpoint molecules and other immunosuppressants. These included PD-L1, which triggers the suppressive PD-1/PD-L1 immune checkpoint pathway and induces the polarization of M1 Mφs into M2 Mφs; CD86, which triggers the immunosuppressive CTLA-4 pathway in activated CD8⁺ T cells; immunomodulatory drug LEF which inhibits activated T cell and Mφ proliferation; and a pro-regulatory cytokine TGF-β1 mimetic peptide, which binds to the TGβII receptor to suppress the activation of CD8⁺ T cells and polarize M1 Mφs to M2 Mφs.

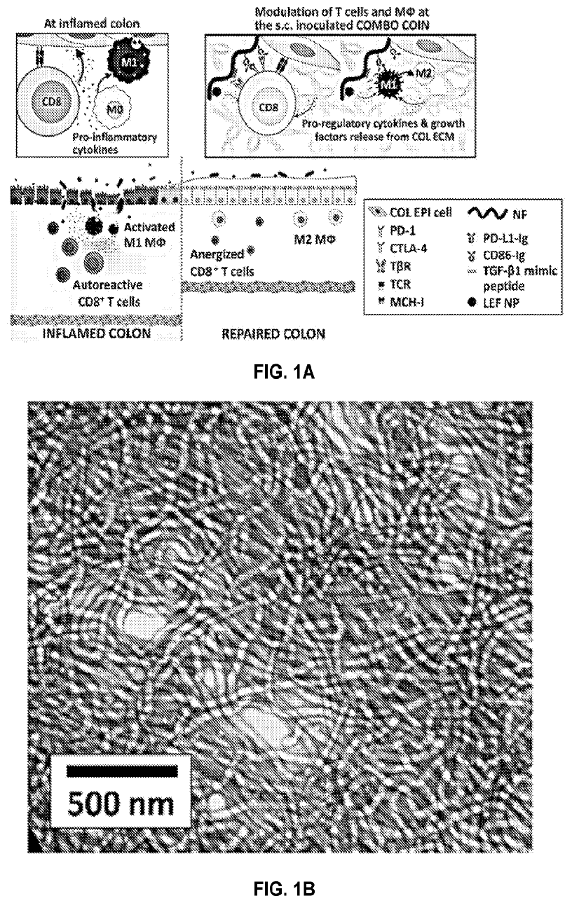

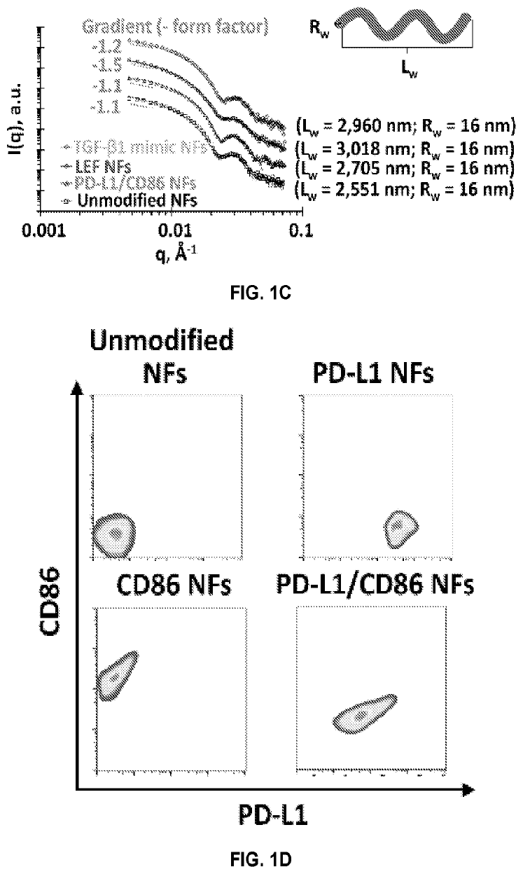

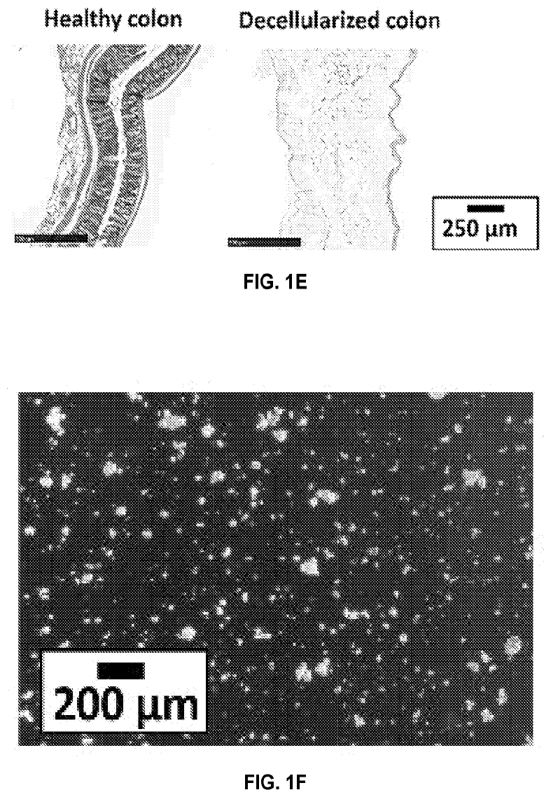

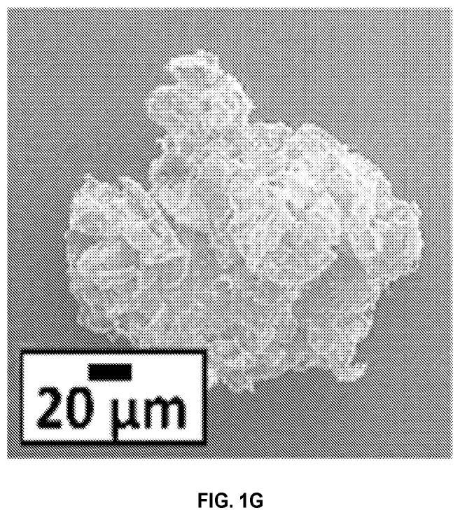

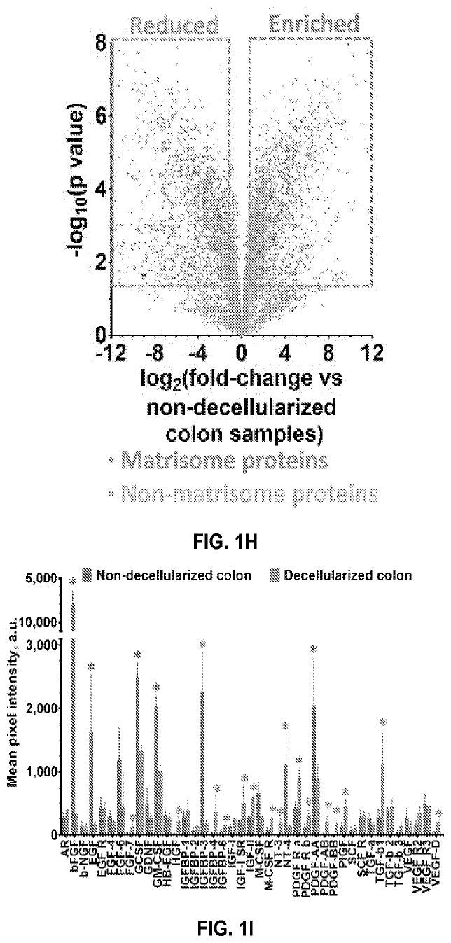

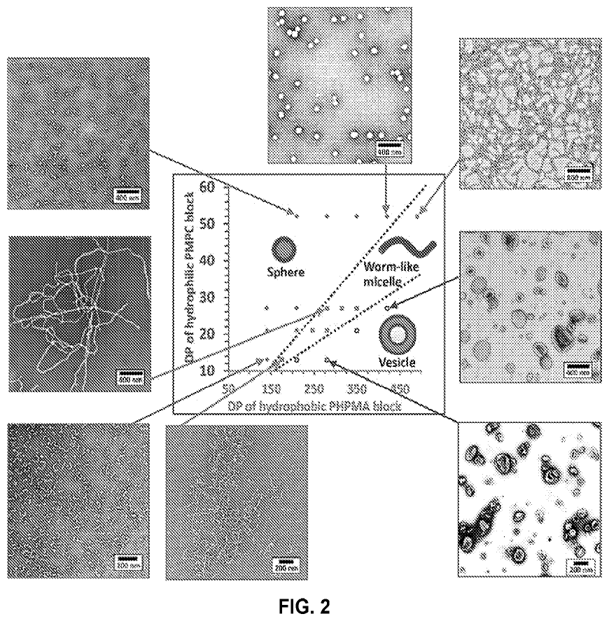

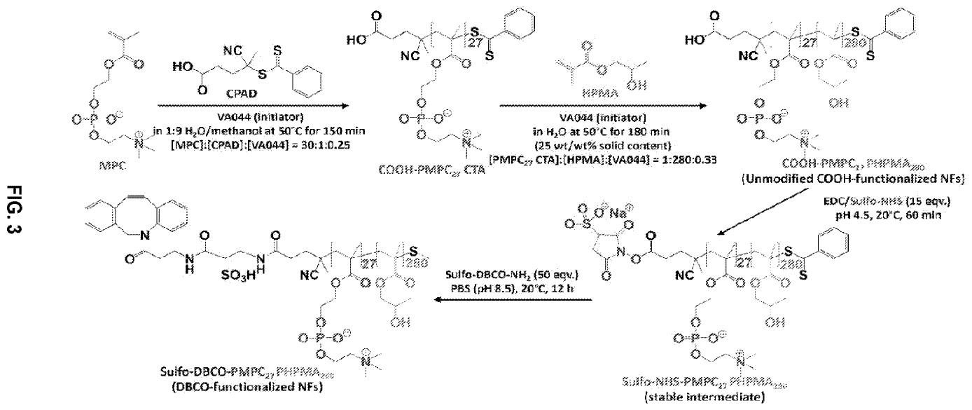

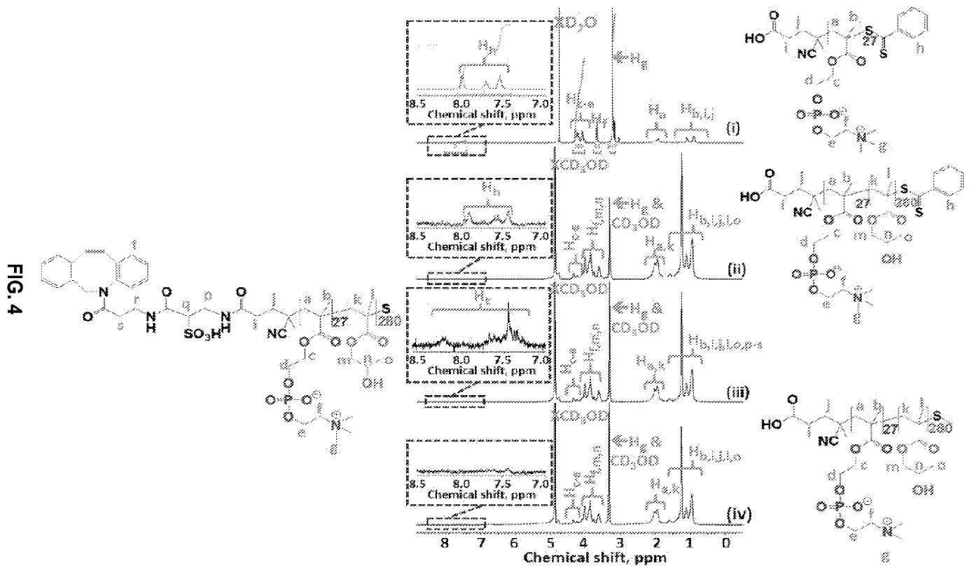

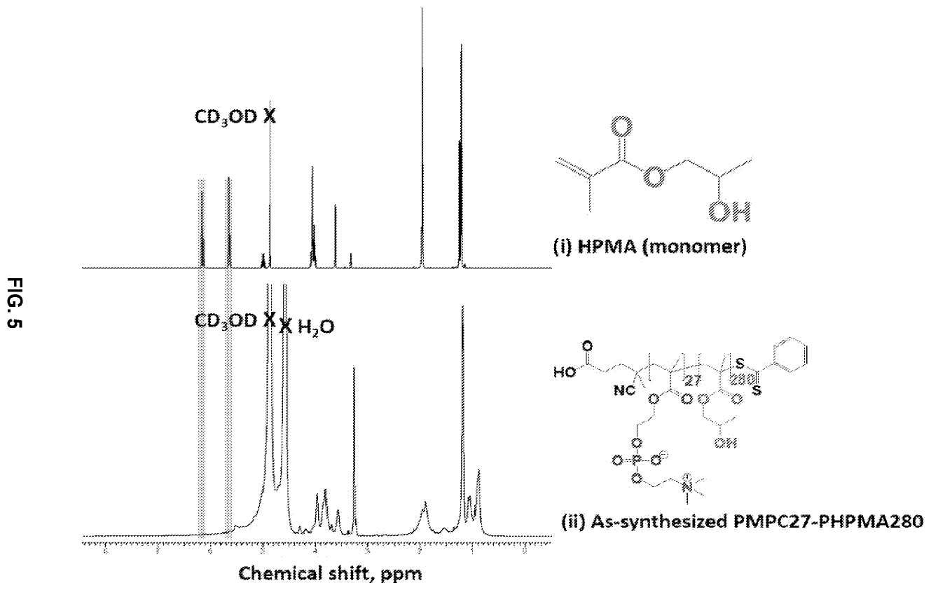

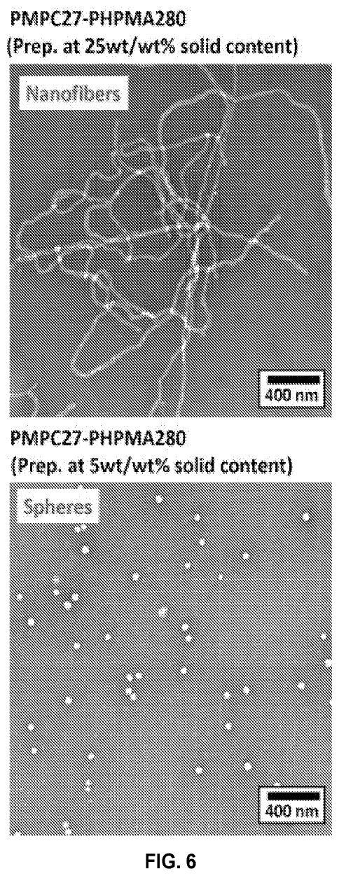

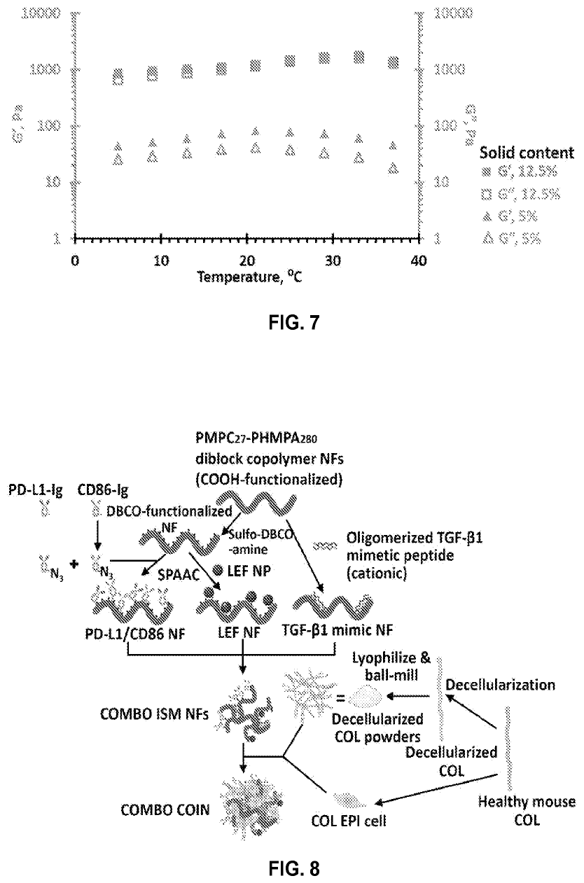

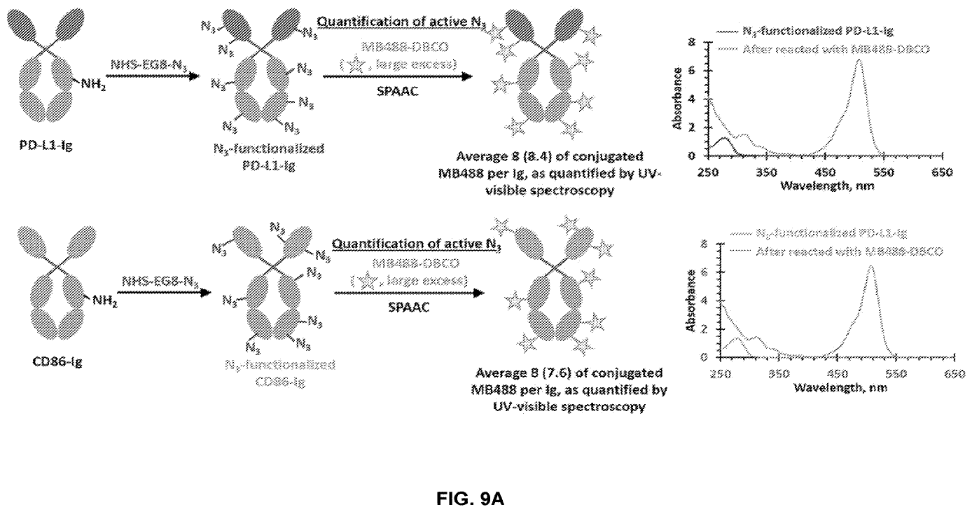

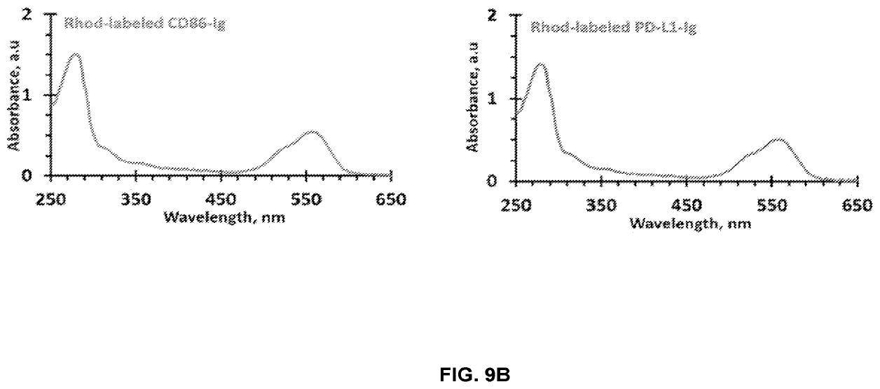

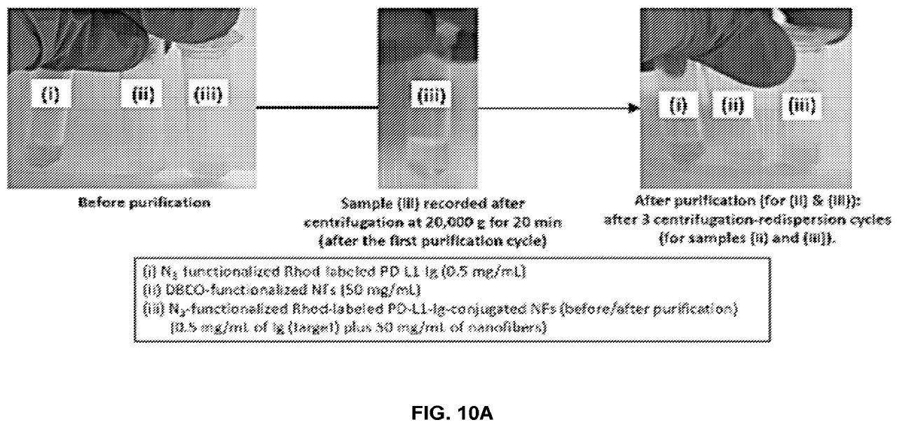

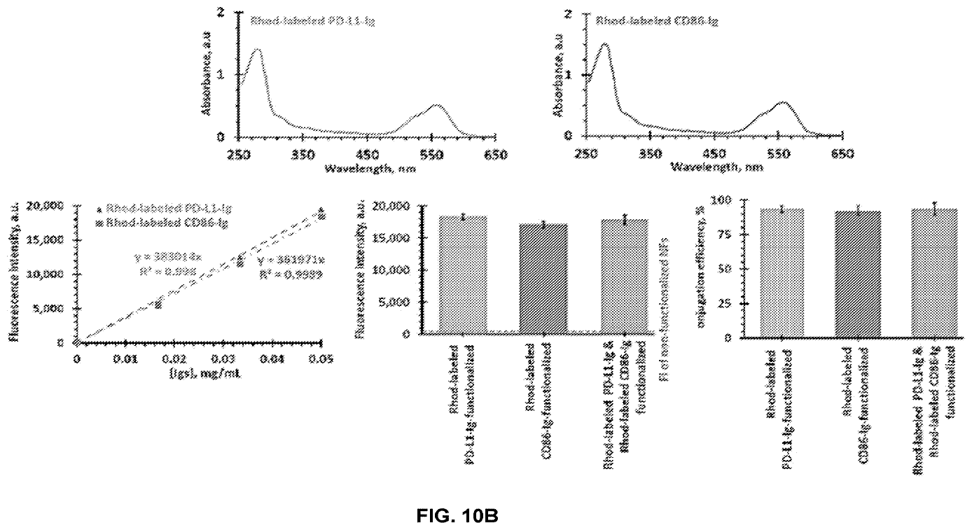

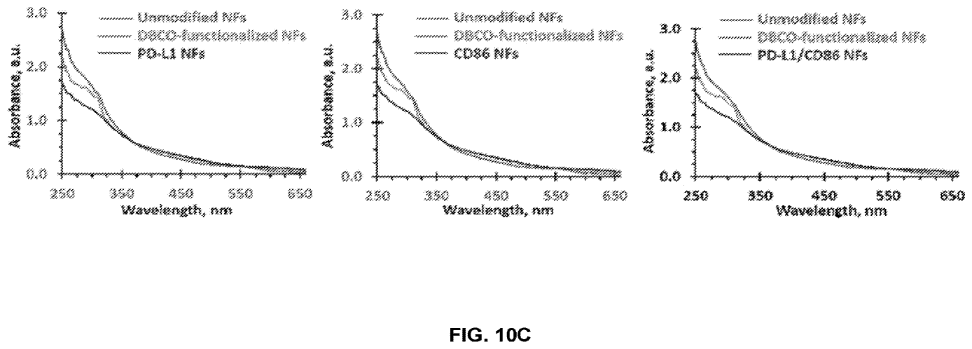

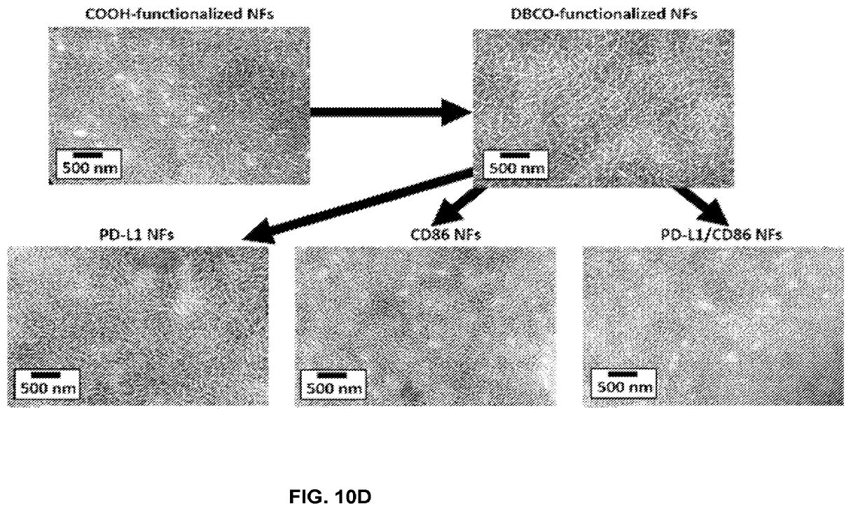

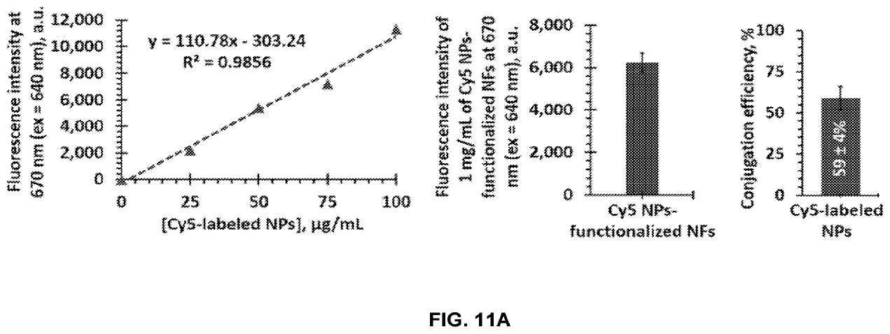

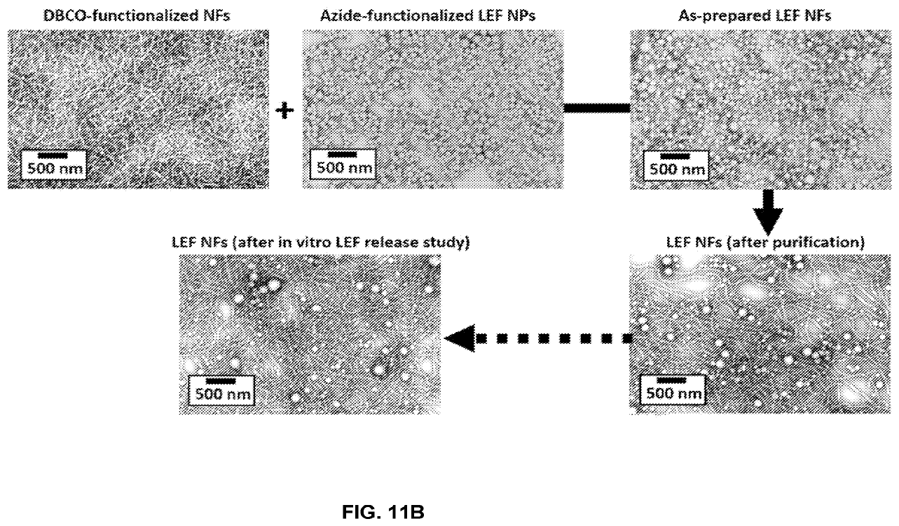

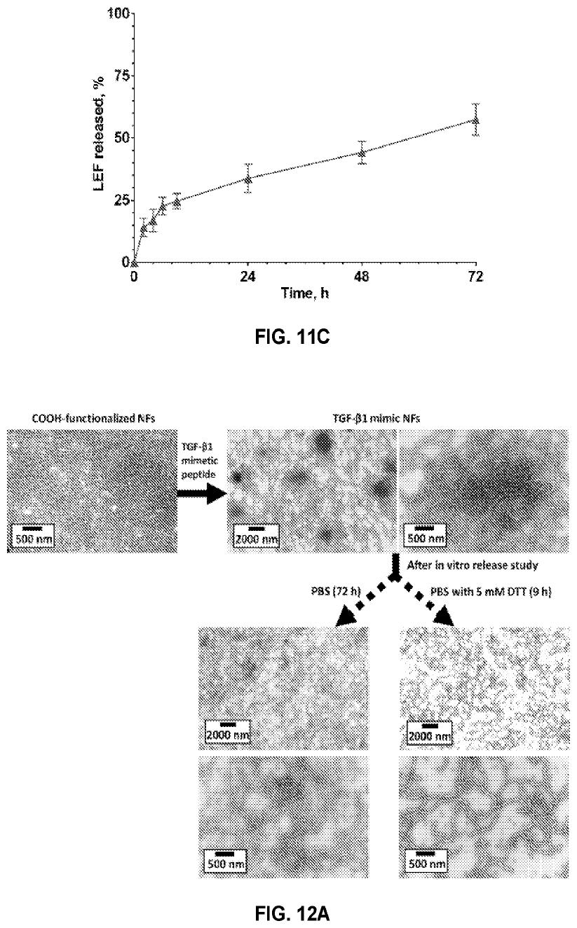

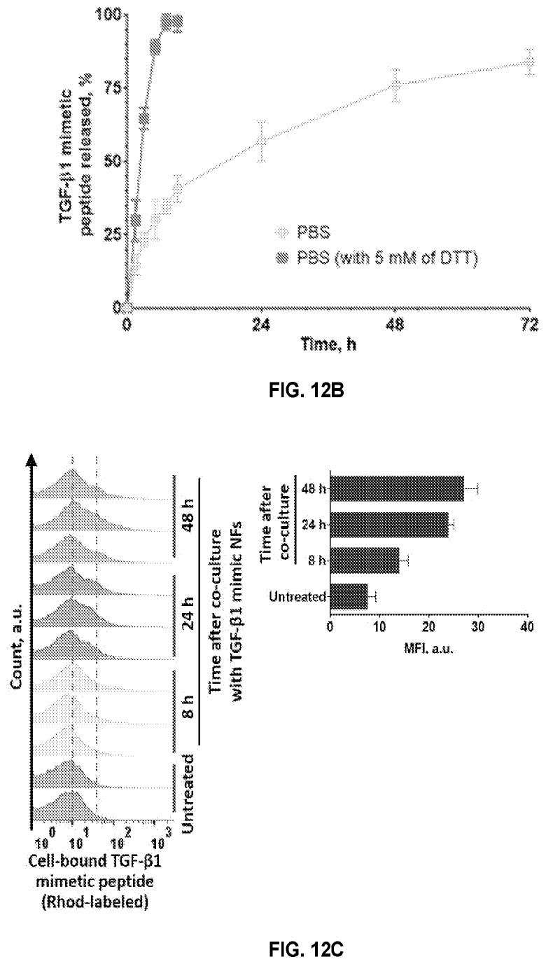

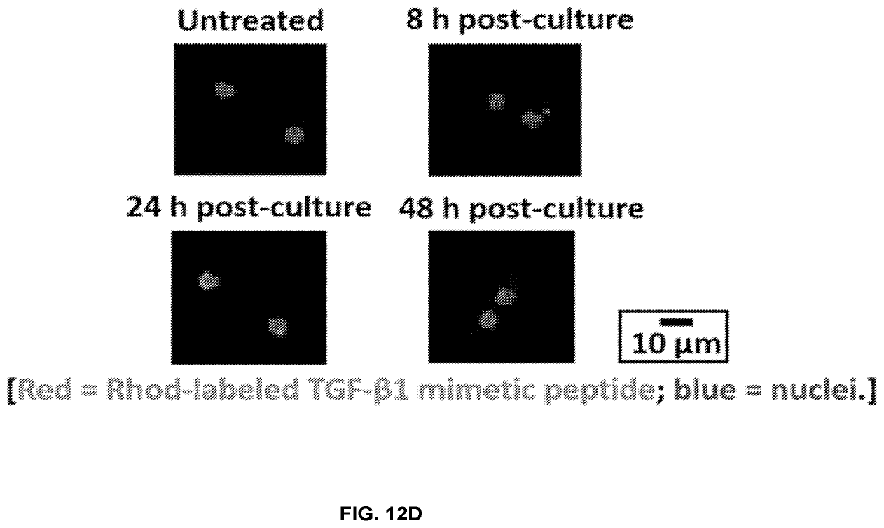

Modular-based ISM NFs were engineered using poly(2-(methacryloyloxy)ethyl phosphorylcholine)-poly(2-hydroxypropyl methacrylate) (PMPC-PHPMA) diblock copolymer as a scaffold. The diblock copolymer was synthesized through a two-step reversible addition-fragmentation chain transfer (RAFT) polymerization, and self-assembled into rod-shaped, fiber-like micelles in situ (FIG. 1B, FIG. 2-7, and Tables 1-2). The carboxylic acid (COOH) terminus of the RAFT reagent's alpha-end group on the NF surface was converted into a dibenzocyclooctyne (DBCO) group via an N-hydroxysulfosuccinimide (sulfo-NHS)/l-ethyl-3-(3-dimethylaminopropyl)carbodiimide (EDC) coupling reaction with the sulfo-DBCO amine (FIG. 3). PD-L1 and CD86 mono- and dual-functionalized NFs (PD-L1 and/or CD86 NFs) and LEF-encapsulated NFs (LEF NFs) were prepared from DBCO-functionalized NFs by strain-promoted azide-alkyne cycloaddition with the corresponding azide-functionalized immune checkpoint ligands (FIG. 8, FIG. 9A-9B, and FIG. 10A-10D) and azide-functionalized LEF-encapsulated poly(ethylene glycol)-poly(lactide-co-glycolide) nanoparticles (LEF NPs) (FIG. 11A-11C). TGF-β1 mimetic peptide-encapsulated NFs (TGF-β1 mimic NFs) were engineered through the ionic adsorption of the oligomerized cationic TGF-β1 mimetic peptide onto anionic carboxylic acid-functionalized NFs (FIG. 12A-12D). These non-thermal responsive NFs (FIG. 7) can be manufactured on industrial scale and stored at 4° C. before further use.

TABLE 1
Characterization of different PMPC-PHPMA diblock copolymers with
different PMPC and PHPMA DPs synthesized at different solid contents,
as determined by dynamic light scattering (DLS) and TEM.

| | Average intensity-average diameter | |
|---|---|---|
| | (Dₕ), nm (average polydispersity | |
| | index (PDI))^{II} | Morphology\hat{ } |
| PMPC27-PHPMA140 | 28 ± 1 (0.080 ± 0.006) | Spheres |
| (prepared at 25 wt/wt % | | |
| solid content) | | |
| PMPC27-PHPMA210 | 47 ± 1 (0.175 ± 0.008) | Spheres + short |
| (prepared at 25 wt/wt % | | nanofibers |
| solid content) | | |
| PMPC27-PHPMA280 | 490 ± 2 (0.532 ± 0.053) | Nanofibers |
| (prepared at 25 wt/wt % | | |
| solid content) | | |
| PMPC27-PHPMA280 | 66 ± 1 (0.049 ± 0.016) | Spheres |
| (prepared at 5 wt/wt % | | |
| solid content) | | |
| PMPC27-PHPMA350 | 597 ± 3 (0.483 ± 0.063) | Nanofibers |
| (prepared at 25 wt/wt % | | |
| solid content) | | |
| PMPC27-PHPMA420 | 320 ± 4 (0.197 ± 0.021) | Nanofibers + |
| (prepared at 25 wt/wt % | | vesicles |
| solid content) | | |

Note:
^{II}sphere-equivalent Dₕ determined by DLS. All DLS measurements were based on the average of 3 independent measurements. All measurements were measured at 25° C.
\hat{ }determined by TEM.

TABLE 2
Characterization of unmodified and different functionalized PMPC27-
PHPMA280-based NFs by DLS and aqueous electrophoresis methods.

| | Average intensity-average diameter | |
|---|---|---|
| | (Dₕ), nm (average polydispersity | Average zeta |
| | index (PDI))\hat{ } | potential, mV\hat{ } |
| Unmodified (COOH- | 442 ± 7 (0.427 ± 0.007)^{II}; | −24.9 ± 0.9 |
| functionalized) NFs | 489 ± 6 (0.437 ± 0.012) | |
| | at 4° C. in 1X PBS; | |
| | 456 ± 5 (0.389 ± 0.012) | |
| | at 25° C. in 1X PBS; | |
| | 454 ± 9 (0.367 ± 0.009) | |
| | at 37° C. in 1X PBS; | |
| | 428 ± 8 (0.356 ± 0.016) | |
| | at 45° C. in 1X PBS. | |
| DBCO-functionalized NFs | 474 ± 10 (0.384 ± 0.067)^{II} | −14.1 ± 2.9 |
| 1:1 mixture of PD-L1-Ig/ | 13 ± 2^{#} | −42.3 ± 2.9 |
| CD86-Ig | | |
| PD-L1-Ig/CD86-Ig dual- | 470 ± 20 (0.333 ± 0.052)^{II} | −23.0 ± 3.6 |
| functionalized NFs | | |
| (PD-L1/CD86 NFs) | | |
| TGF-β1 mimetic peptide | Not applicable | +22.1 ± 3.1 |
| TGF-β1 mimetic peptide- | 503 ± 24 (0.714 ± 0.039)^{II} | −2.6 ± 4.2 |
| functionalized NFs | | |
| (TGF-β1 mimic NFs) | | |
| LEF NPs | 107 ± 1 (0.092 ± 0.003) | −23.9 ± 0.5 |
| LEF NP-functionalized NFs | 477 ± 2 (0.392 ± 0.010)^{II} | −19.5 ± 1.5 |
| (LEF NFs) | | |
| Decellularized COL ECM | Not applicable | −34.5 ± 1.9 |

Note:
^{#}Average number-average diameter (Dₙ);
^{II}sphere-equivalent Dₕ;
\hat{ }all samples, unless specified, were dispersed in 1X PBS, and diluted 100 times with deionized water before DLS and aqueous electrophoresis studies. The pH of the background dispersion was 6.8-8.0. All measurements were based on the average of three independent measurements. All measurements, unless specified, were measured at 25° C.

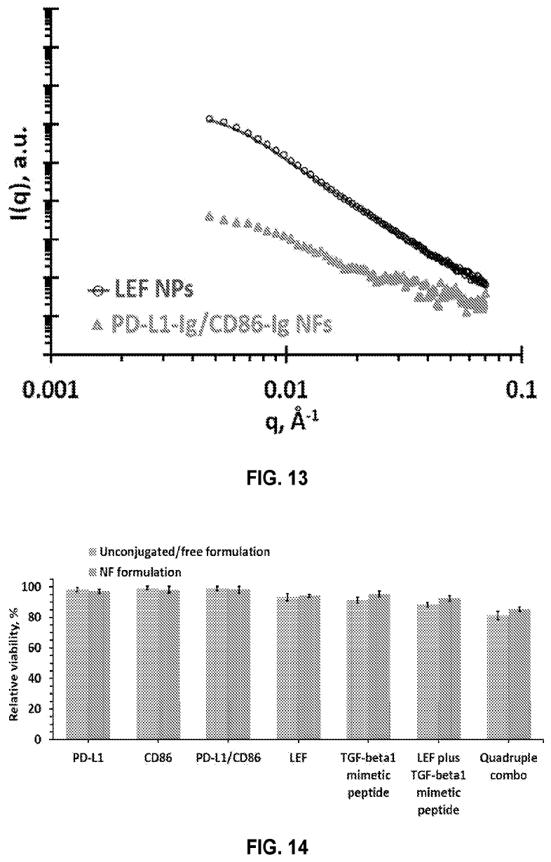

Transmission electron microscopy (TEM) confirmed that the in situ self-assembled PMPC-PHPMA diblock copolymer NFs retained their fiber-like morphology after functionalization (FIG. 11B, FIG. 10A-10D, FIG. 11A-11C, FIG. 12A-12D). Local NF aggregation was observed in the LEE NFs and TGF-β1 mimic NFs since both functionalization components can act as bridges to cross-link the NFs. Furthermore, solution small-angle X-ray scattering (SAX(S) analysis (FIG. 1C, FIG. 13) showed that all functionalized NFs conformed to a core-shell cylinder model with gradients of −1.1 (carboxylic acid-functionalized NFs) to −1.5 (LEF NFs) in the Guinier region. Steeper gradients were recorded for the LEF NFs and TGF-β1 mimic NFs due to their partial aggregation. Nevertheless, the weight-averaged lengths (Lw) of all NFs were approximately 3 μm, and their core weight-averaged radius was 16 nm. In vitro binding studies using the fluorescence-activated cell sorting (FACS) method confirmed that both PD-L1 and CD86 mono-/dual-functionalized NFs were bound selectively to their corresponding fluorescently labeled antibodies (FIG. 1D). A drug release study confirmed the controlled release of the encapsulated LEF and adsorbed TGF-β1 mimetic peptides from the NFs under physiological conditions (FIG. 11A-11C and FIG. 12A-12D). All functionalized NFs showed minimal in vitro toxicity to cultured COL EPI cells (FIG. 14).

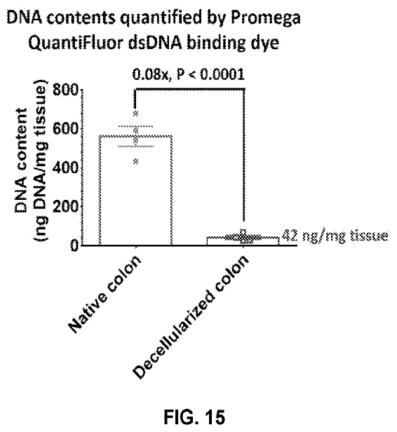

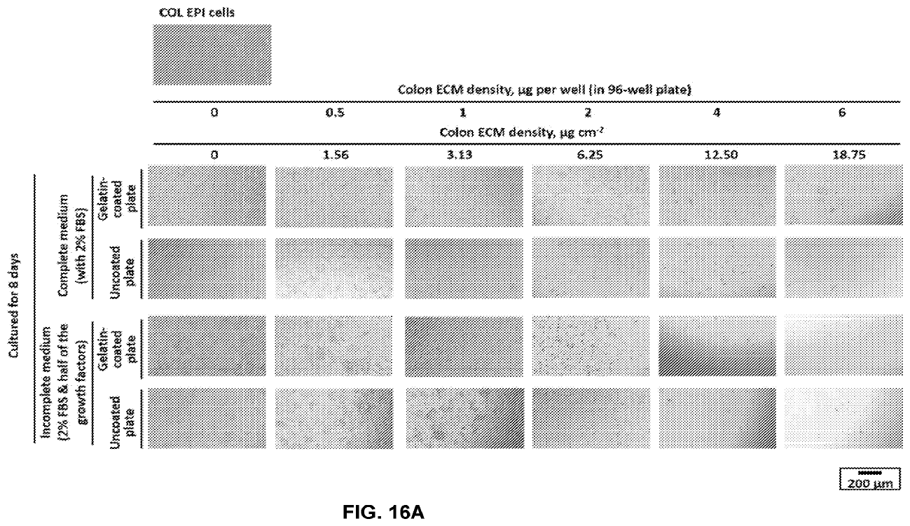

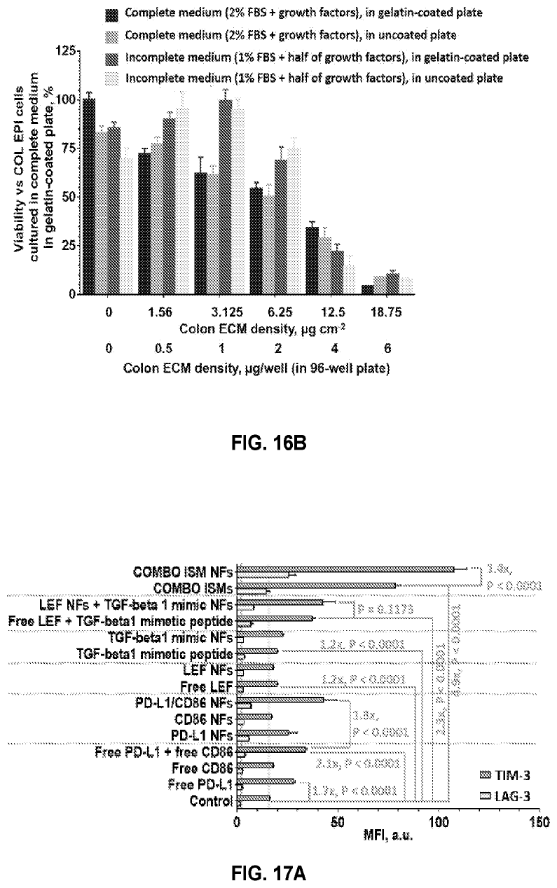

To engineer the COIN, decellularized colons were first generated from preserved mouse colons using a spin-decellularization protocol. Histological analysis confirmed the absence of cells and the presence of collagen in the decellularized colon scaffolds (FIG. 1E), and the DNA content of the colons was reduced by 92% on average (FIG. 15). Ball milling of the lyophilized decellularized colon scaffolds yielded sub-hundred micrometer-sized s.c. injectable fine powders (FIG. 1F-1G). Proteomic analysis confirmed that decellularization preserved key nonmatrisome and matrisome proteins (FIG. 1H). Additionally, the multiplexed growth factor immunoassay showed that the decellularized colon ECM preserved most of the immunomodulatory growth factors (e.g., granulocyte colony-stimulating factor (GCSF), granulocyte-macrophage colony-stimulating factor (GM-CSF), and platelet-derived growth factor (PDGF)) and pro-regulatory cytokines (e.g., TGF-β1) (FIG. 1I). An in vitro proliferation studies were used to identify the optimal concentration of colon ECM (500 μg of colon ECM/10⁶ colon epithelial cells) colon epithelial cell growth in a reduced-serum colon epithelial cell culture medium since high growth factor and cytokine concentrations suppress colon epithelial cell growth (FIG. 16A-16B). Although this study focused on the use of mouse colon epithelial cells and colon ECM to engineer the immune niche, human colon epithelial cells and colon ECM can be isolated via similar methods. More specifically, human colon epithelial cells can be isolated from intestinal crypts collected through endoscopic epithelial biopsy, and human colon ECM can be isolated from preserved donor colon (from colonic disease-free deceased donor) followed by detergent-facilitated decellularization and sterilization. Established protocols to sterilize tissue allografts to prevent disease transmission from the donor can be further used in the disclosed method. Furthermore, the decellularized colon ECM can be replaced by fully synthetic artificial ECM, similar to the artificial spinal cord ECM for the maturation of human stem cell-derived neurons.

### Example 2: ISM NFs Effectively Inhibit Autoreactive CD8+ T Cell Activation and Induce M1 Mφ Polarization In Vitro

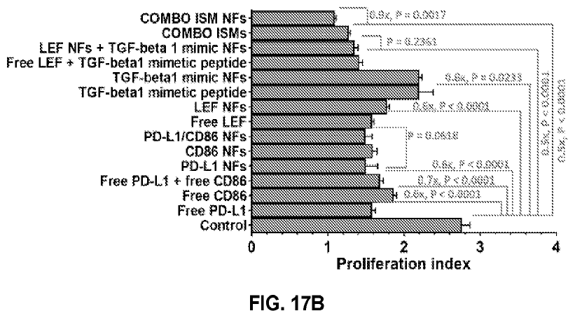

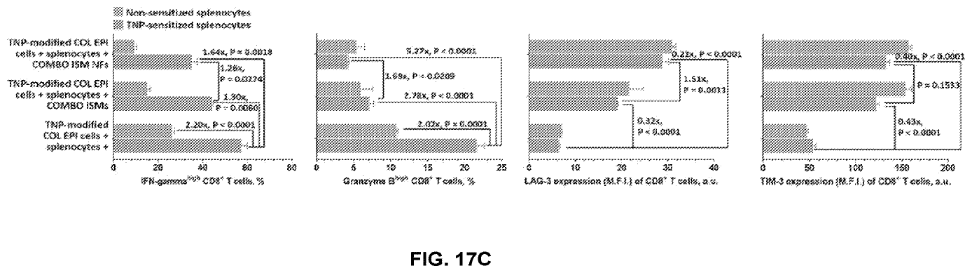

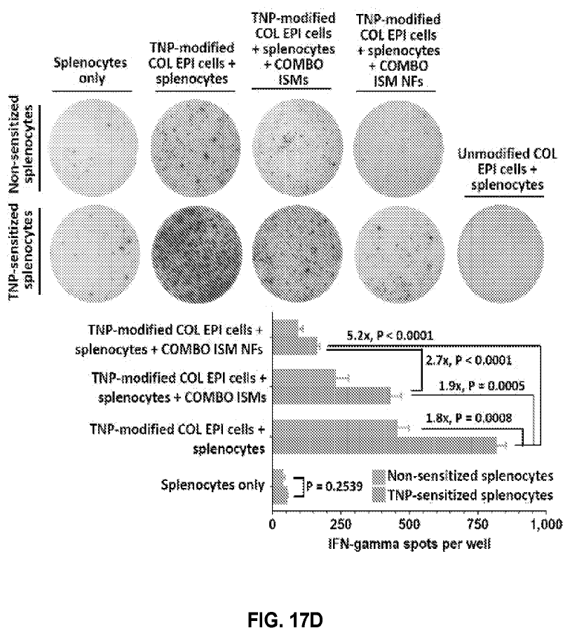

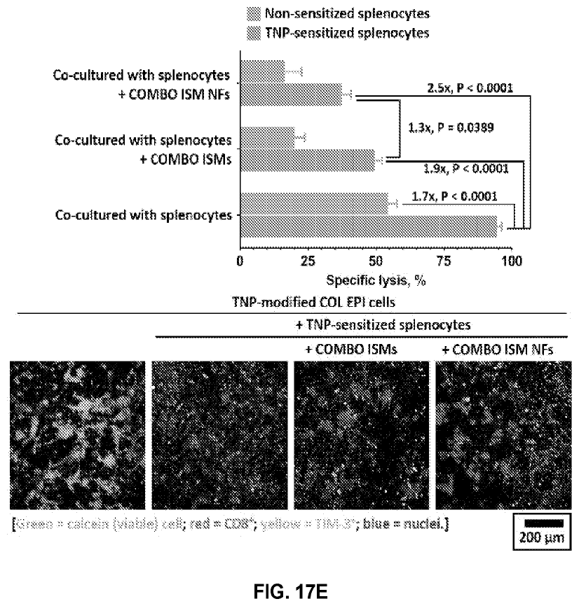

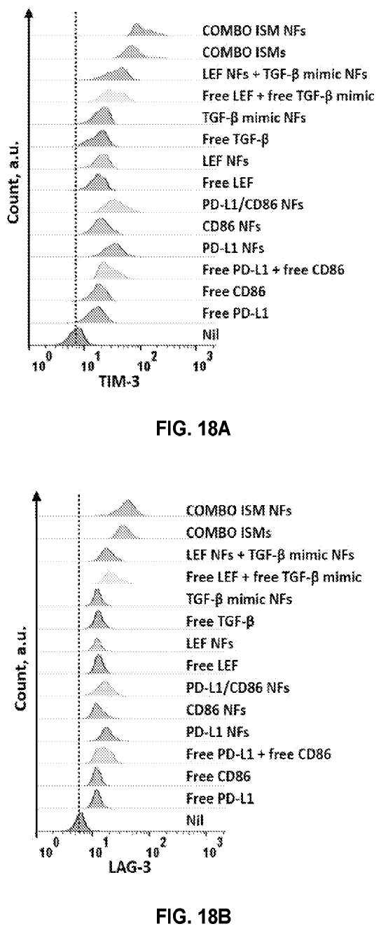

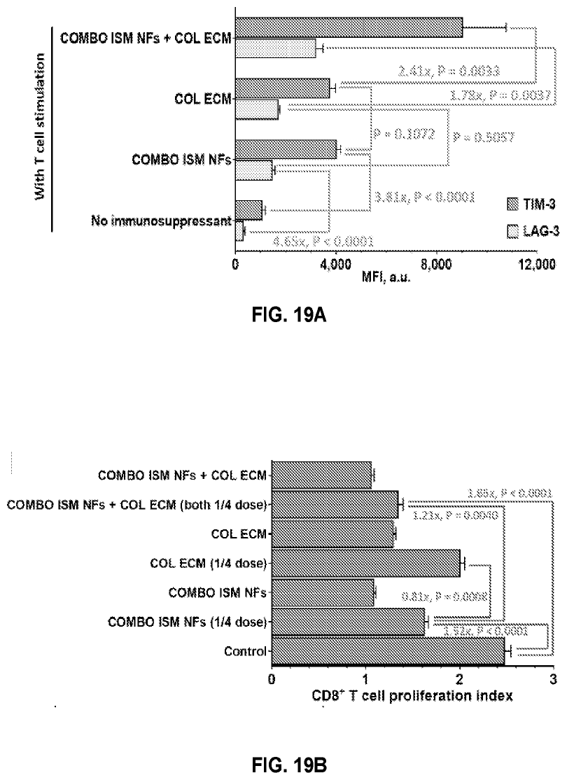

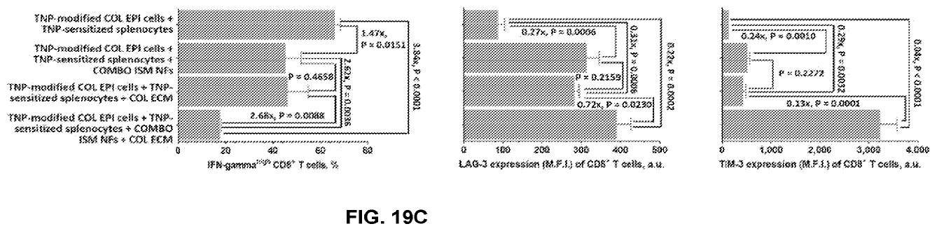

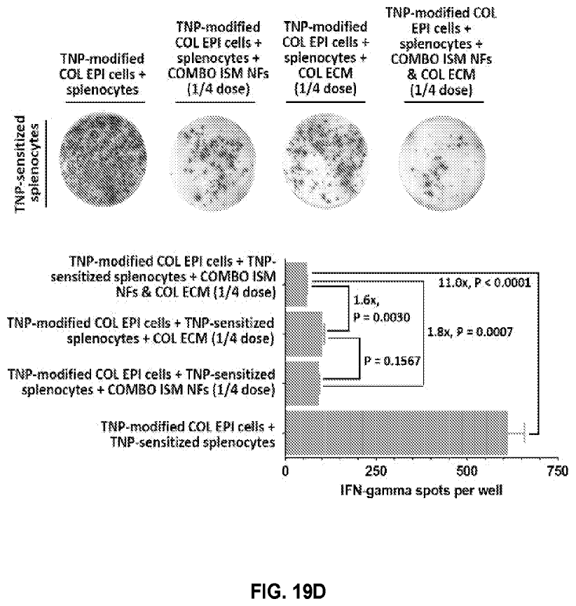

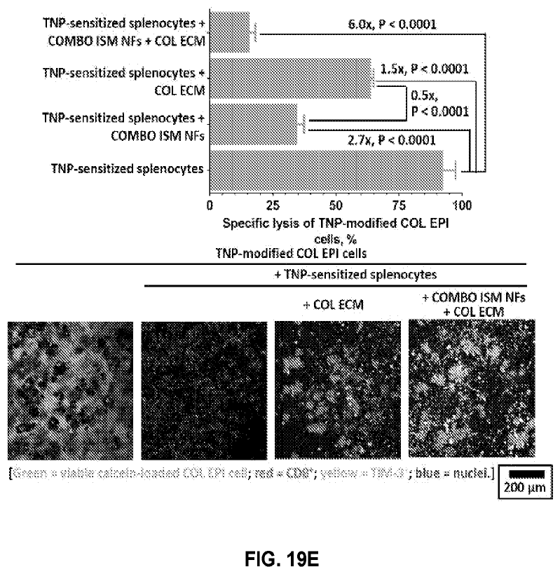

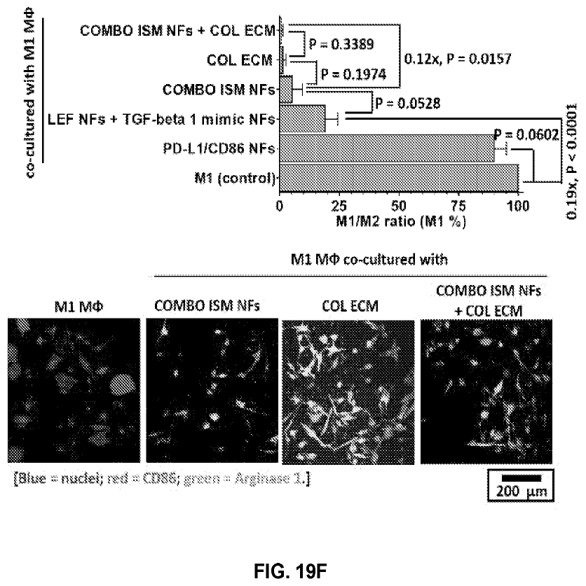

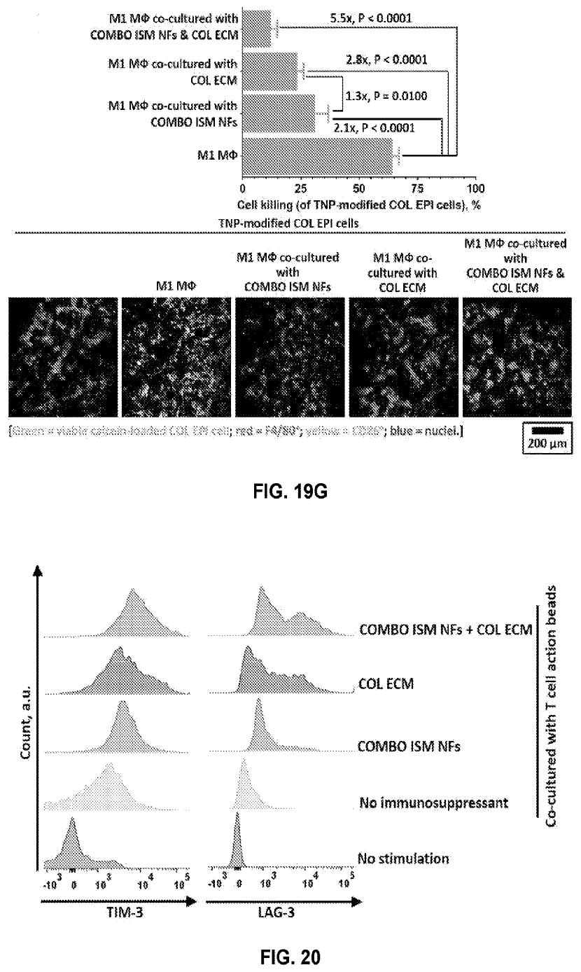

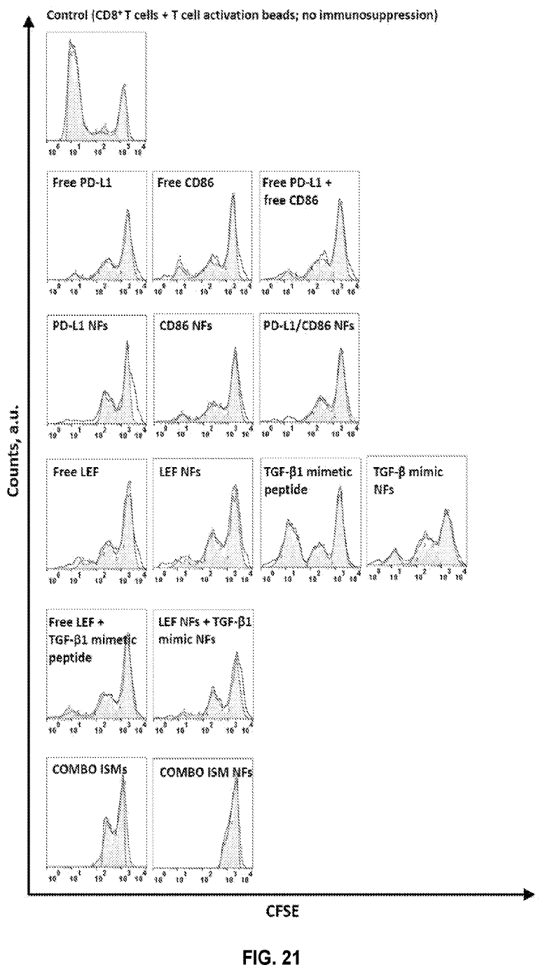

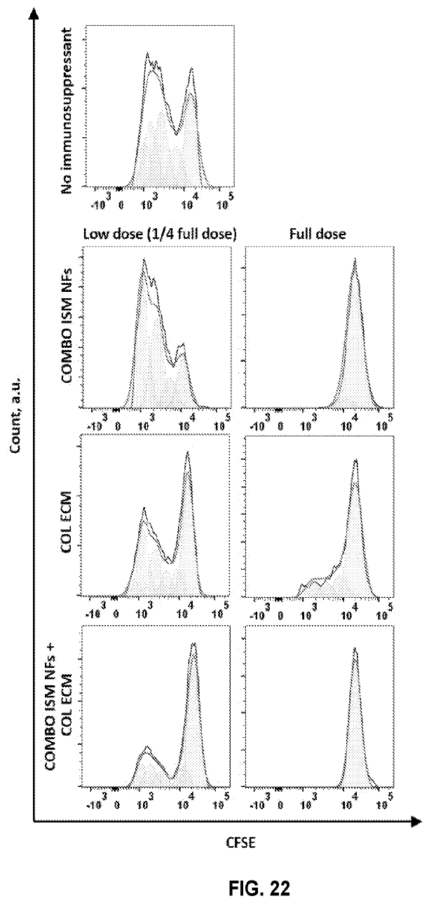

Next, the immunosuppressive effects of NFs formulated with different ISMs and colon ECM were evaluated in vitro. First, their abilities to modulate autoreactive CD8⁺ T cells in vitro were evaluated. The NFs were co-cultured with CD8⁺ T cells with anti-CD3/anti-CD28-functionalized microparticles (Dynabeads™) and interleukin-2 (IL-2). Both unconjugated (“free”) and NF-conjugated PD-L1 and CD86 effectively upregulated T cell exhaustion marker expression (e.g., LAG-3 and TIM-3) in stimulated CD8⁺ T cells. However, the PD-L1 NFs showed a higher inhibitory efficiency than free PD-L1 and CD86 at a 1:1 ratio (FIG. 17A-17E and FIG. 18A-18B). The PD-L1/CD86 NFs more effectively upregulated the inhibitory markers than the free immune checkpoint ligands. Both forms of the TGF-β1 mimetic peptide upregulated inhibitory markers on the stimulated CD8⁺ T cells, with further upregulation observed after coculturing with small-molecule LEF or LEF NFs (FIG. 17A-17E and FIG. 18A-18B). Furthermore, culturing the activated CD8⁺ T cells with the PD-L1, CD86, LEF, and TGF-β1 mimetic peptide combination (COMBO ISMs), especially the COMBO ISM NFs, significantly upregulated LAG-3 and TIM-3 expression by 7- and 26-fold (FIG. 19A, FIG. 17A-17E, FIG. 18A-18B and FIG. 20), respectively. The optimized colon ECM concentration was as effective as COMBO ISM NFs in anergizing stimulated CD8⁺ T cells and upregulating T cell exhaustion markers (FIG. 19A, and FIG. 20) due to the preserved immunomodulatory growth factors and cytokines. Additionally, the combination of COMBO ISM NFs and colon ECM cooperatively upregulated TIM-3 and LAG-3 expressions (FIG. 19A, and FIG. 20). Further in vitro cell proliferation studies indicated that all immunosuppressive NFs, especially the COMBO ISM NFs, and colon ECM effectively inhibited proliferation of carboxyfluorescein succinimidyl ester (CFSE)-labeled CD8⁺ T cells under the same activation conditions (FIG. 19A-19B, FIG. 17A-17E, and FIG. 21-22). The combination of COMBO ISM NFs and colon ECM effectively inhibited the stimulated CD8⁺ T cell proliferation, with less than 5% of the T cells proliferating (FIG. 19B, FIG. 22). The combined inhibition effect was particularly significant when a low concentration of COMBO ISM NFs was combined with a low concentration of colon ECM in the T cell proliferation assay (FIG. 2B, and FIG. 22).

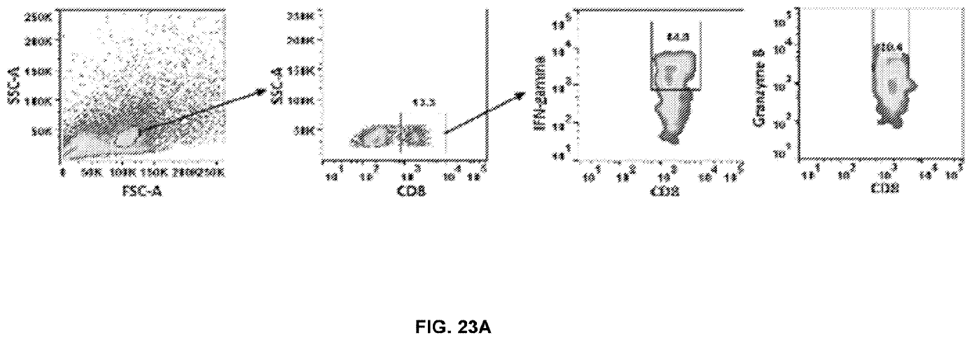

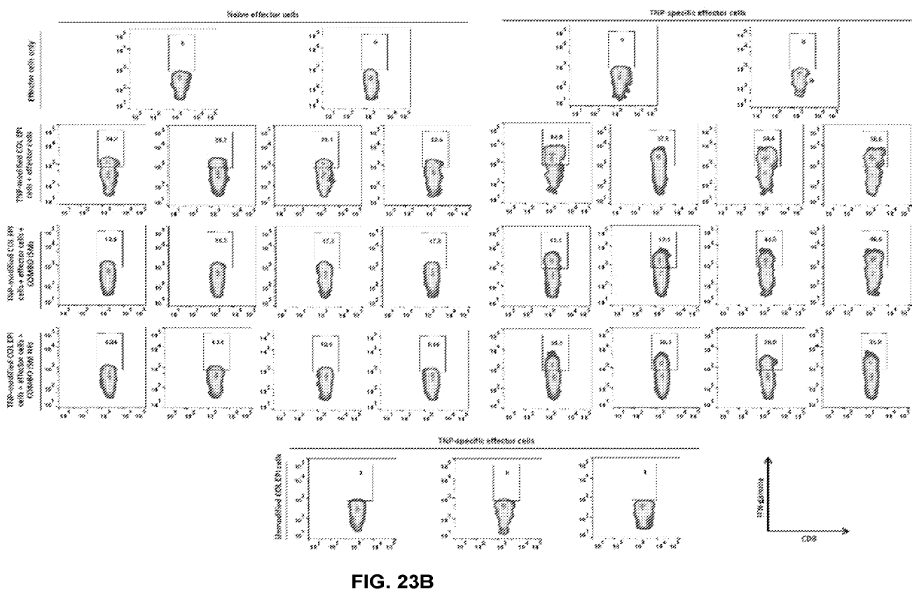

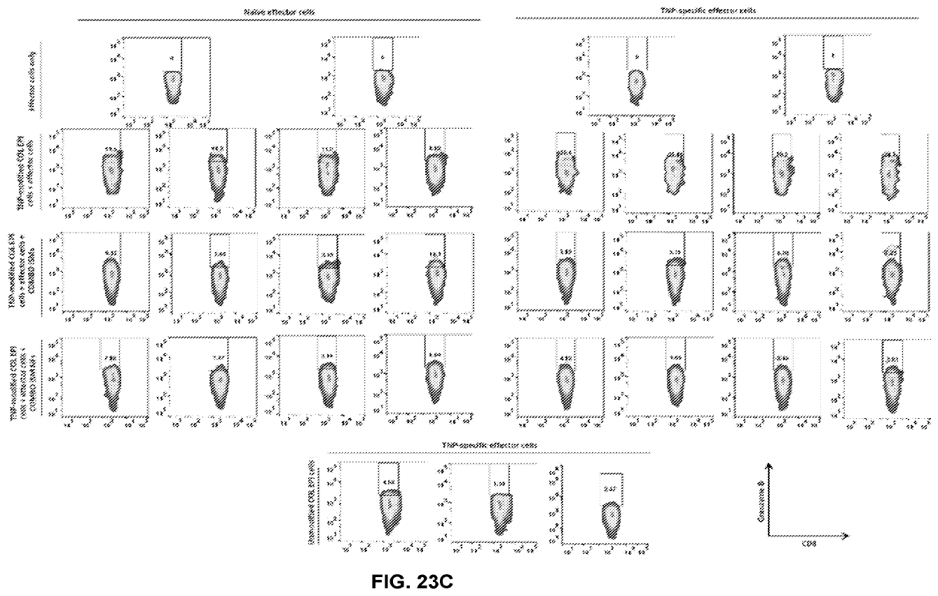

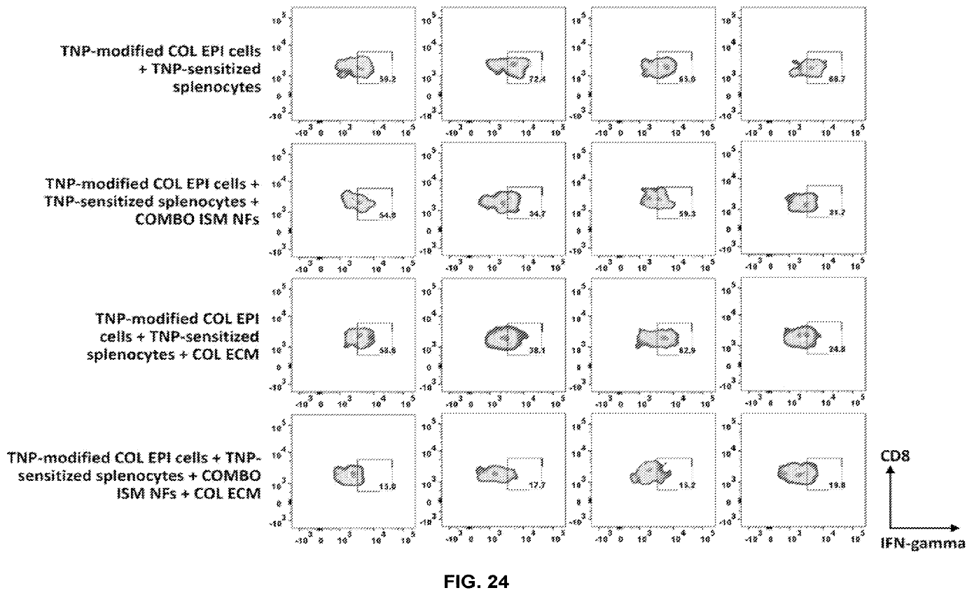

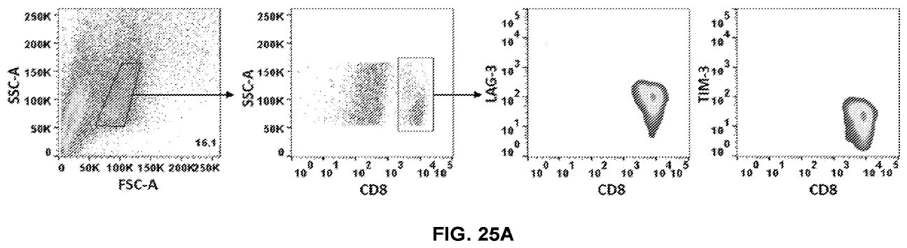

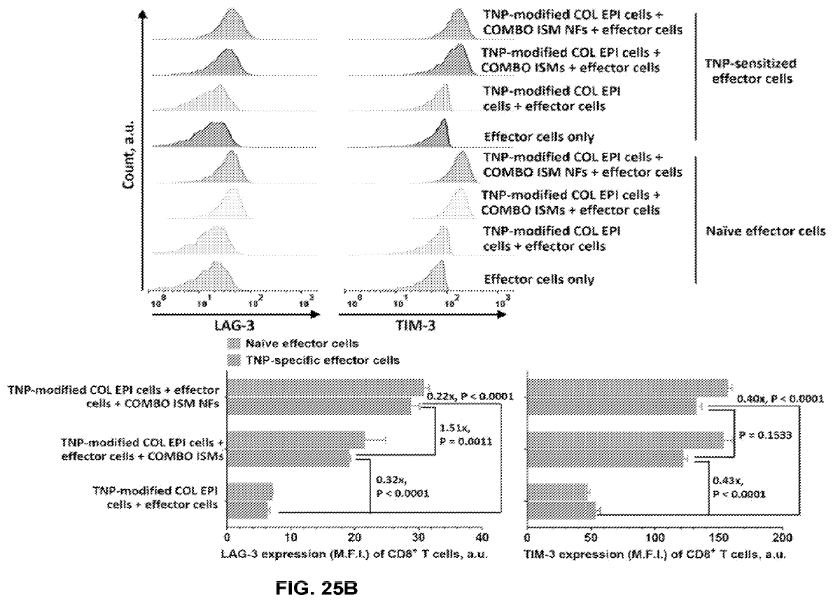

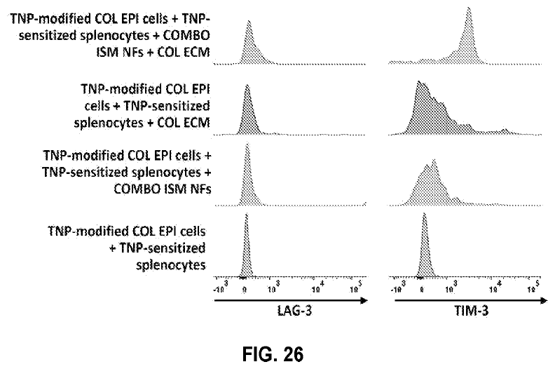

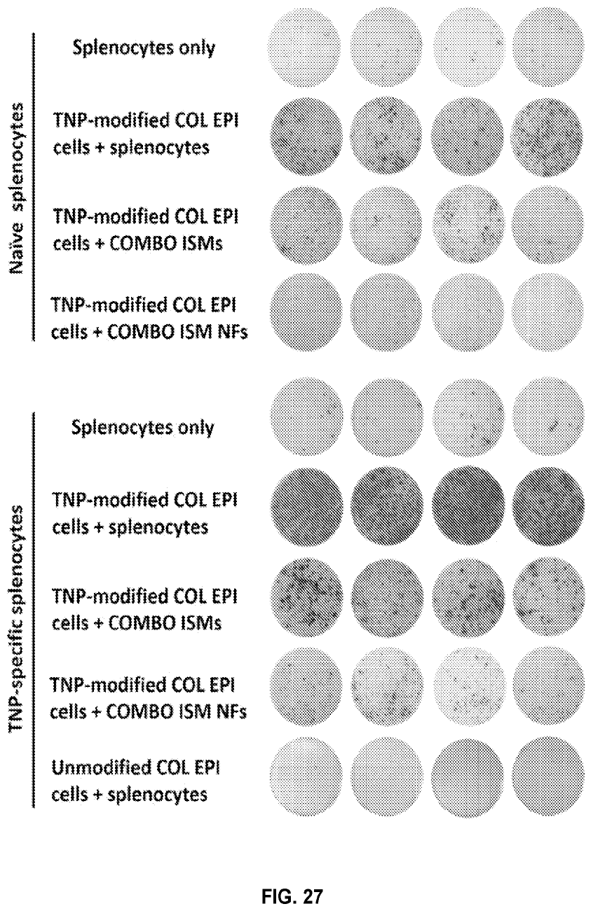

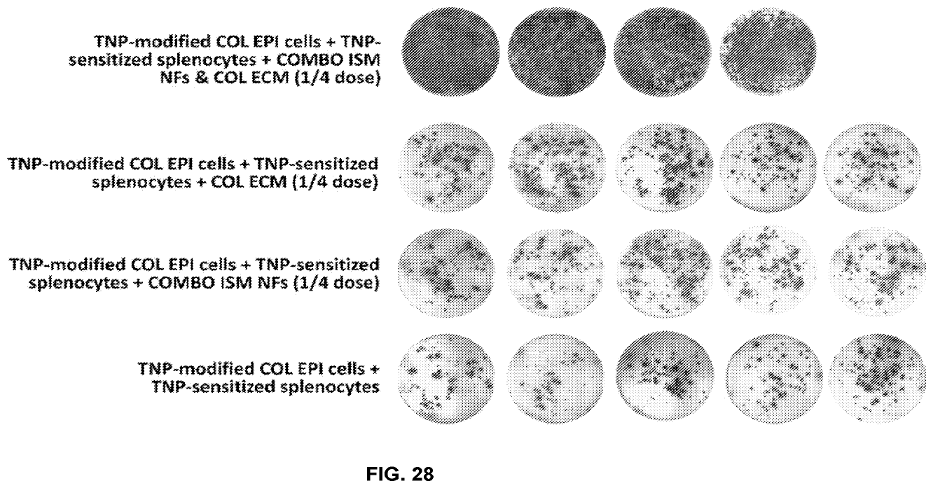

To demonstrate the abilities of the COMBO ISM NFs and colon ECM to inhibit antigen-specific CD8+ T cell activation, CD8+ T cell phenotypes and their cell-killing efficiency after co-culturing with trinitrophenol (TNP)-modified colon epithelial cells were analyzed. Immunogenic TNP-modified colon epithelial cells effectively activated CD8+ T cells in both TNP-sensitized and non-sensitized splenocytes (FIG. 19C, FIG. 17A-17B, and FIG. 23A-23C). The sensitized CD8+ T cells showed significantly higher (over 2-fold) intercellular interferon-gamma (IFN-γ) and granzyme B (GzmB) expressions52 than the non-sensitized CD8+ T cells (FIG. 17A-17B, and FIG. 23A-23C). In contrast, no significant CD8+ T cell activation was observed when the TNP-sensitized splenocytes were cultured with the unmodified colon epithelial cells (FIG. 17A-17B, and FIG. 23A-23C). The co-culturing of the sensitized splenocytes with the TNP-modified colon epithelial cells in the presence of the COMBO ISM NFs or colon ECM markedly reduced the expression of both intercellular T cell activation markers (FIG. 19C, and FIG. 24). A more profound inhibitory effect was observed when the sensitized splenocytes were cultured with the COMBO ISM NFs and colon ECM, where the intercellular IFN-γ expression in the sensitized CD8+ T cells was synergistically reduced by 74% (FIG. 19C, and FIG. 24), whereas COMBO ISM NFs or colon ECM alone reduced the frequencies of IFN-γhigh CD8+ T cells by approximately 30%. Phenotypic analysis showed that co-culturing the TNP-modified colon epithelial cells with COMBO ISM NFs and colon ECM effectively upregulated inhibitory marker expression (LAG-3 and TIM-3) in the sensitized CD8+ T cells (FIG. 19C, FIG. 17A-17B, FIG. 25A-25B, and FIG. 26). Furthermore, the inhibition reduced pro-inflammatory cytokine (e.g., IFN-γ) secretion when the sensitized splenocytes were cocultured with the TNP-modified colon epithelial cells (FIG. 19D, FIG. 17A-17B, and FIG. 27-28). Consequently, the specific lytic activity against the TNP-modified colon epithelial cells was reduced by 91% when the sensitized splenocytes were cocultured with the COMBO ISMs and colon ECM (both at one-quarter of the optimized doses) (FIG. 19E and FIG. 28). Therefore, combining COMBO ISM NFs and colon ECM could effectively inhibit antigen-specific CD8+ T cell lysis.

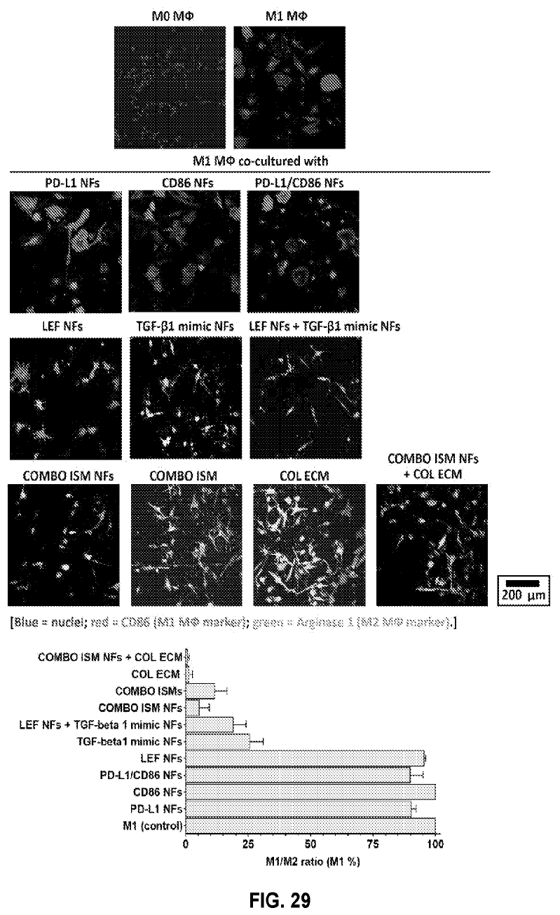

The upregulation of colonic M1 Mφs contributes to ulcerative colitis development. Proinflammatory cytokines (e.g., IFN-γ and tumor necrosis factor-alpha (TNF-α)) produced by colonic CD8⁺ T cells induce the polarization of colonic unpolarized Mφs (M0 Mφs). To demonstrate that COMBO ISM NFs and colon ECM can promote proinflammatory M1 Mφs to differentiate into anti-inflammatory M2 Mφs, the phenotypes of IFN-γ- and lipopolysaccharide (LPS)-induced M1 splenic Mφ s after co-culturing with different immunosuppressive components were analyzed. Similar to free TGF-β1, TGF-β1 mimic NFs effectively polarized approximately 74% of M1 Mφs into M2 Mφs (FIG. 19F, and FIG. 29). Although other mono-functionalized NFs were not adequate to induce M1 Mφ polarization, approximately 94% of M1 Mφs were differentiated into M2 Mφs after co-culturing with COMBO ISM NFs (FIG. 19F, and FIG. 29). Due to the retention of pro-regulatory growth factors and chemokines after colon decellularization (FIG. 1I), the colon ECM effectively induced >98% of M1 Mφs to differentiate into M2 Mφs (FIG. 19F, and FIG. 29). Consequently, combining COMBO ISM NFs and colon ECM effectively converted all M1 Mφs into M2 Mφs (FIG. 19F, and FIG. 29).

To demonstrate that the polarization of M1 Mφs reduces their cell-killing efficiency, co-culture of TNP-modified colon epithelial cells with IFN-γ- and LPS-polarized M1 Mφs with different immunosuppressants were conducted. Due to the immunogenic nature of the TNP-modified cells, M1 Mφs lysed approximately 65% of the COL EPI cells (FIG. 19G). Co-culturing the colon epithelial cells with COMBO COIN inhibited cell killing by approximately half and reduced the CD86+M1 Mφs (FIG. 19G), consistent with the ability of COMBO COIN to polarize M1 Mφs to M2 Mφs. A similar inhibitory effect was observed when the colon epithelial cells were co-cultured with colon ECM. Co-culturing the colon epithelial cells with COMBO COIN and colon ECM significantly inhibited cell killing of M1 Mφs 5.5-fold with <12% of colon epithelial cells being lysed by the M1 Mφs (FIG. 19G). These findings showed that combining COMBO COIN and colon ECM additively inhibited non-specific cell killing by polarizing M1 Mφs into M2 Mφs.

### Example 3: COINs Effectively Ameliorate Colitis in Established Ulcerative Colitis Mouse Models

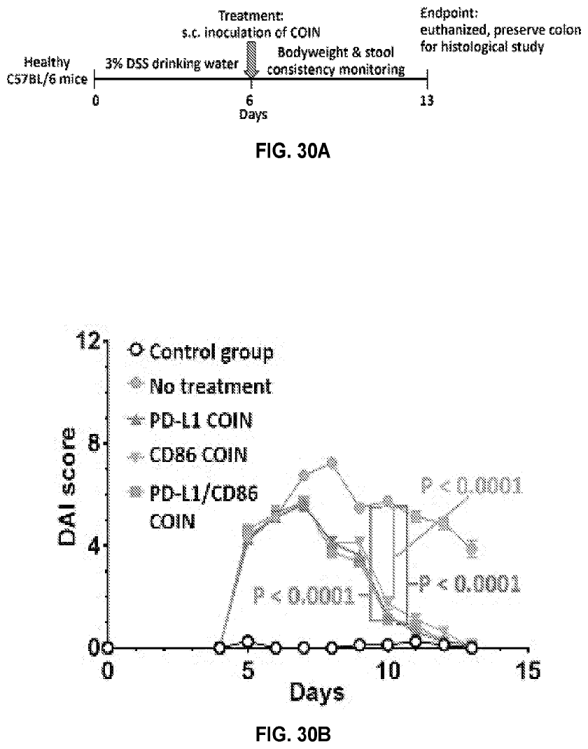

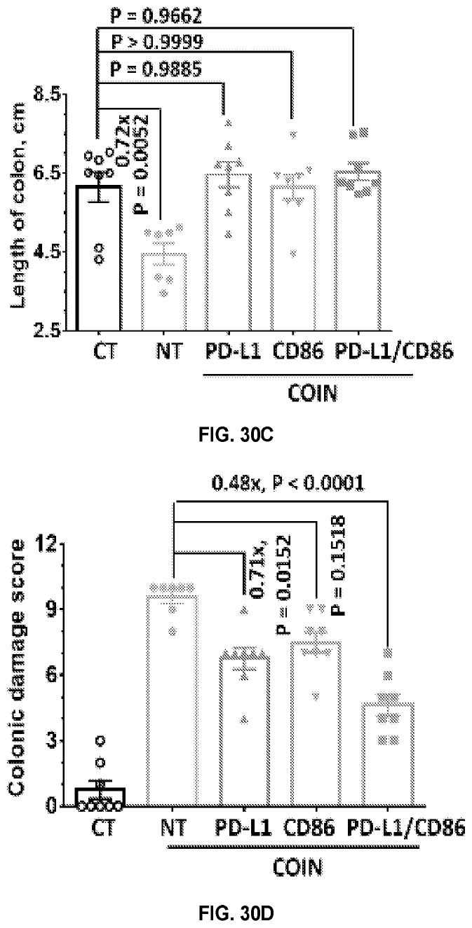

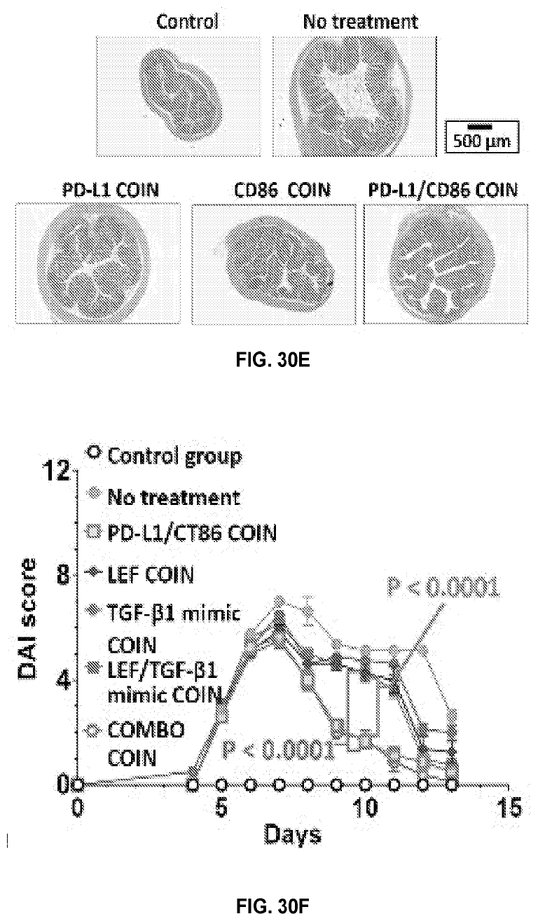

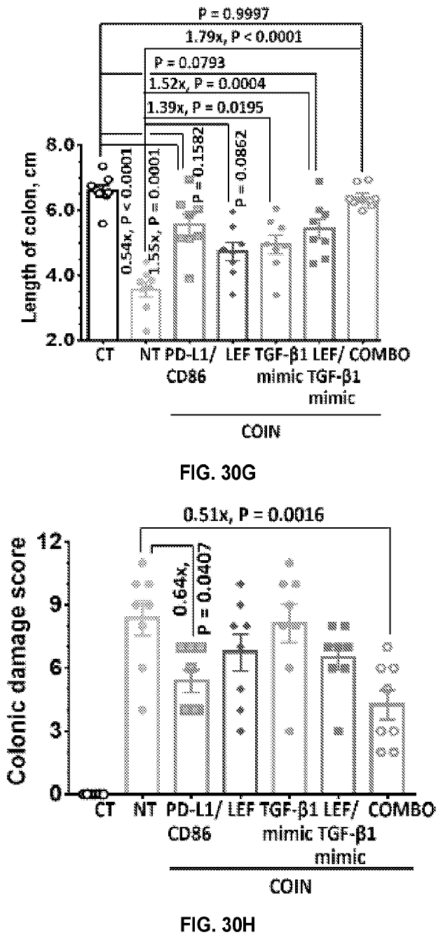

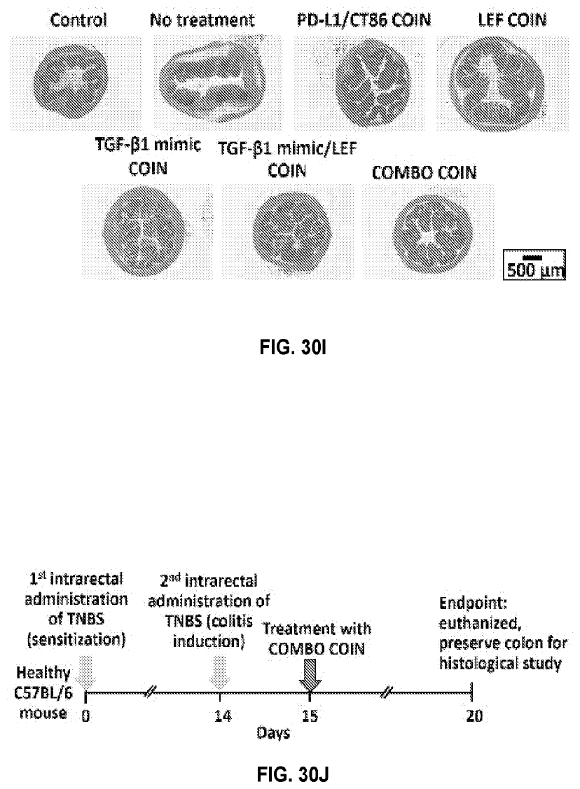

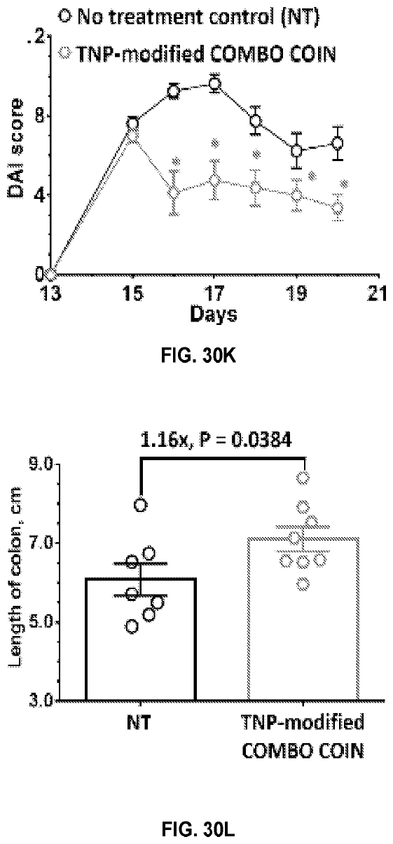

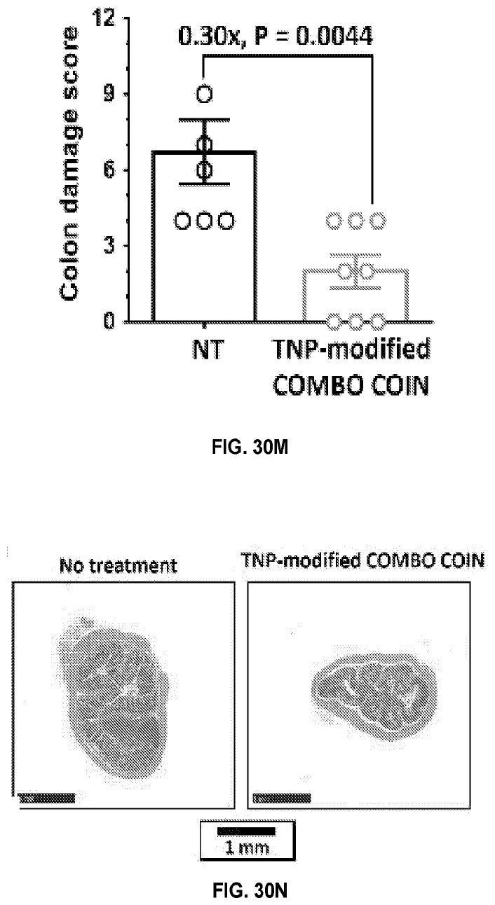

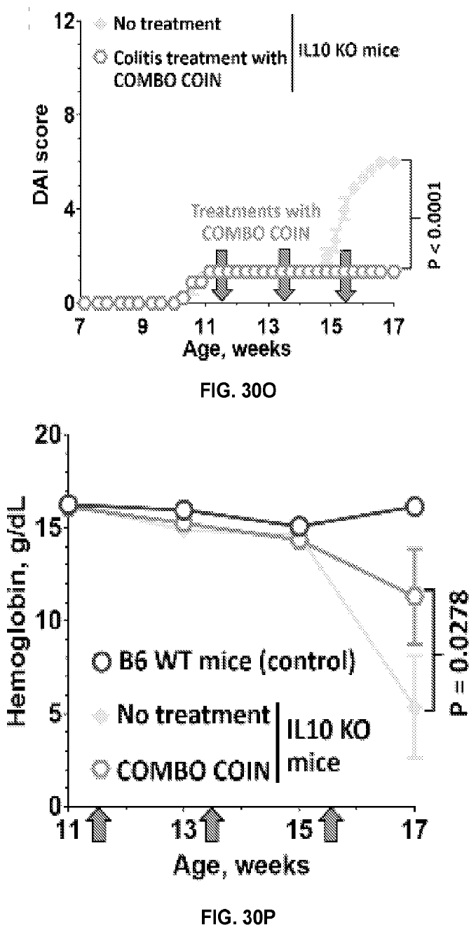

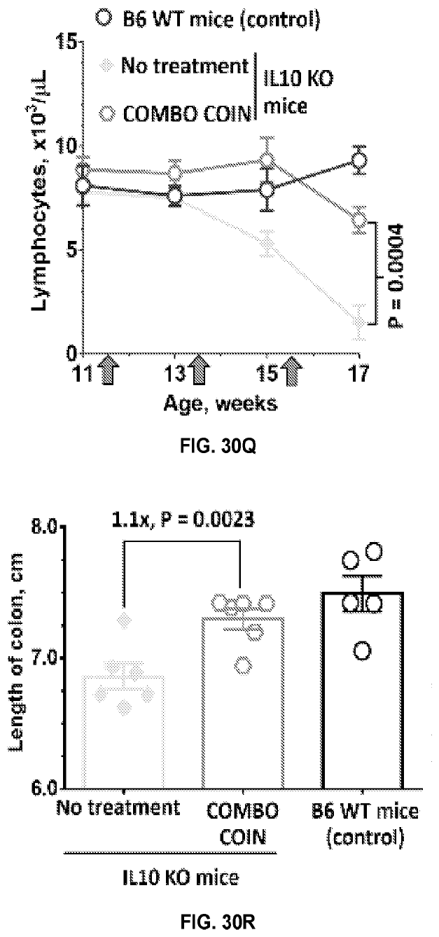

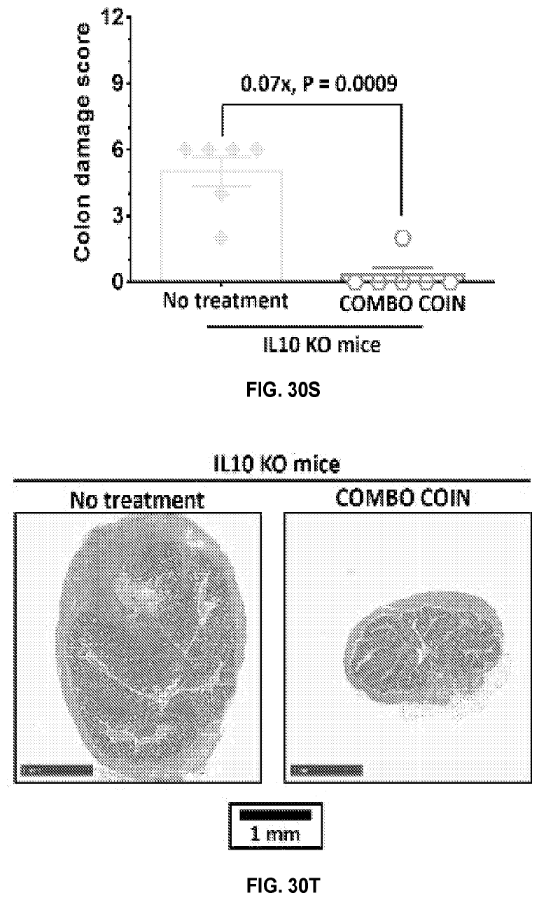

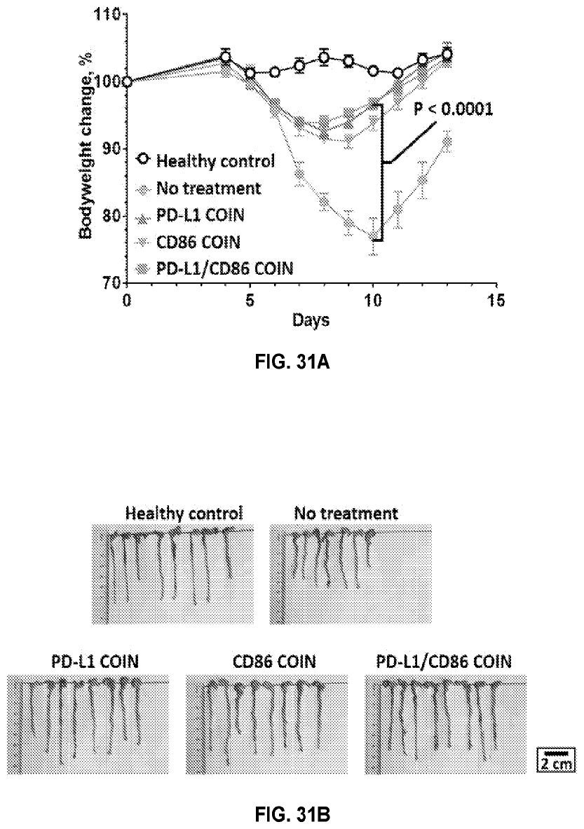

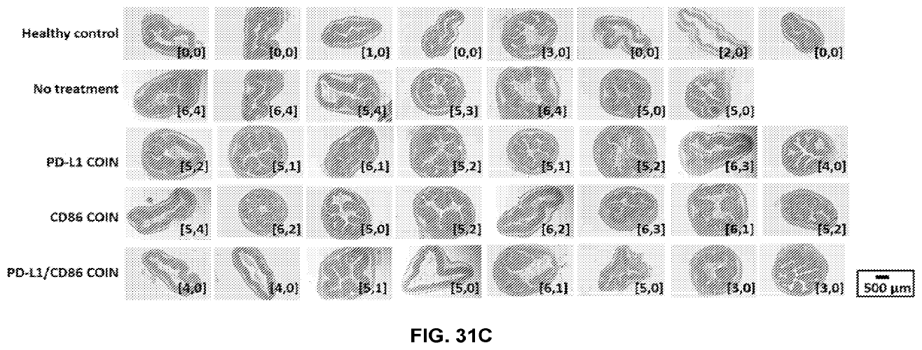

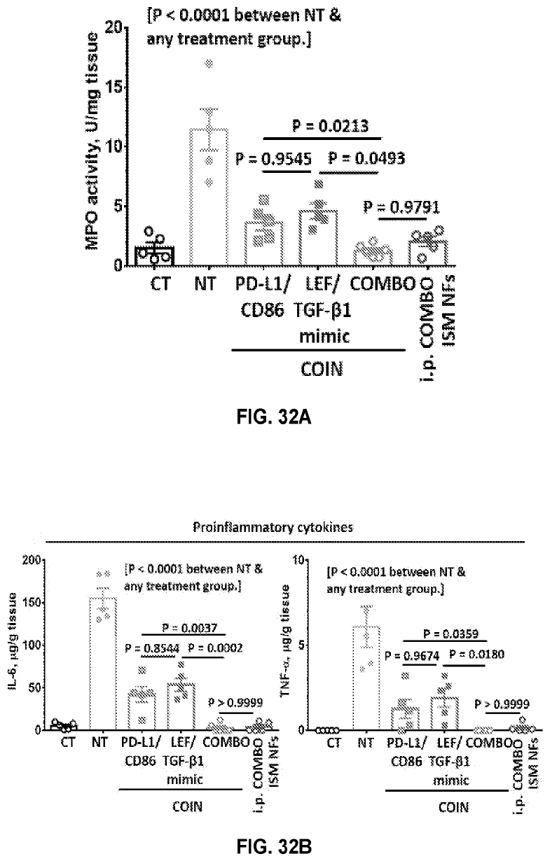

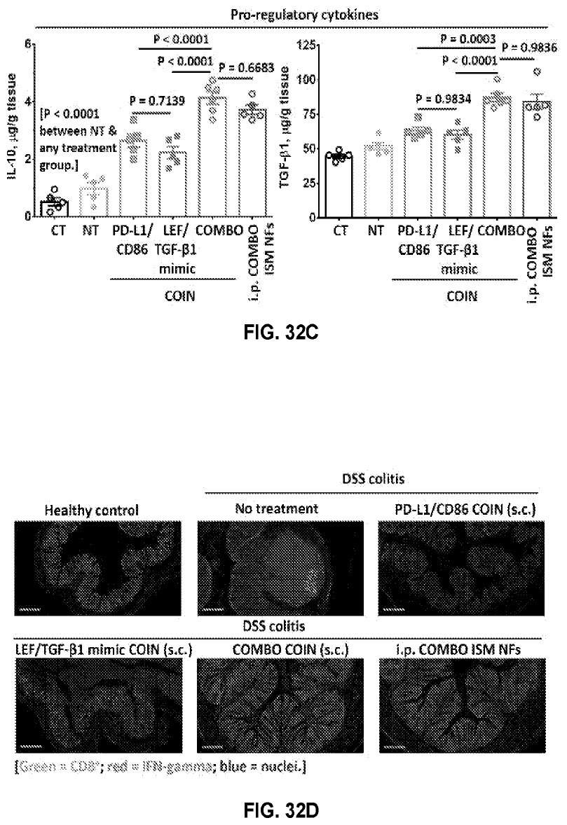

It was further examined whether the s.c. inoculation of COIN could induce organ-specific immunotolerance, ameliorate acute ulcerative colitis symptoms and speed up the recovery of ulcerated colonic mucosa using a well-established dextran sulfate sodium (DSS)-induced ulcerative colitis mouse model. The therapeutic efficacy of immune checkpoint molecule-functionalized COINs were first evaluated (FIG. 30A). It was found that all COINs bioengineered with immune checkpoint molecules effectively suppressed colitis symptoms (e.g., diarrhea and rectal bleeding; FIG. 30B), minimized bodyweight loss (FIG. 31A), and prevented inflammation-associated colon shortening in mice (FIG. 30C, and FIG. 31B). Specifically, at the study endpoint, mice treated with PD-L1/CD86 COIN exhibited complete bodyweight recovery (FIG. 31A), reduced inflammation-associated myeloperoxidase (MPO) activity (FIG. 32A), attenuated local pro-inflammatory cytokine expression (e.g., interleukin-6 (IL-6) and TNF-α) (FIG. 32B), and increased pro-regulatory cytokine expression (e.g., interleukin-10 (IL-10) and TGF-β1; FIG. 32C). Histological analysis confirmed that all treatments with immune checkpoint molecule-functionalized COINs significantly reduced immune cell infiltration into the colonic mucosa and submucosa (FIG. 30D-30E, and FIG. 31C).

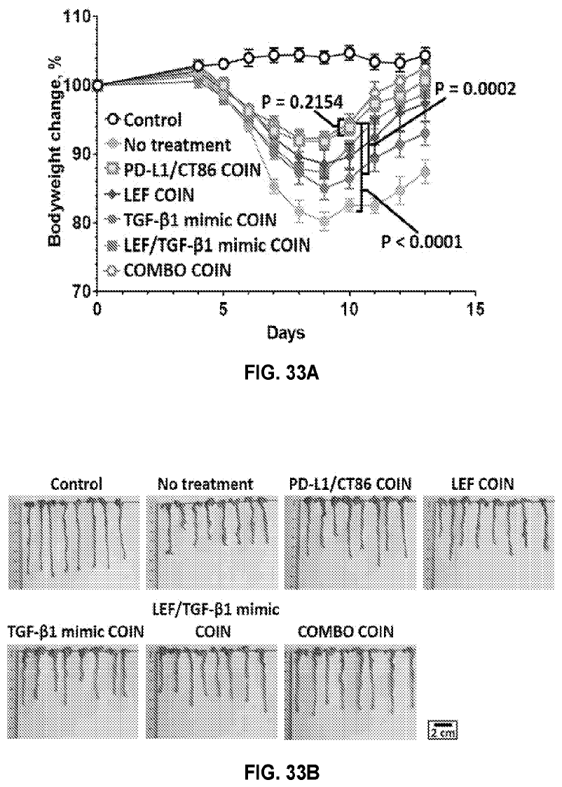

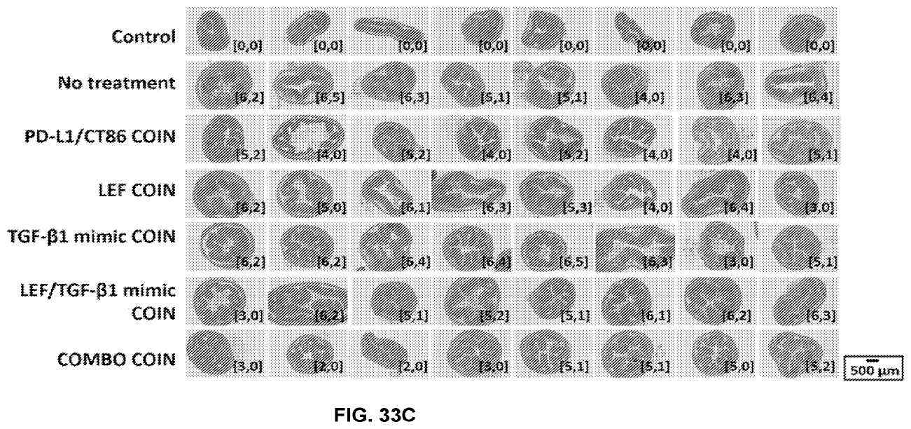

Next, the therapeutic efficiencies of COINs bioengineered with non-immune checkpoint immunomodulators were evaluated. Consistent with the weaker T cell inhibition efficiency observed for the LEF NFs or TGF-β1 mimic NFs in vitro (FIG. 17A-17E, FIG. 18A-18B, FIG. 20, FIG. 21, FIG. 23A-23C, FIG. 25A-25B, and FIG. 27), the LEF COIN and TGF-β1 mimic COIN were less effective in relieving colitis symptoms (FIG. 30F-30G) and promoting repair of colon epithelium damage (FIG. 30H-301) than immune checkpoint immunomodulator-functionalized COINs. However, the colitis mice treated with the LEF/TGF-β1 mimic COIN showed full bodyweight recovery (FIG. 33A), colon length maintenance (FIG. 30G, FIG. 33B), reduced local MPO activity (FIG. 32A), and proinflammatory cytokine expression (FIG. 32B, and FIG. 32D). The improved therapeutic efficiency of LEF/TGF-β1 mimic COIN can be explained by the abilities of both immunosuppressive components to simultaneously inhibit CD8⁺ T cell activation and polarize M1 Mφs through multiple regulatory pathways.

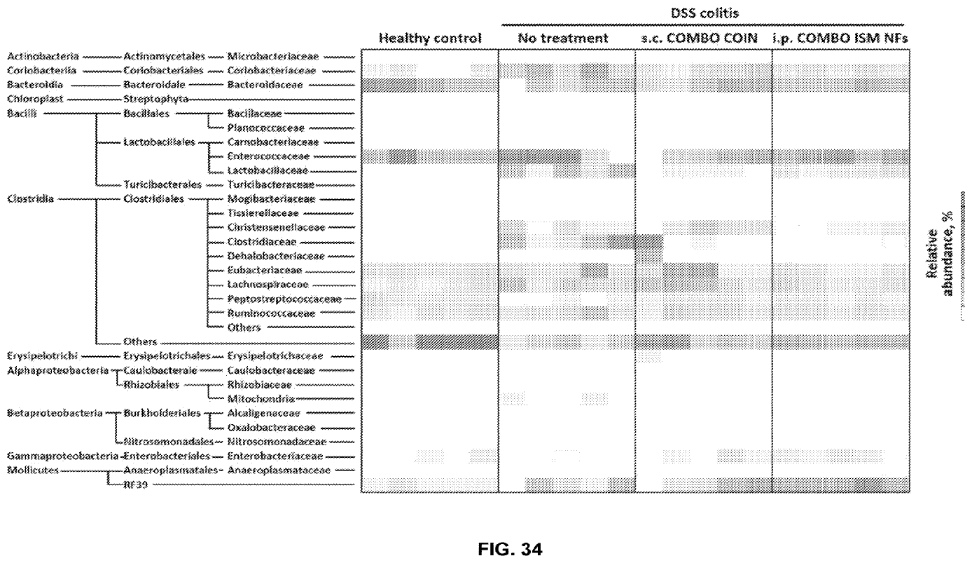

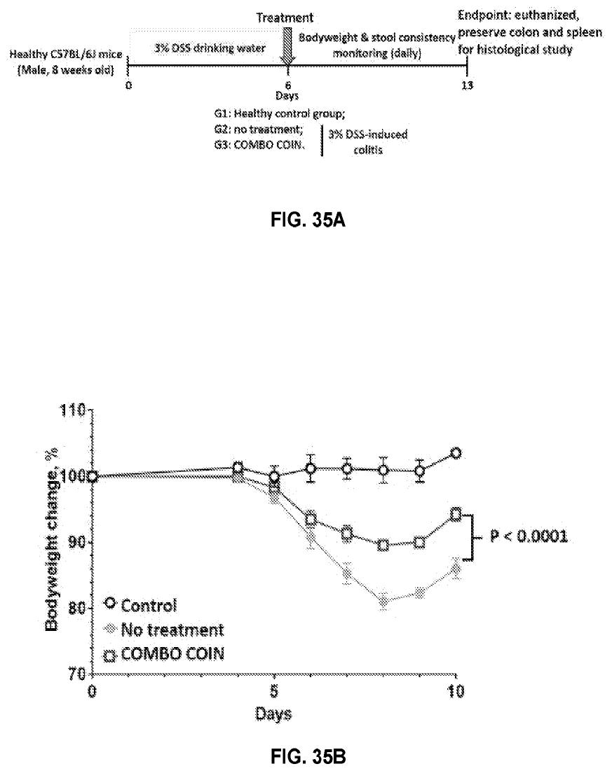

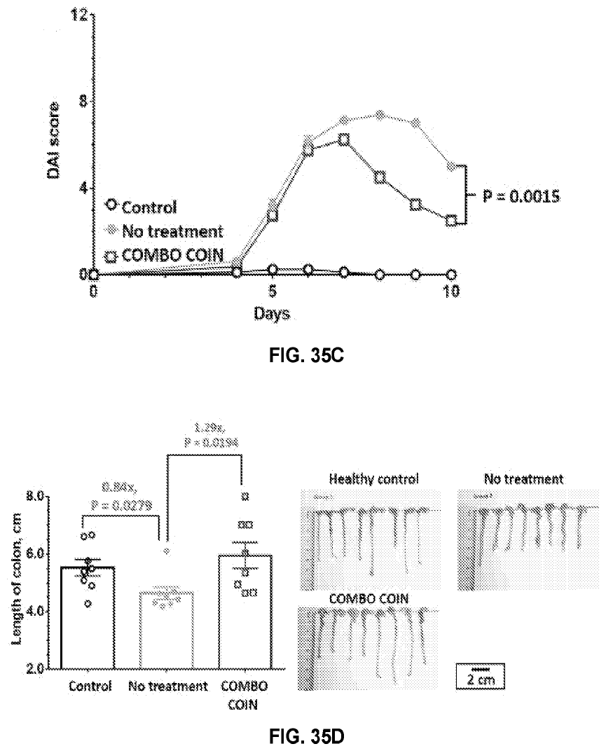

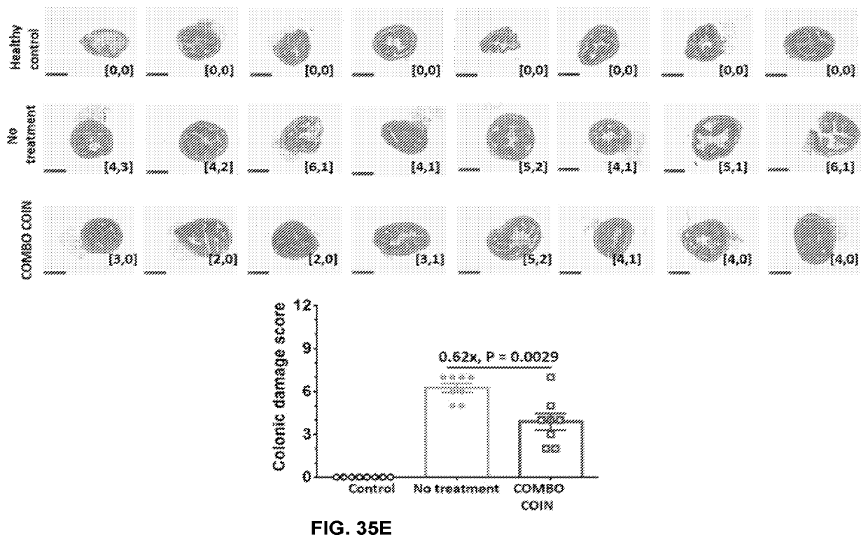

The COIN prepared from the COMBO ISM NFs (COMBO COIN) was further evaluated. Unlike the earlier treatments, s.c. treatment with COMBO COIN accelerated the amelioration of colitis symptoms compared to single immunomodulator-functionalized COINs and promoted complete recovery within 7 days aftertreatment (FIG. 30F, FIG. 33A-33B). The treatment also reduced local MPO activity (FIG. 32A) and proinflammatory cytokine expression to baseline levels (FIG. 32B) and increased the local production of pro-regulatory cytokines by nearly 3-fold compared to healthy mice (FIG. 32C). Further histological analysis showed that COMBO COIN was the only treatment that promoted complete recovery of epithelium damages within 7 days aftertreatment and prevented crypt loss (FIG. 33C). Fecal microbial analysis at the study endpoint indicated that COMBO COIN treatment partly resolved microbiome dysbiosis (FIG. 34, Table 3A-3C). To avoid fighting between male mice after the randomization (just before the colitis treatment) affecting the therapeutic study, most of the therapeutic studies were performed on female mice. Nevertheless, a similar effective therapeutic effect in male colitis mice that were randomized 1 week before the induction of colitis was observed (FIG. 35A-35E). This finding confirmed that the s.c. COMBO COIN treatment effect was sex-independent.

TABLE 3A
Statistical Analysis of Fecal Microbiome Compositions in Healthy control and Colitis, no treatment

| | | | | Colitis, | | P value |
|---|---|---|---|---|---|---|
| | | | Healthy | No | Fold-change | VS |
| | | | control | treatment | VS healthy | healthy |
| | | | Aver- | Aver- | control | control |
| | | | age % | age % | group | group |
| Actinobacteria | Actinomycetales | Microbacteriaceae | 0.0348 | 0.1180 | 3.40 | 0.1053 |
| Coriobacteriia | Coriobacteriales | Coriobacteriaceae | 1.2586 | 11.9506 | 9.50 | 0.0055 |
| Bacteroidia | Bacteroidale | Bacteroidaceae | 21.1180 | 8.0732 | 0.38 | 0.0117 |
| Chloroplast | Streptophyta | | 0.0000 | 0.0007 | N/A | N/A |
| Bacilli | Bacillales | Bacillaceae | 0.0000 | 0.0028 | N/A | N/A |
| | | Planococcaceae | 0.0007 | 0.0000 | 0.00 | 0.1733 |
| | Lactobacillales | Carnobacteriaceae | 0.0022 | 0.0490 | 22.02 | 0.0506 |
| | | Enterococcaceae | 18.0446 | 19.1740 | 1.06 | 0.4481 |
| | | Lactobacillaceae | 0.0128 | 9.0962 | 709.47 | 0.0064 |
| | Turicibacterales | Turicibacteraceae | 0.0000 | 0.0246 | N/A | N/A |
| Clostridia | Others | | 40.7313 | 6.5512 | 0.16 | 0.0016 |
| | Clostridiales | Others | 0.0068 | 0.0060 | 0.88 | 0.3235 |
| | | Mogibacteriaceae | 0.0000 | 0.0000 | N/A | N/A |
| | | Tissierellaceae | 0.0154 | 0.0122 | 0.79 | 0.3346 |
| | | Christensenellaceae | 0.2153 | 3.3284 | 15.46 | 0.0445 |
| | | Clostridiaceae | 0.1178 | 11.1710 | 94.79 | 0.0244 |
| | | Dehalobacteriaceae | 0.0000 | 0.0000 | N/A | N/A |
| | | Eubacteriaceae | 4.8530 | 7.4426 | 1.53 | 0.2546 |
| | | Lachnospiraceae | 2.6404 | 7.6683 | 2.90 | 0.0161 |
| | | Peptostreptococcaceae | 3.3743 | 0.8534 | 0.25 | 0.0008 |
| | | Ruminococcaceae | 4.3504 | 5.7265 | 1.32 | 0.2454 |
| Erysipelotrichi | Erysipelotrichales | Erysipelotrichaceae | 0.0080 | 0.1748 | 21.92 | 0.0250 |
| Alphaproteobacteria | Caulobacterale | Caulobacteraceae | 0.0000 | 0.0007 | N/A | N/A |
| | Rhizobiales | Rhizobiaceae | 0.0148 | 0.0038 | 0.25 | 0.0692 |
| | | Mitochondria | 0.1025 | 0.5846 | 5.70 | 0.0037 |
| Betaproteobacteria | Burkholderiales | Alcaligenaceae | 0.0000 | 0.0018 | N/A | N/A |
| | | Oxalobacteraceae | 0.0006 | 0.0032 | 5.52 | 0.0345 |
| | Nitrosomonadales | Nitrosomonadaceae | 0.0006 | 0.0000 | 0.00 | 0.1733 |
| Gammaproteobacteria | Enterobacteriales | Enterobacteriaceae | 1.0197 | 0.3483 | 0.34 | 0.1271 |
| Mollicutes | Anaeroplasmatales | Anaeroplasmataceae | 0.0593 | 0.0463 | 0.78 | 0.4303 |
| | RF39 | | 2.0172 | 7.5879 | 3.76 | 0.0621 |

TABLE 3B
Statistical Analysis of Fecal Microbiome Compositions in Colitis, COMBO COIN treatment

| | | | | | | Fold- | |
|---|---|---|---|---|---|---|---|
| | | | | Fold- | | change | P value |
| | | | Colitis, | change | P value | VS | VS |
| | | | COMBO | VS | VS | colitis | colitis |
| | | | COIN | healthy | healthy | non- | non- |
| | | | treatment | control | control | treatment | treatment |
| | | | Average % | group | group | group | group |
| Actinobacteria | Actinomycetales | Microbacteriaceae | 0.3270 | 9.41 | 0.0876 | 2.7710 | 0.0454 |
| Coriobacteriia | Coriobacteriales | Coriobacteriaceae | 4.7897 | 3.81 | 0.0384 | 0.4008 | 0.0203 |
| Bacteroidia | Bacteroidale | Bacteroidaceae | 12.6376 | 0.60 | 0.1603 | 1.5654 | 0.0186 |
| Chloroplast | Streptophyta | | 0.0684 | N/A | N/A | 94.7300 | 0.1733 |
| Bacilli | Bacillales | Bacillaceae | 0.1228 | N/A | N/A | 43.8591 | 0.0969 |
| | | Planococcaceae | 0.0478 | 72.89 | 0.1733 | N/A | N/A |
| | Lactobacilles | Carnobacteriaceae | 0.0363 | 16.30 | 0.3897 | 0.7402 | 0.0517 |
| | | Enterococcaceae | 10.6562 | 0.59 | 0.1549 | 0.5558 | 0.4478 |
| | | Lactobacillaceae | 0.7381 | 57.57 | 0.0095 | 0.0811 | 0.0478 |
| | Turicibacterales | Turicibacteraceae | 0.0040 | N/A | N/A | 0.1613 | 0.1611 |
| Clostridia | Others | | 16.1173 | 0.40 | 0.0258 | 2.4602 | 0.0005 |
| | Clostridiales | Others | 0.0448 | 6.56 | 0.0193 | 7.4121 | 0.0014 |
| | | Mogibacteriaceae | 0.0034 | N/A | N/A | N/A | N/A |
| | | Tissierellaceae | 0.0214 | 1.39 | 0.2192 | 1.7596 | 0.3778 |
| | | Christensenellaceae | 5.1024 | 23.69 | 0.2454 | 1.5330 | 0.1149 |
| | | Clostridiaceae | 6.5605 | 55.67 | 0.2855 | 0.5873 | 0.0254 |
| | | Dehalobacteriaceae | 2.7264 | N/A | N/A | N/A | N/A |
| | | Eubacteriaceae | 10.9534 | 2.26 | 0.2924 | 1.4717 | 0.1164 |
| | | Lachnospiraceae | 10.1404 | 3.84 | 0.2423 | 1.3224 | 0.1374 |
| | | Peptostrepto- | 2.6241 | 0.78 | 0.0164 | 3.0748 | 0.0021 |
| | | coccaceae | | | | | |
| | | Ruminococcaceae | 4.3573 | 1.00 | 0.2645 | 0.7609 | 0.3765 |
| Erysipelotrichi | Erysipelotrichales | Erysipelotrichaceae | 0.2698 | 33.84 | 0.3173 | 1.5437 | 0.0432 |
| Alphaproteo- | Caulobacterale | Caulobacteraceae | 0.0529 | N/A | N/A | 77.7055 | 0.1733 |
| bacteria | | | | | | | |
| | Rhizobiales | Rhizobiaceae | 0.0538 | 3.63 | 0.1812 | 14.3443 | 0.2201 |
| | | Mitochondria | 0.2562 | 2.50 | 0.0265 | 0.4383 | 0.0102 |
| Betaproteo- | Burkholderiales | Alcaligenaceae | 0.0434 | N/A | N/A | 24.0390 | 0.1733 |
| bacteria | | | | | | | |
| | | Oxalobacteraceae | 0.0335 | 58.68 | 0.1858 | 10.6260 | 0.0483 |
| | Nitrosomonadales | Nitrosomonadaceae | 0.0316 | 57.23 | 0.1733 | N/A | N/A |
| Gammaproteo- | Enterobacteriales | Enterobacteriaceae | 1.6670 | 1.63 | 0.0885 | 4.7861 | 0.0115 |
| bacteria | | | | | | | |
| Mollicutes | Anaero- | Anaeroplasmataceae | 0.0318 | 0.54 | 0.3853 | 0.6881 | 0.1030 |
| | plasmatales | | | | | | |
| | RF39 | | 9.4453 | 4.68 | 0.3674 | 1.2448 | 0.0031 |

TABLE 3C
Statistical Analysis of Fecal Microbiome Compositions in Colitis, i.p. COMBO ISM NFS

| | | | | | | Fold- | |
|---|---|---|---|---|---|---|---|
| | | | | Fold- | | change | P value |
| | | | Colitis, | change | P value | VS | VS |
| | | | i.p. | VS | VS | colitis | colitis |
| | | | COMBO | healthy | healthy | non- | non- |
| | | | ISM NFs | control | control | treatment | treatment |
| | | | Average % | group | group | group | group |
| Actinobacteria | Actinomycetales | Microbacteriaceae | 0.0008 | 0.02 | 0.0454 | 0.01 | 0.0001 |
| Coriobacteriia | Coriobacteriales | Coriobacteriaceae | 3.9670 | 3.15 | 0.0203 | 0.33 | 0.0022 |
| Bacteroidia | Bacteroidale | Bacteroidaceae | 14.7173 | 0.70 | 0.0186 | 1.82 | 0.0850 |
| Chloroplast | Streptophyta | | 0.0000 | N/A | 0.1733 | 0.00 | N/A |
| Bacilli | Bacillales | Bacillaceae | 0.0000 | N/A | 0.0969 | 0.00 | N/A |
| | | Planococcaceae | 0.0000 | 0.00 | N/A | N/A | 0.1733 |
| | Lactobacillales | Carnobacteriaceae | 0.0024 | 1.08 | 0.0517 | 0.05 | 0.4738 |
| | | Enterococcaceae | 20.1907 | 1.12 | 0.4478 | 1.05 | 0.3560 |
| | | Lactobacillaceae | 3.3325 | 259.92 | 0.0478 | 0.37 | 0.0086 |
| | Turicibacterales | Turicibacteraceae | 0.0012 | N/A | 0.1611 | 0.05 | 0.1733 |
| Clostridia | Others | | 16.8328 | 0.41 | 0.0005 | 2.57 | 0.0094 |
| | Clostridiales | Others | 0.0216 | 3.16 | 0.0014 | 3.58 | 0.0026 |
| | | Mogibacteriaceae | 0.0000 | N/A | N/A | N/A | N/A |
| | | Tissierellaceae | 0.0145 | 0.94 | 0.3778 | 1.19 | 0.4236 |
| | | Christensenellaceae | 1.1799 | 5.48 | 0.1149 | 0.35 | 0.0201 |
| | | Clostridiaceae | 0.2296 | 1.95 | 0.0254 | 0.02 | 0.1719 |
| | | Dehalobacteriaceae | 0.0041 | N/A | N/A | N/A | N/A |
| | | Eubacteriaceae | 2.6135 | 0.54 | 0.1164 | 0.35 | 0.0176 |
| | | Lachnospiraceae | 5.2704 | 2.00 | 0.1374 | 0.69 | 0.0160 |
| | | Peptostrepto- | 2.0520 | 0.61 | 0.0021 | 2.40 | 0.0228 |
| | | coccaceae | | | | | |
| | | Ruminococcaceae | 5.1265 | 1.18 | 0.3765 | 0.90 | 0.1724 |
| Erysipelotrichi | Erysipelotrichales | Erysipelotrichaceae | 0.0331 | 4.15 | 0.0432 | 0.19 | 0.0057 |
| Alphaproteo- | Caulobacterale | Caulobacteraceae | 0.0000 | N/A | 0.1733 | 0.00 | N/A |
| bacteria | | | | | | | |
| | Rhizobiales | Rhizobiaceae | 0.0072 | 0.48 | 0.2201 | 1.91 | 0.1680 |
| | | Mitochondria | 0.1908 | 1.86 | 0.0102 | 0.33 | 0.0256 |
| Betaproteo- | Burkholderiales | Alcaligenaceae | 0.0000 | N/A | 0.1733 | 0.00 | N/A |
| bacteria | | | | | | | |
| | | Oxalobacteraceae | 0.0008 | 1.34 | 0.0483 | 0.24 | 0.3739 |
| | Nitrosomonadales | Nitrosomonadaceae | 0.0000 | 0.00 | N/A | N/A | 0.1733 |
| Gammaproteo- | Enterobacteriales | Enterobacteriaceae | 2.4081 | 2.36 | 0.0115 | 6.91 | 0.0800 |
| bacteria | | | | | | | |
| Mollicutes | Anaero- | Anaeroplasmataceae | 0.1550 | 2.61 | 0.1030 | 3.35 | 0.1594 |
| | plasmatales | | | | | | |
| | RF39 | | 21.6484 | 10.73 | 0.0031 | 2.85 | <0.0001 |

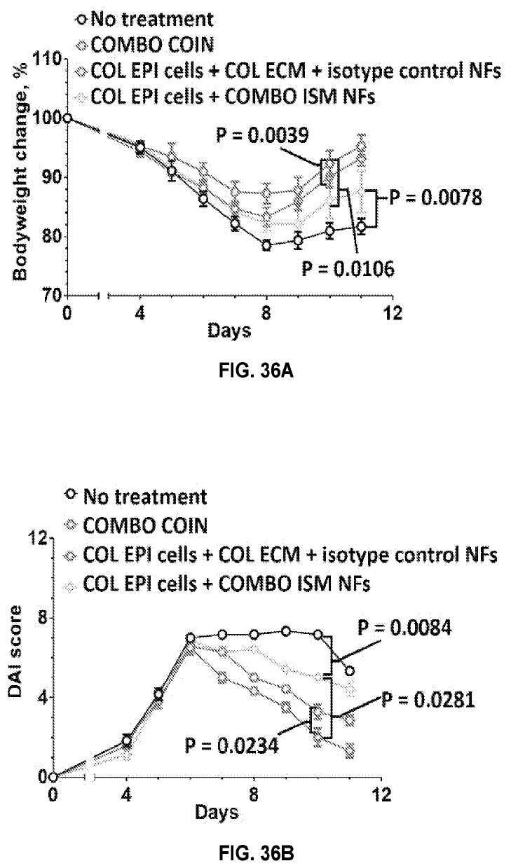

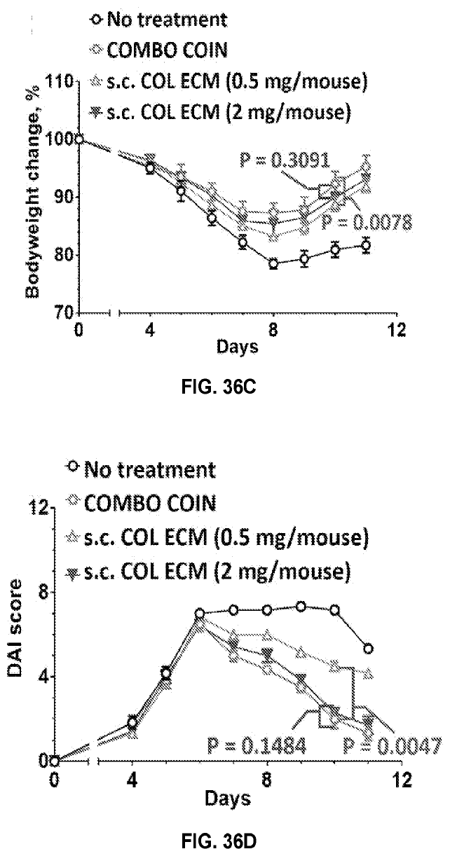

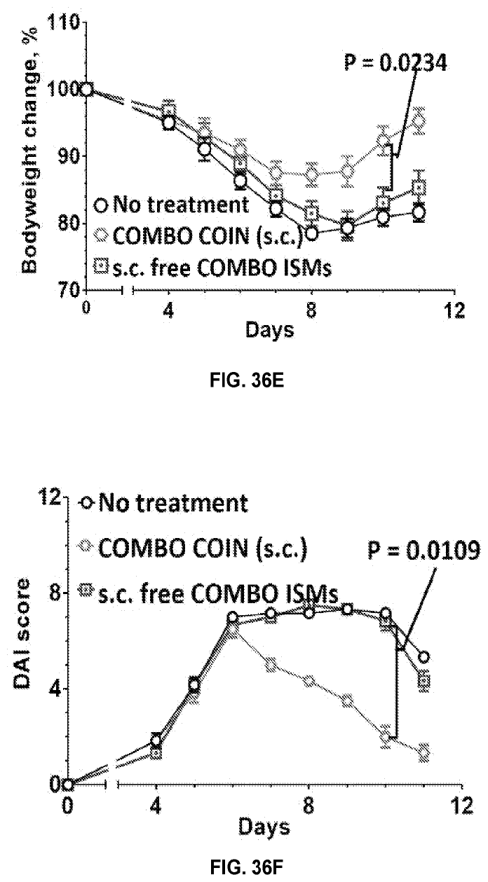

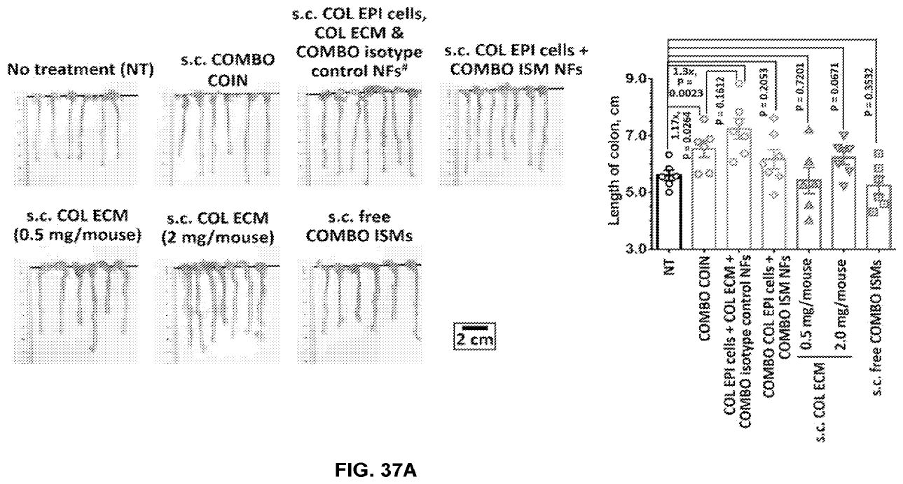

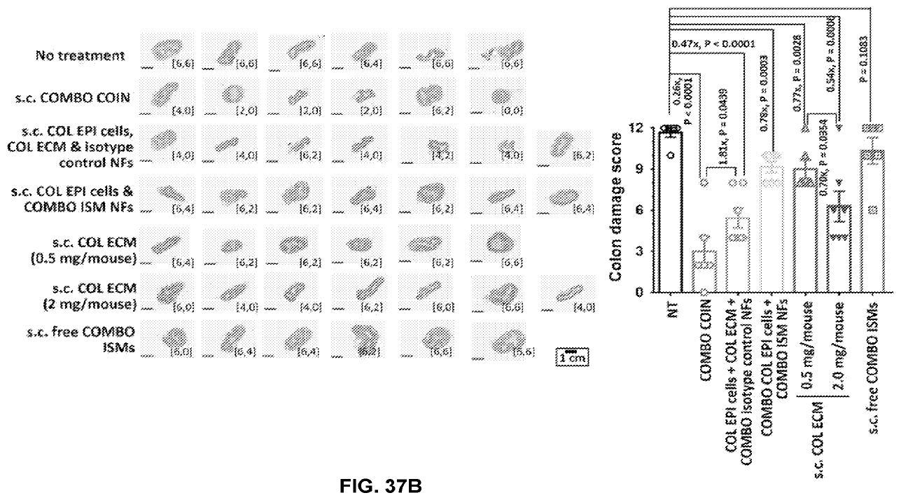

Given the CD8+ T cell anergization and M1 Mφ polarization abilities observed for the colon ECM in vitro (FIG. 19A-19G), control studies were performed to investigate the therapeutic effect of colon ECM. Administrating a therapeutic dose of colon ECM (0.5 mg/treatment, the same quantity as in the COMBO COIN) failed to relieve colitis symptoms (FIG. 36A-36F, and FIG. 37A-37B) and prevent colitis-associated colon epithelium damage and immune cell infiltration (FIG. 36A-36F, and FIG. 37A-37B). Colitis treatment with a high-dose of colon ECM (4 mg/treatment) was as effective as COMBO COIN in ameliorating colitis symptoms (FIG. 36A-36F, and FIG. 37A-37B). However, the treatment did not prevent inflammation-associated colon shortening, colon epithelium damage or immune cell infiltration into the lamina propria (LP). These findings indicated that colon ECM alone was ineffective in treating ulcerative colitis. Obtaining a large quantity of colon tissues from patients with ulcerative colitis to engineer the immune niche would be difficult.

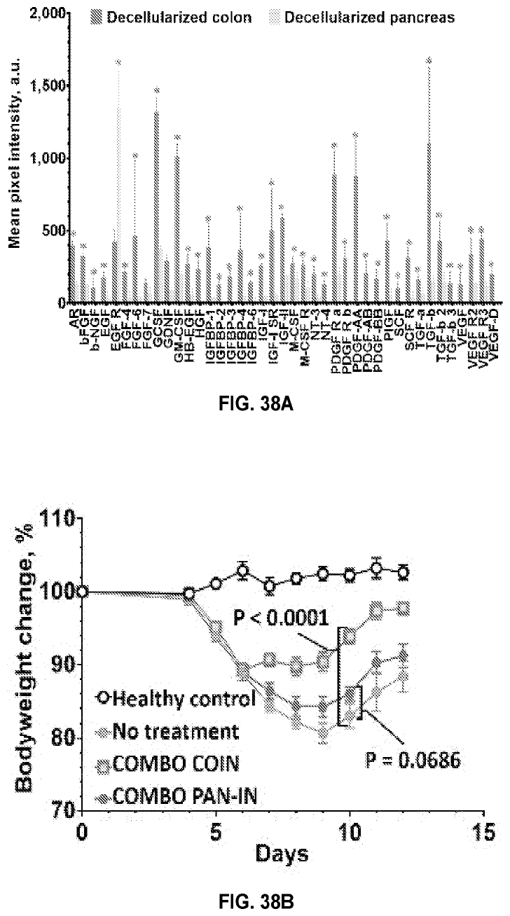

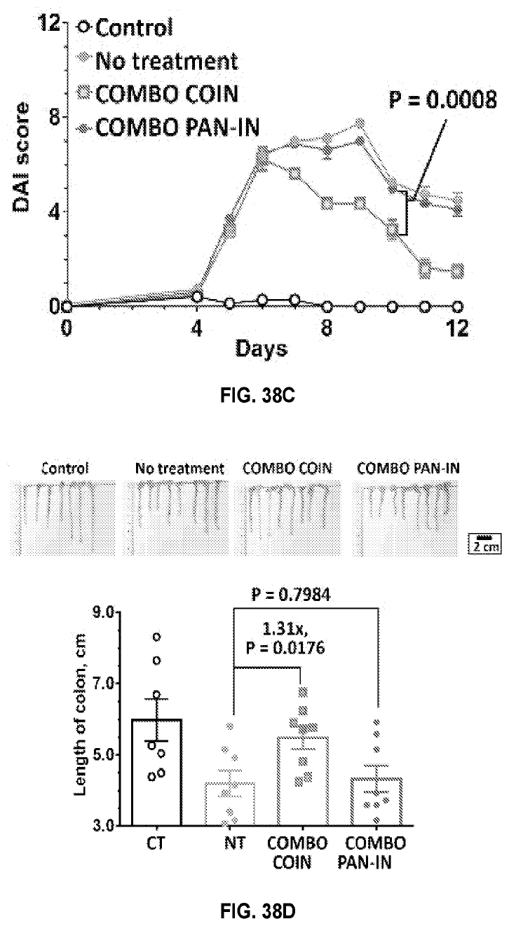

Additional control studies administered immune niches engineered from colon epithelial cells and COMBO ISM NFs (FIG. 36A-36F), pancreatic β cells (MIN6 cells), decellularized pancreas ECM and COMBO ISM NFs (COMBO pancreas immune niche, COMBO PAN-IN). The treatment failed to alleviate the colitis symptoms (FIG. 38A-38D) or prevent inflammation-associated colon shortening (FIG. 38A-38D). This finding confirmed that the colon ECM released pro-regulatory growth factors and cytokines, generating a colon-specific tissue microenvironment to modulate autoreactive immune cells that were observed in vitro (FIG. 19A-19G). Additional control studies administered isotype control COMBO COIN engineered from colon epithelial cells, colon ECM and COMBO isotype control NFs (composed of isotype control mouse IgG-functionalized NFs, drug-free NP-functionalized NFs and non-functionalized NFs). These showed moderate therapeutic effects and relieved the colitis symptoms (FIG. 36A-36F, and FIG. 37A-37B). However, they were less effective in promoting the repair of epithelium damage and preventing immune cell infiltration into the lamina propria (FIG. 36A-36F, and FIG. 37A-37B) than the COMBO COIN treatment. These findings confirmed that COMBO ISM NFs play a key role in ameliorating colitis symptoms.

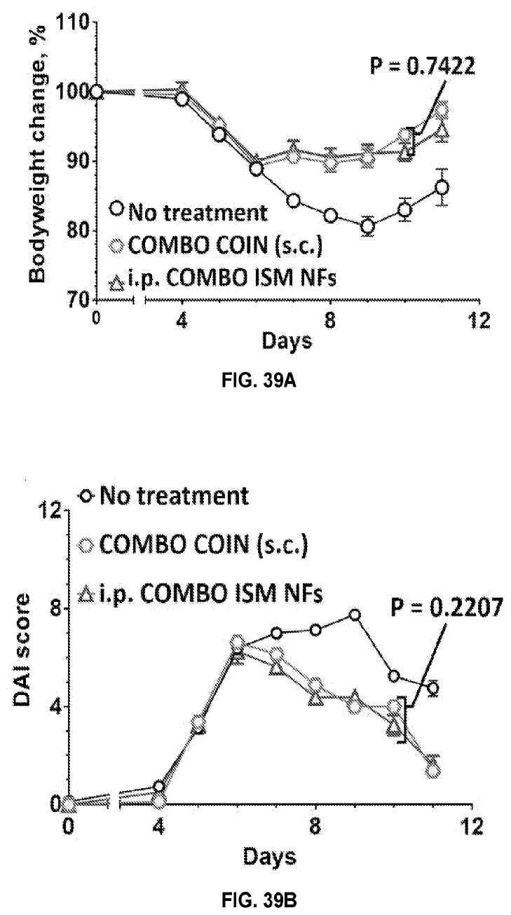

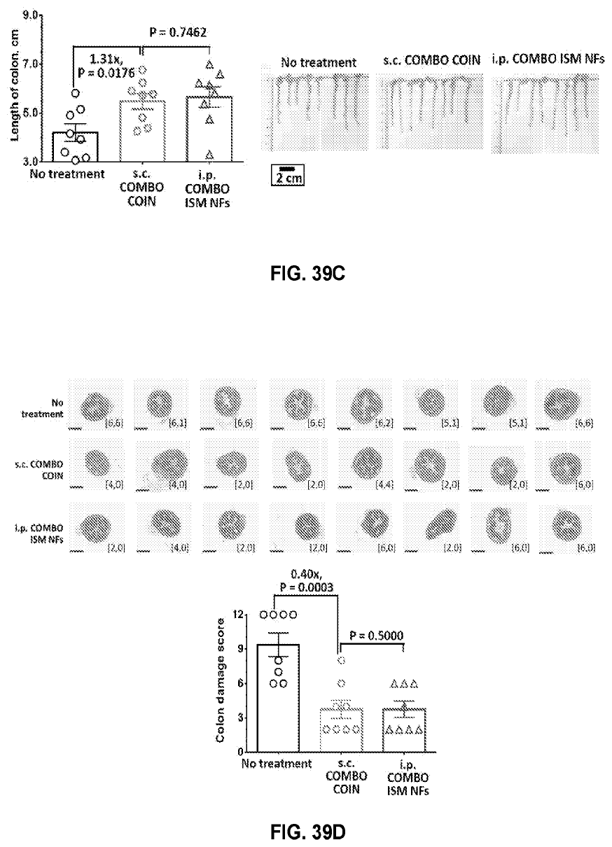

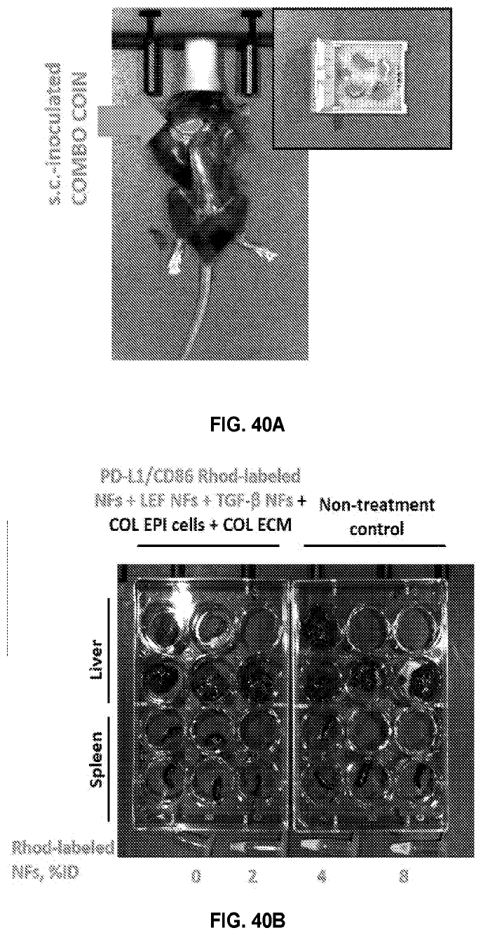

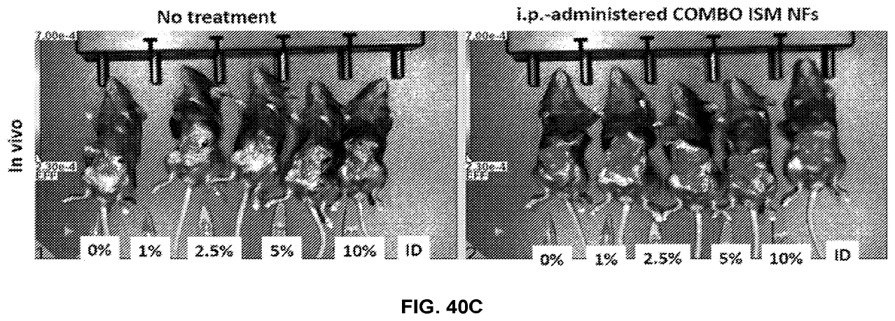

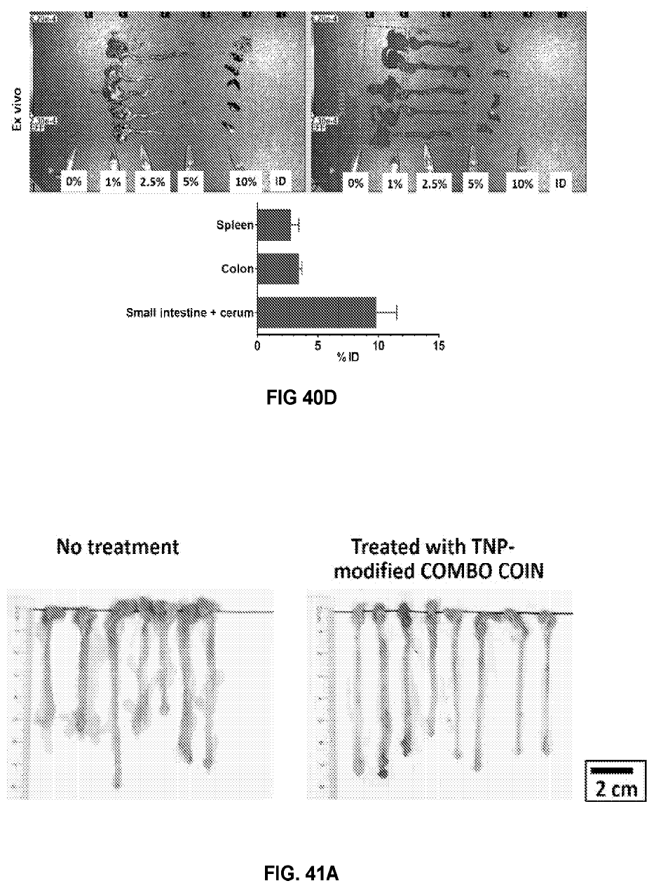

Recognizing the translational challenges for bioengineering COMBO COIN, it was further investigated whether intraperitoneal (i.p.)-administered fully synthetic COMBO ISM NFs could treat UC since they can directly engage autoreactive immune cells in the abdominal cavity. Similar to the s.c. COMBO COIN, i.p. COMBO ISM NFs effectively suppressed colitis symptoms (FIG. 39A-39B), prevented inflammation-associated colon shortening (FIG. 39C), promoted repair of epithelium damage (FIG. 39D), and reduced immune cell infiltration into the colon (FIG. 39D). Fecal microbial analysis showed that the i.p. treatment was more effective than the s.c. COMBO COIN treatment resolved microbiota dysbiosis (FIG. 34, Table 3A-3C) and upregulated several commensal microbiomes (e.g., RF39). An ex vivo biodistribution study performed 5 days after the i.p. treatment showed that most administered rhodamine-labeled NFs were distributed throughout the abdominal cavity (FIG. 40A-40D), whereas the s.c. administered COMBO COIN was retained at the injection site. These findings highlighted the direct therapeutic potential of COMBO COIN when autogenous colon epithelial cells and colon ECM are unavailable.

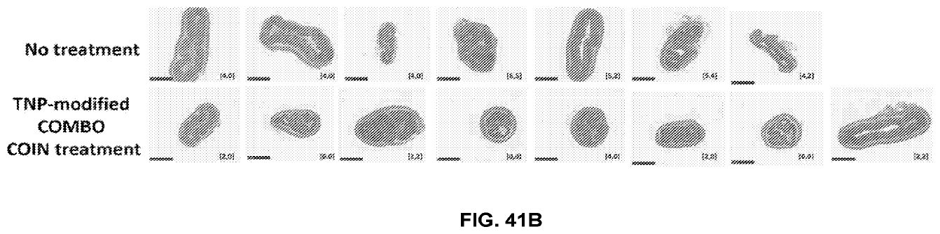

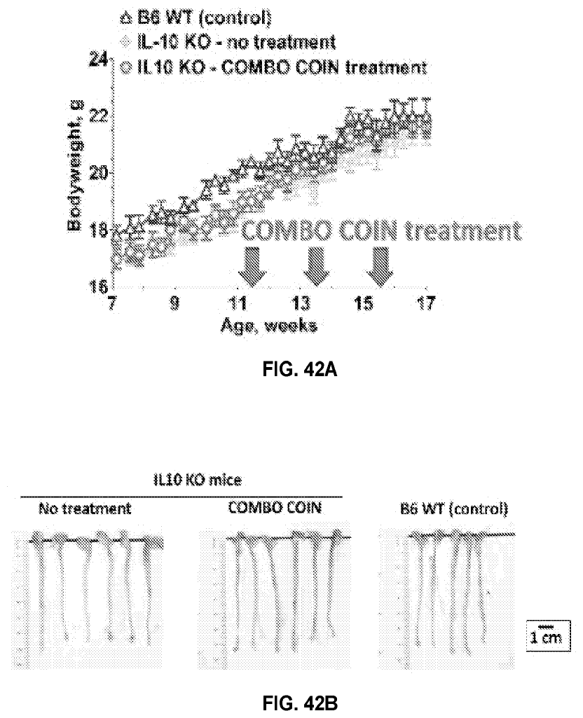

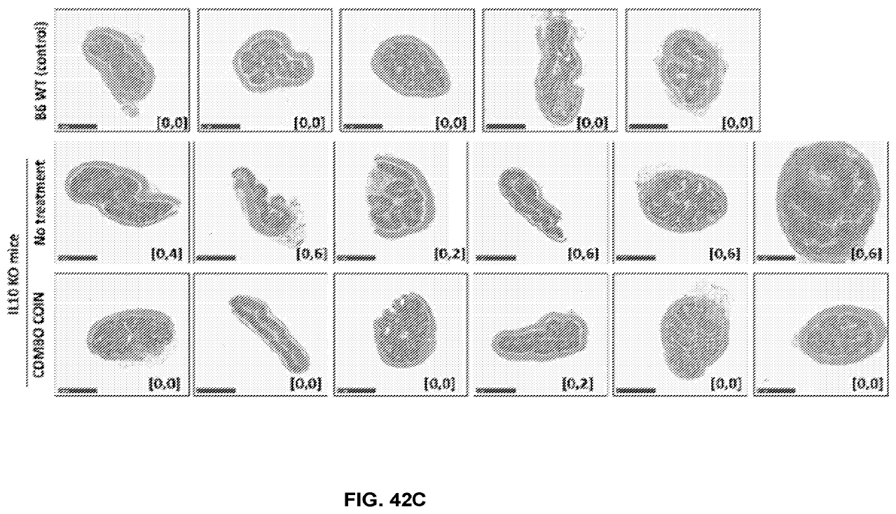

To demonstrate that COMBO COIN can ameliorate different types of colitis, COMBO COIN in the TNP-induced colitis model and IL10 KO mouse model of spontaneous colitis were further evaluated. The TNP-induced colitis model is an established CD8+ T cell-driven relapsing colitis model in which TNP-sensitized CD8+ T cells attack TNP-modified colon epithelial cells. In the TNP-induced colitis mice (FIG. 30J), treatment with a TNP-modified COMBO COIN (a COMBO COIN engineered with TNP-modified colon epithelial cells) significantly relieved the severe colitis symptoms (e.g., rectal bleeding and rectum prolapse) within 24 h after treatment (FIG. 30K), minimized colitis-associated colon shortening (FIG. 30L, FIG. 41A), and promoted repair of the ulcerated epithelium (FIG. 30M-30N, and FIG. 41B). In the IL10 KO mouse model, mice spontaneously develop enterocolitis due to the dysfunction of regulatory T (Treg) cells that cannot produce pro-regulatory IL1055. Repeated treatments with COMBO COINs upon colitis onset prevented disease progression (FIG. 30O), such as hematochezia, colitis-induced anemia (FIG. 30P), lymphocytic colitis (FIG. 30Q), inflammation-induced colon shortening (FIG. 30R, and FIG. 42A-42C), and immune cell infiltration into the LP (FIG. 30S-30T, and FIG. 42A-42C). This finding confirmed that the COMBO COIN treatment strategy was applicable to a broad range of colitis.

### Example 4: COINs Ameliorate Colitis by Inhibiting Autoreactive CD8⁺ T Cell Activation and Polarization of M1 Mφs into M2 Mφs

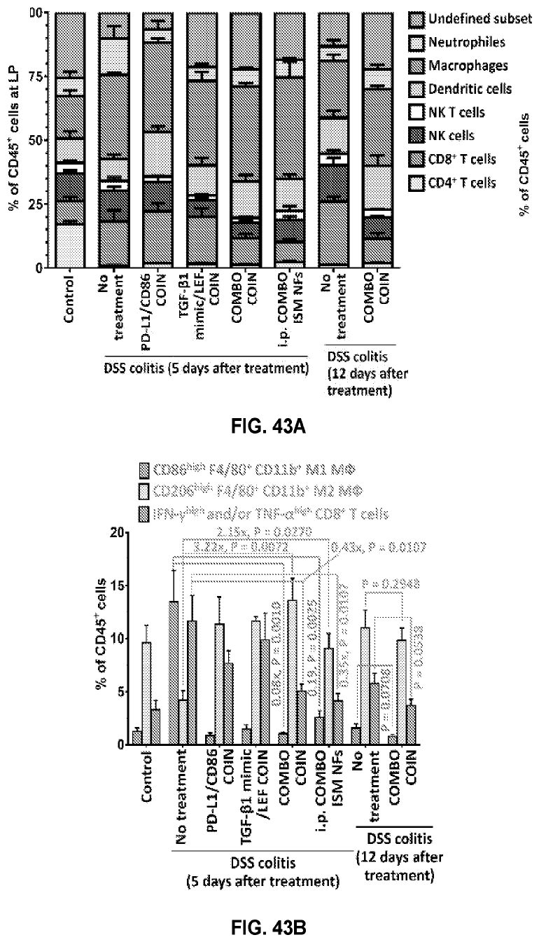

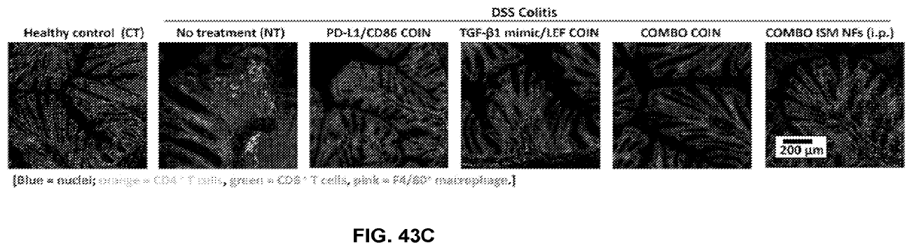

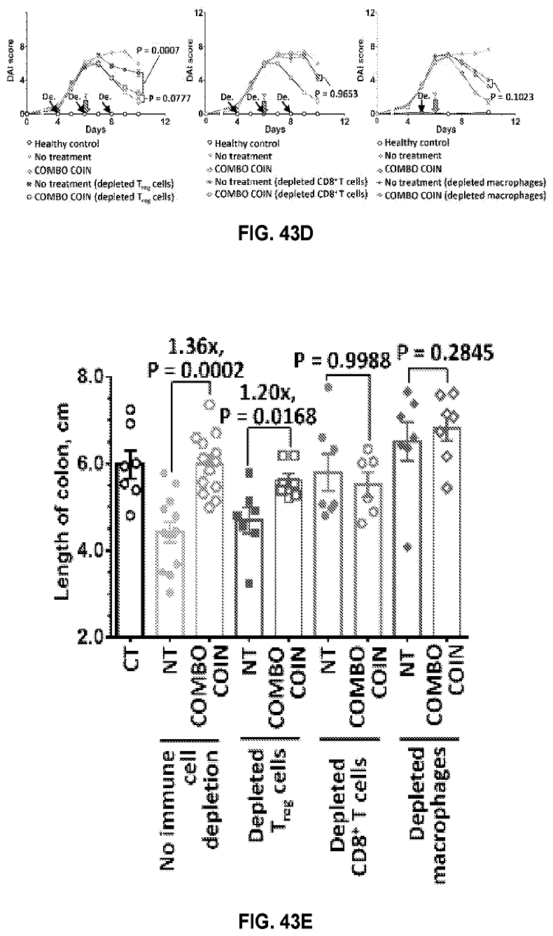

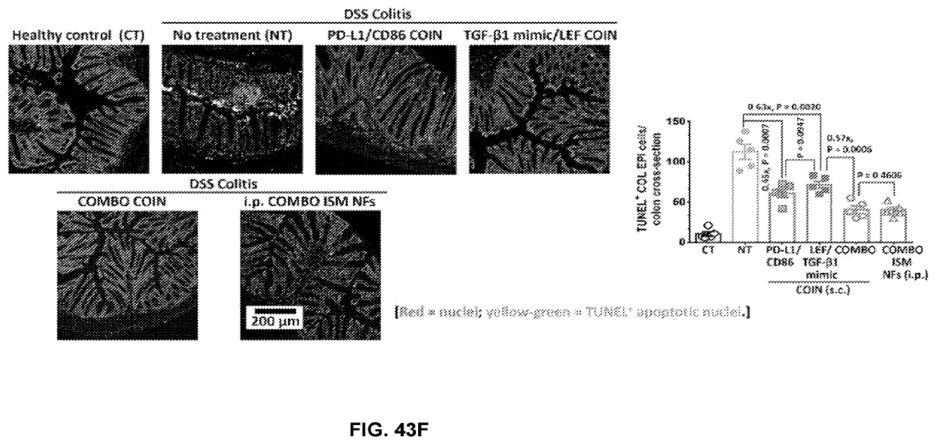

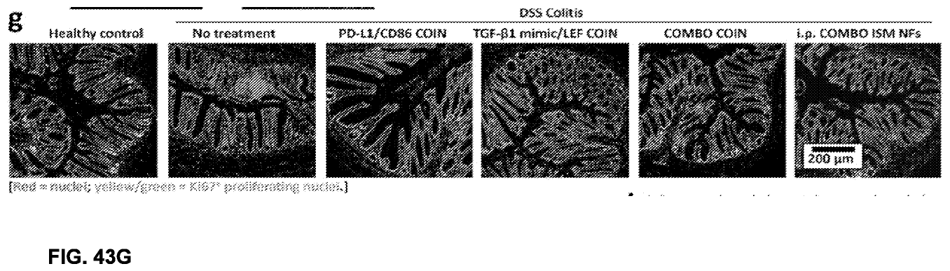

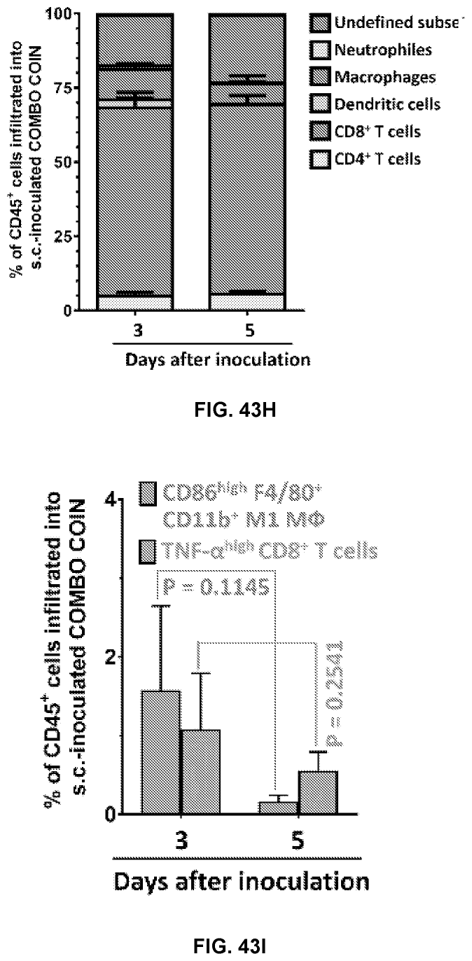

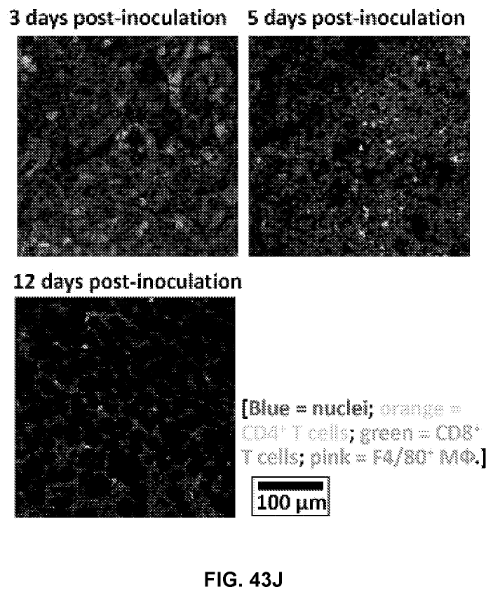

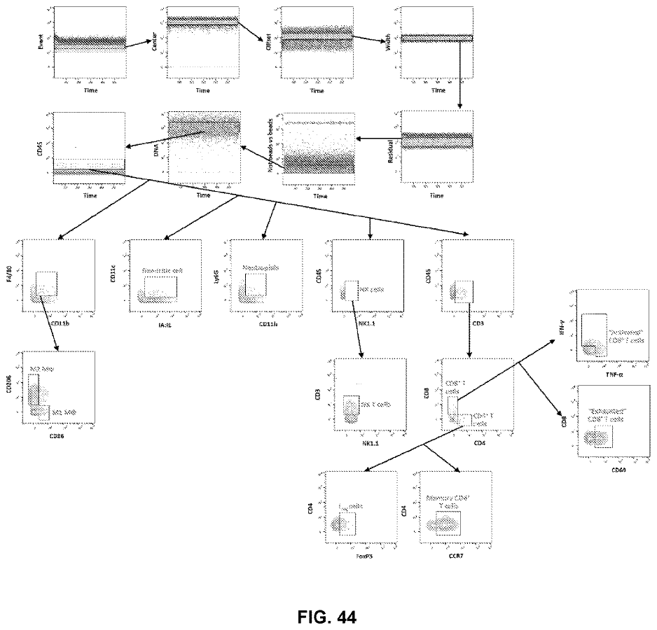

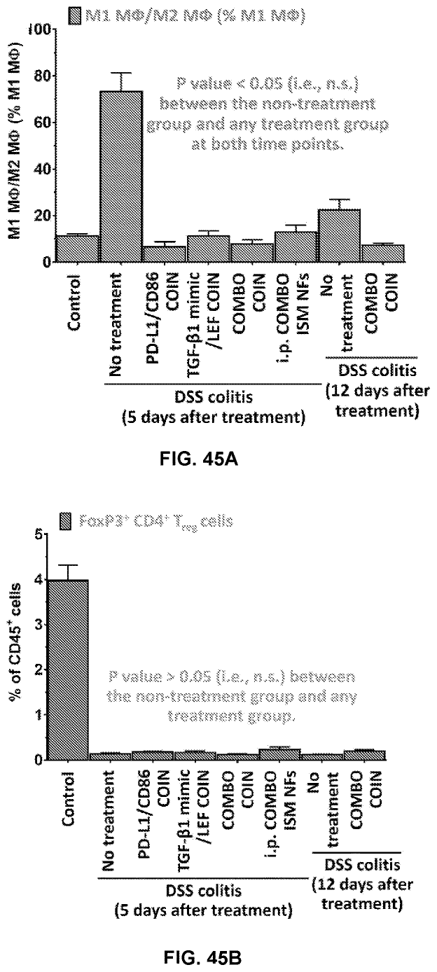

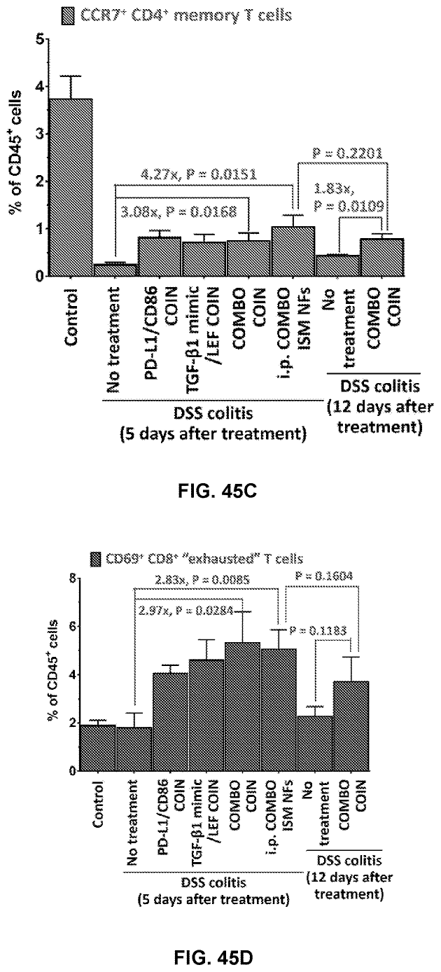

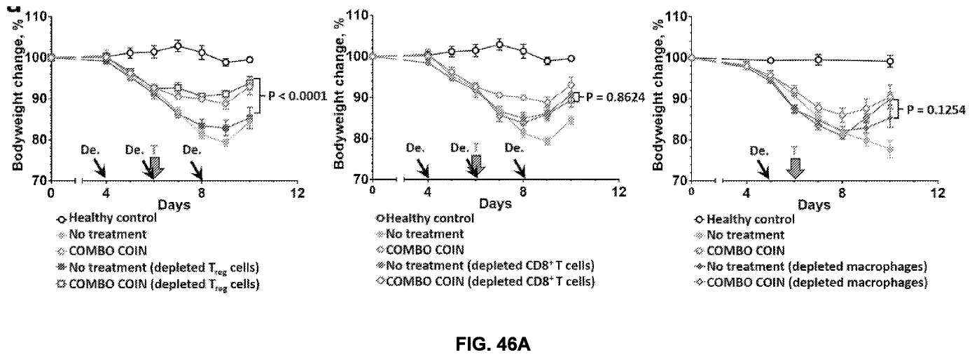

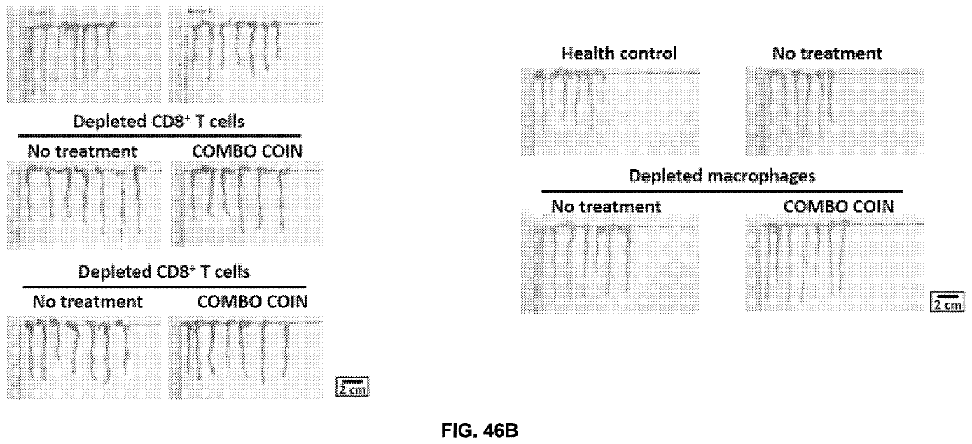

Immune profiling was performed (FIG. 43A, and FIG. 44) to better understand different immune treatment action mechanisms in the DSS-induced colitis model. Colitis significantly increased the frequencies of proinflammatory CD₈₆^{high} M1 Mφs and IFN-γ^{high} and/or TNF-α^{high} CD8+ T cells and decreased the frequencies of CCR7⁺ CD4⁺ memory T cells and pro-regulatory FoxP3⁺ CD4⁺ T_{reg} cells infiltrating the LP compared to those in the healthy colon (FIG. 43A-43B, and FIG. 45A-45D). 5 days after colitis treatment, COMBO COIN significantly reduced the frequencies of CD₈₆^{high} M1 Mφs (thus the M1 Mφ/M2 Mφ ratio; FIG. 43B, and FIG. 45A-45D) and IFN-γ^{high} and TNF-α^{high} CD8+ T cells compared to the non-treatment group (FIG. 43B, and FIG. 45A-45D), normalized the frequency of CD206^{high} M2 Mφs to the background level (FIG. 43B, and FIG. 45A-45D), and increased the frequency of exhausted CD8⁺ T cells (e.g., CD69) (FIG. 45A-45D). The treatment also significantly increased the frequency of CCR7⁺ CD4⁺ memory T cells compared to the non-treatment group (FIG. 45A-45D) but did not normalize the frequency of T_{reg} cells (FIG. 45A-45D). Similar reductions in proinflammatory M1 Mφs and CD8⁺ T cells were observed in the LP of colitis mice receiving i.p. treatment with COMBO ISM NFs (FIG. 43A-43B, and FIG. 45A-45D). This result was consistent with the reduced infiltration of CD8⁺ T cells, especially INF-γ⁺ CD8⁺ T cells, observed in the histological analysis, (FIG. 43C, and FIG. 32D) and the reduced therapeutic efficacy in CD8⁺ T cell- and Mφ-depleted colitis mice (FIG. 43D-43E, and FIG. 46A-46B). The immune cell depletion study supported the anti-colitis effect of COMBO COIN observed in the IL10 KO mice. If the COMBO COIN works by modulating CD4⁺ T cells in both models, any anti-colitis effect in the IL10 KO mice would not be observed because its CD4⁺ T cells cannot produce IL10 to regulate the CD8⁺ T cells and Mφs. Further immune profiling of colons preserved 12 days post-treatment showed a similar reduction of in lamina propria-infiltrated CD8⁺ T cells and M1 Mφs (FIG. 43A-43B, and FIG. 45A-45D). However, the reductions were less significant than those in the non-treatment group (FIG. 43A-43B, and FIG. 45A-45D) due to the recovery from inflammation after feeding with normal drinking water. Nevertheless, this mechanistic study confirmed the hypothesis that the proposed COMBO COIN could directly inhibit CD8⁺ T cell activation and promoted M1 Mφs differentiation into M2 Mφs.

Immunohistological analysis was performed on preserved colon specimens 4 days after treatment to show that reduced CD8⁺ T cell and M1 Mφ infiltration created less inflammatory tissue microenvironments that allow healthy COL EPI stem cells in crypts to repair epithelial damage. Without any colitis treatment, significant apoptosis of colon epithelial cells (TUNEL⁺ cells) in the villi and immune cell invasion were observed (FIG. 43F), and most colon epithelial cells at the crypt were arrested in the Go phase or underwent apoptosis (i.e., Ki67⁻ cells; FIG. 43G). Therapeutic treatments with COINs significantly reduced immune invasion and apoptotic TUNEL⁺ epithelial cells in the villi and increased the frequency of proliferating Ki67⁺ EPI cells at in the crypt (FIG. 43G). Specifically, treatment with COMBO COIN reduced apoptotic TUNEL⁺ epithelial cells in the villi by approximately 65% (FIG. 43F) and nearly completely repaired the damaged colon epithelium. The recovery of colitis-induced EPI damage was observed after i.p. treatment with COMBO ISM NFs.

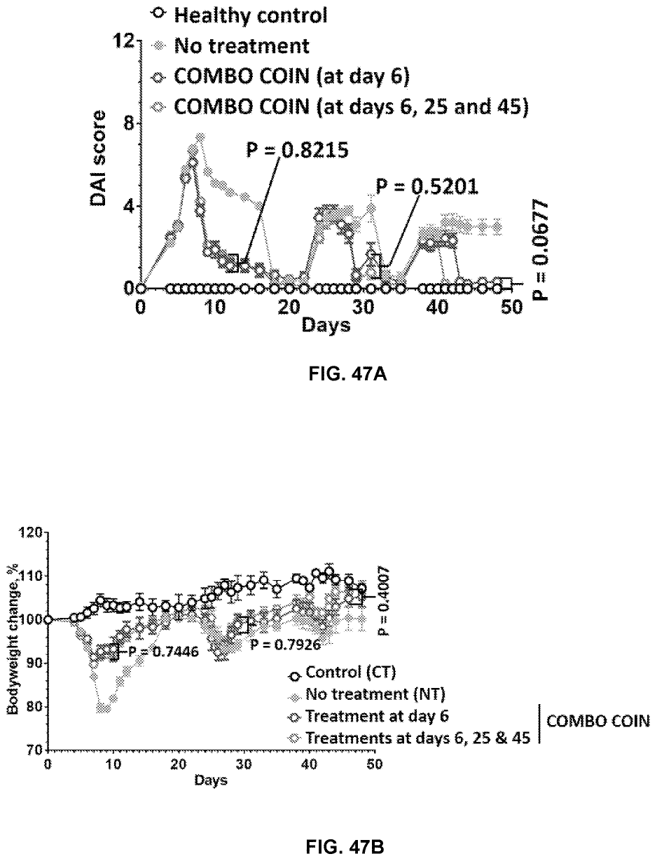

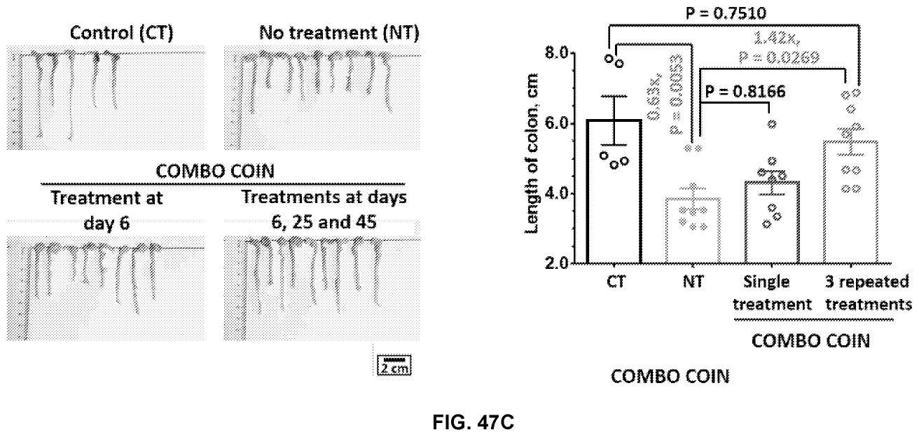

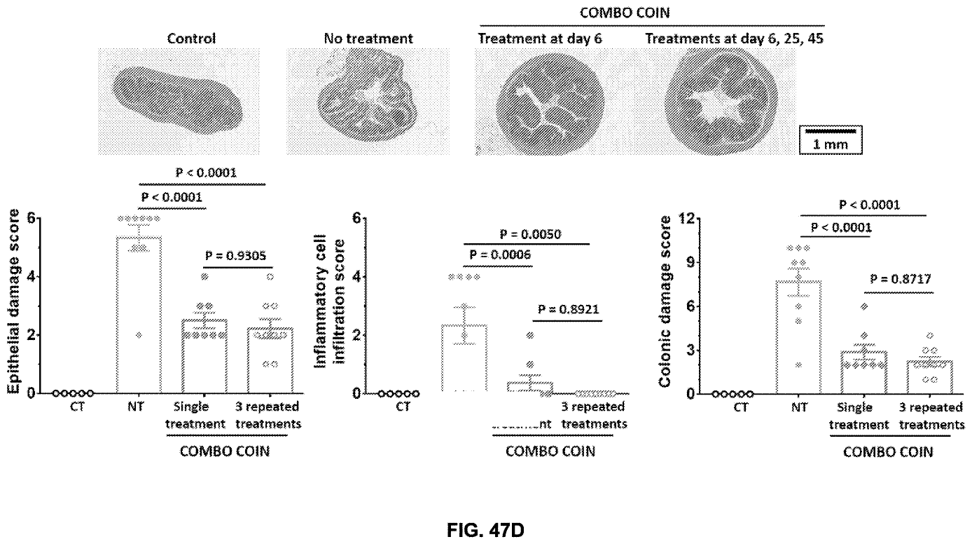

A therapeutic study in a DSS-induced chronic colitis mouse model was performed to demonstrate that the partial restoration of colonic CCR7⁺ CD4⁺ memory T cells after colitis treatment with COMBO COIN induced memory immune responses (FIG. 47A-47D). Similar to the monophasic DSS-induced colitis study, mice that repeatedly received treatment with COMBO COIN showed complete bodyweight recovery (FIG. 47A-47D), maintained colon length (FIG. 47A-47D), and reduced inflammation-associated colon epithelium damage (FIG. 47A-47D) after three colitis induction-recovery cycles. However, another experimental group that received only one treatment with COMBO COIN in the first colitis onset cycle also showed comparable recovery patterns to those who repeatedly received treatment (FIG. 47A-47D). At the study endpoint, both groups of mice showed similar bodyweight recovery (FIG. 47A-47D), colon lengths (FIG. 29B-29C), and colon damage scores (FIG. 47A-47D). Hence, memory T cells likely developed in the first phase of treatment, preventing further colitis development.

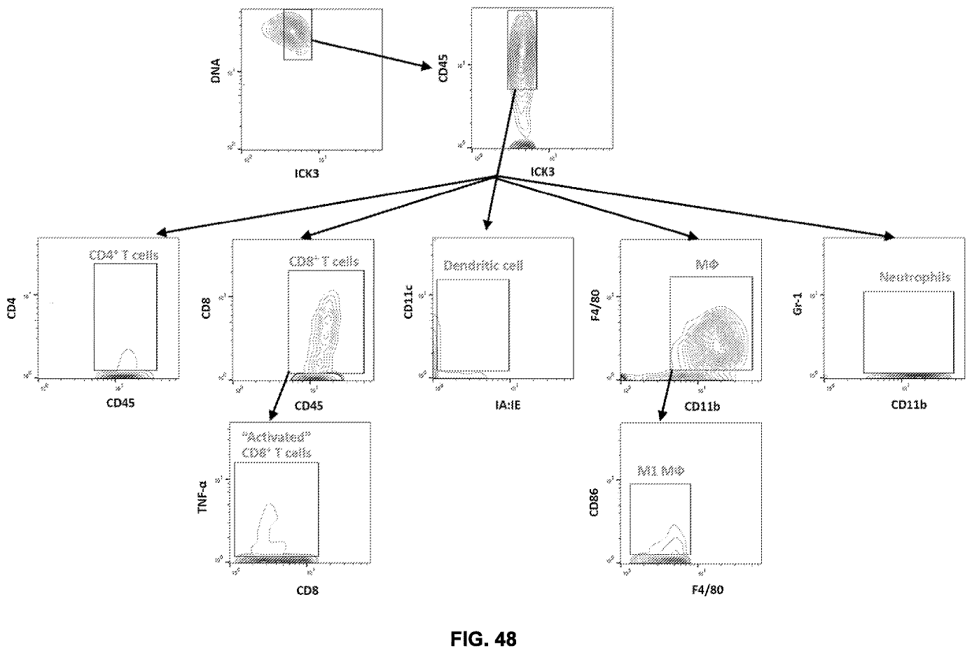

An additional immune profiling study was performed to understand the immune modulation mechanism of the s.c. administered COMBO COIN (FIG. 48). Significant CD8+ T cell and Mφ infiltration was observed 3 days after administration, with approximately 63% and 10% of the graft infiltrated lymphocytes CD8+ and F4/80+ (FIG. 43H), respectively. Specifically, approximately 2% of the infiltrated CD8+ T cells expressed T cell exhaustion markers (e.g., CD69) and 13% of the F4/80+ Mφs highly expressed the CD86 M1 Mφ marker (FIG. 43I). The magnitude of immune cell infiltration was significantly reduced 5 days after inoculation (FIG. 43J), but the relative frequencies of TNF-α+ activated CD8+ T cells and CD86+ M1 Mφ had not significantly change (FIG. 43I). The relatively high number of CD8+ T cells infiltrated into the s.c. graft was consistent with the hypothesis that the COIN acted as a site for autoreactive CD8+ T cell modulation. On the other hand, the relatively low frequency of Mφs detected in the s.c. graft suggested that the anti-inflammatory growth factors and cytokines could be released from the colon ECM and systemically dampened M1 Mφs.

### Example 5: COMBO COIN Ameliorates Colitis-Associated Colorectal Cancer

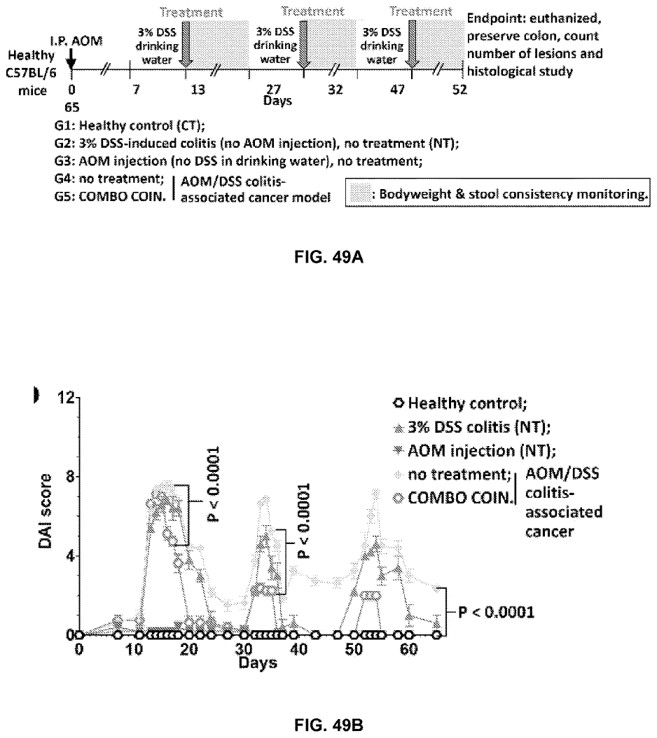

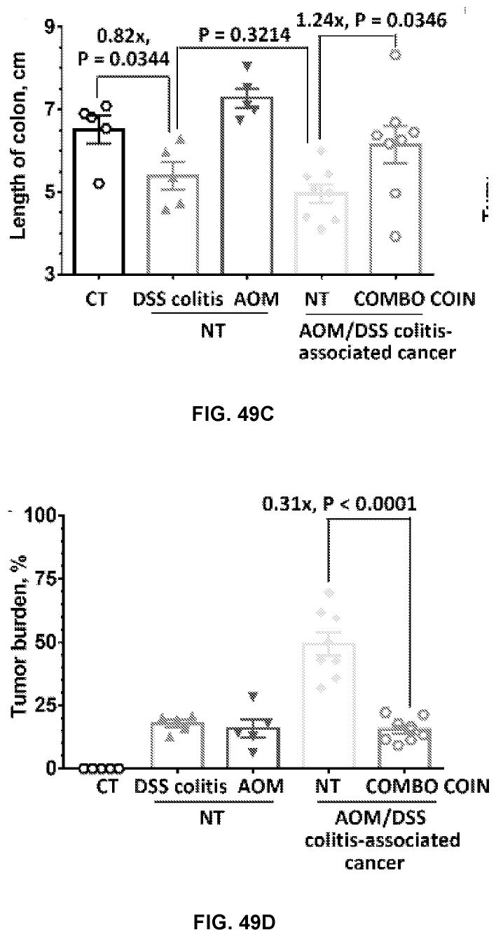

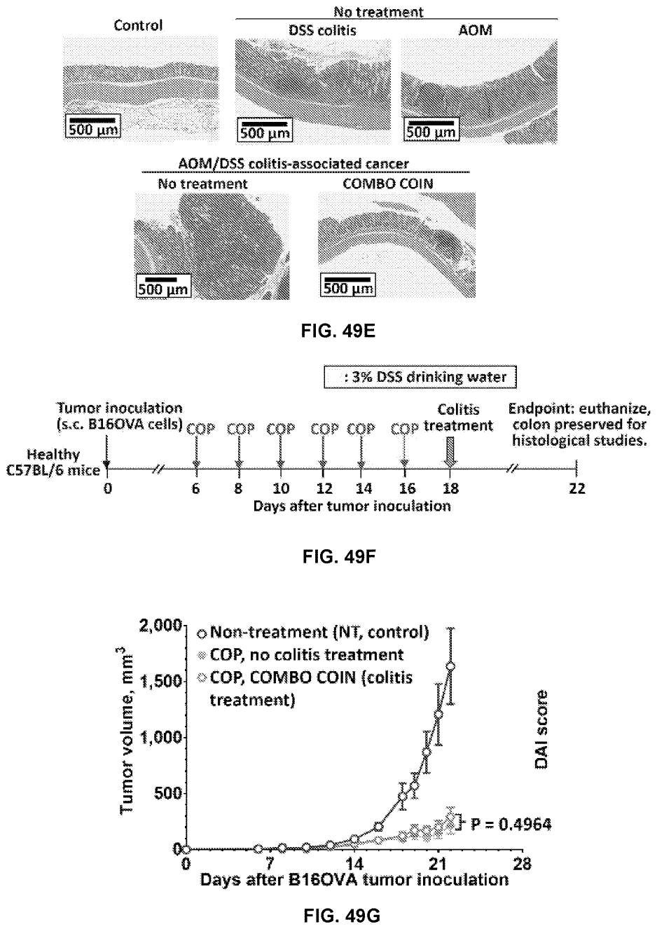

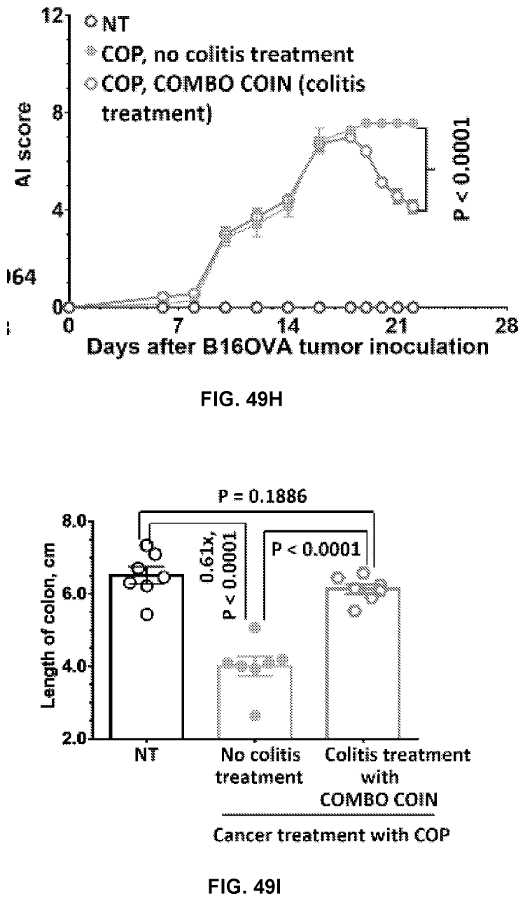

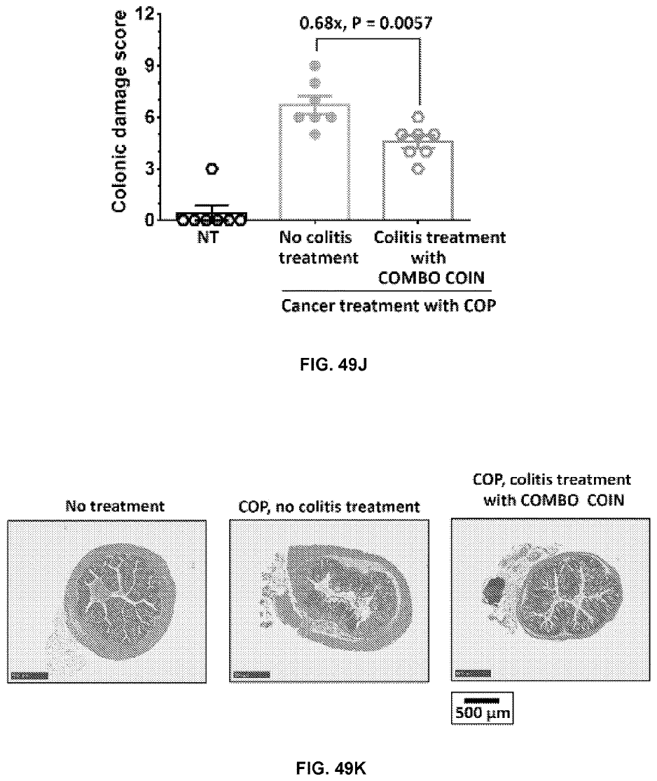

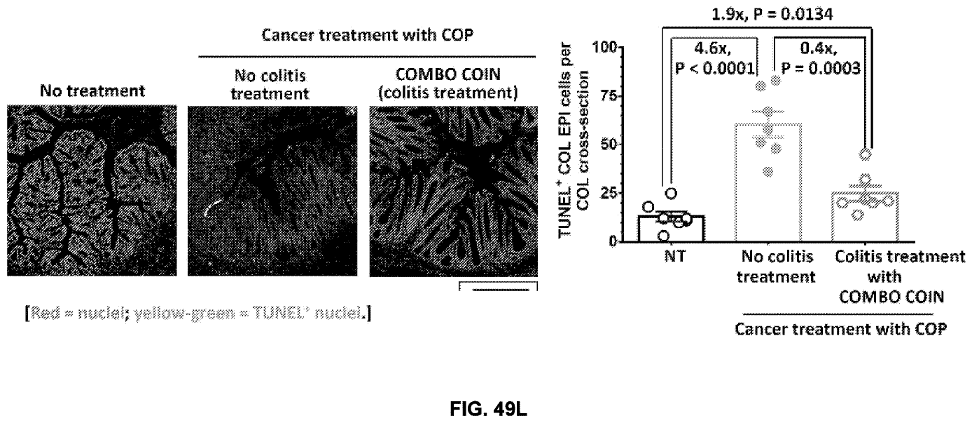

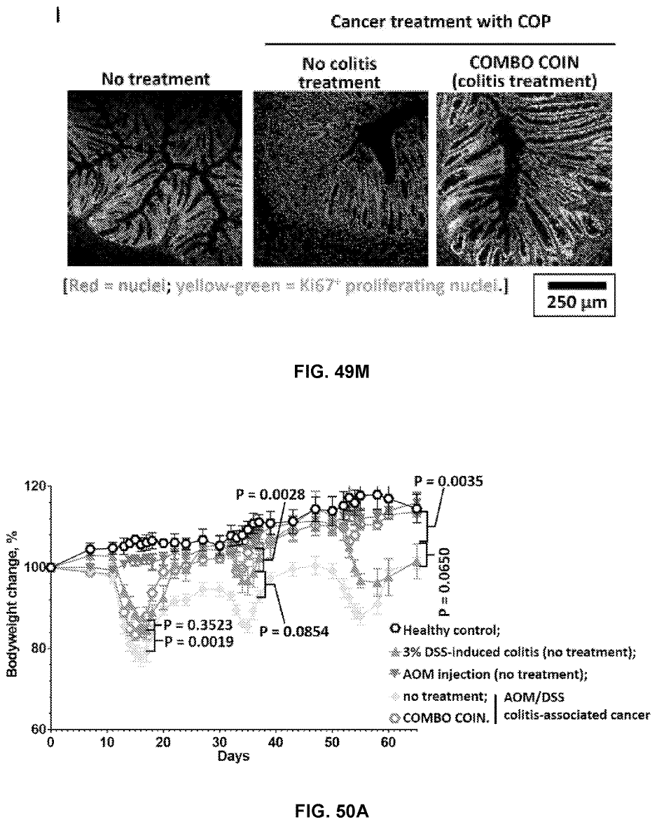

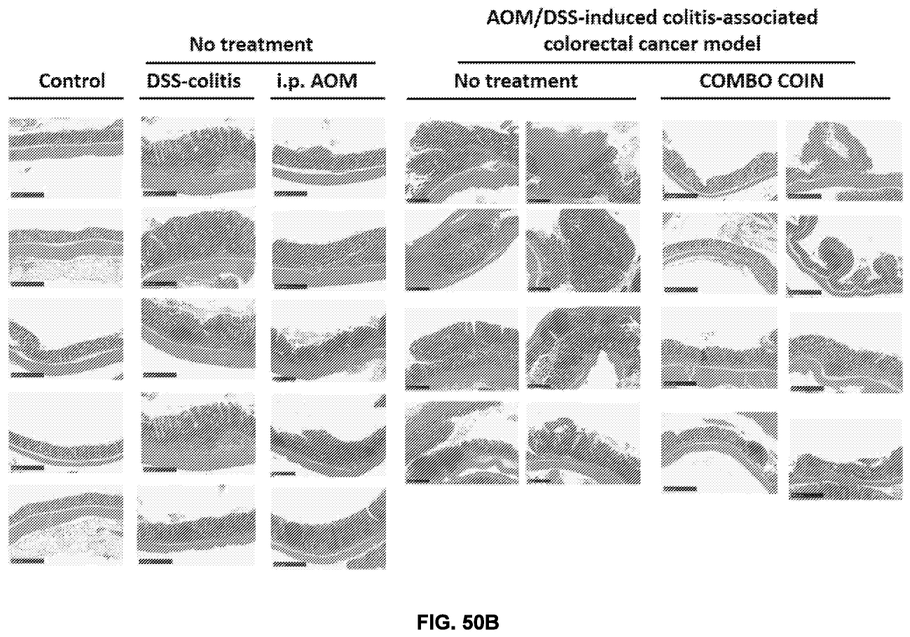

Chronic colonic inflammation increases the risk of developing colorectal cancer. Therefore, whether COMBO COIN could prevent the development of colitis-associated colorectal cancer was assessed in an established azoxymethane (AOM)/DSS model of colorectal cancer (FIG. 49A). Without treatment, AOM/DSS mice experienced more severe colitis and bodyweight loss than DSS colitis mice (FIG. 49B, and FIG. 50A-50B). At the study endpoint, severe colon shortening (FIG. 49C) and higher tumor burdens were evident in the distal colons of AOM/DSS colitis mice (FIG. 49D-49E, and FIG. 50A-50B). In contrast, mice consistently responded to COMBO COIN treatment (FIG. 49B). At the study endpoint, treated mice completely recovered their bodyweight (FIG. 50A-50B) and had 60% lower tumor burdens than the non-treatment group (FIG. 49D-49E, and FIG. 50A-50B). This finding highlighted the potential of using COMBO COIN to prevent colitis-associated colorectal cancer.

### Example 6: COMBO COIN Ameliorates PI3K Inhibition-Associated Colitis in Cell Signaling Pathway-Targeted Cancer Therapy

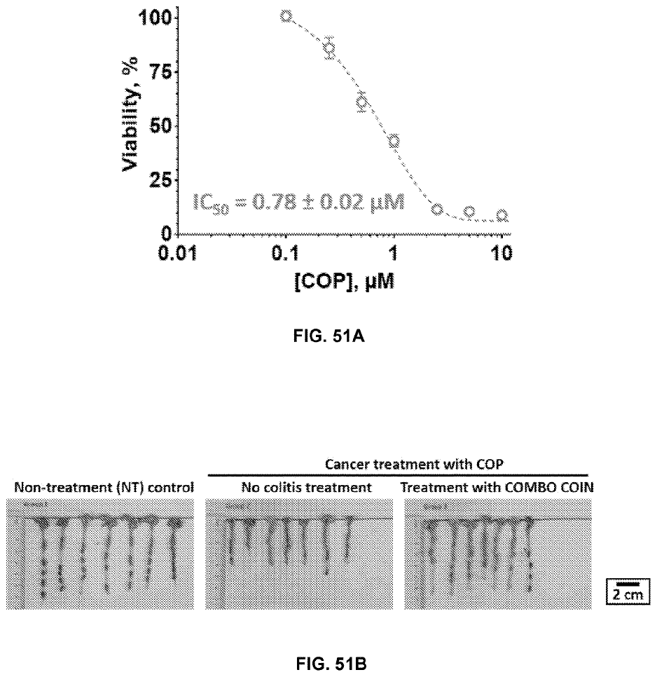

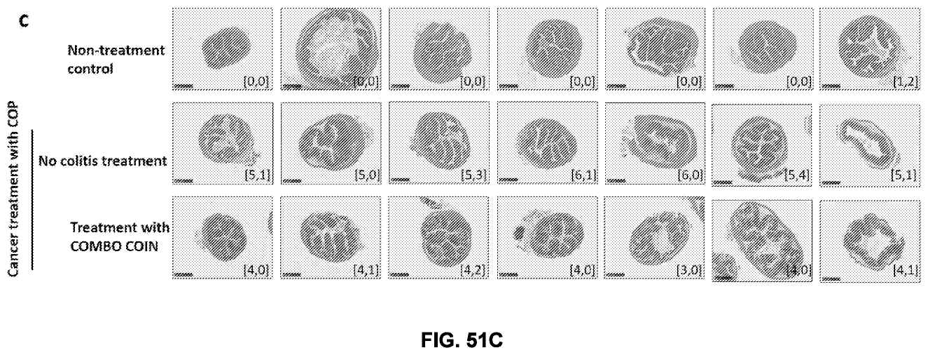

Phosphoinositide 3-kinase (PI3K) inhibitors, such as copanlisib (COP), have been developed to suppress tumor growth by inhibiting the overexpressed PI3K signal pathway in cancer cells. Colitis is one of the most common immune-related adverse events (irARs) associated with PI3K inhibitors due to their ability to deplete T_{reg} cells. The B16OVA melanoma tumor model was used with copanlisib cancer treatment to show that COMBO COIN could attenuate copanlisib-associated colitis without affecting its anticancer activity (FIG. 49F). Copanlisib effectively inhibited B16OVA tumor growth (FIG. 49G) but induced mild colitis symptoms (FIG. 49H). The colitis symptoms worsened after feeding with 3% DSS drinking water (FIG. 49F, and FIG. 49H). Colitis treatment with COMBO COIN effectively relieved colitis symptoms within 24 h after treatment (FIG. 49H). At the study endpoint, COMBO COIN treatment completely prevented inflammation-associated colon shortening (FIG. 49I, and FIG. 51A-51C) and facilitated partial repair of colon epithelium damage (FIG. 49J-49K, and FIG. 51A-51C) without affecting the anticancer activity of copanlisib (FIG. 49G). These findings confirmed the therapeutic potential of COMBO COIN for treating colitis-triggered by T_{reg} cell-depletion cancer therapy.

### Example 7: COMBO COIN Effectively Ameliorates Immune Checkpoint Blockade-Associated Colitis without Impairing Anticancer Efficacy

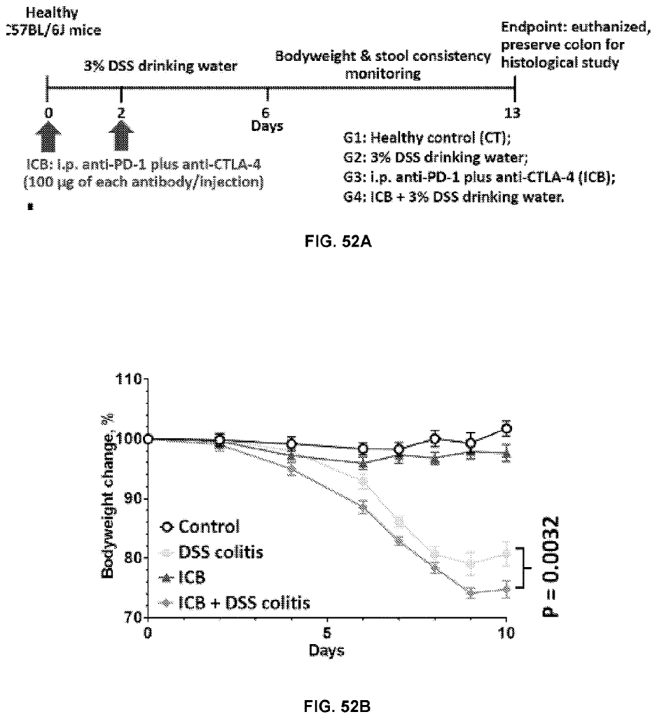

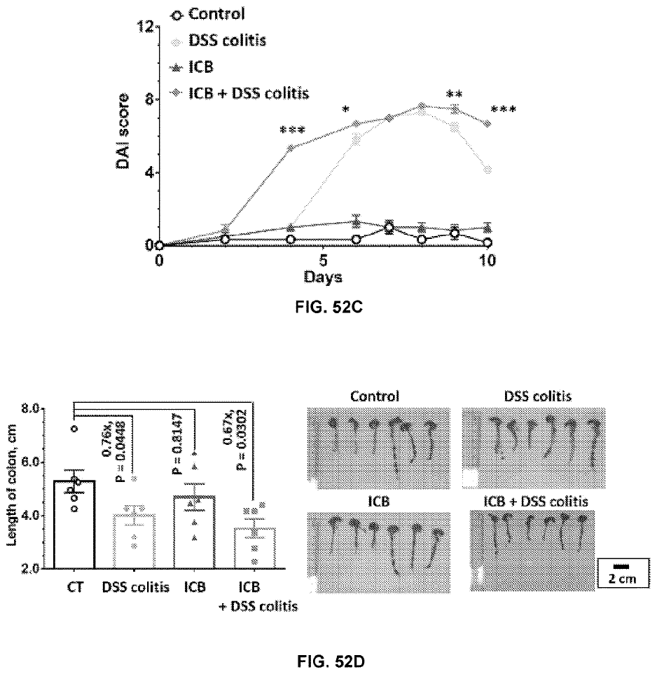

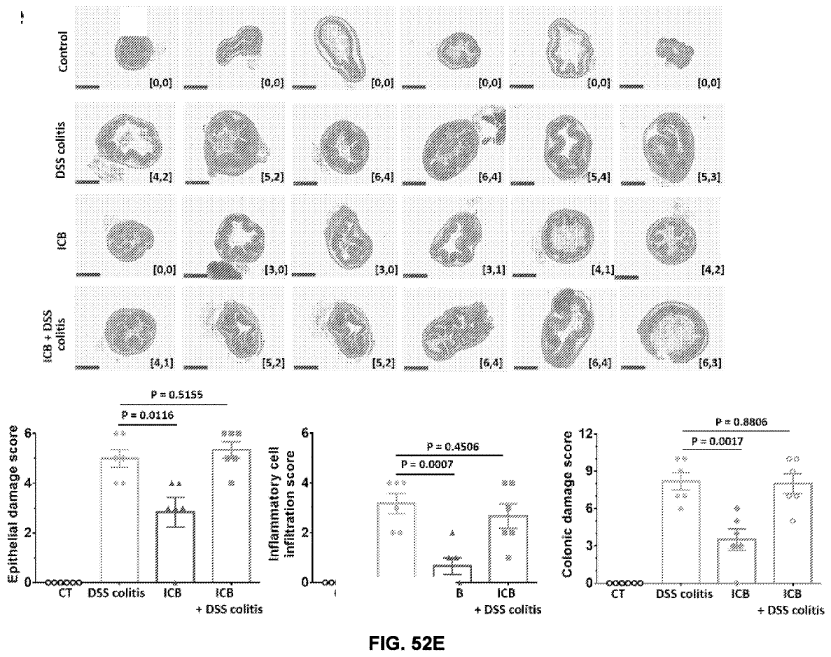

Dual blockade of inhibitory immune checkpoint pathways, such as the PD-(L)1 and CTLA-4 pathways, can synergistically improve cancer immunotherapy efficacy but also increases the chance of irAEs. Acute UC is a common irAE that leads to the discontinuation of cancer treatment. In the DSS-induced colitis mouse model, dual immune checkpoint blockade (ICB) with anti-PD-1 and anti-CTLA-4 antibodies induced profound colitis symptoms and eventually led to severe inflammation-associated COL contraction (33% shorter than healthy mice) and immune cell infiltration into the COL mucosa (FIG. 52A-52E).

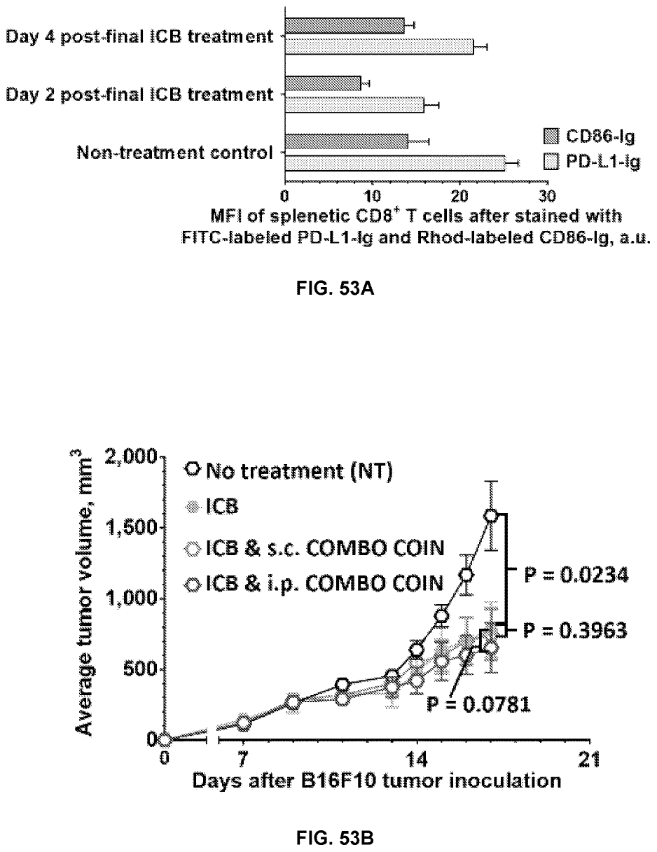

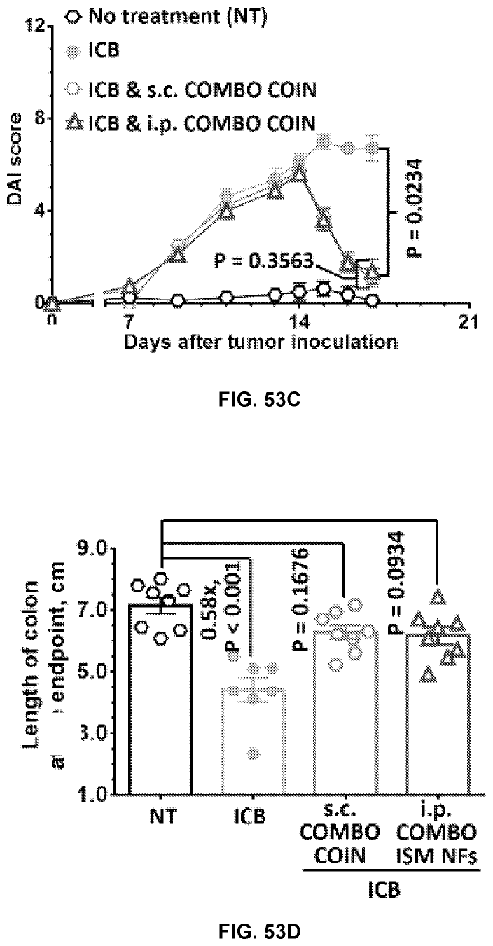

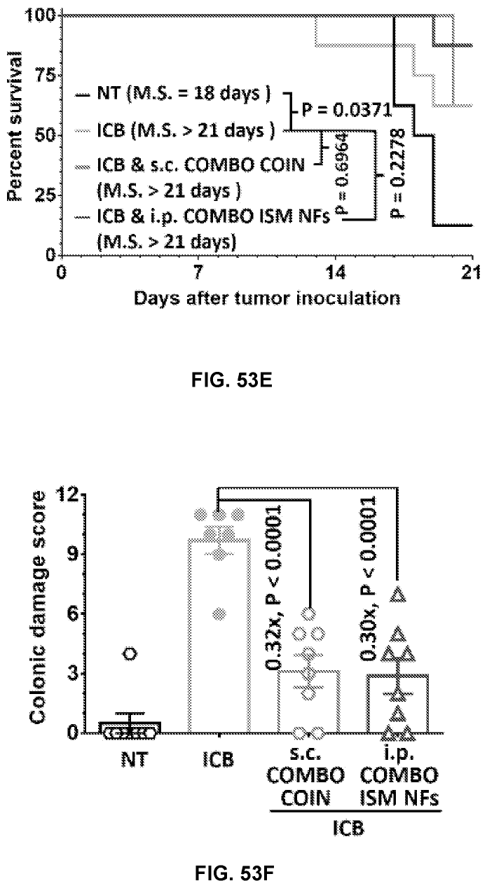

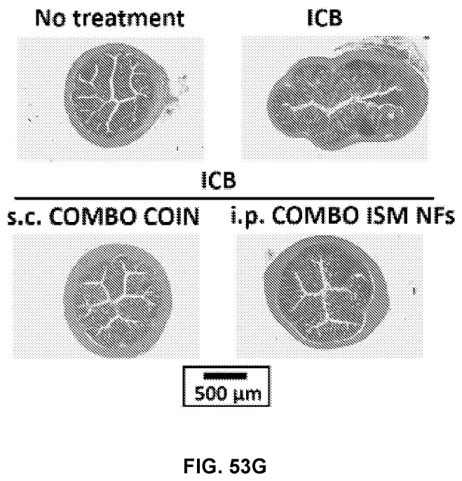

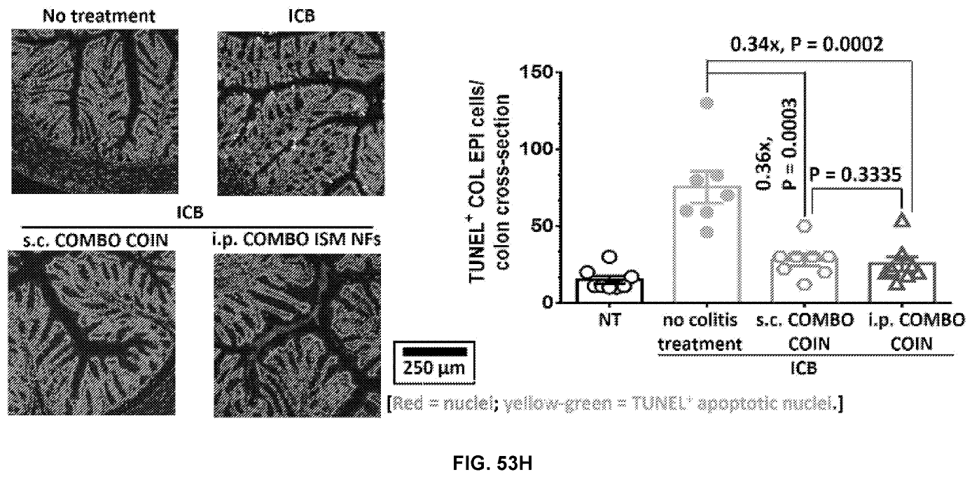

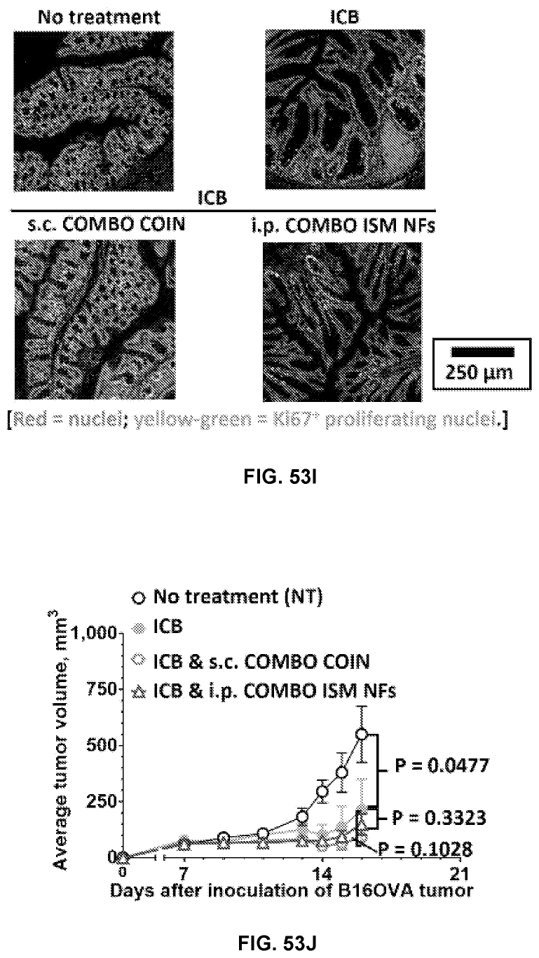

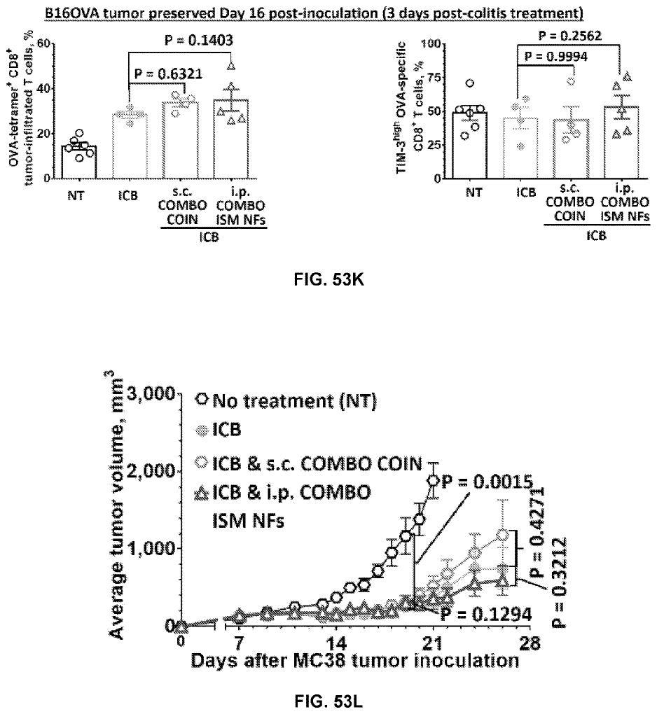

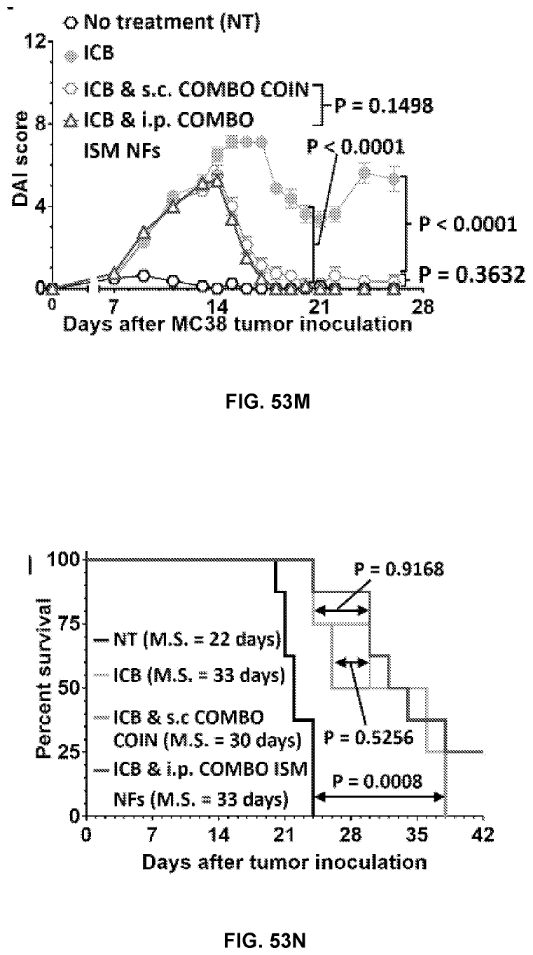

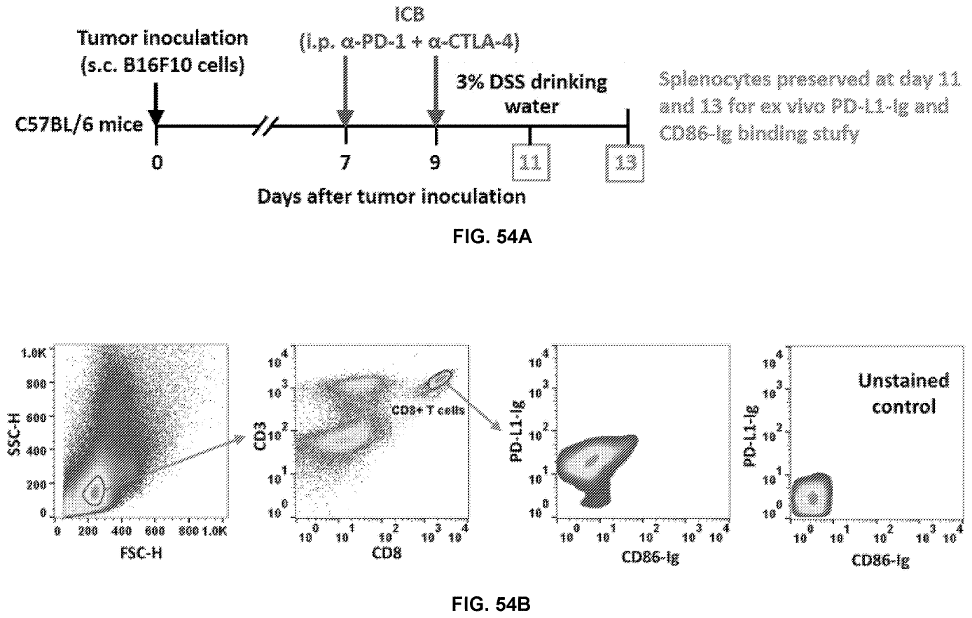

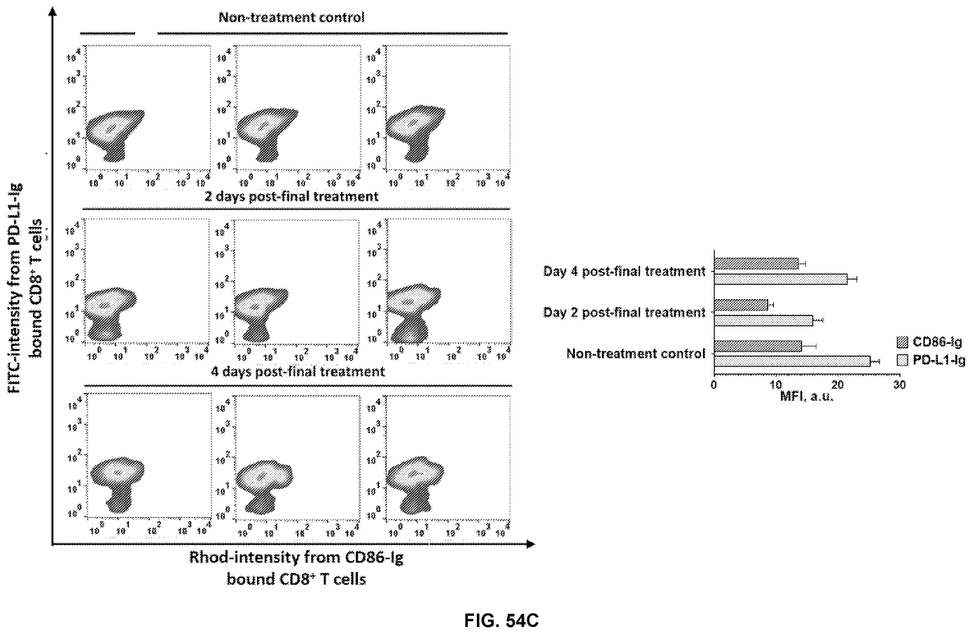

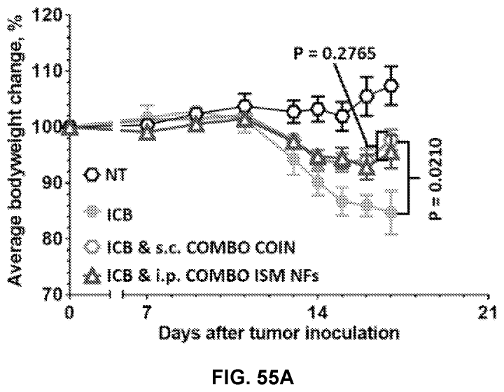

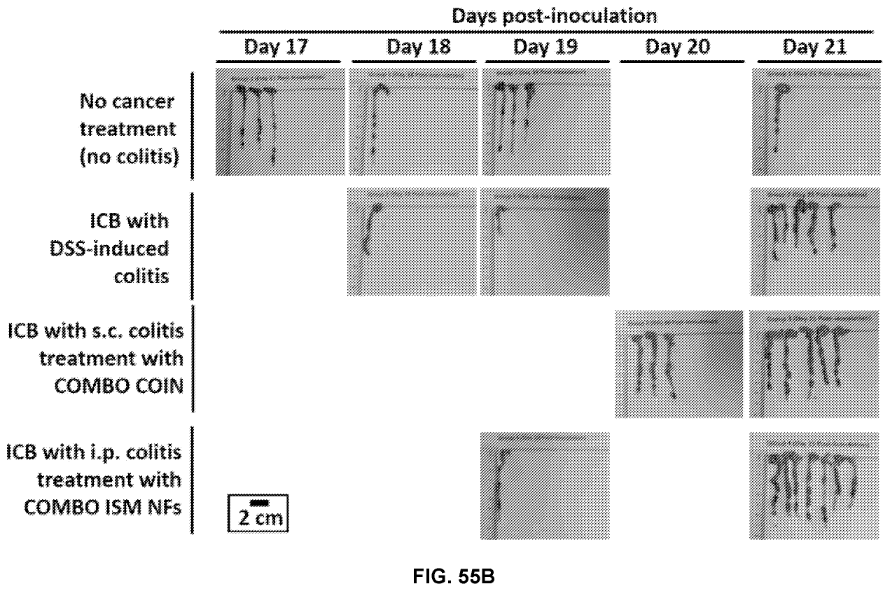

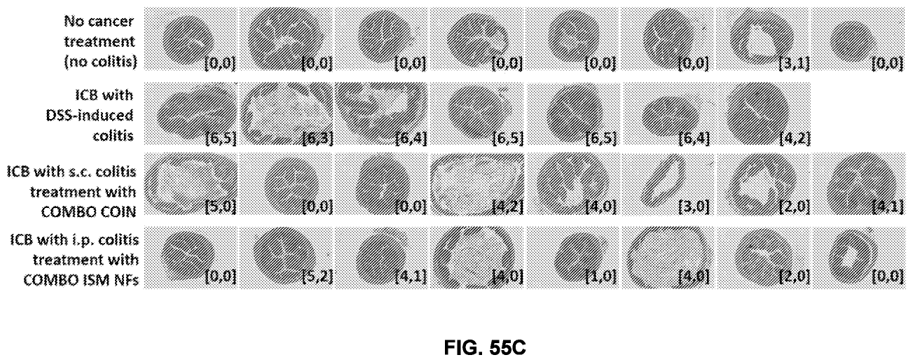

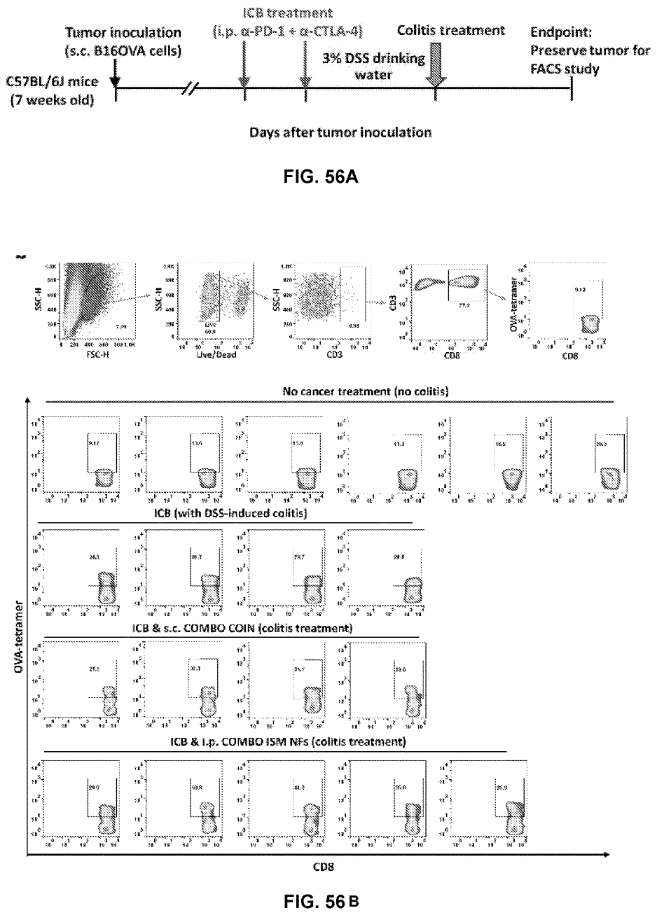

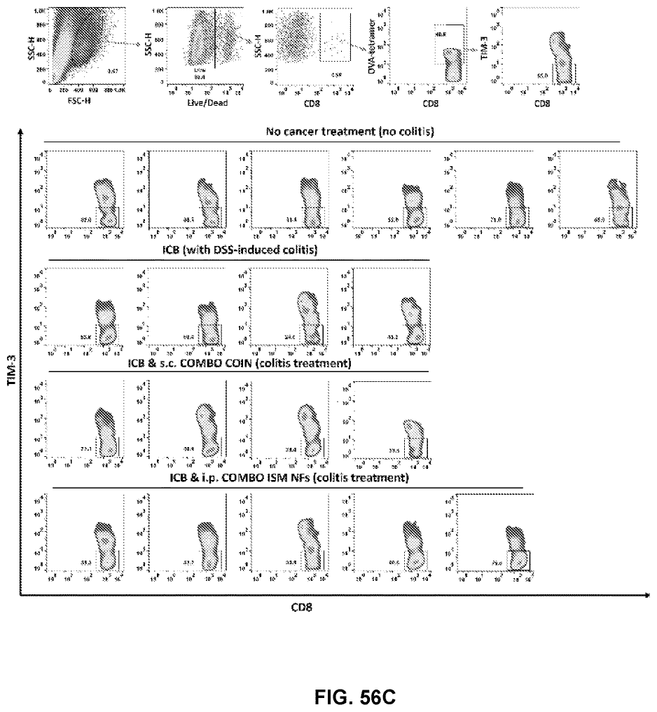

A key concern regarding immunosuppressive treatments for irAEs is their potential interference with ICB treatment. It was hypothesized that an optimized ICB treatment schedule would provide a therapeutic window for colitis treatment without impairing cancer treatment efficacy due to the gradual detachment of anti-PD-1 and anti-CTLA-4 antibodies from T cells (FIG. 53A, and FIG. 54A-54C). Therefore, an in vivo efficacy study was performed in the B16F10 tumor model to investigate whether the organ-specific colitis treatment affected the anticancer efficacy of dual ICB immunotherapy. In the B16F10 melanoma tumor model (FIG. 53C), colitis treatment with COMBO COIN four days after the final dual ICB treatment did not significantly affect cancer treatment efficacy (FIG. 53B). However, it significantly reduced colitis symptoms and colon inflammation at the study endpoint (FIG. 53C-53D, and FIG. 55A-55C). Histological analysis confirmed that the colitis treatment significantly reduced epithelial damage and immune cell infiltration into the colon epithelium (FIG. 53F-531, and FIG. 55A-55C). Similar therapeutic effects were observed for the i.p. treatment with COMBO ISM NFs (FIG. 53C-53D and FIG. 53F-531), and colitis treatment did not affect ICB treatment efficacy (FIG. 53B, and FIG. 53E). Since COMBO COIN ameliorated colitis partially through the immunosuppressive PD-L1/PD-1 and CD86/CTLA-4 immune checkpoint pathways, it was further investigated whether both colitis treatments would affect tumor antigen-specific CD8⁺ T cells. In the B16OVA tumor model (FIG. 53J), the frequency of tumor-infiltrated ovalbumin (OVA)-specific CD8⁺ T cell and expression of TIM-3 in the tumor preserved three days after colitis treatment was comparable with those who had not received colitis treatment (FIG. 53I-53K, and FIG. 56A-56C). These findings showed that colon-specific colitis treatment does not affect the anticancer activities of CD8⁺ T cells.

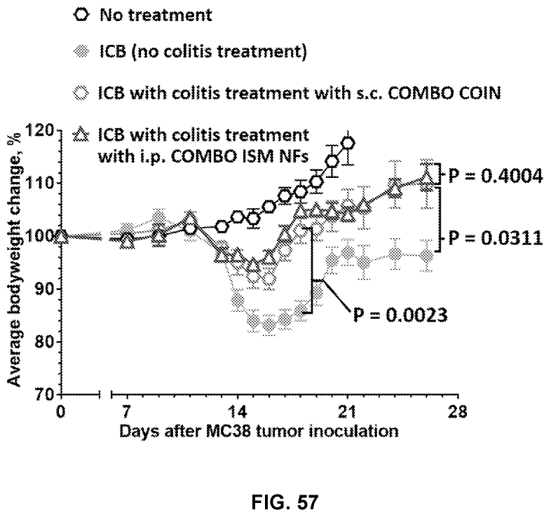

Guided by these promising treatment outcomes, a separate anticancer efficacy study was performed in the MC38 colorectal tumor model (FIG. 53L). Similar to the B16F10 tumor model, dual ICB effectively inhibited tumor growth (FIG. 53L). However, colitis necessitated the discontinuation of ICB treatment (FIG. 53M). Colitis treatment was performed four days after the second ICB treatment and effectively reversed weight loss and ameliorated colitis symptoms (FIG. 53M, and FIG. 57). Although MC38 tumors were derived from colon epithelial cells, colitis treatment with COMBO COIN did not affect the anticancer efficacy of subsequent ICB treatments (FIG. 53L-53M). The colitis treatment protected mice from developing further colitis symptoms during subsequent maintenance ICB immunotherapy (FIG. 53M, and FIG. 57). This was consistent with the development of colon epithelial cell-specific memory T cells observed in the chronic colitis model. Overall, this organ-specific colitis treatment did not affect the anticancer efficacy or survival benefit of ICB treatment (FIG. 53N), that an optimized colitis treatment schedule would be tolerated during ICB treatment.

### SUMMARY OF EXAMPLES

UC is an autoinflammatory disease that affects millions of individuals worldwide and is often refractory to therapeutic intervention. In addition to genetic and environmental factors, ICB can trigger UC during cancer immunotherapy. Poor UC control increases the risk of developing colorectal cancer. In the present disclosure, a clinically translatable protocol for the bioengineering of an immune niche for UC treatment has been successfully developed. The developed s.c. injectable immune niches could simultaneously anergize autoreactive CD8⁺ T cells and polarize proinflammatory M1 Mφs to anti-inflammatory M2 Mφs through multiple immunosuppressive pathways. The immune niche was engineered from colon epithelial cells, decellularized colon ECM, and COMBO ISM NFs. The COMBO ISM NFs were functionalized with two immunosuppressive immune checkpoint ligands (PD-L1 and CD86) to activate two key inhibitory immune checkpoint pathways in CD8⁺ T cells, LEF NFs to inhibit the proliferation of overreactive immune cells, and the pro-regulatory cytokine TGF-β1 mimetic peptide to inhibit CD8⁺ T cell proliferation and polarize M1 Mφs. Comprehensive in vitro studies demonstrated that COMBO ISM NFs effectively anergized CD8⁺ T cells and induced M1 Mφ polarization into M2 Mφs. The immunosuppression effects were further improved in the presence of colon ECM due to its preserved pro-regulatory growth factors and cytokines.

Comprehensive correlative studies in the DSS colitis model confirmed that s.c. treatment with COINs could ameliorate colitis symptoms and speed up colon epithelial stem cells in the crypts in repairing the epithelial damage. The therapeutic effect was also significant for COMBO COIN formulation engineered with four different types of immunosuppressants. The control study showed that colon epithelial cells and colon ECM were essential for recapitulating colon-like tissue microenvironments to modulate autoreactive immune cells. Furthermore, in vivo studies showed that the COMBO COIN could ameliorate colitis symptoms, facilitate the recovery of ulcerated colon epithelium in a TNP-induced colitis model and minimize immune cell infiltration in IL10 KO mice.

Comprehensive immune profiling confirmed that the therapeutic treatment decreased the pro-inflammatory autoreactive CD8⁺ T cells and M1 Mφs in the lamina propria and increased the frequencies of anti-inflammatory M2 Mφs. The histological study confirmed that COIN treatments, especially COMBO COIN treatment, significantly reduced the number of apoptotic colon epithelial cells in the villi and preserved the viability of the colon epithelial stem cells in the crypts. In addition to ulcerative colitis, it was demonstrated that treatment with COMBO COIN could prevent the development of colitis-associated colorectal cancer and ameliorated PI3K inhibitor- and ICB-induced colitis without affecting cancer treatment in established mouse models.

Recognizing the potential challenges of isolating colon epithelial cells and colon ECM from inflamed colon tissue in colitis patients, an i.p. administration of COMBO ISM NFs was further evaluated to treat ulcerative colitis. It was found that the i.p. administration of COMBO ISM NFs was as effective as the s.c. administered COMBO COIN in relieving colitis symptoms in the DSS-induced colitis model. Both immunosuppressive colitis treatments were also equally effective in promoting colon epithelial stem cells to repair the ulcerated colon epithelium. Furthermore, the in vivo study confirmed that i.p. administered COMBO ISM NFs were equally effective in relieving the colitis symptoms triggered by ICB in two established mouse tumor models, without affecting the cancer treatment efficiency. However, the in vivo biodistribution study indicated that the COMBO ISM NFs were rapidly distributed throughout the abdominal cavity after i.p. injection. This challenge can be resolved by directly administrating the COMBO ISM NFs to the inflamed tissues identified through colonoscopy to prevent broad immunosuppression.

The disclosed approach can be used with an autologous source of colonic epithelial to engineer COIN. Further, the methods disclosed herein can be modified based on existing methods and clinical protocols to incorporate COINs as a treatment for UC.

In summary, the disclosed immunosuppressive NFs functionalized with immune checkpoint molecules, immunomodulatory drugs and pro-regulatory cytokines, inhibited the activation of autoreactive CD8+ T cells and polarized M1 Mφs into M2 Mφs in vitro. It was also demonstrated that COMBO COIN bioengineered from colon epithelial cells, decellularized colon ECM, and COMBO ISM NFs effectively ameliorated colitis by inhibiting autoreactive CD8+ T cell activation and allowed healthy COL EPI cells to repair the damaged colon epithelium. Furthermore, the immune niche effectively prevented the development of colitis-associated colorectal cancer and ameliorated colitis-related irAEs in two established syngeneic tumor mouse models. The modular design of the immunosuppressive immune niche enables the on-demand introduction of alternative immunomodulators, targeted cells, and decellularized tissue to treat other autoimmune diseases.

What is claimed is:

1. A method of making a colon-specific immune niche (COIN), the method comprising combining diblock copolymer nanofibers (NFs), with colon epithelial (EPI) cells, and decellularized colon extracellular matrix (ECM) to form the COIN.

2. The method of claim 1, wherein the NFs, EPI, ECM, or any combination thereof is functionalized with an immunomodulator.

3. The method of claim 1, wherein the diblock copolymer comprises carboxylic acid-functionalized poly(2-(methacryloyloxy)ethyl phosphorylcholine)-poly(2-hydroxypropyl methacrylate) (PMPC-PHPMA).

4. The method of any one of the claims 1-3, wherein the NFs are further functionalized with sulfo DBCO-amine via amine-NHS ester coupling reaction (DBCO-NFs) or anionic carboxylic acid (COOH-NFs).

5. The method of any one of the claims 1-4, wherein the immunomodulator comprises a PD-L1 antibody, a CD86 antibody, leflunomide (LEF), HVEM (Herpesvirus entry mediator) or BTLA (B and T lymphocyte attenuator) agonist, Fas ligand, FGL-1, CD47, Gal-9, TGF-β1 mimetic peptide, or any combination thereof.

6. The method of any one of the claims 1-5, wherein the immunomodulator comprises a PD-L1 antibody, a CD86 antibody, or any combination thereof, and the immunomodulator is functionalized onto NFs by strain-promoted azide-alkyne cycloaddition with the azide-functionalized antibody at about 1-20 μg per mg of DBCO-NFs.

7. The method of any one of the claims 1-4, wherein the immunomodulator comprises LEF, and the immunomodulator is functionalized onto NFs by combining azide-functionalized LEF-encapsulated poly(ethylene glycol)-poly(lactide-co-glycolide) nanoparticles (LEF-NPs) with DBCO-NFs at about 1-400 μg of azide-functionalized LEF NPs per milligram of DBCO-NFs.

8. The method of any of the claims 1-5, wherein the immunomodulator is functionalized onto the ECM, EPI, or any combination thereof.

9. The method of any one of the claims 1-5, wherein the immunomodulator comprises TGF-β1 mimetic peptide, and the immunomodulator is functionalized onto NFs by ionic adsorption of the oligomerized cationic TGF-β1 mimetic peptide onto COOH-NFs at about 1 μg to about 100 μg mimetic peptide per milligram of COOH-NFs.

10. The method of any one of the claims 1-9, wherein the colon EPI cells and/or decellularized colon ECM are syngeneic, allogenic, or autologous.

11. The method of any one of the claims 1-10, wherein the combining comprises mixing together, NFs at a concentration of about 0.1 mg to about 100 mg, decellularized colon ECM at concentration of about 0.05 mg to about 5 mg, and colon EPI cells at a concentration of about 1×10² to about 1×10¹⁰ cells.

12. The method of any one of claims 1-10, wherein the combining comprises mixing together, NFs at a concentration of about 1 mg, decellularized colon ECM at a concentration of about 0.5 mg, and colon EPI cells at a concentration of about 1×10⁶ cells.

13. A composition comprising the COIN of any one of the preceding claims.

14. The composition of claim 13, wherein the COIN comprises a combination of NFs functionalized with a PD-L1 antibody, a CD86 antibody, leflunomide (LEF), and a TGF-β1 mimetic peptide.

15. The composition of any one of claims 13-14, wherein the composition is formulated for infusion, implantation, subcutaneous injection, or intraperitoneal injection.

16. A method of preventing, treating, or reducing the risk of a condition or disease of large intestinal tract tissue in a subject comprising: administering to a subject in need thereof an effective amount of a composition comprising COIN of any one of the claims 13-15.

17. A method of preventing, treating, or reducing the risk of a condition or disease of large intestinal tract tissue associated cancer in a subject, comprising: administering to a subject in need thereof an effective amount of a composition comprising COIN of any one of the claims 13-15.

18. A method of preventing, treating, or reducing the risk of a condition or disease of large intestinal tract tissue associated with administration of a Phosphoinositide 3-kinase (PI3K) inhibitor in a subject, comprising: administering to a subject in need thereof an effective amount of a composition comprising COIN of any one of the claims 13-15.

19. A method of preventing, treating, or reducing the risk of a condition or disease of large intestinal tract tissue associated with immune-related adverse events (irAEs) during immune checkpoint blockade therapy in a subject, comprising: administering to a subject in need thereof an effective amount of a composition comprising COIN of any one of the claims 13-15.

20. The method of any one of claims 16-19, wherein the effective amount of the COIN is about 1 μg to about 100 mg.

21. The method of any one of claims 16-19, wherein the administration results in reduced CD8⁺ T cell activation, pro-inflammatory cytokine expression, hematochezia, colitis-induced anemia, lymphocytic colitis, inflammation-induced colon shortening, epithelium damage immune cell infiltration into the lamina propria, tumor burden, rectum prolapse, microbiome dysbiosis, or any combination thereof.

22. The method of any one of claims 16-19, wherein the administration results in enhanced M1 Mφ differentiation into M2 Mφ, pro-regulatory cytokine expression, repair of epithelium damage, recovery of body weight, colon length maintenance, commensal microbiomes, or any combination thereof.

23. The method of any one of claims 16-19, wherein the condition or disease of large intestinal tract tissue comprises colitis, inflammatory bowel disease, Crohn's Disease, ulcerative colitis, familial adenomatous polyposis, Hirschsprungs, proctitits, celiac disease, gastrointestinal mucositis, or fistula.

24. The method of claim 19, wherein the subject is undergoing or planning to undergo cancer treatment with immune checkpoint blockade therapy.

25. The method of claim 24, wherein the immune checkpoint blockade therapy comprises administration of an inhibitor of PD-1, PD-L1, TIM-3, LAG-3, CTLA-4, CSF-1R, or any combinations thereof.

26. The method of claim 18, wherein the PI3K inhibitor is copanlisib, idelalisib, umbralisib, duvelisib, alpelisib, anti-PI3K antibody, CAL-120/GS-9820, Wortmannin, demethoxyviridin, LY294002, perifosine, CAL-101, PX-866, IPI-145, BAY 80-6946, BEZ-235, RP-6503, TGR-1202, SF-1126, INK-1117, GDC-0941, BKM-120, XL-147, XL-765, Palomid 529, GSK-1059615, ZSTK-474, PWT-33597, IC-87114, TG100-115, CAL-263, RP-6530, PI-103, GNE-477, CUDC-907, AEZS-136, or any combination thereof.

27. A kit comprising COIN of any one of the claims 1-15.
